(19) United States
(12) Patent Application Publication
(10) Pub. No.: US 2026/0092179 A1
(43) Pub. Date: Apr. 2, 2026

(54) GENETICALLY MODIFIED HOST CELLS PRODUCING VIOLACEIN, ANALOGUES, AND DERIVATIVES THEREOF

(71) Applicant: OCTARINE BIO APS, Copenhagen (DK)

(72) Inventors: Nicholas Stuart William MILNE, Copenhagen (DK); Annette Munch NIELSEN, Copenhagen (DK); Javier Fuentes IZQUIERDO, Copenhagen (DK); Leticia MISSAGIA DE MARCO, Copenhagen (DK); Camilla Knudsen BADEN, Copenhagen (DK)

(21) Appl. No.: 19/114,533

(22) PCT Filed: Sep. 21, 2023
(86) PCT No.: PCT/EP2023/076106

§ 371 Date: Mar. 24, 2025

(30) Foreign Application Priority Data
Sep. 26, 2022 (EP) 22197700.2

(51) Int. Cl. C09B 7/02; C07D 403/14; C07H 17/02; C12N 9/08; C12N 9/10; C12N 9/16; C12N 9/88; C12N 15/52; C12N 15/81; C12P 19/18; C12P 19/44; D06P 1/22; D06P 1/92; D06P 3/14; D06P 3/24; D06P 3/52; D06P 3/60

(52) U.S. Cl. CPC C09B 7/02; C07D 403/14; C07H 17/02; C12N 9/0065; C12N 9/1048; C12N 9/16; C12N 9/88; C12N 15/52; C12N 15/81; C12P 19/18; C12P 19/445; C12Y 111/01006; C12Y 301/03001; C12Y 401/01023; C12Y 401/01028; D06P 1/228; D06P 1/928; C12N 2800/102; D06P 3/14; D06P 3/243; D06P 3/52; D06P 3/60

(57) ABSTRACT

The present invention relates to methods for making compounds of formula (I) including violacein, violacein analogues and derivatives thereof, and to compositions, cells, and fermentation liquids comprising the compounds resulting from these methods.

### FIELD

The present invention relates to methods for making compounds of formula (I) including violacein, violacein analogues and derivatives thereof, and to compositions, cells, and fermentation liquids comprising the compounds resulting from these methods.

### BACKGROUND

Violacein is a purple-colored, natural indole derivative that has previously been biosynthesized by the condensation of two tryptophan molecules in several bacterial genera in response to quorum-sensing signals. Its chemical structure consists of three structural units, a 5-hydroxy indole, an oxindole, and a 2-pyrrolidone. The biosynthetic pathway of violacein from L-tryptophan has previously been elucidated, involving five enzymes (VioA, B, E, D, and C).

Violacein was first isolated from C. violaceum, the most studied bacteria particularly for its potential for violacein production. Apart from being a quorum-sensing metabolite, violacein tends to have a broad range of biological activities, including anti-tumoral, bacteriostatic and antibiotic potential, antifungal, anti-protozoan, anti-cancer, and antiviral properties. Meanwhile, the structural derivative deoxyviolacein (synthesized from L-tryptophan by VioA, B, E, and C) shows stronger antifungal properties than antimicrobial properties as compared to violacein. Moreover, violacein and its analogues are of immense industrial importance and have applications in cosmetics, textiles, agriculture, and drug discovery.

The commercially and scientifically significant properties of violacein and its analogues, has increased the demand for an industrially viable preparation method. However, an industrially viable preparation method of violacein and its analogues is lacking in the art.

### SUMMARY

The present inventors have developed a surprisingly effective biosynthesis of a compound of formula (I) including violacein, analogues and glycosides thereof using a genetically modified host cell.

Hence, the biosynthesis of the present disclosure provides a commercially viable access to previously unavailable compounds.

In one aspect, a method is provided for producing a compound of formula (I):

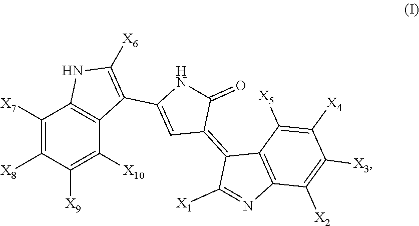

or a tautomer thereof, wherein any one of X₁, X₂, X₃, X₄, X₅, X₆, X₇, X₈, X₉, and X₁₀ are independently of each other selected from the group consisting of: H, R₁, R₂, O, OH, OR₁, NH, NO₂, NH₂, NHR₁, NHR₂, SR₁, F, Cl, Br, I, and SH; wherein R₁ and R₂ are independently of each other selected from the group consisting of a C₁₋₈ alkyl, C₁₋₈ alkenyl, C₁₋₈ alkoyl, C₁₋₈ aryl, and C₁₋₈ aroyl, and R₁ and R₂ are optionally covalently linked to form a ring; wherein the method comprises providing an indole of formula (II):

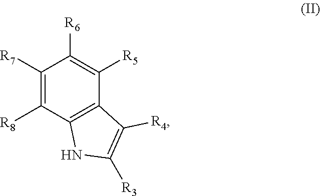

wherein
R₃, R₅, R₆, R₇, and R₈ are independently of each other selected from the group consisting of: H, R₁, R₂, O, OH, OR₁, NH, NH₂, NHR₁, NHR₂, NO₂, SR₁, F, Cl, Br, I and SH; wherein R₁ and R₂ are independently of each other selected from the group consisting of a C₁₋₈ alkyl, C₁₋₈ alkenyl, C₁₋₈ alkoyl, C₁₋₈ aryl, and C₁₋₈ aroyl, and R₁ and R₂ are optionally covalently linked to form a ring; and wherein
R₄ is a chemical handle for enzymatic conversion toward one or more intermediates leading to the compound of formula (I), including the compound of formula (I), and further comprises contacting the indole of formula (II) with one or more enzymes, optionally wherein the one or more enzymes are from an operative biosynthetic pathway for producing violacein.

In one aspect, a compound is provided of formula (I):

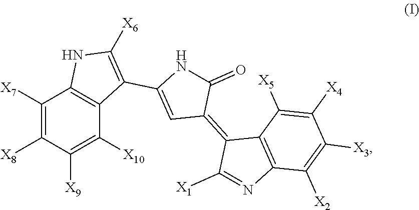

or a tautomer thereof, wherein any one of X₁, X₂, X₃, X₄, X₅, X₆, X₇, X₈, X₉, and X₁₀ are independently of each other selected from the group consisting of: H, R₁, R₂, O, OH, OR₁, NH, NO₂, NH₂, NHR₁, NHR₂, SR₁, F, Cl, Br, I, and SH; wherein R₁ and R₂ are independently of each other selected from the group consisting of a C₁₋₈ alkyl, C₁₋₈ alkenyl, C₁₋₈ alkoyl, C₁₋₈ aryl, and C₁₋₈ aroyl, and R₁ and R₂ are optionally covalently linked to form a ring.

In one aspect, a microbial host cell genetically modified to perform any method disclosed herein is provided to produce a compound of formula (I),

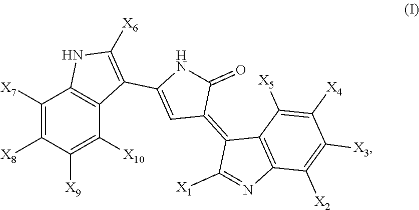

or a tautomer thereof, wherein any one of X₁, X₂, X₃, X₄, X₅, X₆, X₇, X₈, X₉, and X₁₀ are independently of each other selected from the group consisting of: H, R₁, R₂, O, OH, OR₁, NH, NO₂, NH₂, NHR₁, NHR₂, SR₁, F, Cl, Br, I, and SH; wherein R₁ and R₂ are independently of each other selected from the group consisting of a C₁₋₈ alkyl, C₁₋₈ alkenyl, C₁₋₈ alkoyl, C₁₋₈ aryl, and C₁₋₈ aroyl, and R₁ and R₂ are optionally covalently linked to form a ring;
wherein the host cell expresses one or more heterologous genes encoding the one or more enzymes.

In one aspect, a cell culture is provided, comprising a host cell as defined herein and a growth medium.

In one aspect, a fermentation liquid is provided comprising the compound of formula (I) comprised in the cell culture defined herein.

In one aspect, a composition is provided comprising the fermentation liquid defined herein, and/or the compound of formula (I) and one or more agents, additives and/or excipients.

In one aspect, a method for in-situ extraction of the compound of formula (I) or the glycosylated compound of formula (I) is provided, comprising:
a) Providing a host cell as defined herein or the cell culture as defined herein comprising the compound of formula (I) or the glycosylated compound of formula (I) in an aqueous phase; b) Subjecting the aqueous phase to extraction with an extractant, optionally wherein the extractant is a non-ionic surfactant.

In one aspect, a method is provided for dyeing a textile material, comprising:
a) providing an optionally dried composition of one or more compounds as defined herein, for example violacein, proviolacein, prodeoxyviolacein, and/or deoxyviolacein; and subsequently preparing a dye solution by suspending said composition in a liquid, such as an alcohol, for example ethanol; or
b) providing a colored fermentation extract comprising an extractant and one or more compounds as defined herein, for example violacein, proviolacein, prodeoxyviolacein, and/or deoxyviolacein
c) contacting a textile material with said dye solution or said colored fermentation extract, optionally for a predetermined duration, thereby dyeing the textile material.

In one aspect, a dyed textile material comprising the compound as defined herein is provided.

In one aspect, a method of colouring a beverage is provided, comprising:
a) providing an optionally dried composition of one or more compounds as defined herein, for example glycosylated violacein, glycosylated proviolacein, glycosylated prodeoxyviolacein, and/or glycosylated deoxyviolacein; and optionally subsequently preparing a dye solution by suspending said composition in a liquid, such as an alcohol or water; and
b) contacting a beverage with said dye solution or said composition, optionally for a predetermined duration, thereby colouring the beverage.

In one aspect, a method is provided for enhancing the antimicrobial properties of a textile material, such as clothing or a wound dressing, or a beverage, comprising dyeing the textile material or colouring the beverage with the compound as defined herein, or with an extractant comprising the compound thereby enhancing the antimicrobial properties of the textile material or beverage.

In one aspect, a method is provided for enhancing the antioxidant properties of a textile material, such as clothing, or a beverage comprising dyeing the textile material or colouring the beverage with the compound as defined herein, or with an extractant comprising the compound thereby enhancing the antioxidant properties of the textile material or beverage.

In one aspect, a method is provided for enhancing the UV resistance of a textile material, such as clothing, or of a beverage comprising dyeing the textile material or colouring the beverage with the compound as defined herein, or with an extractant comprising the compound thereby enhancing the UV resistance of the textile material or beverage.

In one aspect, a beverage is provided comprising the comprising a glycoside of the compound as defined herein.

In one aspect, a nanocellulose is provided comprising a compound as defined herein.

In one aspect, method for dyeing nanocellulose is provided, comprising
a. providing a compound as defined herein, optionally in a dye bath comprising an alcohol and optionally a surfactant;
b. providing nanocellulose, such as bacterial nanocellulose (BNC) or nanofabricated cellulose (NFC);
c. incubating the cellulose with the compound, optionally in the dye bath, at a predefined temperature until the nanocellulose takes on the color of the compound, thereby providing dyed nanocellulose.

In one aspect, a dyed product is provided comprising the nanocellulose as defined herein.

In one aspect, a method is provided for dyeing a product, comprising:
a) Providing a nanocellulose as defined herein;
b) Providing a product;
c) Contacting the nanocellulose with the product, optionally incubating the product with the nanocellulose for a duration.

In one aspect, a method of producing a dye bath is provided, the method comprising the steps of:
a) cultivating a host cell as defined herein in growth medium to produce the compound as defined herein, such as engineered S. cerevisiae production strains producing at least one of violacein, deoxyviolacein, prodeoxyviolacein, and proviolacein;
b) adding an extractant to the growth medium thereby providing a compound enriched extractant;
c) optionally collecting the compound enriched extractant and adding further extractant to the growth medium;
d) optionally repeating step c a number of times to provide a collection of compound enriched extractants,
e) diluting the compound enriched extractant or the collection of compound enriched extractants with a liquid, such as an organic solvent, thereby providing a dye bath.

In one aspect, a dye bath obtainable using the method of the present disclosure is provided.

In one aspect, a method for dyeing a product is provided, comprising the steps of:
a) adding a product to a dye bath comprising a compound of formula (I) as defined herein, and a liquid and optionally an extractant;
b) optionally pre/post-treating the product to modify its pH;
c) optionally dyeing the product at a predetermined temperature for a predetermined time to obtain a dyed product, optionally in a dyeing machine;
d) washing the dyed product with water; and
e) optionally drying the product.

In one aspect, a method of recycling a used dye bath is provided, comprising the steps of:
a) subjecting a used dye bath comprising i) a liquid, ii) an extractant, and iii) a compound of formula (I) as defined herein to evaporation, optionally in vacuo to remove the liquid, wherein the dye bath has been used for dyeing a product;
b) passing the remaining extractant and compound from step a through silica to obtain a recycled dye bath.

### DRAWINGS AND FIGURES

FIG. 1 shows a biosynthetic pathway for production of violacein and natural violacein derivatives in S. cerevisiae. Overexpressed genes are shown in italics, relevant gene deletions involved in the biosynthetic pathway are indicated with a Δ. Dashed arrows indicate multiple enzymatic steps, solid arrows indicate a single enzymatic step. Metabolites of the biosynthetic pathway are shown in boxes. Violacein biosynthetic enzymes (CvVioA,C,D,E) natively produce the acidic form of their respective violacin derivative (e.g. violaceinic acid), for simplicity the spontaneous decarboxylation produce (e.g. violacein is shown). Glc: D-Glucose, G6P: D-glucopyranose 6-phosphate, Ru5P: D-ribulose 5-phosphate, F6P: D-fructose 6-phosphate, E4P: D-erythrose 4-phosphate, ACP: Acetyl phosphate, ACC: Acetyl-CoA, ACE: Acetate, PEP: Phosphoenol pyruvate, DAHP: 3-deoxy-D-arabino-heptulosonate 7-phosphate, SHM: Shikimate, S3P: Shikimate 3-phosphate, ES3P: 5-enolpyruvoyl-shikimate 3-phosphate, CHM: Chorismate, ANT: Anthranilate, Rib5P: Ribose 5-phosphate, PRPP: 5-phospho-α-D-ribose 1-diphosphate, NPA: N-(5-phosphoribosyl)-anthranilate, CDP: 1-(o-carboxyphenylamino)-1′-deoxyribulose 5′-phosphate, IGP: (1S,2R)-1-C-(indol-3-yl)glycerol 3-phosphate, SER: Serine, TRP: Tryptophan, TRYP: Tryptamine, I3E: Indole 3-ethanol, IPA: Indole 3-pyruvic acid imine (IPA imine), HEME: Ferroheme b, GLY: Glycine, IPAD: IPA imine dimer, BIL: Biliverdin, PDV: Prodeoxyviolacein, PVIO: Proviolacein, DVIO: Deoxyviolacein, VIO: Violacein.

FIG. 2 shows a HPLC chromatogram of violacein and violacein derivative producing S. cerevisiae strains after cultivation at 30° c. for 3 days and intracellular extraction. Dotted line (a) Authentic Violacein analytical standard (RT 5.32 min at 575 nm), Solid lines: (b) SC-144 (deoxyviolacein RT 5.6 min at 575 nm); (c) SC-141 (violacein RT 5.32 min); (d) SC-139 (prodeoxyviolacein RT 5.8 min at 230 nm) and (e) SC-145 (proviolacein RT 5.6 min at 230 nm).

FIG. 3 shows chemical structures of exemplary Proviolacein-5-O-β-glycoside and Violacein-5-O-β-glycoside produced by a glycosyltransferase enzyme.

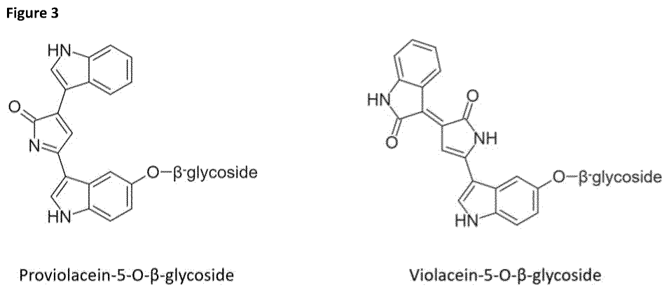

FIG. 4 shows exemplary overviews of native production of tryptophan in a microbial cell (a), and production of substituted tryptophan derivatives (b) useful for the production of violacein and substituted violacein derivatives, respectively.

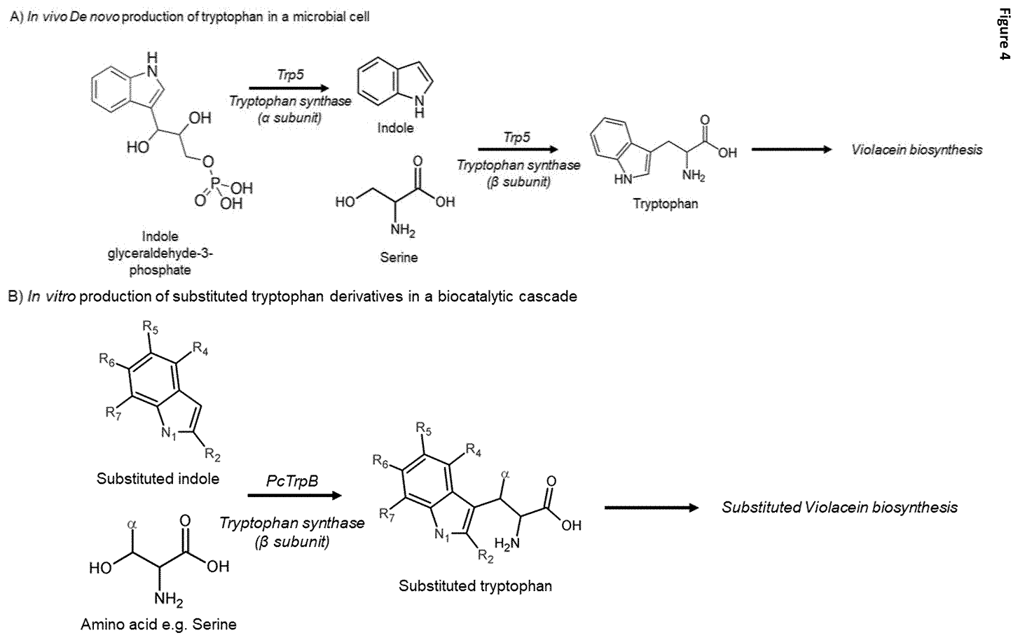

FIG. 5 shows a schematic overview of a consolidated process for the production of compounds of formula (I), such as violacein and violacein derivatives and their direct dyeing onto fabrics, yarns, and fibers. (1) Fermentation of engineered strains with in situ product recover (ISPR). (2) Product rich extractant phase is used directly as input for a dye bath by diluting in an organic or aqueous solvent. (3) Fabrics, yarns or fibers are dyed in dye bath for 15 minutes at room temperature. (4) Once dye is exhausted the individual components are recycled by a combination of cloud point extraction or gel silica chromatography and evaporation of volatile solvents. Alternatively, a dry powder can be obtained through the same DSP process to be used as input for the dye bath.

### INCORPORATION BY REFERENCE

All publications, patents, and patent applications referred to herein are incorporated by reference to the same extent as if each individual publication, patent, or patent application was specifically and individually indicated to be incorporated by reference. In the event of a conflict between a term herein and a term in an incorporated reference, the term herein prevails and controls.

### DETAILED DESCRIPTION

### Definitions

The terms “heterologous” or “recombinant” or “genetically modified” and their grammatical equivalents as used herein interchangeably refers to entities “derived from a different species or cell”. For example, a heterologous or recombinant polynucleotide gene is a gene in a host cell not naturally containing that gene, i.e. the gene is from a different species or cell type than the host cell. The terms as used herein about microbial host cells refers to microbial host cells comprising and expressing heterologous or recombinant polynucleotide genes.

The term “pathway” as used herein is intended to mean two or more enzymes acting sequentially in a live cell to convert chemical substrate(s) into chemical product(s). Enzymes are characterized by having catalytic activity, which can change the chemical structure of the substrate(s).

An enzyme may have more than one substrate and produce more than one product. The enzyme may also depend on cofactors or “pathway molecules”, which can be iganic chemical compounds or organic compounds such as proteins for example enzymes (co-enzymes). In the context of the present disclosure, the pathway molecules are in some embodiments FAD, HEME, and NADPH. The term “operative biosynthetic pathway” refers to a pathway that occurs in a live recombinant host, as described herein i.e. the microbial host cell.

The term “in vivo”, as used herein refers to within a living cell or organism, including, for example animal, a plant or a microorganism.

The term “in vitro”, as used herein refers to outside a living cell or organism, including, without limitation, for example, in a microwell plate, a tube, a flask, a beaker, a tank, a reactor and the like.

The term “in planta”, as used herein refers to within a plant or plant cell.

The term “substrate” or “precursor”, as used herein refers to any compound that can be converted into a different compound. For example, the indole of formula (II) can be a substrate for one or more enzymes and can be converted into one or more intermediates leading to the compound of formula (I), including the compound of formula (I). For clarity, substrates and/or precursors include both compounds generated in situ by a enzymatic reaction in a cell or exogenously provided compounds, such as exogenously provided organic molecules which the host cell can metabolize into a desired compound.

Term “endogenous” or “native” as used herein refers to a gene or a polypeptide in a host cell which originates from the same host cell.

The term “deletion” as used herein refers to manipulation of a gene so that it is no longer expressed in a host cell.

The term “disruption” as used herein refers to manipulation of a gene or any of the machinery participating in the expression the gene, so that it is no longer expressed in a host cell.

The term “attenuation” as used herein refers to manipulation of a gene or any of the machinery participating in the expression the gene, so that it the expression of the gene is reduced as compared to expression without the manipulation.

The terms “substantially” or “approximately” or “about”, as used herein refers to a reasonable deviation around a value or parameter such that the value or parameter is not significantly changed. These terms of deviation from a value should be construed as including a deviation of the value where the deviation would not negate the meaning of the value deviated from. For example, in relation to a reference numerical value the terms of degree can include a range of values plus or minus 10% from that value. For example, deviation from a value can include a specified value plus or minus a certain percentage from that value, such as plus or minus 9%, 8%, 7%, 6%, 5%, 4%, 3%, 2%, or 1% from the specified value.

The term “and/or” as used herein is intended to represent an inclusive “or”. The wording X and/or Y is meant to mean both X or Y and X and Y. Further the wording X, Y and/or Z is intended to mean X, Y and Z alone or any combination of X, Y, and Z.

The term “isolated” as used herein about a compound, refers to any compound, which by means of human intervention, has been put in a form or environment that differs from the form or environment in which it is found in nature. Isolated compounds include but is no limited to compounds of the disclosure for which the ratio of the compounds relative to other constituents with which they are associated in nature is increased or decreased. In an important embodiment the amount of compound is increased relative to other constituents with which the compound is associated in nature. In an embodiment the compound of the disclosure may be isolated into a pure or substantially pure form. In this context a substantially pure compound means that the compound is separated from other extraneous or unwanted material present from the onset of producing the compound or generated in the manufacturing process. Such a substantially pure compound preparation contains less than 10%, such as less than 8%, such as less than 6%, such as less than 5%, such as less than 4%, such as less than 3%, such as less than 2%, such as less than 1%, such as less than 0.5% by weight of other extraneous or unwanted material usually associated with the compound when expressed natively or recombinantly. In an embodiment the isolated compound is at least 90% pure, such as at least 91% pure, such as at least 92% pure, such as at least 93% pure, such as at least 94% pure, such as at least 95% pure, such as at least 96% pure, such as at least 97% pure, such as at least 98% pure, such as at least 99% pure, such as at least 99.5% pure, such as 100% pure by weight.

The term “non-naturally occurring” as used herein about a substance, refers to any substance that is not normally found in nature or natural biological systems. In this context the term “found in nature or in natural biological systems” does not include the finding of a substance in nature resulting from releasing the substance to nature by deliberate or accidental human intervention. Non-naturally occurring substances may include substances completely or partially synthetized by human intervention and/or substances prepared by human modification of a natural substance.

The term “% identity” is used herein about the relatedness between two amino acid sequences or between two nucleotide sequences.

The term “% identity” as used herein about amino acid sequences refers to the degree of identity in percent between two amino acid sequences obtained when using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are gap open penalty of 10, gap extension penalty of 0.5, and the EBLOSUM62 (EMBOSS version of BLOSUM62) substitution matrix. The output of Needle labeled “longest identity” (obtained using the -nobrief option) is used as the percent identity and is calculated as follows:

$$\frac{identical amino acid residues}{Length of alignment - total number of gaps in alignment} \times 100$$

The term “% identity” as used herein about nucleotide sequences refers to the degree of identity in percent between two nucleotide sequences obtained when using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 5.0.0 or later. The parameters used are gap open penalty of 10, gap extension penalty of 0.5, and the EDNAFULL (EMBOSS version of NCBI NUC4.4) substitution matrix. The output of Needle labeled “longest identity” (obtained using the -nobrief option) is used as the percent identity and is calculated as follows:

$$\frac{identical deoxyribonucleotides}{Length of alignment - total number of gaps in alignment} \times 100$$

The protein sequences of the present disclosure can further be used as a “query sequence” to perform a search against sequence databases, for example to identify other family members or related sequences. Such searches can be performed using the BLAST programs. Software for performing BLAST analyses is publicly available through the National Center for Biotechnology Information (http://www.ncbi.nlm.nih.gov). BLASTP is used for amino acid sequences and BLASTN for nucleotide sequences. The BLAST program uses as defaults:
Cost to open gap: default=5 for nucleotides/11 for proteins
Cost to extend gap: default=2 for nucleotides/1 for proteins
Penalty for nucleotide mismatch: default=−3
Reward for nucleotide match: default=1
Expect value: default=10
Wordsize: default=11 for nucleotides/28 for megablast/3 for proteins.
   Furthermore, the degree of local identity between the amino acid sequence query or nucleic acid sequence query and the retrieved homologous sequences is determined by the BLAST program. However only those sequence segments are compared that give a match above a certain threshold. Accordingly, the program calculates the identity only for these matching segments. Therefore, the identity calculated in this way is referred to as local identity.

The term “mature polypeptide” or “mature enzyme” as used herein refers to a polypeptide in its final active form following translation and any post-translational modifications, such as N-terminal processing, C-terminal truncation, glycosylation, phosphorylation, etc. It is known in the art that a host cell may produce a mixture of two of more different mature polypeptides (i.e., with a different C-terminal and/or N-terminal amino acid) expressed by the same polynucleotide.

The term “cDNA” refers to a DNA molecule that can be prepared by reverse transcription from a mature, spliced, mRNA molecule obtained from a eukaryotic or prokaryotic cell. cDNA lacks intron sequences that may be present in the corresponding genomic DNA. The initial, primary RNA transcript is a precursor to mRNA that is processed through a series of steps, including splicing, before appearing as mature spliced mRNA.

The term “coding sequence” refers to a nucleotide sequence, which directly specifies the amino acid sequence of a polypeptide. The boundaries of the coding sequence are generally determined by an open reading frame, which begins with a start codon such as ATG, GTG, or TTG and ends with a stop codon such as TAA, TAG, or TGA. The coding sequence may be a genomic DNA, cDNA, synthetic DNA, or a combination thereof.

The term “control sequence” as used herein refers to a nucleotide sequence necessary for expression of a polynucleotide encoding a polypeptide. A control sequence may be native (i.e., from the same gene) or heterologous or foreign (i.e., from a different gene) to the polynucleotide encoding the polypeptide. Control sequences include, but are not limited to leader sequences, polyadenylation sequence, pro-peptide coding sequence, promoter sequences, signal peptide coding sequence, translation terminator (stop) sequences and transcription terminator (stop) sequences. To be operational control sequences usually must include promoter sequences, transcriptional and translational stop signals. Control sequences may be provided with linkers for the purpose of introducing specific restriction sites facilitating ligation of the control sequences with a coding region of a polynucleotide encoding a polypeptide.

The term “expression” includes any step involved in the production of a polypeptide including, but not limited to, transcription, post-transcriptional modification, translation, post-translational modification, and secretion.

The term “expression vector” refers to a DNA molecule, either single- or double stranded, either linear or circular, which comprises a polynucleotide encoding a polypeptide and is operably linked to control sequences that provide for its expression. Expression vectors include expression cassettes for the integration of genes into a host cell as well as plasmids and/or chromosomes comprising such genes.

The term “host cell” refers to any cell type that is susceptible to transformation, transfection, transduction, or the like with a nucleic acid construct or expression vector comprising a polynucleotide of the present disclosure. Host cell encompasses any progeny of a parent cell that is not identical to the parent cell due to mutations that occur during replication.

The term “polynucleotide construct” refers to a polynucleotide, either single- or double stranded, which is isolated from a naturally occurring gene or is modified to contain segments of nucleic acids in a manner that would not otherwise exist in nature or which is synthetic, and which comprises a polynucleotide encoding a polypeptide and one or more control sequences.

The term “operably linked” refers to a configuration in which a control sequence is placed at an appropriate position relative to the coding polynucleotide such that the control sequence directs expression of the coding polynucleotide.

The terms “nucleotide sequence and “polynucleotide” are used herein interchangeably.

The term “comprise” and “include” as used throughout the specification and the accompanying items as well as variations such as “comprises”, “comprising”, “includes” and “including” are to be interpreted inclusively. These words are intended to convey the possible inclusion of other elements or integers not specifically recited, where the context allows.

The articles “a” and “an” are used herein refers to one or to more than one (i.e. to one or at least one) of the grammatical object of the article. By way of example, “an element” may mean one element or more than one element.

Terms like “preferably”, “commonly”, “particularly”, and “typically” are not utilized herein to limit the scope of the itemed disclosure or to imply that certain features are critical, essential, or even important to the structure or function of the itemed disclosure. Rather, these terms are merely intended to highlight alternative or additional features that can or cannot be utilized in a particular embodiment of the present disclosure.

The term “cell culture” as used herein refers to a culture medium comprising a plurality of host cells of the disclosure. A cell culture may comprise a single strain of host cells or may comprise two or more distinct host cell strains. The culture medium may be any medium that may comprise a recombinant host, e.g., a liquid medium (i.e., a culture broth) or a semi-solid medium, and may comprise additional components, e.g., a carbon source such as dextrose, sucrose, glycerol, or acetate; a nitrogen source such as ammonium sulfate, urea, or amino acids; a phosphate source; vitamins; trace elements; salts; amino acids; nucleobases; yeast extract; aminoglycoside antibiotics such as G418 and hygromycin B.

The term “a chemical handle for enzymatic conversion toward one or more intermediates leading to the compound of formula (I), including the compound of formula (I)” is a molecular group or a hydrogen, which can be recognized for conversion by the one or more enzymes of the present disclosure when situated on the specific position, R₄. The chemical handle can thus be converted by the one or more enzymes to provide the one or more intermediates. Furthermore, the term “a chemical handle for enzymatic conversion toward one or more intermediates leading to the compound of formula (I), including the compound of formula (I)” refers to a molecular group or a hydrogen situated at the specific position, R4. This chemical handle acts as a specific functional group, designed to be selectively and efficiently targeted or recognized by the corresponding enzymes described in the present disclosure. As a targeted point of interaction, the chemical handle facilitates the enzymatic modification, ensuring that the conversion takes place precisely at the R₄ position.

In some embodiments, the chemical handle also comprises an indole, for example in the case of the IPA dimer, where a second molecule derived from tryptophan or another indole-containing species constitutes the chemical handle R₄ connected to the compound of formula (II) disclosed herein.

The term “alkoyl” as used herein refers to the combination of “alk” and “oyl” meaning a combination of alkyl and a carbonyl. The term is equivalent to acyl.

The term “in-situ extraction” as used herein generally refers to extraction happening during cultivation/fermentation of a host cell but could also happen after completion of the fermentation. In-situ extraction is possible by using an extractant that is non-toxic to the cells and has the physicochemical properties rendering it able to reach the compound of formula (I) being produced intracellularly and extract the compound without killing the cells.

The term “Cx-Cy ester”, as used herein, refers to an ester compound containing a carbon chain length that ranges from x to y carbon atoms, inclusive. This includes, without limitation, ester compounds where x is the minimum number of carbon atoms and y is the maximum number of carbon atoms in the chain. For example, a C2-C20 ester encompasses ester compounds with carbon chain lengths ranging from 2 to 20 carbon atoms.

The term “Cx-Cy alcohol”, as used herein, refers to an alcohol compound with a carbon chain length that spans from x to y carbon atoms, inclusive. This includes, without limitation, alcohol compounds where x is the smallest number of carbon atoms and y is the largest number of carbon atoms in the chain. For instance, a C2-C20 alcohol includes alcohol compounds with carbon chain lengths between 2 and 20 carbon atoms.

The term “textile material”, as used herein, refers to any material derived from the interlacing, entwining, bonding of yarns, fibers, or filaments, or resulting from the polymerization of monomers. This encompasses, without limitation, fabrics in their various finished or unfinished forms, individual yarns, fibers (both natural and synthetic), and polymeric materials whether formed through addition or condensation polymerization. The term aims to cover a broad range of materials from raw fiber to finished fabric, inclusive of intermediary products like threads and yarns, as well as synthetic materials originating from polymerization processes. Moreover, the term “textile material”, as used herein, refers to any material, whether originating from the interlacing, entwining, bonding of yarns, fibers, or filaments, or resulting from the polymerization of monomers, that is amenable to dyeing processes. The defining characteristic of these textile materials in this context is their ability to be dyed, either through inherent properties or through subsequent treatments. The term is intended to cover a broad range of dyeable materials, from raw fiber to finished fabric, including intermediary products like threads and yarns, as well as synthetic materials suitable for dyeing that originate from polymerization processes.

### Methods of Producing a Compound of Formula (I)

The present disclosure provides a method which either in vivo, in vitro, or a combination thereof enables formation of the compounds of interest, namely compounds of formula (I) using one or more enzymes of the present disclosure.

In one embodiment, a method is provided for producing a compound of formula (I):

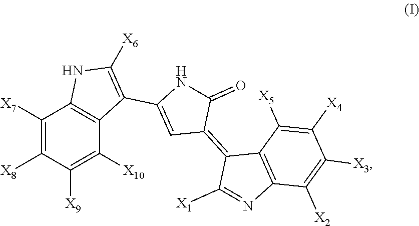

or a tautomer thereof, wherein any one of X₁, X₂, X₃, X₄, X₅, X₆, X₇, X₈, X₉, and X₁₀ are independently of each other selected from the group consisting of: H, R₁, R₂, O, OH, OR₁, NH, NO₂, NH₂, NHR₁, NHR₂, SR₁, F, Cl, Br, I, and SH; wherein R₁ and R₂ are independently of each other selected from the group consisting of a C₁₋₈ alkyl, C₁₋₈ alkenyl, C₁₋₈ alkoyl, C₁₋₈ aryl, and C₁₋₈ aroyl, and R₁ and R₂ are optionally covalently linked to form a ring; wherein the method comprises providing an indole of formula (II):

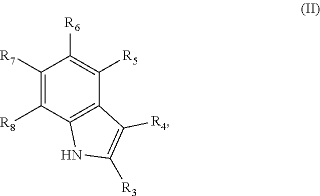

wherein
R₃, R₅, R₆, R₇, and Rₛ are independently of each other selected from the group consisting of: H, R₁, R₂, O, OH, OR₁, NH, NH₂, NHR₁, NHR₂, NO₂, SR₁, F, Cl, Br, I and SH; wherein R₁ and R₂ are independently of each other selected from the group consisting of a C₁₋₈ alkyl, C₁₋₈ alkenyl, C₁₋₈ alkoyl, C₁₋₈ aryl, and C₁₋₈ aroyl, and R₁ and R₂ are optionally covalently linked to form a ring; and wherein
R₄ is a chemical handle for enzymatic conversion toward one or more intermediates leading to the compound of formula (I), including the compound of formula (I), and further comprises contacting the indole of formula (II) with one or more enzymes, optionally wherein the one or more enzymes are from an operative biosynthetic pathway for producing violacein.

In one embodiment, a method is provided for producing a compound of formula (I) selected from the group consisting of:

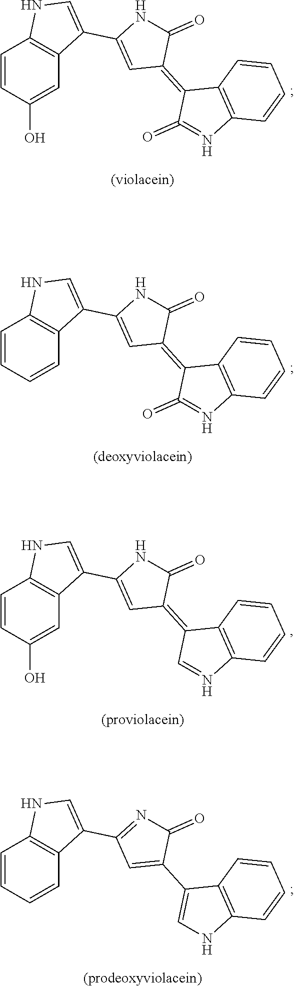

and a tautomer thereof; wherein the method comprises providing an indole of formula (II):

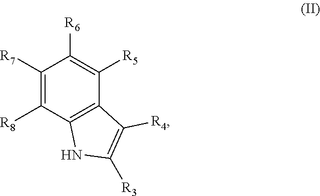

wherein
R₃, R₅, R₆, R₇, and R₈ are independently of each other selected from the group consisting of: H, O, or OH; and wherein R₄ is selected from the group consisting of: H, glycerol-3-phosphate;

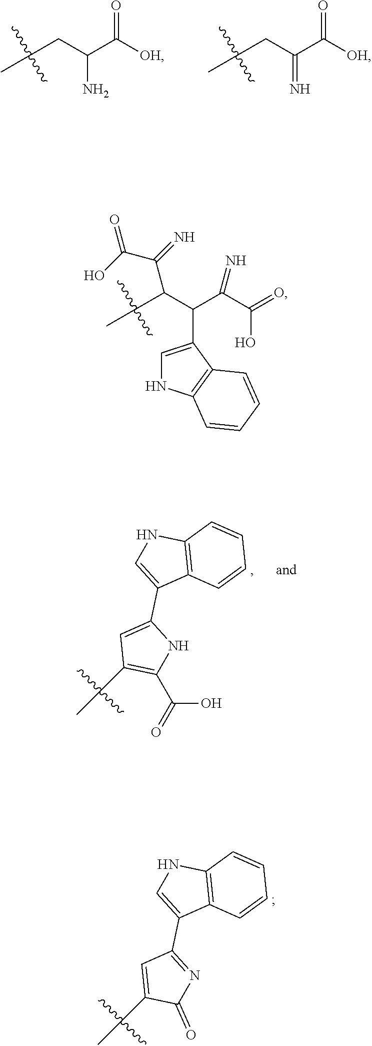 [SMILES: *CC(N)C(=O)O ; *CC(=N)C(=O)O ; *C(C(=N)C(=O)O)C(C(=N)C(=O)O)c1c[nH]c2ccccc12 ; *c1cc(-c2c[nH]c3ccccc23)[nH]c1C(=O)O ; *C1=CC(c2c[nH]c3ccccc23)=NC1=O]

and further comprising contacting the indole of formula (II) with one or more enzymes from an operative biosynthetic pathway for producing violacein comprising, wherein the method involves at least:
a) a tryptophan oxidase having at least 70% identity to the sequence comprised in SsStaO (SEQ ID NO: 80), NlInkO (SEQ ID NO: 84), and/or AmAtmO (SEQ ID NO: 88); and/or
b) an IPA imine dimer synthase has at least 70% identity to the sequence comprised in LaRebD (SEQ ID NO: 78), SsStaD (SEQ ID NO: 82), NlInkD (SEQ ID NO: 86), and/or AmAtmD (SEQ ID NO: 90).

In one embodiment, the chemical handle for enzymatic conversion toward one or more intermediates leading to the compound of formula (I) is selected from the group consisting of: H, and a phosphoric ester of glycerol, such as glycerol-3-phosphate;

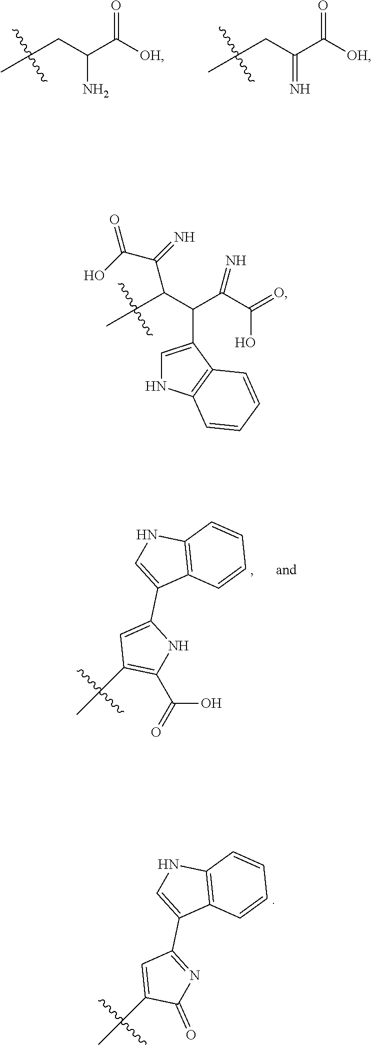 [SMILES: *CC(N)C(=O)O ; *CC(=N)C(=O)O ; *C(C(=N)C(=O)O)C(C(=N)C(=O)O)c1c[nH]c2ccccc12 ; *c1cc(-c2c[nH]c3ccccc23)[nH]c1C(=O)O ; *C1=CC(c2c[nH]c3ccccc23)=NC1=O]

In one embodiment, the one or more intermediates is selected from the group consisting of: TRP, IPA, IPAD, PDV, and PVIO, and substituted analogues thereof bearing substituents corresponding to X₁, X₂, X₃, X₄, X₅, X₆, X₇, X₈, X₉, and/or X₁₀ as defined for the compound of formula (I).

In one embodiment, the one or more intermediates is selected from the group consisting of: tryptophan, indole-3-pyruvic acid imine, indole-3-pyruvic acid imine dimer, protodeoxyviolaceinic acid, and protoviolaceinic acid, and substituted analogues thereof bearing substituents corresponding to X₁, X₂, X₃, X₄, X₅, X₆, X₇, X₈, X₉, and/or X₁₀ as defined for the compound of formula (I).

In one embodiment, the method further involves a non-enzymatic step of oxidative decarboxylation to provide the compound of formula (I). In some embodiments, the reaction happens spontaneously.

In one embodiment, the method comprises contacting the compound of formula (II) with an amino acid, such as a proteinogenic amino acid, for example serine, in the presence of the one or more enzymes.

In one embodiment, the method comprises contacting the compound of formula (II) with one or more pathway molecules selected from: FAD, HEME, and NADPH.

In one embodiment, the compound of formula (I) is selected from the group consisting of:

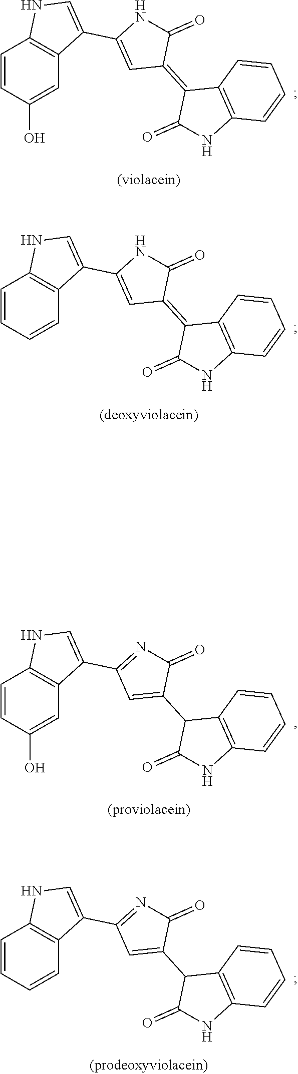 [SMILES: O=C1NC(c2c[nH]c3ccc(O)cc23)=C/C1=C1\C(=O)Nc2ccccc21 ; O=C1NC(c2c[nH]c3ccccc23)=C/C1=C1\C(=O)Nc2ccccc21 ; O=C1N=C(c2c[nH]c3ccc(O)cc23)C=C1C1C(=O)Nc2ccccc21 ; O=C1N=C(c2c[nH]c3ccccc23)C=C1C1C(=O)Nc2ccccc21]

and tautomers thereof.

In one embodiment R₄ is H, and the compound of formula (II) has been prepared in vitro or in vivo.

In one embodiment, at least one of R₃, R₅, R₆, R₇, and R₈ is not H. In one embodiment, R₄ is H, and the compound of formula (II) has been prepared in vitro or in vivo, and at least one of R₃, R₅, R₆, R₇, and R₈ is not H.

In one embodiment, R₄ is glycerol-3-phosphate and the compound of formula (II) has been prepared in vivo.

In one embodiment, the compound of formula (I) is prepared from glucose.

In one embodiment, the indole of formula (II) is selected from indole, and indole-3-glycerol phosphate.

In one embodiment, the compound of formula (I) is contacted with a glycosyl donor comprising a glycosyl group.

In one embodiment, the method comprises a glycosylation step of the compound of formula (I) to provide a glycosylated compound of formula (I), wherein the glycosylated compound of formula (I) comprises the compound of formula (I) covalently attached to the glycosyl group.

In one embodiment, the method comprises a de-glycosylation step such that the glycosylated compound of formula (I) is de-glycosylated to provide the compound of formula (I). The present disclosure enables glycosylation such as to export the compound of formula (I) from the cell to increase isolation yield of the final de-glycosylated compound of formula (I).

In one embodiment, the de-glycosylation step is facilitated by a glycosidase, such as a β-glycosidase.

In one embodiment, the de-glycosylation step is facilitated by a glucosidase, such as a β-glucosidase.

In one embodiment, the glycosyl group of the glycosyl donor comprises one or more of glucose, galactose, xylose, mannose, galactofuranose, arabinose, rhamnose, apiose, fucose, glucosamine, galactosamine, N-acetylglucosamine, N-acetylgalactosamine, xylosamine, mannosamine, arabinosamine, rhamnosamine, apiosamine, fucosamine, glucuronate, galacturonate, mannuronate, arabinate, apionate or a combination thereof.

In one embodiment, the glycosylation step comprises an O-glycosylation, such as a β-O-glycosylation.

In one embodiment, the glycosyl donor is a nucleotide glycoside.

In one embodiment, the nucleotide glycoside is NTP-glycoside, NDP-glycoside or NMP-glycoside.

In one embodiment, the nucleoside of the nucleotide glycoside is selected from Uridine, Adenosin, Guanosin, Cytidin and deoxythymidine.

In one embodiment, the nucleotide glycoside is selected from UDP-glycosides, ADP-glycosides, CDP-glycosides, CMP-glycosides, dTDP-glycosides and GDP-glycosides.

In one embodiment, the nucleotide glycoside is selected from UDP-D-glucose (UDP-Glc); UDP-galactose (UDP-Gal); UDP-D-xylose (UDP-Xyl); UDP-N-acetyl-D-glucosamine (UDP-GlcNAc); UDP-N-acetyl-D-galactosamine (UDP-GalNAc); UDP-D-glucuronic acid (UDP-GlcA); UDP -D-galactofuranose (UDP-Galf); UDP-arabinose; UDP-rhamnose, UDP-apiose; UDP-2-acetamido-2-deoxy-α-D-mannuronate; UDP-N-acetyl-D-galactosamine 4-sulfate; UDP-N-acetyl-D-mannosamine; UDP-2,3-bis(3-hydroxytetradecanoyl)-glucosamine; UDP-4-deoxy-4-formamido-β-L-arabinopyranose; UDP-2,4-bis(acetamido)-2,4,6-trideoxy-α-D-glucopyranose; UDP-galacturonate; UDP-3-amino-3-deoxy-α-D-glucose; guanosine diphospho-D-mannose (GDP-Man); guanosine diphospho-L-fucose (GDP-Fuc); guanosine diphospho-L-rhamnose (GDP-Rha); cytidine monophospho-N-acetylneuraminic acid (CMP-Neu5Ac); cytidine monophospho-2-keto-3-deoxy-D-mannooctanoic acid (CMP-Kdo); and ADP-glucose.

In one embodiment, the one or more enzymes are selected from glycosyltransferases, synthases, kinases, transketolases, transaldolase, phosphoketolases, phosphotransketolases, dehydratases, dehydrogenases, carboxyvinyltransferases, phosphoribosyl transferases, isomerases, oxidases, dimerases, and monooxygenases.

In one embodiment, the glycosyltransferase is derived from a plant or a fungus. In some embodiments, the glycosyltransferase is derived from a plant, a fungus, or a bacterium.

In one embodiment, the plant is selected from Oryza sativa, Crocus sativus, Nicotiana tabacum, Stevia rebaudiana, Nicotiana benthatamiana and Arabidopsis thaliana.

In some embodiments, the plant is selected from Oryza sativa, Crocus sativus, Nicotiana tabacum, Stevia rebaudiana, Nicotiana benthatamiana, Arabidopsis thaliana, Helianthus annuus, and Populus trichocarpa.

In some embodiments, the glycosyltransferase is derived from Bacillus subtilis.

In one embodiment, the glycosyl transferase is an O-glycoside transferase and/or a C-glycoside transferase.

In one embodiment, the glycosyl transferase is an aglycone O-glycosyltransferase.

In one embodiment, the glycosyl transferase is a glycoside O-glycosyltransferase.

In one embodiment, the glycosyl transferase is an aglycone O-glucosyltransferase.

In one embodiment, the glycosyl transferase is an aglycone O-rhamnosyltransferase.

In one embodiment, the glycosyl transferase is an aglycone O-xylosyltransferase.

In one embodiment, the glycosyl transferase is an aglycone O-arabinosyltransferase.

In one embodiment, the glycosyl transferase is an aglycone O—N-acetylgalactosaminyltransferase.

In one embodiment, the glycosyl transferase is an aglycone O—N-acetylglucosaminyltransferase.

In one embodiment, the glycosyl transferase is an aglycone/glycoside mono-O-glycosyltransferase.

In one embodiment, the glycosyl transferase is an aglycone/glycoside di-O-glycosyltransferase.

In one embodiment, the glycosyl transferase is an aglycone/glycoside tri-O-glycosyltransferase.

In one embodiment, the glycosyl transferase is an aglycone/glycoside tetra-O-glycosyltransferase.

In one embodiment, the glycosyl transferase is a hydroxytryptophan glycosyltransferase.

In one embodiment, the glycosyl transferase comprises the sequence of Pt73Y (SEQ ID: NO 64); and/or (SEQ ID NO: 66).

In some embodiments, the glycosyl transferase comprises the sequence of Pt73Y (SEQ ID: NO 64); (SEQ ID NO: 66); Bs109_1 (SEQ ID NO: 68); Bs109A1 (SEQ ID NO: 70); Cp73B (SEQ ID NO: 72); Cs73Y (yeast c/o) (SEQ ID NO: 92); Ha88B_2 (yeast c/o) (SEQ ID NO: 94); and/or Pt73Y (yeast c/o) (SEQ ID NO: 96).

In some embodiments, the glycosyl transferase has at least 70%, such as at least 75%, such as at least 80%, such as at least 90%, such as at least 95%, such as at least 99%, such as 100% identity to the glycosyl transferase comprised in anyone of Pt73Y (SEQ ID: NO 64); (SEQ ID NO: 66); Bs109_1 (SEQ ID NO: 68); Bs109A1 (SEQ ID NO: 70); Cp73B (SEQ ID NO: 72); Cs73Y (yeast c/o) (SEQ ID NO: 92); Ha88B_2 (yeast c/o) (SEQ ID NO: 94); and/or Pt73Y (yeast c/o) (SEQ ID NO: 96).

In one embodiment, the glycosyl transferase has at least 70%, such as at least 75%, such as at least 80%, such as at least 90%, such as at least 95%, such as at least 99%, such as 100% identity to the glycosyl transferase comprised in anyone of SEQ ID NO: 64, and 66.

In one embodiment, the synthase is selected from the group consisting of: a Chorismate synthase, an Anthranilate synthase, an Indole-3-glycerol phosphate synthase, a Tryptophan synthase, a Prodeoxyviolacein synthase, and a Violacein Synthase.

In one embodiment, the Chorismate synthase has at least 70%, such as at least 75%, such as at least 80%, such as at least 90%, such as at least 95%, such as at least 99%, such as 100% identity to the synthase comprised in SEQ ID NO: 12.

In one embodiment, the Anthranilate synthase has at least 70%, such as at least 75%, such as at least 80%, such as at least 90%, such as at least 95%, such as at least 99%, such as 100% identity to the synthase comprised in SEQ ID NO: 14.

In one embodiment, the Indole-3-glycerol phosphate synthase has at least 70%, such as at least 75%, such as at least 80%, such as at least 90%, such as at least 95%, such as at least 99%, such as 100% identity to the synthase comprised in SEQ ID NO: 22.

In one embodiment, the Tryptophan synthase has at least 70%, such as at least 75%, such as at least 80%, such as at least 90%, such as at least 95%, such as at least 99%, such as 100% identity to the synthase comprised in SEQ ID NO: 24 (TRP5) and/or 63 (PcTrpB).

In one embodiment, the synthase has at least 70% identity to the synthase comprised in SEQ ID NO: 24 (TRP5) and the synthase is contacted with the compound of formula (II) in vivo.

In one embodiment, the compound of formula (II) is indole-3-glycerol phosphate.

In one embodiment, the synthase has at least 70% identity to the synthase comprised in SEQ ID NO: 63 (TRP5) and the synthase is contacted with the compound of formula (II) in vitro.

In one embodiment, the compound of formula (II) is indole.

In one embodiment, the Prodeoxyviolacein synthase has at least 70%, such as at least 75%, such as at least 80%, such as at least 90%, such as at least 95%, such as at least 99%, such as 100% identity to the synthase comprised in SEQ ID NO: 30.

In one embodiment, the Violacein Synthase has at least 70%, such as at least 75%, such as at least 80%, such as at least 90%, such as at least 95%, such as at least 99%, such as 100% identity to the synthase comprised in SEQ ID NO: 34.

In one embodiment, the kinase is a Shikimate kinase, a Ribose-phosphate pyrophosphokinase, and/or a NADH kinase.

In one embodiment, the Shikimate kinase has at least 70%, such as at least 75%, such as at least 80%, such as at least 90%, such as at least 95%, such as at least 99%, such as 100% identity to the sequence comprised in SEQ ID NO: 10.

In one embodiment, the Ribose-phosphate pyrophosphokinase has at least 70%, such as at least 75%, such as at least 80%, such as at least 90%, such as at least 95%, such as at least 99%, such as 100% identity to the sequence comprised in SEQ ID NO: 16.

In one embodiment, the NADH kinase has at least 70%, such as at least 75%, such as at least 80%, such as at least 90%, such as at least 95%, such as at least 99%, such as 100% identity to the sequence comprised in SEQ ID NO: 51.

In one embodiment, the one or more enzymes is an Anthranilate phosphoribosyl transferase having at least 70%, such as at least 75%, such as at least 80%, such as at least 90%, such as at least 95%, such as at least 99%, such as 100% identity to the sequence comprised in SEQ ID NO: 18.

In one embodiment, the one or more enzymes is a Flavin-dependent L-tryptophan oxidase having at least 70%, such as at least 75%, such as at least 80%, such as at least 90%, such as at least 95%, such as at least 99%, such as 100% identity to the sequence comprised in SEQ ID NO: 26.

In one embodiment, the one or more enzymes is a 2-imino-3-(indol-3-yl)propanoate dimerase having at least 70%, such as at least 75%, such as at least 80%, such as at least 90%, such as at least 95%, such as at least 99%, such as 100% identity to the sequence comprised in SEQ ID NO: 28.

In one embodiment, the one or more enzymes is a Protodeoxyviolaceinate monooxygenase having at least 70%, such as at least 75%, such as at least 80%, such as at least 90%, such as at least 95%, such as at least 99%, such as 100% identity to the sequence comprised in SEQ ID NO: 32.

In one embodiment, the one or more enzymes is a transaldolase having at least 70%, such as at least 75%, such as at least 80%, such as at least 90%, such as at least 95%, such as at least 99%, such as 100% identity to the sequence comprised in SEQ ID NO: 38.

In one embodiment, the one or more enzymes is a Transketolase having at least 70%, such as at least 75%, such as at least 80%, such as at least 90%, such as at least 95%, such as at least 99%, such as 100% identity to the sequence comprised in SEQ ID NO: 40.

In one embodiment, the one or more enzymes is a GTP cyclohydrolase II having at least 70%, such as at least 75%, such as at least 80%, such as at least 90%, such as at least 95%, such as at least 99%, such as 100% identity to the sequence comprised in SEQ ID NO: 42.

In one embodiment, the one or more enzymes is Mitochondrial flavin adenine dinucleotide transporter having at least 70%, such as at least 75%, such as at least 80%, such as at least 90%, such as at least 95%, such as at least 99%, such as 100% identity to the sequence comprised in SEQ ID NO: 44.

In one embodiment, the one or more enzymes is Porphobilinogen deaminase having at least 70%, such as at least 75%, such as at least 80%, such as at least 90%, such as at least 95%, such as at least 99%, such as 100% identity to the sequence comprised in SEQ ID NO: 47.

In one embodiment, the sequence identity is at least 90%, such as at least 91%, such as at least 92%, such as at least 93%, such as at least 94%, such as at least 95%, such as at least 96%, such as at least 97%, such as at least 98%, such as at least 99%, such as 100%.

In some embodiments, the one or more enzymes is a tryptophan oxidase having at least 70%, such as at least 75%, such as at least 80%, such as at least 90%, such as at least 95%, such as at least 99%, such as 100% identity to the sequence comprised in: SEQ ID NO: 80, SEQ ID NO: 84, and/or SEQ ID NO: 88.

In some embodiments, the one or more enzymes is a 2-imino-3-(indol-3-yl)propanoate dimerase having at least 70%, such as at least 75%, such as at least 80%, such as at least 90%, such as at least 95%, such as at least 99%, such as 100% identity to the sequence comprised in SEQ ID NO: 28.

In some embodiments, the one or more enzymes is a 2-imino-3-(indol-3-yl)propanoate dimerase Prodeoxyviolacein synthase fusion protein having at least 70%, such as at least 75%, such as at least 80%, such as at least 90%, such as at least 95%, such as at least 99%, such as 100% identity to the sequence comprised in SEQ ID NO: 74 or SEQ ID NO: 76.

In some embodiments, the one or more enzymes is an IPA imine dimer synthase having: a) at least 70% identity, such as at least 75%, such as at least 80%, such as at least 90%, such as at least 95%, such as at least 99%, such as 100% identity to LaRebD (SEQ ID NO: 78); b) at least 70% identity, such as at least 75%, such as at least 80%, such as at least 90%, such as at least 95%, such as at least 99%, such as 100% identity to SsStaD (SEQ ID NO: 82); c) at least 70% identity, such as at least 75%, such as at least 80%, such as at least 90%, such as at least 95%, such as at least 99%, such as 100% identity to NlInkD (SEQ ID NO: 86); or d) at least 70% identity, such as at least 75%, such as at least 80%, such as at least 90%, such as at least 95%, such as at least 99%, such as 100% identity to AmAtmD (SEQ ID NO: 90).

### Further Method Steps

In one embodiment, the method further comprises one or more steps selected from:
a) converting an indole or indole derivative into tryptophan or a tryptophan derivative;
b) adding an isolated indole of formula (II) to a microbial host cell;
c) converting an indole of formula (II) into tryptophan or a tryptophan derivative;
d) converting tryptophan or tryptophan derivative into the compound of formula (I);
e) converting the compound of formula (I) into a glycosylated compound thereof which is the glycosylated compound of formula (I), optionally in vivo;
f) extraction of the compound of formula (I) or the glycosylated compound of formula (I) using an extractant, such as a surfactant, optionally at a concentration above the extractant's cloud point; and
g) recovering the compound of formula (I) from a extractant phase.

In one embodiment, the method further comprises extraction of the glycosylated compound of formula (I).

In one embodiment, the method further comprises de-glycosylation of the glycosylated compound of formula (I) by a β-glycosidase to provide the compound of formula (I), and optionally further isolating the compound of formula (I).

In one embodiment, the method is provided wherein the extractant is a surfactant, such as a non-ionic surfactant. In some embodiments, the extractant is a surfactant, such as a non-ionic surfactant; or a lipophilic extractant. In some embodiments, the extractant is non-miscible with water.

In one embodiment, method is provided wherein the extractant is isopropyl myristate, (1,1,3,3-Tetramethylbutyl)phenyl-polyethylene glycol, Polyethylene glycol tert-octylphenyl ether (Triton X-114), or polydimethylsiloxane (such as Antifoam A).

In one embodiment, the method is provided according to the “Further method steps” section disclosed herein, wherein the steps are performed in vitro or in vivo.

In one embodiment, the method is provided wherein the conversion of the indole into the tryptophan or tryptophan derivative comprises contacting the indole with a tryptophan synthase enzyme, optionally a tryptophan synthase which has at least 70%, such as at least 75%, such as at least 80%, such as at least 90%, such as at least 95%, such as at least 99%, such as 100% identity to the tryptophan synthase comprised in SEQ ID NO: 24 and/or 63.

In one embodiment, the method is provided comprising in vitro enzymatic reaction steps and/or optionally in vivo enzymatic reaction steps.

In one embodiment, the method comprises expressing a glycosyl transferase in yeast, such as in S. cerevisiae and performing in vivo glycosylation of the compound of formula (I).

In one embodiment, the method is provided wherein the glycosyl transferase has at least 70% sequence identity to the polypeptide sequence comprised in sequence of Pt73Y according to SEQ ID NO: 66, such as at least 75%, such as at least 80%, such as at least 85%, such as at least 90%, such as at least 95%, such as 100%.

In one embodiment, the method comprises expressing a glycosyl transferase in yeast, such as in S. cerevisiae or Pichia pastoris and performing in vitro glycosylation of the compound of formula (I).

In one embodiment, the method comprises expressing a glycosyl transferase in E. coli and performing in vitro glycosylation of the compound of formula (I).

In one embodiment, the glycosyl transferase has at least 70% sequence identity to the polypeptide sequence comprised in sequence of Pt73Y according to SEQ ID NO: 66, such as at least 75%, such as at least 80%, such as at least 85%, such as at least 90%, such as at least 95%, such as 100%.

In some embodiments, the glycosyl transferase has at least 70% sequence identity to any one of the polypeptide sequences comprised in sequence of: Pt73Y (SEQ ID: NO 64); (SEQ ID NO: 66); Bs109_1 (SEQ ID NO: 68); Bs109A1 (SEQ ID NO: 70); Cp73B (SEQ ID NO: 72); Cs73Y (yeast c/o) (SEQ ID NO: 92); Ha88B_2 (yeast c/o) (SEQ ID NO: 94); and/or Pt73Y (yeast c/o) (SEQ ID NO: 96).

In some embodiments, the glycosyl transferase has at least 70% sequence identity to any one of the polypeptide sequences comprised in sequence of: Pt73Y (SEQ ID: NO 64); Cs73Y (yeast c/o) (SEQ ID NO: 92); and/or Pt73Y (yeast c/o) (SEQ ID NO: 96), such as at least 75%, such as at least 80%, such as at least 85%, such as at least 90%, such as at least 95%, such as 100%.

In some embodiments, the glycosyl transferase has at least 70% sequence identity to any one of the polypeptide sequences comprised in sequence of: Pt73Y (SEQ ID: NO 64); (SEQ ID NO: 66); Bs109_1 (SEQ ID NO: 68); Bs109A1 (SEQ ID NO: 70); Cp73B (SEQ ID NO: 72); Cs73Y (yeast c/o) (SEQ ID NO: 92); Ha88B_2 (yeast c/o) (SEQ ID NO: 94); and/or Pt73Y (yeast c/o) (SEQ ID NO: 96).

### Compounds of Formula (I)

In one embodiment, the present disclosure provides a compound of formula (I):

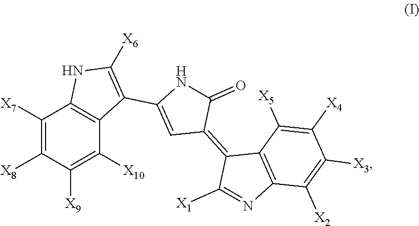

or a tautomer thereof, wherein any one of X₁, X₂, X₃, X₄, X₅, X₆, X₇, X₈, X₉, and X₁₀ are independently of each other selected from the group consisting of: H, R₁, R₂, O, OH, OR₁, NH, NO₂, NH₂, NHR₁, NHR₂, SR₁, F, Cl, Br, I, and SH; wherein R₁ and R₂ are independently of each other selected from the group consisting of a C₁₋₈ alkyl, C₁₋₈ alkenyl, C₁₋₈ alkoyl, C₁₋₈ aryl, and C₁₋₈ aroyl, and R₁ and R₂ are optionally covalently linked to form a ring.

In one embodiment, the compound is selected from the group consisting of:

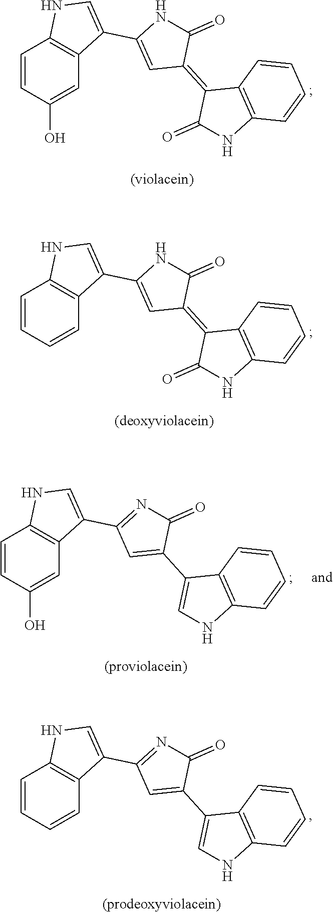 [SMILES: O=C1NC(c2c[nH]c3ccc(O)cc23)=C/C1=C1\C(=O)Nc2ccccc21 ; O=C1NC(c2c[nH]c3ccccc23)=C/C1=C1\C(=O)Nc2ccccc21 ; O=C1N=C(c2c[nH]c3ccc(O)cc23)C=C1c1c[nH]c2ccccc12 ; O=C1N=C(c2c[nH]c3ccccc23)C=C1c1c[nH]c2ccccc12]

and tautomers thereof.

### Glycosides of Formula (I)

In one embodiment, the compound is provided according to formula (I) or according to violacein and its analogues above further covalently linked to a saccharide, preferably by a glycosidic linkage.

In one embodiment, the compound referred to above is in enol form covalently linked to a saccharide via an enol oxygen, preferably by a glycosidic linkage.

In one embodiment, the compound is of formula (III) or formula (IV):

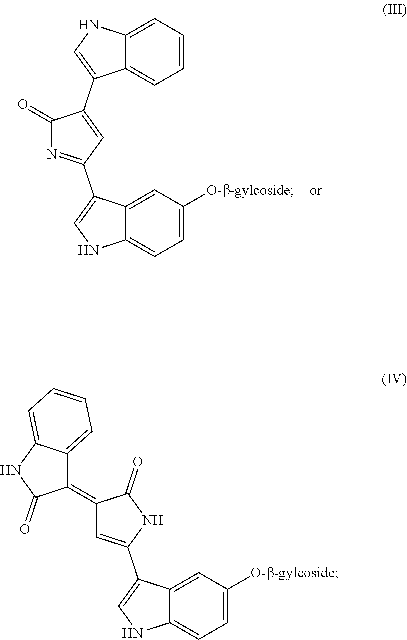

wherein “β-glycoside” is a saccharide linked by a β-glycosidic bond to the remainder of the molecule.

In one embodiment, the compound is provided wherein the saccharide is a monosaccharide, a disaccharide, a trisaccharide, or a tetrasaccharide.

In one embodiment, the monosaccharide is selected from the group consisting of: glucose, fructose, galactose, mannose, arabinose, xylose, ribulose, xylulose, ribose, desoxyribose, desoxygalactose, fucose, and rhamnose, preferably wherein the monosaccharide is glucose, such as D-glucose.

### Microbial Host Cell

In one embodiment, a microbial host cell is provided genetically modified to perform any of the methods disclosed herein and produce a compound of formula (I),

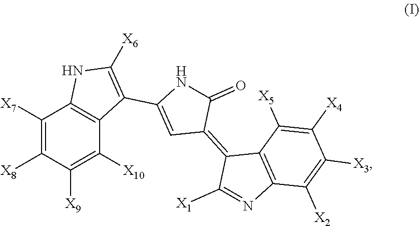

or a tautomer thereof, wherein any one of X₁, X₂, X₃, X₄, X₅, X₆, X₇, X₈, X₉, and X₁₀ are independently of each other selected from the group consisting of: H, R₁, R₂, O, OH, OR₁, NH, NO₂, NH₂, NHR₁, NHR₂, SR₁, F, Cl, Br, I, and SH; wherein R₁ and R₂ are independently of each other selected from the group consisting of a C₁₋₈ alkyl, C₁₋₈ alkenyl, C₁₋₈ alkoyl, C₁₋₈ aryl, and C₁₋₈ aroyl, and R₁ and R₂ are optionally covalently linked to form a ring;
wherein the host cell expresses one or more heterologous genes encoding the one or more enzymes.

In one embodiment, a microbial host cell is provided genetically modified to perform any of the methods disclosed herein and produce a compound of formula (I) selected from the group consisting of:

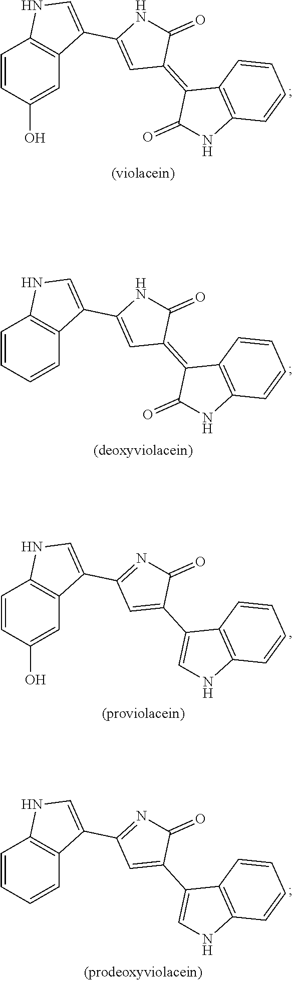 [SMILES: O=C1NC(c2c[nH]c3ccc(O)cc23)=C/C1=C1\C(=O)Nc2ccccc21 ; O=C1NC(c2c[nH]c3ccccc23)=C/C1=C1\C(=O)Nc2ccccc21 ; O=C1N=C(c2c[nH]c3ccc(O)cc23)C=C1c1c[nH]c2ccccc12 ; O=C1N=C(c2c[nH]c3ccccc23)C=C1c1c[nH]c2ccccc12]

and a tautomer thereof; wherein the host cell expresses one or more heterologous genes encoding the one or more enzymes, and wherein the microbial host cell comprises at least:
a) a tryptophan oxidase having at least 70% identity to the sequence comprised in SsStaO (SEQ ID NO: 80), NlInkO (SEQ ID NO: 84), and/or AmAtmO (SEQ ID NO: 88); and/or
b) an IPA imine dimer synthase has at least 70% identity to the sequence comprised in LaRebD (SEQ ID NO: 78), SsStaD (SEQ ID NO: 82), NlInkD (SEQ ID NO: 86), and/or AmAtmD (SEQ ID NO: 90); and wherein the microbial host cell is Saccharomyces cerevisiae.

In one embodiment, the host cell is provided which further comprises an operative biosynthetic pathway for producing violacein, wherein the host cell expresses one or more pathway genes encoding polypeptides selected from:
a) one or more enzymes capable of converting glucose to fructose-6-phosphate;
b) one or more enzymes capable of converting glucose to D-ribulose-5-phosphate;
c) a transketolase capable of converting xylulose-5-phosphate and ribose-5-phosphate to glyceraldehyde-3-phosphate and sedoheptulose-7-phosphate, such as the transketolase TKL1;
d) a transaldolase capable of converting glyceraldehyde 3-phosphate and sedoheptulose 7-phosphate to erythrose 4-phosphate and fructose 6-phosphate, such as the transaldolase TAL1;
e) a fructose-6-phosphate phosphoketolase capable of converting fructose-6-phosphate to Erythrose-4-phosphate and acetyl phosphate, such as the phosphoketolase BfXfpk;
f) a Phosphotransacetylase capable of converting Acetyl phosphate to Acetyl-CoA, such as the phosphotransacetylase CkPTa;
g) one or more enzymes capable of converting Fructose-6-phosphate to Phosphoenolpyruvate;
h) a 3-deoxy-D-arabino-heptulosonate 7-phosphate synthase (DAHP synthase) capable of converting Phosphoenolpyruvate and Erythrose-4-phosphate to 3-deoxy-D-arabino-heptulosonate-7-phosphate (DAHP), such as the synthase ARO4(K229L);
i) a 3-dehydroquinate synthase capable of converting 3-deoxy-D-arabino-heptulosonate 7-phosphate to 3-dehydroquinate, such as the synthase ARO1;
j) a 3-dehydroquinate dehydratase capable of converting 3-dehydroquinate to 3-dehydroshikimate, such as the dehydratase ARO1;
k) a Shikimate dehydrogenase capable of converting 3-dehydroshikimate to Shikimate, such as the dehydrogenase ARO1;
l) a Shikimate kinase capable of converting Shikimate to Shikimate-3-phosphate, such as the kinase ARO1 and/or EcAroL;
m) a 3-phosphoshikimate 1-carboxyvinyltransferase capable of converting Shikimate-3-phosphate and Phosphoenolpyruvate to 5-enolpyruvoyl-shikimate 3-phosphate, such as the transferase ARO1;
n) a Chorismate synthase capable of converting 5-enolpyruvoyl-shikimate 3-phosphate to Chorismate, such as the synthase ARO2;
o) an Anthranilate synthase capable of converting Chorismate to Anthranilate, such as the synthase TRP2(S65R, S76L);
p) a Ribose-phosphate pyrophosphokinase capable of converting Ribose-5-phosphate to Phospho-alpha-D-ribosyl-1-pyrophosphate, such as the pyrophosphokinase BsPrs;
q) an Anthranilate phosphoribosyl transferase capable of converting Anthranilate and Phospho-alpha-D-ribosyl-1-pyrophosphate to N-(5-phosphoribosyl)-anthranilate, such as the transferase TRP4;
r) a N′(5′-phosphoribosyl)-anthranilate isomerase capable of converting N-(5-phosphoribosyl)-anthranilate to 1-(o-carboxyphenylamino′-1′-deoxyribulos' 5′-phosphate, such as the isomerase TRP1;
s) a Indole-3-glycerol phosphate synthase capable of converting 1-(o-carboxyphenylamino′-1-deoxyribulos' 5-phosphate to (1S,2R)-1-C-(indol-3-yl)glycerol 3-phosphate, such as the synthase TRP3
t) a Tryptophan synthase capable of converting (1S,2R)-1-C-(indol-3-yl)glycerol 3-phosphate and Serine to L-Tryptophan, such as the synthase TRP5;
u) a tryptophan synthase capable of converting Indole and Serine to L-Tryptophan, such as the synthase TRP5;
v) a Flavin-dependent L-tryptophan oxidase capable of converting L-Tryptophan to IPA imine, such as CvVioA;
w) a 2-imino-3-(indol-3-yl)propanoate dimerase capable of converting IPA imine to IPA imine dimer, such as the dimerase CvVioB;
x) a Prodeoxyviolacein synthase capable of converting IPA imine dimer to Protodeoxyviolaceinic acid, such as the synthase CvVioE;
y) a Protodeoxyviolaceinate monooxygenase synthase capable of converting Protodeoxyviolaceinic acid to Protoviolaceinic acid, such as the synthase CvVioD; and z) a Violacein synthase capable of converting Protoviolaceinic acid to Violaceinic acid, such as CvVioC.

In some embodiments, a host cell is provided further comprising an operative biosynthetic pathway for producing violacein, wherein the host cell expresses one or more pathway genes encoding polypeptides selected from:
a) one or more enzymes capable of converting glucose to fructose-6-phosphate;
b) one or more enzymes capable of converting glucose to D-ribulose-5-phosphate;
c) a transketolase capable of converting xylulose-5-phosphate and ribose-5-phosphate to glyceraldehyde-3-phosphate and sedoheptulose-7-phosphate, such as the transketolase TKL1;
d) a transaldolase capable of converting glyceraldehyde 3-phosphate and sedoheptulose 7-phosphate to erythrose 4-phosphate and fructose 6-phosphate, such as the transaldolase TAL1;
e) a fructose-6-phosphate phosphoketolase capable of converting fructose-6-phosphate to Erythrose-4-phosphate and acetyl phosphate, such as the phosphoketolase BfXfpk;
f) a Phosphotransacetylase capable of converting Acetyl phosphate to Acetyl-CoA, such as the phosphotransacetylase CkPTa;
g) one or more enzymes capable of converting Fructose-6-phosphate to Phosphoenolpyruvate;
h) a 3-deoxy-D-arabino-heptulosonate 7-phosphate synthase (DAHP synthase) capable of converting Phosphoenolpyruvate and Erythrose-4-phosphate to 3-deoxy-D-arabino-heptulosonate-7-phosphate (DAHP), such as the synthase ARO4(K229L);
i) a 3-dehydroquinate synthase capable of converting 3-deoxy-D-arabino-heptulosonate 7-phosphate to 3-dehydroquinate, such as the synthase ARO1;
j) a 3-dehydroquinate dehydratase capable of converting 3-dehydroquinate to 3-dehydroshikimate, such as the dehydratase ARO1;
k) a Shikimate dehydrogenase capable of converting 3-dehydroshikimate to Shikimate, such as the dehydrogenase ARO1;
l) a Shikimate kinase capable of converting Shikimate to Shikimate-3-phosphate, such as the kinase ARO1 and/or EcAroL;
m) a 3-phosphoshikimate 1-carboxyvinyltransferase capable of converting Shikimate-3-phosphate and Phosphoenolpyruvate to 5-enolpyruvoyl-shikimate 3-phosphate, such as the transferase ARO1;
n) a Chorismate synthase capable of converting 5-enolpyruvoyl-shikimate 3-phosphate to Chorismate, such as the synthase ARO2;
o) an Anthranilate synthase capable of converting Chorismate to Anthranilate, such as the synthase TRP2(S65R, S76L);
p) a Ribose-phosphate pyrophosphokinase capable of converting Ribose-5-phosphate to Phospho-alpha-D-ribosyl-1-pyrophosphate, such as the pyrophosphokinase BsPrs;
q) an Anthranilate phosphoribosyl transferase capable of converting Anthranilate and Phospho-alpha-D-ribosyl-1-pyrophosphate to N-(5-phosphoribosyl)-anthranilate, such as the transferase TRP4;
r) a N-(5′-phosphoribosyl)-anthranilate isomerase capable of converting N-(5-phosphoribosyl)-anthranilate to 1-(o-carboxyphenylamino)-1′-deoxyribulose 5′-phosphate, such as the isomerase TRP1;
s) a Indole-3-glycerol phosphate synthase capable of converting 1-(o-carboxyphenylamino)-1′-deoxyribulose 5′-phosphate to (1S,2R)-1-C-(indol-3-yl)glycerol 3-phosphate, such as the synthase TRP3;
t) a Tryptophan synthase capable of converting (1S,2R)-1-C-(indol-3-yl)glycerol 3-phosphate and Serine to L-Tryptophan, such as the synthase TRP5;
u) a tryptophan synthase capable of converting Indole and Serine to L-Tryptophan, such as the synthase TRP5;
v) a Flavin-dependent L-tryptophan oxidase capable of converting L-Tryptophan to IPA imine, such as CvVioA;
w) a tryptophan oxidase, such as SsStaO, NlInkO, or AmAtmO;
x) a 2-imino-3-(indol-3-yl)propanoate dimerase capable of converting IPA imine to IPA imine dimer, such as the dimerase CvVioB;
y) an IPA imine dimer synthase, such as LaRebD, SsStaD, NlInkD, and/or AmAtmD;
z) a Prodeoxyviolacein synthase capable of converting IPA imine dimer to Protodeoxyviolaceinic acid, such as the synthase CvVioE;
aa) a Protodeoxyviolaceinate monooxygenase synthase capable of converting Protodeoxyviolaceinic acid to Protoviolaceinic acid, such as the synthase CvVioD; and
bb) a Violacein synthase capable of converting Protoviolaceinic acid to Violaceinic acid and capable of converting Protodeoxyviolaceinic acid to Protoviolaceinic acid, such as CvVioC.

In one embodiment, the host cell further comprising an operative biosynthetic pathway for heme biosynthesis, wherein the host cell expresses one or more pathway genes encoding polypeptides selected from:
a) one or more enzymes capable of converting glucose to glycine;
b) one or more enzymes capable of converting glycine to porphobilinogen;
c) a Porphobilinogen deaminase capable of converting Porphobilinogen to Hydroxymethylbilane, such as the deaminase HEM3; and
d) one or more enzymes capable of converting Hydroxymethylbilane to Ferroheme b.

In one embodiment, the host cell of the present disclosure further comprises an operative biosynthetic pathway for flavin biosynthesis, wherein the host cell expresses one or more pathway genes encoding polypeptides selected from:
a) a GTP cyclohydrolase II capable of converting GTP to 2,5-diamino-6-ribosylamino-4(3H)-pyrimidinone 5′-phosphate, such as the cyclohydrolase RIB1; and
b) one or more enzymes capable of converting 2,5-diamino-6-ribosylamino-4(3H)-pyrimidinone 5′-phosphate to FAD.

In one embodiment, the host cell further expresses one or more genes encoding catalytic or non-catalytic polypeptides selected from:
a) a NADH kinase capable of converting NADH and ATP to NADPH and ADP, such as the kinase POS5; and
b) a Mitochondrial flavin adenine dinucleotide transporter, such as FLX1.

In one embodiment, the host cell is provided wherein one or more genes has been attenuated, disrupted and/or deleted, said one or more genes encoding catalytic or non-catalytic polypeptides selected from:
a) a Heme oxygenase capable of converting Ferroheme b to Biliverdin, such as the oxygenase HMX1;
b) a Heme-responsive transcription factor, such as HAP1;
c) a mRNA-binding ubiquitin-specific protease, such as UBP3;
d) a Cis-Golgi network transporter protein, such as RIC1; and
e) a Heme-dependent repressor of hypoxic genes, such as ROX1.

In one embodiment, the host cell is provided, wherein the corresponding:
a) transketolase capable of converting xylulose-5-phosphate and ribose-5-phosphate to glyceraldehyde-3-phosphate and sedoheptulose-7-phosphate has at least 70% identity to the sequence comprised in SEQ ID NO: 40;
b) transaldolase capable of converting glyceraldehyde 3-phosphate and sedoheptulose 7-phosphate to erythrose 4-phosphate and fructose 6-phosphate has at least 70% identity to the sequence comprised in SEQ ID NO: 38;
c) fructose-6-phosphate phosphoketolase capable of converting fructose-6-phosphate to Erythrose-4-phosphate and acetyl phosphate has at least 70% identity to the sequence comprised in SEQ ID NO: 2;
d) Phosphotransacetylase capable of converting Acetyl phosphate to Acetyl-CoA has at least 70% identity to the sequence comprised in SEQ ID NO: 4;
e) 3-deoxy-D-arabino-heptulosonate 7-phosphate synthase (DAHP synthase) capable of converting Phosphoenolpyruvate and Erythrose-4-phosphate to 3-deoxy-D-arabino-heptulosonate-7-phosphate (DAHP) has at least 70% identity to the sequence comprised in SEQ ID NO: 6;
f) 3-dehydroquinate synthase capable of converting 3-deoxy-D-arabino-heptulosonate 7-phosphate to 3-dehydroquinate has at least 70% identity to the sequence comprised in SEQ ID NO: 8;
g) 3-dehydroquinate dehydratase capable of converting 3-dehydroquinate to 3-dehydroshikimate has at least 70% identity to the sequence comprised in SEQ ID NO: 8; h) Shikimate dehydrogenase capable of converting 3-dehydroshikimate to Shikimate has at least 70% identity to the sequence comprised in SEQ ID NO: 8;
i) Shikimate kinase capable of converting Shikimate to Shikimate-3-phosphate has at least 70% identity to the sequence comprised in SEQ ID NO: 8; and/or at least 70% identity to the sequence comprised in SEQ ID NO: 10;
j) 3-phosphoshikimate 1-carboxyvinyltransferase capable of converting Shikimate-3-phosphate and Phosphoenolpyruvate to 5-enolpyruvoyl-shikimate 3-phosphate has at least 70% identity to the sequence comprised in SEQ ID NO: 8;
k) Chorismate synthase capable of converting 5-enolpyruvoyl-shikimate 3-phosphate to Chorismate has at least 70% identity to the sequence comprised in SEQ ID NO: 12;
l) Anthranilate synthase capable of converting Chorismate to Anthranilate has at least 70% identity to the sequence comprised in SEQ ID NO: 14;
m) Ribose-phosphate pyrophosphokinase capable of converting Ribose-5-phosphate to Phospho-alpha-D-ribosyl-1-pyrophosphate has at least 70% identity to the sequence comprised in SEQ ID NO: 16;
n) Anthranilate phosphoribosyl transferase capable of converting Anthranilate and Phospho-alpha-D-ribosyl-1-pyrophosphate to N-(5-phosphoribosyl)-anthranilate has at least 70% identity to the sequence comprised in SEQ ID NO: 18;
o) N-(5′-phosphoribosyl)-anthranilate isomerase capable of converting N-(5-phosphoribosyl)-anthranilate to 1-(o-carboxyphenylamino)-1′-deoxyribulose 5′-phosphate has at least 70% identity to the sequence comprised in SEQ ID NO: 20;
p) Indole-3-glycerol phosphate synthase capable of converting 1-(o-carboxyphenylamino)-1′-deoxyribulose 5′-phosphate to (1S,2R)-1-C-(indol-3-yl)glycerol 3-phosphate has at least 70% identity to the sequence comprised in SEQ ID NO: 22;
q) Tryptophan synthase capable of converting (1S,2R)-1-C-(indol-3-yl)glycerol 3-phosphate and Serine to L-Tryptophan has at least 70% identity to the sequence comprised in SEQ ID NO: 24;
r) Flavin-dependent L-tryptophan oxidase capable of converting L-Tryptophan to IPA imine has at least 70% identity to the sequence comprised in SEQ ID NO: 26;
s) 2-imino-3-(indol-3-yl)propanoate dimerase capable of converting IPA imine to IPA imine dimer has at least 70% identity to the sequence comprised in SEQ ID NO: 28;
t) Prodeoxyviolacein synthase capable of converting IPA imine dimer to Protodeoxyviolaceinic acid has at least 70% identity to the sequence comprised in SEQ ID NO: 30;
u) Protodeoxyviolaceinate monooxygenase synthase capable of converting Protodeoxyviolaceinic acid to Protoviolaceinic acid has at least 70% identity to the sequence comprised in SEQ ID NO: 32; and/or
v) Violacein synthase capable of converting Protoviolaceinic acid to Violaceinic acid has at least 70% identity to the sequence comprised in SEQ ID NO: 34.

In some embodiments, the host cell is provided wherein the corresponding:
a) transketolase capable of converting xylulose-5-phosphate and ribose-5-phosphate to glyceraldehyde-3-phosphate and sedoheptulose-7-phosphate has at least 70% identity to the sequence comprised in SEQ ID NO: 40;
b) transaldolase capable of converting glyceraldehyde 3-phosphate and sedoheptulose 7-phosphate to erythrose 4-phosphate and fructose 6-phosphate has at least 70% identity to the sequence comprised in SEQ ID NO: 38;
c) fructose-6-phosphate phosphoketolase capable of converting fructose-6-phosphate to Erythrose-4-phosphate and acetyl phosphate has at least 70% identity to the sequence comprised in SEQ ID NO: 2;
d) Phosphotransacetylase capable of converting Acetyl phosphate to Acetyl-CoA has at least 70% identity to the sequence comprised in SEQ ID NO: 4;
e) 3-deoxy-D-arabino-heptulosonate 7-phosphate synthase (DAHP synthase) capable of converting Phosphoenolpyruvate and Erythrose-4-phosphate to 3-deoxy-D-arabino-heptulosonate-7-phosphate (DAHP) has at least 70% identity to the sequence comprised in SEQ ID NO: 6;
f) 3-dehydroquinate synthase capable of converting 3-deoxy-D-arabino-heptulosonate 7-phosphate to 3-dehydroquinate has at least 70% identity to the sequence comprised in SEQ ID NO: 8;
g) 3-dehydroquinate dehydratase capable of converting 3-dehydroquinate to 3-dehydroshikimate has at least 70% identity to the sequence comprised in SEQ ID NO: 8;
h) Shikimate dehydrogenase capable of converting 3-dehydroshikimate to Shikimate has at least 70% identity to the sequence comprised in SEQ ID NO: 8;
i) Shikimate kinase capable of converting Shikimate to Shikimate-3-phosphate has at least 70% identity to the sequence comprised in SEQ ID NO: 8; and/or at least 70% identity to the sequence comprised in SEQ ID NO: 10;
j) 3-phosphoshikimate 1-carboxyvinyltransferase capable of converting Shikimate-3-phosphate and Phosphoenolpyruvate to 5-enolpyruvoyl-shikimate 3-phosphate has at least 70% identity to the sequence comprised in SEQ ID NO: 8;
k) Chorismate synthase capable of converting 5-enolpyruvoyl-shikimate 3-phosphate to Chorismate has at least 70% identity to the sequence comprised in SEQ ID NO: 12;
l) Anthranilate synthase capable of converting Chorismate to Anthranilate has at least 70% identity to the sequence comprised in SEQ ID NO: 14;
m) Ribose-phosphate pyrophosphokinase capable of converting Ribose-5-phosphate to Phospho-alpha-D-ribosyl-1-pyrophosphate has at least 70% identity to the sequence comprised in SEQ ID NO: 16;
n) Anthranilate phosphoribosyl transferase capable of converting Anthranilate and Phospho-alpha-D-ribosyl-1-pyrophosphate to N-(5-phosphoribosyl)-anthranilate has at least 70% identity to the sequence comprised in SEQ ID NO: 18;
o) N-(5′-phosphoribosyl)-anthranilate isomerase capable of converting N-(5-phosphoribosyl)-anthranilate to 1-(o-carboxyphenylamino)-1′-deoxyribulose 5′-phosphate has at least 70% identity to the sequence comprised in SEQ ID NO: 20;
p) Indole-3-glycerol phosphate synthase capable of converting 1-(o-carboxyphenylamino)-1′-deoxyribulose 5′-phosphate to (1S,2R)-1-C-(indol-3-yl)glycerol 3-phosphate has at least 70% identity to the sequence comprised in SEQ ID NO: 22;
q) Tryptophan synthase capable of converting (1S,2R)-1-C-(indol-3-yl)glycerol 3-phosphate and Serine to L-Tryptophan has at least 70% identity to the sequence comprised in SEQ ID NO: 24;
r) Flavin-dependent L-tryptophan oxidase capable of converting L-Tryptophan to IPA imine has at least 70% identity to the sequence comprised in SEQ ID NO: 26;
s) tryptophan oxidase has at least 70% identity to the sequence comprised in SsStaO (SEQ ID NO: 80), NlInkO (SEQ ID NO: 84), and/or AmAtmO (SEQ ID NO: 88);
t) 2-imino-3-(indol-3-yl)propanoate dimerase capable of converting IPA imine to IPA imine dimer has at least 70% identity to the sequence comprised in SEQ ID NO: 28;
u) IPA imine dimer synthase has at least 70% identity to the sequence comprised in LaRebD (SEQ ID NO: 78), SsStaD (SEQ ID NO: 82), NlInkD (SEQ ID NO: 86), and/or AmAtmD (SEQ ID NO: 90);
v) Prodeoxyviolacein synthase capable of converting IPA imine dimer to Protodeoxyviolaceinic acid has at least 70% identity to the sequence comprised in SEQ ID NO: 30;
w) Protodeoxyviolaceinate monooxygenase synthase capable of converting Protodeoxyviolaceinic acid to Protoviolaceinic acid has at least 70% identity to the sequence comprised in SEQ ID NO: 32; and/or
x) Violacein synthase capable of converting Protoviolaceinic acid to Violaceinic acid and capable of converting Protodeoxyviolaceinic acid to Protoviolaceinic acid has at least 70% identity to the sequence comprised in SEQ ID NO: 34.

In one embodiment, the host cell is provided wherein the one or more expressed genes are selected from:
a) genes encoding a transketolase capable of converting xylulose-5-phosphate and ribose-5-phosphate to glyceraldehyde-3-phosphate and sedoheptulose-7-phosphate, said genes being at least 70% identical to the polynucleotide sequence comprised in SEQ ID NO: 39 or genomic DNA thereof;
b) genes encoding a transaldolase capable of converting glyceraldehyde 3-phosphate and sedoheptulose 7-phosphate to erythrose 4-phosphate and fructose 6-phosphate, said genes being at least 70% identical to the polynucleotide sequence comprised in SEQ ID NO: 37 or genomic DNA thereof;
c) genes encoding a fructose-6-phosphate phosphoketolase capable of converting fructose-6-phosphate to Erythrose-4-phosphate and acetyl phosphate, said genes being at least 70% identical to the polynucleotide sequence comprised in SEQ ID NO: 1 or genomic DNA thereof;
d) genes encoding a Glycerol-1-phosphatase capable of converting Acetyl phosphate to Acetate, said genes being at least 70% identical to the polynucleotide sequence comprised in SEQ ID NO: 59 or genomic DNA thereof;
e) genes encoding a Phosphotransacetylase capable of converting Acetyl phosphate to Acetyl-CoA, said genes being at least 70% identical to the polynucleotide sequence comprised in SEQ ID NO: 3 or genomic DNA thereof;
f) genes encoding a 3-deoxy-D-arabino-heptulosonate 7-phosphate synthase (DAHP synthase) capable of converting Phosphoenolpyruvate and Erythrose-4-phosphate to 3-deoxy-D-arabino-heptulosonate-7-phosphate (DAHP), said genes being at least 70% identical to the polynucleotide sequence comprised in SEQ ID NO: 5 or genomic DNA thereof;
g) genes encoding a 3-dehydroquinate synthase capable of converting 3-deoxy-D-arabino-heptulosonate 7-phosphate to 3-dehydroquinate, said genes being at least 70% identical to the polynucleotide sequence comprised in SEQ ID NO: 7 or genomic DNA thereof;
h) genes encoding a 3-dehydroquinate dehydratase capable of converting 3-dehydroquinate to 3-dehydroshikimate, said genes being at least 70% identical to the polynucleotide sequence comprised in SEQ ID NO: 7 or genomic DNA thereof;
i) genes encoding a Shikimate dehydrogenase capable of converting 3-dehydroshikimate to Shikimate, said genes being at least 70% identical to the polynucleotide sequence comprised in SEQ ID NO: 7 or genomic DNA thereof;
j) genes encoding a Shikimate kinase capable of converting Shikimate to Shikimate-3-phosphate, said genes being at least 70% identical to the polynucleotide sequence comprised in SEQ ID NO: 7 or genomic DNA thereof and/or at least 70% identical to the polynucleotide sequence comprised in SEQ ID NO: 9 or genomic DNA thereof;
k) genes encoding a 3-phosphoshikimate 1-carboxyvinyltransferase capable of converting Shikimate-3-phosphate and Phosphoenolpyruvate to 5-enolpyruvoyl-shikimate 3-phosphate, said genes being at least 70% identical to the polynucleotide sequence comprised in SEQ ID NO: 7 or genomic DNA thereof;
l) genes encoding a Chorismate synthase capable of converting 5-enolpyruvoyl-shikimate 3-phosphate to Chorismate, said genes being at least 70% identical to the polynucleotide sequence comprised in SEQ ID NO: 11 or genomic DNA thereof;
m) genes encoding an Anthranilate synthase capable of converting Chorismate to Anthranilate, said genes being at least 70% identical to the polynucleotide sequence comprised in SEQ ID NO: 13 or genomic DNA thereof;
n) genes encoding a Ribose-phosphate pyrophosphokinase capable of converting Ribose-5-phosphate to Phospho-alpha-D-ribosyl-1-pyrophosphate, said genes being at least 70% identical to the polynucleotide sequence comprised in SEQ ID NO: 15 or genomic DNA thereof;
o) genes encoding an Anthranilate phosphoribosyl transferase capable of converting Anthranilate and Phospho-alpha-D-ribosyl-1-pyrophosphate to N-(5-phosphoribosyl)-anthranilate, said genes being at least 70% identical to the polynucleotide sequence comprised in SEQ ID NO: 17 or genomic DNA thereof;
p) genes encoding a N-(5′-phosphoribosyl)-anthranilate isomerase capable of converting N-(5-phosphoribosyl)-anthranilate to 1-(o-carboxyphenylamino)-1′-deoxyribulose 5′-phosphate, said genes being at least 70% identical to the polynucleotide sequence comprised in SEQ ID NO: 19 or genomic DNA thereof;
q) genes encoding a Indole-3-glycerol phosphate synthase capable of converting 1-(o-carboxyphenylamino)-1′-deoxyribulose 5′-phosphate to (1S,2R)-1-C-(indol-3-yl)glycerol 3-phosphate, said genes being at least 70% identical to the polynucleotide sequence comprised in SEQ ID NO: 21 or genomic DNA thereof;
r) genes encoding a Tryptophan synthase capable of converting (1S,2R)-1-C-(indol-3-yl)glycerol 3-phosphate and Serine to L-Tryptophan, said genes being at least 70% identical to the polynucleotide sequence comprised in SEQ ID NO: 23 or genomic DNA thereof;
s) genes encoding a Flavin-dependent L-tryptophan oxidase capable of converting L-Tryptophan to IPA imine, said genes being at least 70% identical to the polynucleotide sequence comprised in SEQ ID NO: 25 or genomic DNA thereof;
t) genes encoding a 2-imino-3-(indol-3-yl)propanoate dimerase capable of converting IPA imine to IPA imine dimer, said genes being at least 70% identical to the polynucleotide sequence comprised in SEQ ID NO: 27 or genomic DNA thereof;
u) genes encoding a Prodeoxyviolacein synthase capable of converting IPA imine dimer to Protodeoxyviolaceinic acid, said genes being at least 70% identical to the polynucleotide sequence comprised in SEQ ID NO: 29 or genomic DNA thereof;
v) genes encoding a Protodeoxyviolaceinate monooxygenase synthase capable of converting Protodeoxyviolaceinic acid to Protoviolaceinic acid, said genes being at least 70% identical to the polynucleotide sequence comprised in SEQ ID NO: 31 or genomic DNA thereof;
w) genes encoding a Violacein synthase capable of converting Protoviolaceinic acid to Violaceinic acid, said genes being at least 70% identical to the polynucleotide sequence comprised in SEQ ID NO: 33 or genomic DNA thereof.

In one embodiment, the host cell is provided, wherein the corresponding:
a) Porphobilinogen deaminase capable of converting Porphobilinogen to Hydroxymethylbilane has at least 70% identity to the sequence comprised in SEQ ID NO: 47.

In one embodiment, the host cell is provided, wherein the one or more expressed genes are selected from:
a) genes encoding Porphobilinogen deaminase capable of converting Porphobilinogen to Hydroxymethylbilane, said genes being at least 70% identical to the polynucleotide sequence comprised in SEQ ID NO: 46 or genomic DNA thereof.

In one embodiment, the host cell is provided wherein the corresponding:
a) GTP cyclohydrolase II capable of converting GTP to 2,5-diamino-6-ribosylamino-4(3H)-pyrimidinone 5′-phosphate has at least 70% identity to the sequence comprised in SEQ ID NO: 42.

In one embodiment, the host cell is provided wherein the one or more expressed genes are selected from:
a) genes encoding GTP cyclohydrolase II capable of converting GTP to 2,5-diamino-6-ribosylamino-4(3H)-pyrimidinone 5′-phosphate, said genes being at least 70% identical to the polynucleotide sequence comprised in SEQ ID NO: 41 or genomic DNA thereof.

In one embodiment, the host cell is provided wherein the corresponding:
a) NADH kinase capable of converting NADH and ATP to NADPH and ADP has at least 70% identity to the sequence comprised in SEQ ID NO: 51;
b) Mitochondrial flavin adenine dinucleotide transporter has at least 70% identity to the sequence comprised in SEQ ID NO: 44;
c) Tryptophan synthase has at least 70% identity to the sequence comprised in SEQ ID NO: 63; and/or
d) Glycosyltransferase has at least 70% identity to the sequence comprised in SEQ ID NO: 66, SEQ ID NO: 68, SEQ ID NO: 70, SEQ ID NO: 72, SEQ ID NO: 92, SEQ ID NO: 94, and/or SEQ ID NO:96.

In one embodiment, the host cell is provided wherein the one or more expressed genes are selected from:
a) genes encoding a NADH kinase capable of converting NADH and ATP to NADPH and ADP, said genes being at least 70% identical to the polynucleotide sequence comprised in SEQ ID NO: 50 or genomic DNA thereof;
b) genes encoding a Mitochondrial flavin adenine dinucleotide transporter, said genes being at least 70% identical to the polynucleotide sequence comprised in SEQ ID NO: 43 or genomic DNA thereof;
c) genes encoding a Tryptophan synthase, said genes being at least 70% identical to the polynucleotide sequence comprised in SEQ ID NO: 62 or genomic DNA thereof; and
d) genes encoding a Glycosyltransferase, said genes being at least 70% identical to the polynucleotide sequence comprised in SEQ ID NO: 64, SEQ ID NO: 65, SEQ ID NO: 67, SEQ ID NO: 69, SEQ ID NO: 71, SEQ ID NO: 91, SEQ ID NO: 93, and/or SEQ ID NO: 95, or genomic DNA thereof.

In some embodiments, the host cell is provided wherein the one or more expressed genes are selected from:
a) genes encoding a transketolase capable of converting xylulose-5-phosphate and ribose-5-phosphate to glyceraldehyde-3-phosphate and sedoheptulose-7-phosphate, said genes being at least 70% identical to the polynucleotide sequence comprised in SEQ ID NO: 39 or genomic DNA thereof;
b) genes encoding a transaldolase capable of converting glyceraldehyde 3-phosphate and sedoheptulose 7-phosphate to erythrose 4-phosphate and fructose 6-phosphate, said genes being at least 70% identical to the polynucleotide sequence comprised in SEQ ID NO: 37 or genomic DNA thereof;
c) genes encoding a fructose-6-phosphate phosphoketolase capable of converting fructose-6-phosphate to Erythrose-4-phosphate and acetyl phosphate, said genes being at least 70% identical to the polynucleotide sequence comprised in SEQ ID NO: 1 or genomic DNA thereof;
d) genes encoding a Glycerol-1-phosphatase capable of converting Acetyl phosphate to Acetate, said genes being at least 70% identical to the polynucleotide sequence comprised in SEQ ID NO: 59 or genomic DNA thereof;
e) genes encoding a Phosphotransacetylase capable of converting Acetyl phosphate to Acetyl-CoA, said genes being at least 70% identical to the polynucleotide sequence comprised in SEQ ID NO: 3 or genomic DNA thereof;
f) genes encoding a 3-deoxy-D-arabino-heptulosonate 7-phosphate synthase (DAHP synthase) capable of converting Phosphoenolpyruvate and Erythrose-4-phosphate to 3-deoxy-D-arabino-heptulosonate-7-phosphate (DAHP), said genes being at least 70% identical to the polynucleotide sequence comprised in SEQ ID NO: 5 or genomic DNA thereof;
g) genes encoding a 3-dehydroquinate synthase capable of converting 3-deoxy-D-arabino-heptulosonate 7-phosphate to 3-dehydroquinate, said genes being at least 70% identical to the polynucleotide sequence comprised in SEQ ID NO: 7 or genomic DNA thereof;
h) genes encoding a 3-dehydroquinate dehydratase capable of converting 3-dehydroquinate to 3-dehydroshikimate, said genes being at least 70% identical to the polynucleotide sequence comprised in SEQ ID NO: 7 or genomic DNA thereof;
i) genes encoding a Shikimate dehydrogenase capable of converting 3-dehydroshikimate to Shikimate, said genes being at least 70% identical to the polynucleotide sequence comprised in SEQ ID NO: 7 or genomic DNA thereof;
j) genes encoding a Shikimate kinase capable of converting Shikimate to Shikimate-3-phosphate, said genes being at least 70% identical to the polynucleotide sequence comprised in SEQ ID NO: 7 or genomic DNA thereof and/or at least 70% identical to the polynucleotide sequence comprised in SEQ ID NO: 9 or genomic DNA thereof;
k) genes encoding a 3-phosphoshikimate 1-carboxyvinyltransferase capable of converting Shikimate-3-phosphate and Phosphoenolpyruvate to 5-enolpyruvoyl-shikimate 3-phosphate, said genes being at least 70% identical to the polynucleotide sequence comprised in SEQ ID NO: 7 or genomic DNA thereof;
l) genes encoding a Chorismate synthase capable of converting 5-enolpyruvoyl-shikimate 3-phosphate to Chorismate, said genes being at least 70% identical to the polynucleotide sequence comprised in SEQ ID NO: 11 or genomic DNA thereof;
m) genes encoding an Anthranilate synthase capable of converting Chorismate to Anthranilate, said genes being at least 70% identical to the polynucleotide sequence comprised in SEQ ID NO: 13 or genomic DNA thereof;
n) genes encoding a Ribose-phosphate pyrophosphokinase capable of converting Ribose-5-phosphate to Phospho-alpha-D-ribosyl-1-pyrophosphate, said genes being at least 70% identical to the polynucleotide sequence comprised in SEQ ID NO: 15 or genomic DNA thereof;
o) genes encoding an Anthranilate phosphoribosyl transferase capable of converting Anthranilate and Phospho-alpha-D-ribosyl-1-pyrophosphate to N-(5-phosphoribosyl)-anthranilate, said genes being at least 70% identical to the polynucleotide sequence comprised in SEQ ID NO: 17 or genomic DNA thereof;
p) genes encoding a N-(5′-phosphoribosyl)-anthranilate isomerase capable of converting N-(5-phosphoribosyl)-anthranilate to 1-(o-carboxyphenylamino)-1′-deoxyribulose 5′-phosphate, said genes being at least 70% identical to the polynucleotide sequence comprised in SEQ ID NO: 19 or genomic DNA thereof;
q) genes encoding a Indole-3-glycerol phosphate synthase capable of converting 1-(o-carboxyphenylamino)-1′-deoxyribulose 5′-phosphate to (1S,2R)-1-C-(indol-3-yl)glycerol 3-phosphate, said genes being at least 70% identical to the polynucleotide sequence comprised in SEQ ID NO: 21 or genomic DNA thereof;
r) genes encoding a Tryptophan synthase capable of converting (1S,2R)-1-C-(indol-3-yl)glycerol 3-phosphate and Serine to L-Tryptophan, said genes being at least 70% identical to the polynucleotide sequence comprised in SEQ ID NO: 23 or genomic DNA thereof;
s) genes encoding a Flavin-dependent L-tryptophan oxidase capable of converting L-Tryptophan to IPA imine, said genes being at least 70% identical to the polynucleotide sequence comprised in SEQ ID NO: 25 or genomic DNA thereof;
t) genes encoding a tryptophan oxidase, said genes being at least 70% identical to the polynucleotide sequence comprised in any one of SEQ ID NO: 79, SEQ ID NO: 83, SEQ ID NO: 87, or genomic DNA thereof;
u) genes encoding a 2-imino-3-(indol-3-yl)propanoate dimerase capable of converting IPA imine to IPA imine dimer, said genes being at least 70% identical to the polynucleotide sequence comprised in SEQ ID NO: 27 or genomic DNA thereof;
v) genes encoding a IPA imine dimer synthase, said genes being at least 70% identical to the polynucleotide sequence comprised in any one of SEQ ID NO: 77, SEQ ID NO: 81, SEQ ID NO: 85, SEQ ID NO: 89, or genomic DNA thereof;
w) genes encoding a Prodeoxyviolacein synthase capable of converting IPA imine dimer to Protodeoxyviolaceinic acid, said genes being at least 70% identical to the polynucleotide sequence comprised in SEQ ID NO: 29 or genomic DNA thereof;
x) genes encoding a Protodeoxyviolaceinate monooxygenase synthase capable of converting Protodeoxyviolaceinic acid to Protoviolaceinic acid, said genes being at least 70% identical to the polynucleotide sequence comprised in SEQ ID NO: 31 or genomic DNA thereof;
y) genes encoding a Violacein synthase capable of converting Protoviolaceinic acid to Violaceinic acid, said genes being at least 70% identical to the polynucleotide sequence comprised in SEQ ID NO: 33 or genomic DNA thereof.

In one embodiment, the host cell is provided wherein the sequence identity is least 90%, such as at least 91%, such as at least 92%, such as at least 93%, such as at least 94%, such as at least 95%, such as at least 96%, such as at least 97%, such as at least 98%, such as at least 99%, such as 100%.

In one embodiment, the host cell is provided wherein the sequence identity is at least 99%, such as 100%.

In one embodiment, the host cell is provided comprising at least two copies of one or more of the heterologous genes encoding the one or more enzymes of the pathway genes.

In one embodiment, the host cell is provided wherein one or more of the heterologous genes encoding the one or more enzymes are overexpressed.

In one embodiment, the host cell is provided which is further genetically modified to provide an increased amount of a substrate for at least one polypeptide of the violacein pathway.

In one embodiment, the host cell is further genetically modified to exhibit increased tolerance towards one or more substrates, intermediates, or product molecules from the indole acceptor pathway.

### Host Cell Origin

In one embodiment, the host cell is a eukaryotic, prokaryotic or archaic host cell.

In one embodiment, the host cell is a eukaryote cell selected from the group consisting of a mammalian, insect, plant, or fungal host cell.

In one embodiment, the host cell is a fungal host cell selected from phylas consisting of Ascomycota, Basidiomycota, Neocallimastigomycota, Glomeromycota, Blastocladiomycota, Chytridiomycota, Zygomycota, Oomycota and Microsporidia.

In one embodiment, the fungal host cell is a yeast host cell selected from the group consisting of ascosporogenous yeast (Endomycetales), basidiosporogenous yeast, and Fungi Imperfecti yeast (Blastomycetes).

In one embodiment, the yeast host cell is selected from the genera consisting of Saccharomyces, Kluveromyces, Candida, Pichia, Debaromyces, Hansenula, Yarrowia, Zygosaccharomyces, and Schizosaccharomyces.

In one embodiment, the host cell is provided, wherein the yeast host cell is selected from the species consisting of Kluyveromyces lactis, Saccharomyces carlsbergensis, Saccharomyces cerevisiae, Saccharomyces diastaticus, Saccharomyces douglasii, Saccharomyces kluyveri, Saccharomyces norbensis, Saccharomyces oviformis, Saccharomyces boulardii, Pichia pastoris and Yarrowia lipolytica.

In one embodiment, the host cell is a filamentous fungus host cell.

In one embodiment, the filamentous fungal host cell is selected from the phylas consisting of Ascomycota, Eumycota and Oomycota.

In one embodiment, the filamentous fungal host cell is selected from the genera consisting of Acremonium, Aspergillus, Aureobasidium, Bjerkandera, Ceriporiopsis, Chrysosporium, Coprinus, Corio/us, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, and Trichoderma.

In one embodiment, the filamentous fungal host cell is selected from the species consisting of Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporiuminops, Chrysosporiumkeratinophilum, Chrysosporium lucknowense, Chrysosporium merdarium, Chrysosporium pannicola, Chrysosporium queenslandicum, Chrysosporium tropicum, Chrysosporium zonatum, Coprinus cinereus, Coriolus hirsutus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Thielavia terrestris, Trametes villosa, Trametes versicolor, Trichoderma harzianum, Trichoderma koningii, Trichoderma longibrachiatum, Trichoderma reesei, and Trichoderma viride.

In one embodiment, the host cell is a prokaryotic cell.

In one embodiment, the host cell is E. coli.

In one embodiment, the host cell is an archaic cell.

In one embodiment, the archaic cell is an algae.

Modifications of Genes and/or Polypeptides

In one embodiment, the host cell is provided, wherein one or more native genes are attenuated, disrupted and/or deleted.

In one embodiment, the host cell is a yeast strain modified by attenuating, disrupting and/or deleting one or more native genes selected from:
a) The ARO10 gene comprised in anyone of SEQ ID NO: 49 or any of its paralogs or orthologs having at least 70% identity to SEQ ID NO: 49;
b) The PDC5 gene comprised in anyone of SEQ ID NO: 48 or any of its paralogs or orthologs having at least 70% identity to SEQ ID NO: 48;
c) The UBP3 gene comprised in anyone of SEQ ID NO: 57 or any of its paralogs or orthologs having at least 70% identity to SEQ ID NO: 57;
d) The RIC1 gene comprised in anyone of SEQ ID NO: 58 or any of its paralogs or orthologs having at least 70% identity to SEQ ID NO: 58;
e) The GPP1 gene comprised in anyone of SEQ ID NO: 59 or any of its paralogs or orthologs having at least 70% identity to SEQ ID NO: 59;
f) The ROX1 gene comprised in anyone of SEQ ID NO: 59 or any of its paralogs or orthologs having at least 70% identity to SEQ ID NO: 60;
g) The HMX1 gene comprised in anyone of SEQ ID NO: 59 or any of its paralogs or orthologs having at least 70% identity to SEQ ID NO: 45; and
h) The HAP1 gene comprised in anyone of SEQ ID NO: 59 or any of its paralogs or orthologs having at least 70% identity to SEQ ID NO: 61.

In one embodiment, the host cell is a yeast strain modified by overexpressing one or more genes selected from:
a) The ARO1 gene comprised in SEQ ID NO: 7 or any of its paralogs or orthologs having at least 70% identity to SEQ ID NO: 7;
b) The ARO2 gene comprised in SEQ ID NO: 11 or any of its paralogs or orthologs having at least 70% identity to SEQ ID NO:11;
c) The TRP4 gene comprised in SEQ ID NO: 17 or any of its paralogs or orthologs having at least 70% identity to SEQ ID NO: 17;
d) The TRP1 gene comprised in SEQ ID NO: 19 or any of its paralogs or orthologs having at least 70% identity to SEQ ID NO: 19;
e) The TRP3 gene comprised in SEQ ID NO: 21 or any of its paralogs or orthologs having at least 70% identity to SEQ ID NO: 21;
f) The TRP5 gene comprised in SEQ ID NO: 23 or any of its paralogs or orthologs having at least 70% identity to SEQ ID NO: 23;
g) The TAL1 gene comprised in SEQ ID NO: 37 or any of its paralogs or orthologs having at least 70% identity to SEQ ID NO: 37;
h) The TKL1 gene comprised in SEQ ID NO: 39 or any of its paralogs or orthologs having at least 70% identity to SEQ ID NO: 39;
i) The RIB1 gene comprised in SEQ ID NO: 41 or any of its paralogs or orthologs having at least 70% identity to SEQ ID NO: 41;
j) The FLX1 gene comprised in SEQ ID NO: 43 or any of its paralogs or orthologs having at least 70% identity to SEQ ID NO: 43;
k) The POS5 gene comprised in SEQ ID NO: 50 or any of its paralogs or orthologs having at least 70% identity to SEQ ID NO: 50; and
l) The HEM3 gene comprised in SEQ ID NO: 46 or any of its paralogs or orthologs having at least 70% identity to SEQ ID NO: 46.

In one embodiment, the host cell is a yeast strain modified by overexpressing one or more genes selected from:
a) The K229L modified ARO4 gene, ARO4(K229L) comprised in SEQ ID NO: 5 or any of its paralogs or orthologs having at least 70% identity to SEQ ID NO: 5; and
b) The (S65R, S76L) modified TRP2 gene, TRP2(S65R, S76L) comprised in SEQ ID NO: 13 or any of its paralogs or orthologs having at least 70% identity to SEQ ID NO: 13.

In one embodiment, the host cell is a yeast strain modified by heterologous gene overexpressing of one or more genes selected from:
a) CvVioA encoding comprised in SEQ ID NO: 25 or any of its paralogs or orthologs having at least 70% identity to SEQ ID NO: 25;
b) CvVioB comprised in SEQ ID NO: 27 or any of its paralogs or orthologs having at least 70% identity to SEQ ID NO: 27;
c) CvVioC comprised in SEQ ID NO: 33 or any of its paralogs or orthologs having at least 70% identity to SEQ ID NO: 33;
d) CvVioD comprised in SEQ ID NO: 31 or any of its paralogs or orthologs having at least 70% identity to SEQ ID NO: 31;
e) CvVioE comprised in SEQ ID NO: 29 or any of its paralogs or orthologs having at least 70% identity to SEQ ID NO: 29;
f) BfXfpk comprised in SEQ ID NO: 1 or any of its paralogs or orthologs having at least 70% identity to SEQ ID NO: 1;
g) CkPta comprised in SEQ ID NO: 3 or any of its paralogs or orthologs having at least 70% identity to SEQ ID NO: 3;
h) EcAroL comprised in SEQ ID NO: 9 or any of its paralogs or orthologs having at least 70% identity to SEQ ID NO: 9; and
i) BsPrs comprised in SEQ ID NO: 15 or any of its paralogs or orthologs having at least 70% identity to SEQ ID NO: 15.

In some embodiments, the host cell is a yeast strain modified by heterologous gene overexpressing of one or more genes selected from:
a) CvVioA encoding comprised in SEQ ID NO: 25 or any of its paralogs or orthologs having at least 70% identity to SEQ ID NO: 25;
b) CvVioB comprised in SEQ ID NO: 27 or any of its paralogs or orthologs having at least 70% identity to SEQ ID NO: 27;
c) CvVioC comprised in SEQ ID NO: 33 or any of its paralogs or orthologs having at least 70% identity to SEQ ID NO: 33;
d) CvVioD comprised in SEQ ID NO: 31 or any of its paralogs or orthologs having at least 70% identity to SEQ ID NO: 31;
e) CvVioE comprised in SEQ ID NO: 29 or any of its paralogs or orthologs having at least 70% identity to SEQ ID NO: 29;
f) CvVioB-E fusion GGGGS3 linker comprised in SEQ ID NO:73 or any of its paralogs or orthologs having at least 70% identity to SEQ ID NO: 73;
g) CvVioB-E fusion EAAAK3 linker comprised in SEQ ID NO: 75 or any of its paralogs or orthologs having at least 70% identity to SEQ ID NO: 75;
h) BfXfpk comprised in SEQ ID NO: 1 or any of its paralogs or orthologs having at least 70% identity to SEQ ID NO: 1;
i) CkPta comprised in SEQ ID NO: 3 or any of its paralogs or orthologs having at least 70% identity to SEQ ID NO: 3;
j) EcAroL comprised in SEQ ID NO: 9 or any of its paralogs or orthologs having at least 70% identity to SEQ ID NO: 9; and
k) BsPrs comprised in SEQ ID NO: 15 or any of its paralogs or orthologs having at least 70% identity to SEQ ID NO: 15.

In some embodiments, the host cell is genetically engineered to produce one or more glycosyl transferases, such as one or more UDP-glucuronosyltransferases (UGT's).

In some embodiments, the one or more glycosyl transferases are configured for or capable of glycosylating the compound of formula (I).

In some embodiments, the one or more glycosyl transferases have at least 70% sequence identity to the polypeptide sequence comprised in the sequence of Pt73Y according to SEQ ID NO: 66, such as at least 75%, such as at least 80%, such as at least 85%, such as at least 90%, such as at least 95%, such as 100%.

In some embodiments, the one or more glycosyl transferases have at least 70% sequence identity to any one of the polypeptide sequences comprised in the sequence of: Pt73Y (SEQ ID: NO 64); (SEQ ID NO: 66); Bs109_1 (SEQ ID NO: 68); Bs109A1 (SEQ ID NO: 70); Cp73B (SEQ ID NO: 72); Cs73Y (yeast c/o) (SEQ ID NO: 92); Ha88B_2 (yeast c/o) (SEQ ID NO: 94); and/or Pt73Y (yeast c/o) (SEQ ID NO: 96).

In some embodiments, the one or more glycosyl transferases produced by the host cell have at least 70% sequence identity to any one of the polypeptide sequences comprised in sequence of: Pt73Y (SEQ ID: NO 64); Cs73Y (yeast c/o) (SEQ ID NO: 92); and/or Pt73Y (yeast c/o) (SEQ ID NO: 96), such as at least 75%, such as at least 80%, such as at least 85%, such as at least 90%, such as at least 95%, such as 100%.

In some embodiments, said host cell is a yeast, such as S. cerevisiae, and expresses the one or more glycosyl transferases. In some embodiments, said host cell is an E. coli, and expresses the one or more glycosyl transferases.

### Cell Culture

The present disclosure further provides a cell culture including methods involving said cell culture. In one embodiment, a cell culture is provided comprising a host cell as defined herein and a growth medium.

### Methods Involving the Cell Culture

In one embodiment, the method of the present disclosure further comprises:
a) culturing the cell culture of disclosed herein at conditions allowing the host cell to produce the compound of formula (I); and
b) optionally recovering and/or isolating the compound of formula (I).

In one embodiment, the above method further comprising one or more elements selected from:
c) culturing the cell culture in a nutrient growth medium;
d) culturing the cell culture under aerobic or anaerobic conditions
e) culturing the cell culture under agitation;
f) culturing the cell culture at a temperature of between 25 to 50° C.;
g) culturing the cell culture at a pH of between 3-9;
h) culturing the cell culture for between 10 hours to 30 days; and
i) culturing the cell culture under fed-batch, repeated fed-batch, continuous, or semi-continuous conditions.

In one embodiment, the method further comprises feeding one or more exogenous indoles of formula (II) to the cell culture.

In one embodiment, the recovering and/or isolation step comprises separating a liquid phase of host cell or cell culture from a solid phase of host cell or cell culture to obtain a supernatant comprising the compound of formula (I) by one or more steps selected from:
a) disrupting the host cell to release intracellular the compound of formula (I) into the supernatant;
b) separating the supernatant from the solid phase of the host cell, such as by filtration or gravity separation;
c) contacting the supernatant with one or more adsorbent resins in order to obtain at least a portion of the produced compound of formula (I);
d) contacting the supernatant with one or more ion exchange or reversed-phase chromatography columns in order to obtain at least a portion of the compound of formula (I);
e) extracting the compound of formula (I); and
f) precipitating the compound of formula (I) by crystallization or evaporating the solvent of the liquid phase; and optionally isolating the compound of formula (I) by filtration or gravity separation;

thereby recovering and/or isolating the compound of formula (I).

### Fermentation Liquid

In one embodiment, a fermentation liquid comprising the compound of formula (I) comprised in the cell culture disclosed herein is provided.

In one embodiment, the fermentation liquid is provided, wherein at least 50%, such as at least 75%, such as at least 95%, such as at least 99% of the host cells are disrupted.

In one embodiment, the fermentation liquid is provided, wherein at least 50%, such as at least 75%, such as at least 95%, such as at least 99% of solid cellular material has separated from the liquid.

In one embodiment, the fermentation liquid is provided further comprising one or more compounds selected from:
a) precursors or products of the operative biosynthetic pathway producing the compound of formula (I);
b) supplemental nutrients comprising trace metals, vitamins, salts, yeast nitrogen base, YNB, and/or amino acids; and

wherein the concentration of the compound of formula (I) is at least 1 mg/I liquid.

### Composition

In one embodiment, a composition comprising the fermentation liquid defined herein and/or the compound of formula (I) and one or more agents, additives and/or excipients.

In one embodiment, the composition is provided wherein the fermentation liquid and/or the compound of formula (I) have been processed into in a dry solid form, optionally in form of a powder.

In one embodiment, the composition is in a liquid form, optionally in a stabilized liquid form.

### Modification of the Microbial Cell

In one embodiment, a method for modification of a microbial host cell is provided producing the compound formula (I) as defined herein, comprising:
a) Providing a microbial host cell, such as a yeast host cell, such as S. cerevisiae;
b) Engineering the microbial host cell by inserting one or more genes encoding one or more of the enzymes as defined herein.

In one embodiment, the modification of the microbial host cell is provided where the microbial host cell is as defined herein.

### In-Situ Extraction

In one embodiment, a method for in-situ extraction of the compound of formula (I) or the glycosylated compound of formula (I), comprising:
a) Providing a host cell as defined herein or the cell culture as defined herein comprising the compound of formula (I) or the glycosylated compound of formula (I) in an aqueous phase;
b) Subjecting the aqueous phase to extraction with an extractant, optionally wherein the extractant is a non-ionic surfactant, preferably wherein the extraction is performed during cultivation of the host cell.

In one embodiment, the method is provided wherein the extractant is a surfactant. In some embodiments, the extractant is a surfactant or a lipophilic extractant.

In one embodiment, the method is provided wherein the surfactant is a non-ionic or ionic surfactant.

In some embodiments, the method further comprises producing the compound of formula (I) using the method as defined herein.

In one embodiment, the method is provided wherein the extractant is subjected to the aqueous phase to form a liquid media with the aqueous phase such that the concentration of the extractant with respect to the liquid media is at least at the cloud-point of the extractant.

In one embodiment, the method is provided, wherein the extractant is subjected to the aqueous phase to form a liquid media with the aqueous phase such that the concentration of the extractant with respect to the liquid media is at least at the cloud-point of the extractant and below the toxicity level for the host cell. Typically, the toxicity level can be expressed as a LD50 value, or whether the “toxicity level” has been reached can be determined as in Example 10; if the final OD600 of a strain cultivated in the presence of an extractant is similar to the final OD600 of the same strain cultivated without an extractant then it can be considered non-toxic at the given concentration.

In some embodiments, the extractant is a lipophilic extractant, preferably a non-toxic lipophilic extractant. In some embodiments, the extractant is a lipophilic non-volatile extractant. In some embodiments, the lipophilic extractant is selected from the group consisting of: an ester, such as a C₂-C₂₀ ester, an alcohol, such as a C₂-C₂₀ alcohol, and a vegetable oil, such as grapeseed oil, olive oil, sunflower oil, or canola oil.

In one embodiment, the method is provided, wherein the extractant is (1,1,3,3-tetramethylbutyl)phenyl-polyethylene glycol, polyethylene glycol tert-octylphenyl ether (Triton X-114).

In some embodiments, the method is provided wherein the extractant is polydimethylsiloxane (such as Antifoam A). In some embodiments, the extractant is isopropyl myristate.

In some embodiments, the extractant is selected from the group consisting of Antifoam-A, Triton-X 114, isopropyl myristate, isopropyl palmitate, polysorbate 20, ethyl laurate, castor oil, oleyl alcohol, butyl caprilate, grapeseed oil, 2-butyl-1-octanol, and oleic acid, or any combination thereof.

In some embodiments, the method is provided where the extractant is isopropyl myristate.

In some embodiments, the method is provided where the extractant is added such as to provide a concentration of the extractant of at least 1%, such as from 1-20%, such as from 1-2%, such as from 2-3%, such as from 3-4%, such as from 4-5%, such as from 5-6%, such as from 6-7%, such as from 7-8%, such as from 8-9%, such as from 9-10%, such as from 10-11%, such as from 11-12%, such as from 12-13%, such as from 13-14%, such as from 14-15%, such as from 15-16%, such as from 16-17%, such as from 17-18%, such as from 18-19%, such as from 19-20%.

In some embodiments, the method according to the present in situ extraction is provided, wherein the compound of formula (I) is isolated subsequent to extraction.

### Downstream Processing (DSP)

In some embodiments, the present disclosure provides a method involving in-situ extraction as disclosed herein, further comprising one or more steps of:
a) removing biomass by filtration or centrifugation from the aqueous phase or the extractant;
b) separating and recovering the extractant comprising the compound of formula (I) or the glycosylated compound of formula (I) from the aqueous phase;
c) separating and recovering the compound of formula (I) or the glycosylated compound of formula (I) from the extractant by precipitation;
d) recovering the extractant.

In some embodiments, the step b of separating and recovering the extractant involves one or more steps of i) increasing the temperature, ii) adding one or more salts to the mixture of the aqueous phase and extractant, and/or iii) centrifuging the mixture.

In some embodiments, the step b of separating and recovering the extractant involves one or more steps of i) increasing the temperature, ii) adding one or more salts to the mixture of the aqueous phase and extractant, and/or iii) centrifuging the mixture, such that one or more of these steps moves the mixture above its cloud point.

In some embodiments, the step c of separating and recovering the compound of formula (I) or the glycosylated compound of formula (I) involves one or more steps of i) lowering the temperature, ii) adding an alcohol to the extractant, such as ethanol, and/or iii) centrifuging the extractant.

In some embodiments, the step d of recovering the extractant involves one or more steps of i) increasing the temperature, ii) evaporating the alcohol, such as ethanol, iii) adding one or more salts to the extractant, and/or iv) centrifuging.

In some embodiments, the removing of biomass by centrifugation in step a is performed at room temperature.

In some embodiments, the extractant used for the downstream processing involving moving the mixture above its cloud point is a non-ionic surfactant. In some embodiments, the non-ionic surfactant is selected from the group consisting of: antifoam-A, Triton and polysorbate 20.

In some embodiments, the salt added in step ii is a sulfate salt, such as Na₂SO₄. In some embodiments, the alcohol added in step ii is ethanol at a final concentration of from 15% to 30%, such as 25%.

In some embodiments, the precipitation of the compound of formula (I) or the glycosylated compound of formula (I) in step c is accelerated by centrifuging at room temperature. In some embodiments, after the step c referred to herein, the precipitated compound of formula (I) or the glycosylated compound of formula (I) is resuspended in ethanol and subjected to evaporation to remove ethanol.

In some embodiments, the remaining solution after evaporation is further subjected to freeze drying to obtain a dried form of the compound of formula (I) or the glycosylated compound of formula (I).

In some embodiments, the step d of recovering the extractant comprises evaporating the ethanol using a vacuum centrifuge.

In some embodiments, after the step d, the method involves using the recovered extractant in subsequent extractions.

In some embodiments, the method further comprises cultivating the host cell in a growth medium.

In some embodiments, the method further comprises the steps of i) separating the cultivation into distinct phases comprising a biomass phase, an aqueous phase, and an extractant phase; and subsequently ii) collecting the extractant phase comprising the compound of formula (I) or the glycosylated compound of formula (I). In some embodiments, the extractant is added to a final concentration of from 6 to 14%, such as from 8 to 12%, for example 10%.

In some embodiments, the method involves cultivating the host cell for one or more days, such as from 2 to 7 days, for example 3 to 6 days, such as 4 days, wherein the host cell is cultivated at from 25 to 40° C., such as from 25 to 38° C., such as from 26 to 36° C., such as from 28 to 34° C., for example 30° C.

In some embodiments, the extractant is at least one of isopropyl myristate, isopropyl palmitate, antifoam-A, polysorbate, ethyl laurate, and castor oil.

In some embodiments, the present downstream processing method is provided wherein the compound of formula (I) is violacein or deoxyviolacein and the extractant is at least one of Antifoam-A, isopropyl myristate, isopropyl palmitate, ethyl laurate, grapeseed oil, 2-butyl-1-octanol, and oleic acid.

In some embodiments, the method involves loading the extractant comprising the compound of formula (I) or the glycosylated compound of formula (I) onto dry silica to provide an extractant bound to silica. In some embodiments, the dry silica has a pore size ranging from 50 Å to 70 Å, preferably 60 Å, and a particle size ranging from 0.4 mm to 1.2 mm, more preferably 0.5-1 mm. In some embodiments, the binding to silica is done at a weight-to-weight ratio ranging from 0.8:1 to 1.2:1, with a preferable ratio being 1:1 (w/w). In some embodiments, the extractant bound to silica is washed with a volatile solvent one or more times to remove the extractant. In some embodiments, the volatile solvent is selected from the group consisting of dichloromethane, hexane, and ethyl acetate.

In some embodiments, the method further comprises a step of eluting the compound of formula (I) or the glycosylated compound of formula (I) from the silica using a polar protic solvent, such as an alcohol, for example ethanol. In some embodiments, the method further comprises a step of evaporating the polar protic solvent used in elution to obtain the compound of formula (I) or the glycosylated compound of formula (I) in solid form.

In some embodiments, purification of the compound of formula (I) or the glycosylated compound of formula (I) is done using column chromatography. In some embodiments, the column chromatography is gravity or peristaltic pump driven. In some embodiments, the elution is done using a mixture comprising ethyl acetate in hexane or heptane.

In some embodiments, the recovered compound of formula (I) or the glycosylated compound of formula (I) has a residual solvent concentration below 0.1%.

In some embodiments, the downstream processing method disclosed herein involves violacein, deoxyviolacein, proviolacein, and/or prodeoxyviolacein, for example deoxyviolacein.

In some embodiments, the in-situ extraction method discloses herein further comprises the steps of:
a) collecting the extractant comprising the compound of formula (I) or the glycosylated compound of formula (I),
b) subsequently diluting the extractant with an alcohol, such as ethanol to a predefined concentration of the extractant with respect to the alcohol to provide a mixture of extractant and alcohol, and
c) cooling the mixture of extractant and alcohol to a preset temperature, optionally under stirring, to solidify the extractant thereby increasing the concentration of the compound of formula (I) or the glycosylated compound of formula (I) in the alcohol.

In some embodiments, the method further comprises a step of filtration, such that solidified extractant is removed, optionally at the preset temperature.

In some embodiments, the method further comprises a step of evaporating the alcohol to provide the compound of formula (I) or the glycosylated compound of formula (I) in concentrated form relative to the concentration of the compound of formula (I) or the glycosylated compound of formula (I) in the extractant collected in step a, optionally wherein the concentrated form is a paste.

In some embodiments, the predefined concentration of the extractant with respect to the alcohol is from 20 to 40% extractant, such as from 20 to 21%, such as from 21 to 22%, such as from 22 to 23%, such as from 23 to 24%, such as from 24 to 25%, such as from 25 to 26%, such as from 26 to 27%, such as from 27 to 28%, such as from 28 to 29%, such as from 29 to 30%, such as from 30 to 31%, such as from 31 to 32%, such as from 32 to 33%, such as from 33 to 34%, such as from 34 to 35%, such as from 35 to 36%, such as from 36 to 37%, such as from 37 to 38%, such as from 38 to 39%, such as from 39 to 40%, for example 33%.

In some embodiments, the preset temperature is at the solidification temperature (melting point) of the extractant or less.

In some embodiments, the preset temperature is 20° C. or less, such as 19° C. or less, such as 18° C. or less, such as 17° C. or less, such as 16° C. or less, such as 15° C. or less, such as 14° C. or less, such as 13° C. or less, such as 12° C. or less, such as 11° C. or less, such as 10° C. or less, such as 9° C. or less, such as 8° C. or less, such as 7° C. or less, such as 6° C. or less, such as 5° C. or less, such as 4° C. or less, such as 3° C. or less, such as 2° C. or less, such as 1° C. or less, such as 0° C. or less, such as −1° C. or less, such as −2° C. or less, such as −3° C. or less, such as −4° C. or less, such as −5° C. or less, such as −6° C. or less, such as −7° C. or less, such as −8° C. or less, such as −9° C. or less, such as −10° C. or less.

In some embodiments, the preset temperature is from 20° C. to −5° C., such as from 19° C. to −5° C., such as from 18° C. to −5° C., such as from 17° C. to −5° C., such as from 16° C. to −5° C., such as from 15° C. to −5° C., such as from 14° C. to −5° C., such as from 13° C. to −5° C., such as from 12° C. to −5° C., such as from 11° C. to −5° C., such as from 10° C. to −5° C., such as from 9° C. to −5° C., such as from 8° C. to −5° C., such as from 7° C. to −5° C., such as from 6° C. to −5° C., such as from 5° C. to −5° C.

### Methods of Dyeing

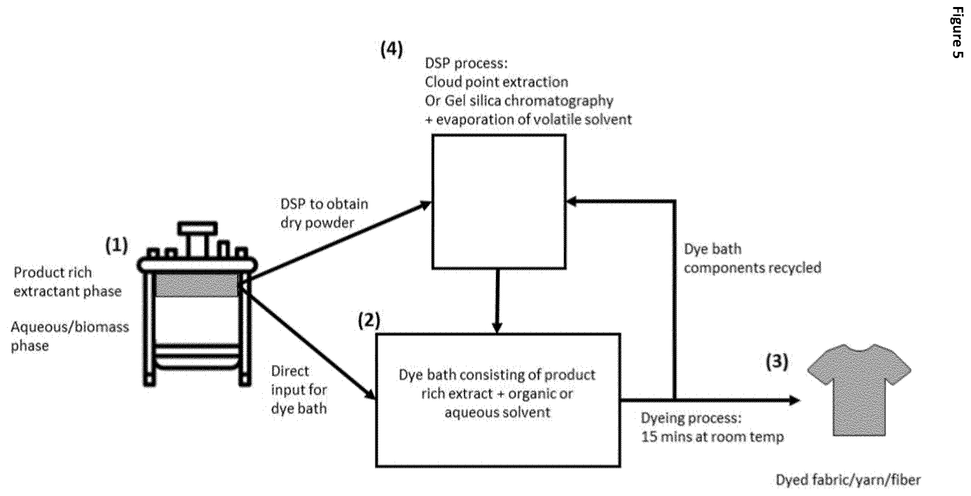

The present disclosure provides a series of alternative methods for dyeing textile materials as shown e.g. in FIG. 5.

In some embodiments, a method is provided for dyeing a textile material, comprising:
a) providing an optionally dried composition of one or more compounds as defined herein, for example violacein, proviolacein, prodeoxyviolacein, and/or deoxyviolacein; and subsequently preparing a dye solution by suspending said composition in a liquid, such as an alcohol, for example ethanol; or
b) providing a colored fermentation extract comprising an extractant and one or more compounds as defined herein, for example violacein, proviolacein, prodeoxyviolacein, and/or deoxyviolacein,
c) contacting a textile material with said dye solution or said colored fermentation extract, optionally for a predetermined duration, thereby dyeing the textile material.

In some embodiments, the method for dyeing further comprises a step d) of removing the textile material from said dye solution or colored fermentation extract and washing with water to remove any excess dye.

In some embodiments, the method for dyeing further comprises a step e) of drying the dyed textile material without the use of pre-treatments, mordants, or other chemical processing steps, and wherein the textile material retains a color change indicative of dyeing.

In some embodiments, the liquid is at least 90% ethanol, such as 100% ethanol.

In some embodiments, the textile material is selected from the group consisting of nylon 6,6, diacetate, polyester, cotton, such as bleached cotton, wool, hemp rayon, denim, viscose, and silk.

In some embodiments, the predetermined duration is from 10 minutes to 2 hours, such as 30 minutes. In some embodiments, the dyeing imparts both color and bioactivity to the textile material.

In some embodiments, the composition employed in the method of dyeing according to the present disclosure is derived from the host cell as defined herein. In some embodiments, the composition is in the form of a purified fermentation extract.

In some embodiments, the colored fermentation extract is obtainable by the in-situ extraction method as defined herein.

In some embodiments, the method further comprises providing a colored fermentation extract using the method disclosed herein and further comprising a step of diluting the colored fermentation extract in a liquid to provide a dye bath.

In some embodiments, the method comprises diluting the colored fermentation extract to an extractant concentration of from 2% to 30%, such as from 2 to 4%, such as from 4 to 6%, such as from 6 to 8%, such as from 8 to 10%, such as from 10 to 12%, such as from 12 to 14%, such as from 14 to 16%, such as from 16 to 18%, such as from 18 to 20%, such as from 20 to 22%, such as from 22 to 24%, such as from 24 to 26%, such as from 26 to 28%, such as from 28 to 30%, for example to a concentration of 10% extractant in 90% of the liquid.

In some embodiments, the liquid is a polar protic solvent, such as an alcohol or water, for example ethanol.

In some embodiments, the extractant is a lipophilic non-volatile solvent.

In some embodiments, the extractant is selected from the group consisting of: Antifoam-A, Triton-X 114, isopropyl myristate, isopropyl palmitate, polysorbate, ethyl laurate, castor oil, oleyl alcohol, butyl caprilate, grapeseed oil, 2-butyl-1-octanol, and oleic acid, for example isopropyl myristate.

### Coloured Aqueous Dilutions

In some embodiments, the method comprises diluting the colored fermentation extract with water, optionally at room temperature, until a single phase is produced between the colored fermentation extract and the water.

In some embodiments, the method comprises dilution until the concentration of the extractant is below its cloud point at room temperature.

In some embodiments, the method involving diluting the colored fermentation extract provides a colored aqueous suspension.

In some embodiments, method comprises dyeing textile material by contacting the textile material with the aqueous suspension and incubating at room temperature.

In some embodiments, the extractant is a non-ionic surfactant, such as Antifoam-A.

In some embodiments, the textile material is selected from the group consisting of: nylon 6,6, diacetate, polyester, cotton, such as bleached cotton, wool, hemp rayon, denim, viscose, and silk, for example nylon 6,6.

In some embodiments, the liquid is water and the one or more compounds are glycosides as defined herein, for example glycosides of violacein, proviolacein, deoxyviolacein, or prodeoxyviolacein. In particular embodiments, the compound is a glycoside of violacein or proviolacein.

In some embodiments, the method further comprises the steps of:
a) incubating the textile material in a dye bath comprising the one or more compounds covalently linked to a saccharide as defined herein and water; and
b) adding a glucosidase, such as a beta-glucosidase to the dye bath to de-glycosylate the one or more compounds thereby providing a dyed textile material.

In some embodiments, the method comprises incubating for from 15 minutes to 24 hours, such as from 15 minutes to 30 minutes, such as from 30 minutes to 45 minutes, such as from 45 minutes to 1 hour, such as from 1 hour to 2 hours, such as from 2 hours to 3 hours, such as from 3 hours to 4 hours, such as from 4 hours to 5 hours, such as from 5 hours to 6 hours, such as from 6 hours to 7 hours, such as from 7 hours to 8 hours, such as from 8 hours to 9 hours, such as from 9 hours to 10 hours, such as from 10 hours to 11 hours, such as from 11 hours to 12 hours, such as from 12 hours to 13 hours, such as from 13 hours to 14 hours, such as from 14 hours to 15 hours, such as from 15 hours to 16 hours, such as from 16 hours to 17 hours, such as from 17 hours to 18 hours, such as from 18 hours to 19 hours, such as from 19 hours to 20 hours, such as from 20 hours to 21 hours, such as from 21 hours to 22 hours, such as from 22 hours to 23 hours, such as from 23 hours to 24 hours, preferably at room temperature.

In some embodiments, the method further comprises washing the dyed textile material after step b.

### Dyeing in a Growth Medium

It is an aspect of the present disclosure to dye a textile material as defined herein directly in the growth medium wherein the host cell is being cultivated.

In some embodiments, method for dyeing textile material in a growth medium is provided, comprising:
a) Cultivating a microbial host cell as defined herein in a growth medium;
b) Adding textile material to the growth medium to provide a dyed textile material comprising a compound of formula (I), optionally for a predefined duration, optionally during the cultivation process.

In some embodiments, the microbial host cell is cultivated at from 25 to 35° C., such as 30° C. for a number of days, such as for from 2 to 8 days, such as 4 days.

In some embodiments, the method further comprises a step of sterilizing the textile material prior to step b, such as by adding the textile material into an alcohol or a solution of alcohol in water, for example ethanol, such as 75% ethanol in water.

In some embodiments, the method further comprises a step c) of recovering the dyed textile material from the growth medium.

In some embodiments, the host cell is of a strain selected from the group consisting of SC-139, SC-141, SC-144, and SC-145.

In some embodiments, the textile material is dyed without requiring additional downstream processing, pre-treatments, mordants, and/or other chemicals.

In some embodiments, the method involves a step of extracting the compound of formula (I) from the growth medium by recovering the dyed textile material from the growth medium.

In some embodiments, the textile material is selected from the group consisting of diacetate, bleached cotton, nylon 6,6, polyester, acrylic, and wool.

In some embodiments, the method further comprises a step of washing the dyed textile material with water post-cultivation.

Accordingly, the present disclosure also provides a dyed textile material comprising the compound as defined herein. The dyed textile material can be provided using the method disclosed herein. Thus, a dyed textile material obtainable by one or more of the methods disclosed herein is provided.

In some embodiments, the textile material is selected from the group consisting of: Nylon 6,6, Diacetate, Bleached cotton, Polyester, Wool, and Acrylic.

In some embodiments, the dyed textile material is provided comprising the compound selected from the group consisting of: deoxyviolacein, violacein, prodeoxyviolacein, proviolacein, or a combination thereof.

In some embodiments, the dyed textile material is antimicrobial, i.e. has antimicrobial activity.

### Colouring Beverages

The present disclosure also provides methods for colouring beverages and enhancing desirable properties of the beverages.

In some embodiments, a method of colouring a beverage is provided, comprising:
a) providing an optionally dried composition of one or more compounds as defined herein, for example glycosylated violacein, glycosylated proviolacein, glycosylated prodeoxyviolacein, and/or glycosylated deoxyviolacein; and optionally subsequently preparing a dye solution by suspending said composition in a liquid, such as an alcohol or water; and
b) contacting a beverage with said dye solution or said composition, optionally for a predetermined duration, thereby colouring the beverage.

In some embodiments, a method for enhancing the antimicrobial properties of a textile material is provided, such as clothing or a wound dressing, or a beverage, comprising dyeing the textile material or colouring the beverage with the compound as defined herein, or with an extractant comprising the compound thereby enhancing the antimicrobial properties of the textile material or beverage.

In some embodiments, a method for enhancing the UV resistance of a textile materials provided, such as clothing, or of a beverage comprising dyeing the textile material or colouring the beverage with the compound as defined herein, or with an extractant comprising the compound thereby enhancing the UV resistance of the textile material or beverage.

In some embodiments, the present disclosure provides a beverage comprising the compound as defined herein, in particular a glycoside of the compound. Preferably, the present disclosure provides a beverage comprising a glycoside of violacein, deoxyviolacein, proviolacein, and/or prodeoxyviolacein.

### Dyed Nanocellulose

In some embodiments, the present disclosure provides a nanocellulose comprising a compound as defined herein.

In some embodiments, the nanocellulose is selected from the group consisting of bacterial nanocellulose (BNC), nanofabricated cellulose (NFC), cellulose nanocrystals (CNC), cellulose nanofibrils (CNF), and electrospun cellulose nanofibers. In some embodiments, the nanocellulose is bacterial nanocellulose (BNC) or nanofabricated cellulose (NFC).

In some embodiments, the nanocellulose further comprises a non-ionic surfactant, such as Triton-X 100, Tween 20, sodium dodecyl sulfate (SDS), or polyvinyl alcohol (PVA).

In some embodiments, the nanocellulose is derived from a microbial culture.

In some embodiments, the microbial culture comprises one or more of Acetobacter xylinum, Gluconacetobacter hansenii, and Komagataeibacter medellinensis.

In some embodiments, the nanocellulose is derived from a Kombucha starter culture.

In some embodiments, the nanocellulose is derived from a Kombucha starter culture comprising green tea and sucrose.

In some embodiments, the nanocellulose further comprises an additive selected from the group consisting of starch, lignin, and chitosan.

In some embodiments, the nanocellulose comprises one or more of violacein, proviolacein, deoxyviolacein, prodeoxyviolacein, and combinations thereof.

In some embodiments, the compound is deoxyviolacein and the nanocellulose is NFC.

In some embodiments, the nanocellulose is in a form selected from the group consisting of a hydrogel, an aerogel, and a film.

In some embodiments, a method for dyeing nanocellulose is provided, comprising
a) providing a compound as defined herein, optionally in a dye bath comprising an alcohol and optionally a surfactant;
b) providing nanocellulose, such as bacterial nanocellulose (BNC) or nanofabricated cellulose (NFC);
c) incubating the cellulose with the compound, optionally in the dye bath, at a predefined temperature until the nanocellulose takes on the color of the compound, thereby providing dyed nanocellulose.

In some embodiments, the predefined temperature is from 20 to 50° C.

In some embodiments, the predefined temperature is room temperature.

In some embodiments, the dye bath comprises from 70 to 95% alcohol in non-ionic surfactant, such as 90%. In some embodiments, the alcohol is ethanol.

In some embodiments, the extractant is selected from the group consisting of isopropyl myristate, Triton-X 100, Tween-20, and Tween-80.

In some embodiments, the nanocellulose is selected from the group consisting of bacterial nanocellulose (BNC), nanofabricated cellulose (NFC), cellulose nanocrystals (CNC), cellulose nanofibrils (CNF), and electrospun cellulose nanofibers.

In some embodiments, the nanocellulose is selected from the group consisting of bacterial nanocellulose (BNC), and nanofabricated cellulose (NFC).

In some embodiments, the method of dyeing nanocellulose further comprises a step of: d. drying the dyed nanocellulose at room temperature.

In some embodiments, the compound is provided in a dye bath, and wherein the dye bath further comprises a non-ionic surfactant at a concentration of approximately 0.01%, for example Triton-X 100.

### Dyed Products

In some embodiments, a dyed product is provided comprising the nanocellulose as defined herein.

In some embodiments, the product is selected from the group consisting of: a wound healing product, such as a wound dressing, a food packaging, a cosmetic product, a textile fiber, a bio-based paint, a paper, and a textile dye.

In some embodiments, a method for dyeing a product is provided, comprising,
a) Providing a nanocellulose as defined herein;
b) Providing a product;
c) Contacting the nanocellulose with the product, optionally incubating the product with the nanocellulose for a duration.

In some embodiments, the product is selected from the group consisting of: a wound healing product, such as a wound dressing, a food packaging, a cosmetic product, a textile fiber, a bio-based paint, a paper, and a textile dye.

In some embodiments, the product is paper and the nanocellulose comprises NFC.

### Dye Baths

The present disclosure also relates to dye baths for dyeing products and for methods of preparing the dye baths.

In some embodiments, a method of producing a dye bath is provided, the method comprising the steps of:
a) cultivating a host cell as defined herein in growth medium to produce the compound as defined herein, such as engineered S. cerevisiae production strains producing at least one of violacein, deoxyviolacein, prodeoxyviolacein, and proviolacien;
b) adding an extractant to the growth medium thereby providing a compound enriched extractant;
c) optionally collecting the compound enriched extractant and adding further extractant to the growth medium;
d) optionally repeating step c a number of times to provide a collection of compound enriched extractants,
e) diluting the compound enriched extractant or the collection of compound enriched extractants with a liquid, such as an organic solvent, thereby providing a dye bath.

In some embodiments, the extractant is selected from the group consisting of: isopropyl myristate, Antifoam-A, Triton-X 114, isopropyl palmitate, polysorbate, ethyl laurate, castor oil, oleyl alcohol, butyl caprilate, grapeseed oil, 2-butyl-1-octanol, and oleic acid, or any combination thereof.

In some embodiments, the extractant is isopropyl myristate. In some embodiments, the liquid is ethanol.

Accordingly, the present disclosure also provides a dye bath obtainable using the method for producing a dye bath as disclosed herein.

In some embodiments, a method for dyeing a product is provided, comprising the steps of:
a) adding a product to a dye bath comprising a compound of formula (I) as defined herein, and a liquid, and optionally an extractant;
b) optionally pre/post-treating the product to modify its pH;
c) optionally dyeing the product at a predetermined temperature for a predetermined time to obtain a dyed product, optionally in a dyeing machine;
d) washing the dyed product with water; and
e) optionally drying the product.

In some embodiments, the product is selected from the group consisting of: a fabric, a fiber, a yarn, a textile, a filament, a weave, a non-woven material, a twill, a felt, a lace, a mesh, a cord, a tapestry, a tuft, and a batting; for example a fabric, a fiber, or a yarn.

In some embodiments, the product comprises a material selected from the group consisting of nylon 6,6, diacetate, polyester, cotton, such as bleached cotton, wool, hemp rayon, denim, viscose, and silk.

In some embodiments, the predetermined temperature is from 15 to 50° C., such as from 20 to 35° C., for example about 23° C.

In some embodiments, the predetermined temperature is from 80 to 180° C., such as from 85 to 170° C., such as from 90 to 160° C., such as from 95 to 155° C., such as from 100 to 150° C., such as from 105 to 145° C., such as from 110 to 140° C., for example 130° C. Increasing the temperature of the dye bath enables dyeing of some materials which can be otherwise difficult to dye. An example of such material is polyester. Hence, in some embodiments, the predetermined temperature is from 80 to 180° C., such as from 85 to 170° C., such as from 90 to 160° C., such as from 95 to 155° C., such as from 100 to 150° C., such as from 105 to 145° C., such as from 110 to 140° C., for example 130° C.; wherein the product comprises polyester.

In some embodiments, the predetermined time is from 5 minutes to 360 minutes, such as for 10 minutes to 60 minutes, for example 15 minutes.

In some embodiments, the final concentration of the extractant in the dye bath is less than 70%, such as less than 69%, such as less than 68%, such as less than 67%, such as less than 66%, such as less than 65%, such as less than 64%, such as less than 63%, such as less than 62%, such as less than 61%, such as less than 60%, such as less than 59%, such as less than 58%, such as less than 57%, such as less than 56%, such as less than 55%, such as less than 54%, such as less than 53%, such as less than 52%, such as less than 51%, such as less than 50%.

In some embodiments, the final concentration of the extractant in the dye bath is from 20 to 70%, such as from 30 to 69%, such as from 35 to 68%, such as from 40 to 67%, for example from 45 to 66%.

In some embodiments, method of recycling a used dye bath is provided, comprising the steps of:
a) subjecting a used dye bath comprising i) a liquid, ii) an extractant, and iii) a compound of formula (I) as defined herein to evaporation, optionally in vacuo to remove the liquid, wherein the dye bath has been used for dyeing a product;
b) passing the remaining extractant and compound from step a through silica to obtain a recycled dye bath.

In some embodiments, it is beneficial to add a dispersing agent to the dye bath of the present disclosure or to the composition comprising the compound of formula (I) as the dispersing agent has been shown in Example 31 to enhance the dyeing performance.

In some embodiments, the method disclosed herein further comprises a step of adding a dispersing agent, such as a soap. The dispersing agent should be added to the relevant container, dyeing machine or physical space where the dyeing is taking place.

In some embodiments, the dispersing agent is selected from the group consisting of: an anionic surfactant, such as sodium dodecyl sulfate or alkylbenzene sulfonate; a cationic surfactant, such as a quaternary ammonium compound; a non-ionic surfactant, such as an ethoxylated alcohol, an alkylphenol, or a polysorbate; a zwitterionic surfactant, such as cocamidopropyl betaine; a polysaccharide, a cellulose derivative, such as carboxymethylcellulose, or hydroxyethylcellulose; a protein, such as casein, a gum, such as xanthan gum, guar gum, or acacia gum, and lecithin.

In some embodiments, the dispersing agent is added to provide a final concentration of from 0.05 to 3 g/L, for example from 1 to 2 g/L.

### Sequence Listings

The present application contains a Sequence Listing prepared in Patentin included below but also submitted electronically in ST26 format which is hereby incorporated by reference in its entirety.

| | | | | | | |
|---|---|---|---|---|---|---|
| SEQ ID | DNA | sequence | Fructose-6-phosphate | BfXfpk | From | Bifidobacterium |
| NO: 1 | | of | Phosphoketolase | | | breve |
| SEQ ID | Protein | sequence | Fructose-6-phosphate | BfXfpk | From | Bifidobacterium |
| NO: 2 | | of | Phosphoketolase | | | breve |
| SEQ ID | DNA | sequence | Phosphotransacetylase | CkPta | From | Clostridium |
| NO: 3 | | of | | | | kluyveri |
| SEQ ID | Protein | sequence | Phosphotransacetylase | CkPta | From | Clostridium |
| NO: 4 | | of | | | | kluyveri |
| SEQ ID | DNA | sequence | 3-deoxy-D-arabino- | ARO4(K229L) | From | Saccharomyces |
| NO: 5 | | of | heptulosonate 7- | | | cerevisiae |
| | | | phosphate | | | |
| SEQ ID | Protein | sequence | 3-deoxy-D-arabino- | ARO4(K229L) | From | Saccharomyces |
| NO: 6 | | of | heptulosonate 7- | | | cerevisiae |
| | | | phosphate | | | |
| SEQ ID | DNA | sequence | Multiple native | ARO1 | From | Saccharomyces |
| NO: 7 | | of | enzymatic steps | | | cerevisiae |
| SEQ ID | Protein | sequence | Multiple native | ARO1 | From | Saccharomyces |
| NO: 8 | | of | enzymatic steps | | | cerevisiae |
| SEQ ID | DNA | sequence | Shikimate kinase | EcAroL | From | Escherichia coli |
| NO: 9 | | of | | | | |
| SEQ ID | Protein | sequence | Shikimate kinase | EcAroL | From | Escherichia coli |
| NO: 10 | | of | | | | |
| SEQ ID | DNA | sequence | Chorismate synthase | ARO2 | From | Saccharomyces |
| NO: 11 | | of | | | | cerevisiae |
| SEQ ID | Protein | sequence | Chorismate synthase | ARO2 | From | Saccharomyces |
| NO: 12 | | of | | | | cerevisiae |
| SEQ ID | DNA | sequence | Anthranilate synthase | TRP2(S65R, | From | Saccharomyces |
| NO: 13 | | of | | S76L) | | cerevisiae |
| SEQ ID | Protein | sequence | Anthranilate synthase | TRP2(S65R, | From | Saccharomyces |
| NO: 14 | | of | | S76L) | | cerevisiae |
| SEQ ID | DNA | sequence | Ribose-phosphate | BsPrs | From | Bacillus subtilis |
| NO: 15 | | of | pyrophosphokinase | | | |
| SEQ ID | Protein | sequence | Ribose-phosphate | BsPrs | From | Bacillus subtilis |
| NO: 16 | | of | pyrophosphokinase | | | |
| SEQ ID | DNA | sequence | Anthranilate | TRP4 | From | Saccharomyces |
| NO: 17 | | of | phosphoribosyl | | | cerevisiae |
| | | | transferase | | | |
| SEQ ID | Protein | sequence | Anthranilate | TRP4 | From | Saccharomyces |
| NO: 18 | | of | phosphoribosyl | | | cerevisiae |
| | | | transferase | | | |
| SEQ ID | DNA | sequence | N-(5′-phosphoribosyl)- | TRP1 | From | Saccharomyces |
| NO: 19 | | of | anthranilate isomerase | | | cerevisiae |
| SEQ ID | Protein | sequence | N-(5′-phosphoribosyl)- | TRP1 | From | Saccharomyces |
| NO: 20 | | of | anthranilate isomerase | | | cerevisiae |
| SEQ ID | DNA | sequence | Indole-3-glycerol | TRP3 | From | Saccharomyces |
| NO: 21 | | of | phosphate synthase | | | cerevisiae |
| SEQ ID | Protein | sequence | Indole-3-glycerol | TRP3 | From | Saccharomyces |
| NO: 22 | | of | phosphate synthase | | | cerevisiae |
| SEQ ID | DNA | sequence | Tryptophan synthase | TRP5 | From | Saccharomyces |
| NO: 23 | | of | | | | cerevisiae |
| SEQ ID | Protein | sequence | Tryptophan synthase | TRP5 | From | Saccharomyces |
| NO: 24 | | of | | | | cerevisiae |
| SEQ ID | DNA | sequence | Flavin-dependent L- | CvVioA | From | Chromobacterium |
| NO: 25 | | of | tryptophan oxidase | | | violaceum |
| SEQ ID | Protein | sequence | Flavin-dependent L- | CvVioA | From | Chromobacterium |
| NO: 26 | | of | tryptophan oxidase | | | violaceum |
| SEQ ID | DNA | sequence | 2-imino-3-(indol-3- | CvVioB | From | Chromobacterium |
| NO: 27 | | of | yl)propanoate | | | violaceum |
| | | | dimerase | | | |
| SEQ ID | Protein | sequence | 2-imino-3-(indol-3- | CvVioB | From | Chromobacterium |
| NO: 28 | | of | yl)propanoate | | | violaceum |
| | | | dimerase | | | |
| SEQ ID | DNA | sequence | Prodeoxyviolacein | CvVioE | From | Chromobacterium |
| NO: 29 | | of | synthase | | | violaceum |
| SEQ ID | Protein | sequence | Prodeoxyviolacein | CvVioE | From | Chromobacterium |
| NO: 30 | | of | synthase | | | violaceum |
| SEQ ID | DNA | sequence | Protodeoxyviolaceinate | CvVioD | From | Chromobacterium |
| NO: 31 | | of | monooxygenase | | | violaceum |
| SEQ ID | Protein | sequence | Protodeoxyviolaceinate | CvVioD | From | Chromobacterium |
| NO: 32 | | of | monooxygenase | | | violaceum |
| SEQ ID | DNA | sequence | Violacein synthase | CvVioC | From | Chromobacterium |
| NO: 33 | | of | | | | violaceum |
| SEQ ID | Protein | sequence | Violacein synthase | CvVioC | From | Chromobacterium |
| NO: 34 | | of | | | | violaceum |
| SEQ ID | DNA | sequence | Tryptophan | CrTdc | From | Catharanthus |
| NO: 35 | | of | decarboxylase | | | roseus |
| SEQ ID | Protein | sequence | Tryptophan | CrTdc | From | Catharanthus |
| NO: 36 | | of | decarboxylase | | | roseus |
| SEQ ID | DNA | sequence | Transaldolase | TAL1 | From | Saccharomyces |
| NO: 37 | | of | | | | cerevisiae |
| SEQ ID | Protein | sequence | Transaldolase | TAL1 | From | Saccharomyces |
| NO: 38 | | of | | | | cerevisiae |
| SEQ ID | DNA | sequence | Transketolase | TKL1 | From | Saccharomyces |
| NO: 39 | | of | | | | cerevisiae |
| SEQ ID | Protein | sequence | Transketolase | TKL1 | From | Saccharomyces |
| NO: 40 | | of | | | | cerevisiae |
| SEQ ID | DNA | sequence | GTP cyclohydrolase II | RIB1 | From | Saccharomyces |
| NO: 41 | | of | | | | cerevisiae |
| SEQ ID | Protein | sequence | GTP cyclohydrolase II | RIB1 | From | Saccharomyces |
| NO: 42 | | of | | | | cerevisiae |
| SEQ ID | DNA | sequence | Mitochondrial flavin | FLX1 | From | Saccharomyces |
| NO: 43 | | of | adenine dinucleotide | | | cerevisiae |
| | | | transporter | | | |
| SEQ ID | Protein | sequence | Mitochondrial flavin | FLX1 | From | Saccharomyces |
| NO: 44 | | of | adenine dinucleotide | | | cerevisiae |
| | | | transporter | | | |
| SEQ ID | DNA | sequence | ER localized heme | HMX1 | From | Saccharomyces |
| NO: 45 | | of | oxygenase | | | cerevisiae |
| SEQ ID | DNA | sequence | Porphobilinogen | HEM3 | From | Saccharomyces |
| NO: 46 | | of | deaminase | | | cerevisiae |
| SEQ ID | Protein | sequence | Porphobilinogen | HEM3 | From | Saccharomyces |
| NO: 47 | | of | deaminase | | | cerevisiae |
| SEQ ID | DNA | sequence | Pyruvate | PDC5 | From | Saccharomyces |
| NO: 48 | | of | decarboxylase | | | cerevisiae |
| SEQ ID | DNA | sequence | Phenylpyruvate | ARO10 | From | Saccharomyces |
| NO: 49 | | of | decarboxylase | | | cerevisiae |
| SEQ ID | DNA | sequence | NADH kinase | POS5 | From | Saccharomyces |
| NO: 50 | | of | | | | cerevisiae |
| SEQ ID | Protein | sequence | NADH kinase | POS5 | From | Saccharomyces |
| NO: 51 | | of | | | | cerevisiae |
| SEQ ID | DNA | sequence | Flavin-dependent L- | CvVioA | From | Chromobacterium |
| NO: 52 | | of | tryptophan oxidase | | | violaceum |
| SEQ ID | DNA | sequence | 2-imino-3-(indol-3- | CvVioB | From | Chromobacterium |
| NO: 53 | | of | yl)propanoate | | | violaceum |
| | | | dimerase | | | |
| SEQ ID | DNA | sequence | Violacein synthase | CvVioC | From | Chromobacterium |
| NO: 54 | | of | | | | violaceum |
| SEQ ID | DNA | sequence | Protodeoxyviolaceinate | CvVioD | From | Chromobacterium |
| NO: 55 | | of | monooxygenase | | | violaceum |
| SEQ ID | DNA | sequence | Prodeoxyviolacein | CvVioE | From | Chromobacterium |
| NO: 56 | | of | synthase | | | violaceum |
| SEQ ID | DNA | sequence | mRNA-binding | UBP3 | From | Saccharomyces |
| NO: 57 | | of | ubiquitin-specific | | | cerevisiae |
| | | | protease | | | |
| SEQ ID | DNA | sequence | cis-Golgi network | RIC1 | From | Saccharomyces |
| NO: 58 | | of | transporter protein | | | cerevisiae |
| SEQ ID | DNA | sequence | DL-glycerol-3- | GPP1 | From | Saccharomyces |
| NO: 59 | | of | phosphate | | | cerevisiae |
| | | | phosphatase | | | |
| SEQ ID | DNA | sequence | Heme-dependent | ROX1 | From | Saccharomyces |
| NO: 60 | | of | repressor of hypoxic | | | cerevisiae |
| | | | genes | | | |
| SEQ ID | DNA | sequence | Heme responsive gene | HAP1 | From | Saccharomyces |
| NO: 61 | | of | regulator | | | cerevisiae |
| SEQ ID | DNA | sequence | Tryptophan synthase | PcTrpB | From | Psilocybe |
| NO: 62 | | of | | | | cubensis |
| SEQ ID | Protein | sequence | Tryptophan synthase | PcTrpB | From | Psilocybe |
| NO: 63 | | of | | | | cubensis |
| SEQ ID | DNA | sequence | Glycosyltransferase | Pt73Y | From | Populus |
| NO: 64 | | of | | | | trichocarpa |
| SEQ ID | DNA | sequence | Glycosyltransferase | Pt73Y | From | Populus |
| NO: 65 | | of | | | | trichocarpa |
| SEQ ID | Protein | sequence | Glycosyltransferase | Pt73Y | From | Populus |
| NO: 66 | | of | | | | trichocarpa |
| SEQ ID | DNA | sequence | Glycosyltransferase | Bs109_1 | From | Bacillus subtilis |
| NO: 67 | | of | | | | |
| SEQ ID | Protein | sequence | Glycosyltransferase | Bs109_1 | From | Bacillus subtilis |
| NO: 68 | | of | | | | |
| SEQ ID | DNA | sequence | Glycosyltransferase | Bs109A1 | From | Bacillus subtilis |
| NO: 69 | | of | | | | |
| SEQ ID | Protein | sequence | Glycosyltransferase | Bs109A1 | From | Bacillus subtilis |
| NO: 70 | | of | | | | |
| SEQ ID | DNA | sequence | Glycosyltransferase | Cp73B | From | Carica papaya |
| NO: 71 | | of | | | | |
| SEQ ID | Protein | sequence | Glycosyltransferase | Cp73B | From | Carica papaya |
| NO: 72 | | of | | | | |
| SEQ ID | DNA | sequence | 2-imino-3-(indol-3- | CvVioB-E | From | Chromobacterium |
| NO: 73 | | of | yl)propanoate | fusion | | violaceum |
| | | | dimerase | GGGGS3 | | |
| | | | Prodeoxyviolacein | linker | | |
| | | | synthase fusion | | | |
| SEQ ID | Protein | sequence | 2-imino-3-(indol-3- | CvVioB-E | From | Chromobacterium |
| NO: 74 | | of | yl)propanoate | fusion | | violaceum |
| | | | dimerase | GGGGS3 | | |
| | | | Prodeoxyviolacein | linker | | |
| | | | synthase fusion | | | |
| SEQ ID | DNA | sequence | 2-imino-3-(indol-3- | CvVioB-E | From | Chromobacterium |
| NO: 75 | | of | yl)propanoate | fusion | | violaceum |
| | | | dimerase | EAAAK3 | | |
| | | | Prodeoxyviolacein | linker | | |
| | | | synthase fusion | | | |
| SEQ ID | Protein | sequence | 2-imino-3-(indol-3- | CvVioB-E | From | Chromobacterium |
| NO: 76 | | of | yl)propanoate | fusion | | violaceum |
| | | | dimerase | EAAAK3 | | |
| | | | Prodeoxyviolacein | linker | | |
| | | | synthase fusion | | | |
| SEQ ID | DNA | sequence | IPA imine dimer | LaRebD | From | Lentzea |
| NO: 77 | | of | synthase | | | aerocolonigenes |
| SEQ ID | Protein | sequence | IPA imine dimer | LaRebD | From | Lentzea |
| NO: 78 | | of | synthase | | | aerocolonigenes |
| SEQ ID | DNA | sequence | Tryptophan oxidase | SsStaO | From | Streptomyces sp. |
| NO: 79 | | of | | | | TP-A0274 |
| SEQ ID | Protein | sequence | Tryptophan oxidase | SsStaO | From | Streptomyces sp. |
| NO: 80 | | of | | | | TP-A0274 |
| SEQ ID | DNA | sequence | IPA imine dimer | SsStaD | From | Streptomyces sp. |
| NO: 81 | | of | synthase | | | TP-A0274 |
| SEQ ID | Protein | sequence | IPA imine dimer | SsStaD | From | Streptomyces sp. |
| NO: 82 | | of | synthase | | | TP-A0274 |
| SEQ ID | DNA | sequence | Tryptophan oxidase | NlInkO | From | Nonomuraea |
| NO: 83 | | of | | | | longicatena |
| SEQ ID | Protein | sequence | Tryptophan oxidase | NlInkO | From | Nonomuraea |
| NO: 84 | | of | | | | longicatena |
| SEQ ID | DNA | sequence | IPA imine dimer | NlInkD | From | Nonomuraea |
| NO: 85 | | of | synthase | | | longicatena |
| SEQ ID | Protein | sequence | IPA imine dimer | NlInkD | From | Nonomuraea |
| NO: 86 | | of | synthase | | | longicatena |
| SEQ ID | DNA | sequence | Tryptophan oxidase | AmAtmO | From | Actinomadura |
| NO: 87 | | of | | | | melliaura |
| SEQ ID | Protein | sequence | Tryptophan oxidase | AmAtmO | From | Actinomadura |
| NO: 88 | | of | | | | melliaura |
| SEQ ID | DNA | sequence | IPA imine dimer | AmAtmD | From | Actinomadura |
| NO: 89 | | of | synthase | | | melliaura |
| SEQ ID | Protein | sequence | IPA imine dimer | AmAtmD | From | Actinomadura |
| NO: 90 | | of | synthase | | | melliaura |
| SEQ ID | DNA | sequence | Glycosyltransferase | Cs73Y (yeast | From | Crocus sativus |
| NO: 91 | | of | | c/o) | | |
| SEQ ID | Protein | sequence | Glycosyltransferase | Cs73Y (yeast | From | Crocus sativus |
| NO: 92 | | of | | c/o) | | |
| SEQ ID | DNA | sequence | Glycosyltransferase | Ha88B_2 | From | Helianthus |
| NO: 93 | | of | | (yeast c/o) | | annuus |
| SEQ ID | Protein | sequence | Glycosyltransferase | Ha88B_2 | From | Helianthus |
| NO: 94 | | of | | (yeast c/o) | | annuus |
| SEQ ID | DNA | sequence | Glycosyltransferase | Pt73Y (yeast | From | Populus |
| NO: 95 | | of | | c/o) | | trichocarpa |
| SEQ ID | Protein | sequence | Glycosyltransferase | Pt73Y (yeast | From | Populus |
| NO: 96 | | of | | c/o) | | trichocarpa |

### ITEMS

1. A method for producing a compound of formula (I):

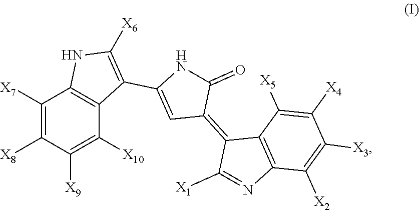

or a tautomer thereof, wherein any one of X₁, X₂, X₃, X₄, X₅, X₆, X₇, X₈, X₉, and X₁₀ are independently of each other selected from the group consisting of: H, R₁, R₂, O, OH, OR₁, NH, NO₂, NH₂, NHR₁, NHR₂, SR₁, F, Cl, Br, I, and SH; wherein R₁ and R₂ are independently of each other selected from the group consisting of a C₁₋₈ alkyl, C₁₋₈ alkenyl, C₁₋₈ alkoyl, C₁₋₈ aryl, and C₁₋₈ aroyl, and R₁ and R₂ are optionally covalently linked to form a ring; wherein the method comprises providing an indole of formula (II):

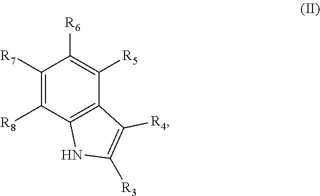

wherein
R₃, R₅, R₆, R₇, and R₈ are independently of each other selected from the group consisting of: H, R₁, R₂, O, OH, OR₁, NH, NH₂, NHR₁, NHR₂, NO₂, SR₁, F, Cl, Br, I and SH; wherein R₁ and R₂ are independently of each other selected from the group consisting of a C₁₋₈ alkyl, C₁₋₈ alkenyl, C₁₋₈ alkoyl, C₁₋₈ aryl, and C₁₋₈ aroyl, and R₁ and R₂ are optionally covalently linked to form a ring; and wherein
R₄ is a chemical handle for enzymatic conversion toward one or more intermediates leading to the compound of formula (I), including the compound of formula (I), and further comprises contacting the indole of formula (II) with one or more enzymes, optionally wherein the one or more enzymes are from an operative biosynthetic pathway for producing violacein.
2. The method according to item 1, wherein the chemical handle for enzymatic conversion toward one or more intermediates leading to the compound of formula (I) is selected from the group consisting of: H, and a phosphoric ester of glycerol, such as glycerol-3-phosphate;

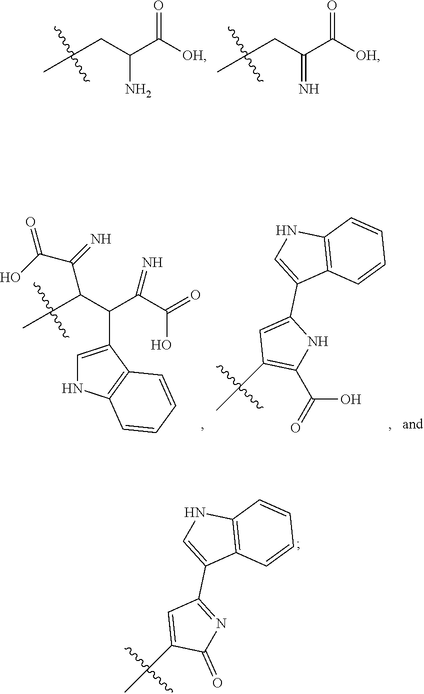 [SMILES: *CC(N)C(=O)O ; *CC(=N)C(=O)O ; *C(C(=N)C(=O)O)C(C(=N)C(=O)O)c1c[nH]c2ccccc12 ; *c1cc(-c2c[nH]c3ccccc23)[nH]c1C(=O)O ; *C1=CC(c2c[nH]c3ccccc23)=NC1=O]

3. The method according to any of the preceding items, wherein the one or more intermediates is selected from the group consisting of: tryptophan, indole-3-pyruvic acid imine, indole-3-pyruvic acid imine dimer, protodeoxyviolaceinic acid, and protoviolaceinic acid, and substituted analogues thereof bearing substituents corresponding to X₁, X₂, X₃, X₄, X₅, X₆, X₇, X₈, X₉, and/or X₁₀ as defined for the compound of formula (I).
4. The method according to any of the preceding items, wherein the method comprises contacting the compound of formula (II) with an amino acid, such as a proteinogenic amino acid, for example serine, in the presence of the one or more enzymes.
5. The method according to any of the preceding items, wherein the method comprises contacting the compound of formula (II) with one or more pathway molecules selected from: FAD, HEME, and NADPH.
6. The method according to any of the preceding items, wherein the compound of formula (I) is selected from the group consisting of:

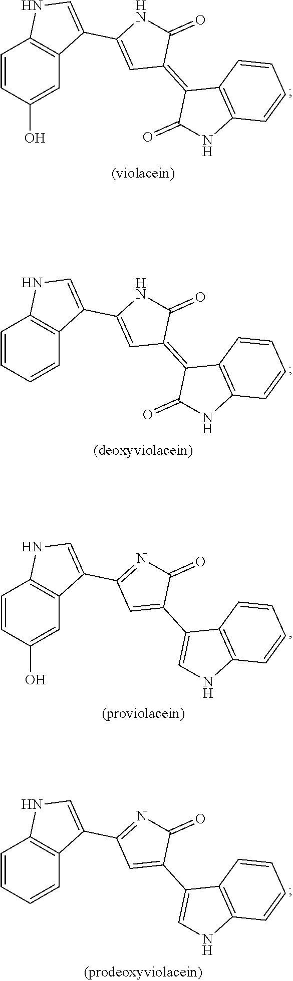 [SMILES: O=C1NC(c2c[nH]c3ccc(O)cc23)=C/C1=C1\C(=O)Nc2ccccc21 ; O=C1NC(c2c[nH]c3ccccc23)=C/C1=C1\C(=O)Nc2ccccc21 ; O=C1N=C(c2c[nH]c3ccc(O)cc23)C=C1c1c[nH]c2ccccc12 ; O=C1N=C(c2c[nH]c3ccccc23)C=C1c1c[nH]c2ccccc12]

and tautomers thereof.
7. The method according to any of the preceding items, wherein R₄ is H, and the compound of formula (II) has been prepared in vitro or in vivo.
8. The method according to item 7, wherein at least one of R₃, R₅, R₆, R₇, and R₈ is not H.
9. The method according to any of the preceding items, wherein R₄ is glycerol-3-phosphate and the compound of formula (II) has been prepared in vivo.
10. The method according to item 9, wherein the compound of formula (I) is prepared from glucose.
11. The method according to any of the preceding items, wherein the indole of formula (II) is selected from indole, and indole-3-glycerol phosphate.
12. The method according to any preceding item, wherein the compound of formula (I) is contacted with a glycosyl donor comprising a glycosyl group.
13. The method according to item 12, wherein the method comprises a glycosylation step of the compound of formula (I) to provide a glycosylated compound of formula (I), wherein the glycosylated compound of formula (I) comprises the compound of formula (I) covalently attached to the glycosyl group.
14. The method according to any of items 12-13, wherein the method comprises a de-glycosylation step such that the glycosylated compound of formula (I) is de-glycosylated to provide the compound of formula (I).
15. The method according to item 14, wherein the de-glycosylation step is facilitated by a glycosidase, such as a β-glycosidase.
16. The method according to item 14, wherein the de-glycosylation step is facilitated by a glucosidase, such as a β-glucosidase.
17. The method according to any of items 12-14, wherein the glycosyl group of the glycosyl donor comprises one or more of glucose, galactose, xylose, mannose, galactofuranose, arabinose, rhamnose, apiose, fucose, glucosamine, galactosamine, N-acetylglucosamine, N-acetylgalactosamine, xylosamine, mannosamine, arabinosamine, rhamnosamine, apiosamine, fucosamine, glucuronate, galacturonate, mannuronate, arabinate, apionate or a combination thereof.
18. The method according to any of items 13-17, wherein the glycosylation step comprises an 0-glycosylation, such as a β-O-glycosylation.
19. The method according to any of items 12-18, wherein the glycosyl donor is a nucleotide glycoside.
20. The method of item 19, wherein the nucleotide glycoside is NTP-glycoside, NDP-glycoside or NMP-glycoside.
21. The method of item 20, wherein the nucleoside of the nucleotide glycoside is selected from Uridine, Adenosin, Guanosin, Cytidin and deoxythymidine.
22. The method of item 21, wherein the nucleotide glycoside is selected from UDP-glycosides, ADP-glycosides, CDP-glycosides, CMP-glycosides, dTDP-glycosides and GDP-glycosides.
23. The method of item 22, wherein the nucleotide glycoside is selected from UDP-D-glucose (UDP-Glc); UDP-galactose (UDP-Gal); UDP-D-xylose (UDP-Xyl); UDP-N-acetyl-D-glucosamine (UDP-GlcNAc); UDP-N-acetyl-D-galactosamine (UDP-GalNAc); UDP-D-glucuronic acid (UDP-GlcA); UDP-D-galactofuranose (UDP-Galf); UDP-arabinose; UDP-rhamnose, UDP-apiose; UDP-2-acetamido-2-deoxy-α-D-mannuronate; UDP-N-acetyl-D-galactosamine 4-sulfate; UDP-N-acetyl-D-mannosamine; UDP-2,3-bis(3-hydroxytetradecanoyl)-glucosamine; UDP-4-deoxy-4-formamido-β-L-arabinopyranose; UDP-2,4-bis(acetamido)-2,4,6-trideoxy-α-D-glucopyranose; UDP-galacturonate; UDP-3-amino-3-deoxy-α-D-glucose; guanosine diphospho-D-mannose (GDP-Man); guanosine diphospho-L-fucose (GDP-Fuc); guanosine diphospho-L-rhamnose (GDP-Rha); cytidine monophospho-N-acetylneuraminic acid (CMP-Neu5Ac); cytidine monophospho-2-keto-3-deoxy-D-mannooctanoic acid (CMP-Kdo); and ADP-glucose.
24. The method of any preceding item, wherein the one or more enzymes are selected from glycosyltransferases, synthases, kinases, transketolases, transaldolase, phosphoketolases, phosphotransketolases, dehydratases, dehydrogenases, carboxyvinyltransferases, phosphoribosyl transferases, isomerases, oxidases, dimerases, and monooxygenases.
25. The method of item 24, wherein the glycosyltransferase is derived from a plant, a fungus, or a bacterium.
26. The method of item 25, wherein the plant is selected from Oryza sativa, Crocus sativus, Nicotiana tabacum, Stevia rebaudiana, Nicotiana benthatamiana, Arabidopsis thaliana, Helianthus annuus, and Populus trichocarpa.
27. The method of item 25, wherein the bacterium is Bacillus subtilis.
28. The method of item 24 to 26, wherein the glycosyl transferase is an O-glycoside transferase and/or a C-glycoside transferase.
29. The method of item 28, wherein the glycosyl transferase is an aglycone O-glycosyltransferase.
30. The method of item 28, wherein the glycosyl transferase is a glycoside O-glycosyltransferase.
31. The method of item 28, wherein the glycosyl transferase is an aglycone O-glucosyltransferase.
32. The method of item 28, wherein the glycosyl transferase is an aglycone O-rhamnosyltransferase.
33. The method of item 28, wherein the glycosyl transferase is an aglycone O-xylosyltransferase.
34. The method of item 28, wherein the glycosyl transferase is an aglycone O-arabinosyltransferase.
35. The method of item 28, wherein the glycosyl transferase is an aglycone O—N-acetylgalactosaminyltransferase.
36. The method of item 28, wherein the glycosyl transferase is an aglycone O—N-acetylglucosaminyltransferase.
37. The method of item 28, wherein the glycosyl transferase is an aglycone/glycoside mono-O-glycosyltransferase.
38. The method of item 28, wherein the glycosyl transferase is an aglycone/glycoside di-O-glycosyltransferase.
39. The method of item 28, wherein the glycosyl transferase is an aglycone/glycoside tri-O-glycosyltransferase.
40. The method of item 28, wherein the glycosyl transferase is an aglycone/glycoside tetra-O-glycosyltransferase.
41. The method of item 28, wherein the glycosyl transferase is a hydroxytryptophan glycosyltransferase.
42. The method of item 28, wherein the glycosyl transferase comprises the sequence of Pt73Y (SEQ ID: NO 64); (SEQ ID NO: 66); Bs109_1 (SEQ ID NO: 68); Bs109A1 (SEQ ID NO: 70); Cp73B (SEQ ID NO: 72); Cs73Y (yeast c/o) (SEQ ID NO: 92); Ha88B_2 (yeast c/o) (SEQ ID NO: 94); and/or Pt73Y (yeast c/o) (SEQ ID NO: 96).
43. The method of items 24-42, wherein the glycosyl transferase has at least 70%, such as at least 75%, such as at least 80%, such as at least 90%, such as at least 95%, such as at least 99%, such as 100% identity to the glycosyl transferase comprised in anyone of Pt73Y (SEQ ID: NO 64); (SEQ ID NO: 66); Bs109_1 (SEQ ID NO: 68); Bs109A1 (SEQ ID NO: 70); Cp73B (SEQ ID NO: 72); Cs73Y (yeast c/o) (SEQ ID NO: 92); Ha88B_2 (yeast c/o) (SEQ ID NO: 94); and/or Pt73Y (yeast c/o) (SEQ ID NO: 96).
44. The method of item 24, wherein the synthase is selected from the group consisting of: a Chorismate synthase, an Anthranilate synthase, an Indole-3-glycerol phosphate synthase, a Tryptophan synthase, a Prodeoxyviolacein synthase, and a Violacein Synthase.
45. The method of item 44, wherein the Chorismate synthase has at least 70%, such as at least 75%, such as at least 80%, such as at least 90%, such as at least 95%, such as at least 99%, such as 100% identity to the synthase comprised in SEQ ID NO: 12.
46. The method of item 44, wherein the Anthranilate synthase has at least 70%, such as at least 75%, such as at least 80%, such as at least 90%, such as at least 95%, such as at least 99%, such as 100% identity to the synthase comprised in SEQ ID NO: 14.
47. The method of item 44, wherein the Indole-3-glycerol phosphate synthase has at least 70%, such as at least 75%, such as at least 80%, such as at least 90%, such as at least 95%, such as at least 99%, such as 100% identity to the synthase comprised in SEQ ID NO: 22.
48. The method of item 44, wherein the Tryptophan synthase has at least 70%, such as at least 75%, such as at least 80%, such as at least 90%, such as at least 95%, such as at least 99%, such as 100% identity to the synthase comprised in SEQ ID NO: 24 (TRP5) and/or 63 (PcTrpB).
49. The method according to item 48, wherein the synthase has at least 70% identity to the synthase comprised in SEQ ID NO: 24 (TRP5) and the synthase is contacted with the compound of formula (II) in vivo.
50. The method according to item 49, wherein the compound of formula (II) is indole-3-glycerol phosphate.
51. The method according to item 48, wherein the synthase has at least 70% identity to the synthase comprised in SEQ ID NO: 63 (TRP5) and the synthase is contacted with the compound of formula (II) in vitro.
52. The method according to item 51, the compound of formula (II) is indole.
53. The method of item 44, wherein the Prodeoxyviolacein synthase has at least 70%, such as at least 75%, such as at least 80%, such as at least 90%, such as at least 95%, such as at least 99%, such as 100% identity to the synthase comprised in SEQ ID NO: 30.
54. The method of item 44, wherein the Violacein Synthase has at least 70%, such as at least 75%, such as at least 80%, such as at least 90%, such as at least 95%, such as at least 99%, such as 100% identity to the synthase comprised in SEQ ID NO: 34.
55. The method of item 24, wherein the kinase is a Shikimate kinase, a Ribose-phosphate pyrophosphokinase, and/or a NADH kinase.
56. The method of item 55, wherein the Shikimate kinase has at least 70%, such as at least 75%, such as at least 80%, such as at least 90%, such as at least 95%, such as at least 99%, such as 100% identity to the sequence comprised in SEQ ID NO: 10.
57. The method of item 55, wherein the Ribose-phosphate pyrophosphokinase has at least 70%, such as at least 75%, such as at least 80%, such as at least 90%, such as at least 95%, such as at least 99%, such as 100% identity to the sequence comprised in SEQ ID NO: 16.
58. The method of item 55, wherein the NADH kinase has at least 70%, such as at least 75%, such as at least 80%, such as at least 90%, such as at least 95%, such as at least 99%, such as 100% identity to the sequence comprised in SEQ ID NO: 51.
59. The method of any preceding items, wherein the one or more enzymes is an Anthranilate phosphoribosyl transferase having at least 70%, such as at least 75%, such as at least 80%, such as at least 90%, such as at least 95%, such as at least 99%, such as 100% identity to the sequence comprised in SEQ ID NO: 18.
60. The method of any preceding items, wherein the one or more enzymes is a Flavin-dependent L-tryptophan oxidase having at least 70%, such as at least 75%, such as at least 80%, such as at least 90%, such as at least 95%, such as at least 99%, such as 100% identity to the sequence comprised in SEQ ID NO: 26.
61. The method of any preceding items, wherein the one or more enzymes is a tryptophan oxidase having at least 70%, such as at least 75%, such as at least 80%, such as at least 90%, such as at least 95%, such as at least 99%, such as 100% identity to the sequence comprised in: SEQ ID NO: 80, SEQ ID NO: 84, and/or SEQ ID NO: 88.
62. The method of any preceding items, wherein the one or more enzymes is a 2-imino-3-(indol-3-yl)propanoate dimerase having at least 70%, such as at least 75%, such as at least 80%, such as at least 90%, such as at least 95%, such as at least 99%, such as 100% identity to the sequence comprised in SEQ ID NO: 28.
63. The method of any preceding items, wherein the one or more enzymes is a 2-imino-3-(indol-3-yl)propanoate dimerase Prodeoxyviolacein synthase fusion protein having at least 70%, such as at least 75%, such as at least 80%, such as at least 90%, such as at least 95%, such as at least 99%, such as 100% identity to the sequence comprised in SEQ ID NO: 74 or SEQ ID NO: 76.
64. The method of any one of the preceding items, wherein the one or more enzymes is an IPA imine dimer synthase having: a) at least 70% identity, such as at least 75%, such as at least 80%, such as at least 90%, such as at least 95%, such as at least 99%, such as 100% identity to LaRebD (SEQ ID NO: 78); b) at least 70% identity, such as at least 75%, such as at least 80%, such as at least 90%, such as at least 95%, such as at least 99%, such as 100% identity to SsStaD (SEQ ID NO: 82); c) at least 70% identity, such as at least 75%, such as at least 80%, such as at least 90%, such as at least 95%, such as at least 99%, such as 100% identity to NlInkD (SEQ ID NO: 86); or d) at least 70% identity, such as at least 75%, such as at least 80%, such as at least 90%, such as at least 95%, such as at least 99%, such as 100% identity to AmAtmD (SEQ ID NO: 90).
65. The method of any preceding items, wherein the one or more enzymes is a Protodeoxyviolaceinate monooxygenase having at least 70%, such as at least 75%, such as at least 80%, such as at least 90%, such as at least 95%, such as at least 99%, such as 100% identity to the sequence comprised in SEQ ID NO: 32.
66. The method of any preceding items, wherein the one or more enzymes is a transaldolase having at least 70%, such as at least 75%, such as at least 80%, such as at least 90%, such as at least 95%, such as at least 99%, such as 100% identity to the sequence comprised in SEQ ID NO: 38.
67. The method of any preceding items, wherein the one or more enzymes is a Transketolase having at least 70%, such as at least 75%, such as at least 80%, such as at least 90%, such as at least 95%, such as at least 99%, such as 100% identity to the sequence comprised in SEQ ID NO: 40.
68. The method of any preceding items, wherein the one or more enzymes is a GTP cyclohydrolase II having at least 70%, such as at least 75%, such as at least 80%, such as at least 90%, such as at least 95%, such as at least 99%, such as 100% identity to the sequence comprised in SEQ ID NO: 42.
69. The method of any preceding items, wherein the one or more enzymes is Mitochondrial flavin adenine dinucleotide transporter having at least 70%, such as at least 75%, such as at least 80%, such as at least 90%, such as at least 95%, such as at least 99%, such as 100% identity to the sequence comprised in SEQ ID NO: 44.
70. The method of any preceding items, wherein the one or more enzymes is Porphobilinogen deaminase having at least 70%, such as at least 75%, such as at least 80%, such as at least 90%, such as at least 95%, such as at least 99%, such as 100% identity to the sequence comprised in SEQ ID NO:47.
71. The method of items 45 to 70, wherein the sequence identity is at least 90%, such as at least 91%, such as at least 92%, such as at least 93%, such as at least 94%, such as at least 95%, such as at least 96%, such as at least 97%, such as at least 98%, such as at least 99%, such as 100%.
72. The method of any preceding item, further comprising one or more steps selected from:
   a) converting an indole or indole derivative into tryptophan or a tryptophan derivative;
   b) adding an isolated indole of formula (II) to a microbial host cell;
   c) converting an indole of formula (II) into tryptophan or a tryptophan derivative;
   d) converting tryptophan or tryptophan derivative into the compound of formula (I);
   e) converting the compound of formula (I) into a glycosylated compound thereof which is the glycosylated compound of formula (I), optionally in vivo;
   f) extraction of the compound of formula (I) or the glycosylated compound of formula (I) using an extractant, such as a surfactant, optionally at a concentration above the extractant's cloud point; and
   g) recovering the compound of formula (I) from an extractant phase.
73. The method of item 72, wherein the method comprises extraction of the glycosylated compound of formula (I).
74. The method of item 73, wherein the method further comprises de-glycosylation of the glycosylated compound of formula (I) by a β-glycosidase to provide the compound of formula (I), and optionally further isolating the compound of formula (I).
75. The method of items 72-74, wherein the extractant is a surfactant, such as a non-ionic surfactant; or a lipophilic extractant.
76. The method of items 72-74, wherein the extractant is non-miscible with water.
77. The method of item 72, wherein the extractant is isopropyl myristate, (1,1,3,3-Tetramethylbutyl)phenyl-polyethylene glycol, Polyethylene glycol tert-octylphenyl ether (Triton X-114), or polydimethylsiloxane (such as Antifoam A).
78. The method of any of the preceding items, wherein the steps are performed in vitro or in vivo.
79. The method of items 72-78, wherein the conversion of the indole into the tryptophan or tryptophan derivative comprises contacting the indole with a tryptophan synthase enzyme, optionally a tryptophan synthase which has at least 70%, such as at least 75%, such as at least
80%, such as at least 90%, such as at least 95%, such as at least 99%, such as 100% identity to the tryptophan synthase comprised in SEQ ID NO: 24 and/or 63.
80. The method of any preceding item comprising in vitro enzymatic reaction steps and/or optionally in vivo enzymatic reaction steps.
81. The method of any preceding items, comprising expressing a glycosyl transferase in yeast, such as in S. cerevisiae and performing in vivo glycosylation of the compound of formula (I).
82. The method of item 81, wherein the glycosyl transferase has at least 70% sequence identity to the polypeptide sequence comprised in sequence of Pt73Y according to SEQ ID NO: 66, such as at least 75%, such as at least 80%, such as at least 85%, such as at least 90%, such as at least 95%, such as 100%.
83. The method of item 81, wherein the glycosyl transferase has at least 70% sequence identity to any one of the polypeptide sequences comprised in sequence of: Pt73Y (SEQ ID: NO 64); (SEQ ID NO: 66); Bs109_1 (SEQ ID NO: 68); Bs109A1 (SEQ ID NO: 70); Cp73B (SEQ ID NO: 72); Cs73Y (yeast c/o) (SEQ ID NO: 92); Ha88B_2 (yeast c/o) (SEQ ID NO: 94); and/or Pt73Y (yeast c/o) (SEQ ID NO: 96).
84. The method of item 83, wherein the glycosyl transferase has at least 70% sequence identity to any one of the polypeptide sequences comprised in sequence of: Pt73Y (SEQ ID: NO 64); Cs73Y (yeast c/o) (SEQ ID NO: 92); and/or Pt73Y (yeast c/o) (SEQ ID NO: 96), such as at least 75%, such as at least 80%, such as at least 85%, such as at least 90%, such as at least 95%, such as 100%,
85. The method of item 80, comprising expressing a glycosyl transferase in yeast, such as in S. cerevisiae or Pichia pastoris and performing in vitro glycosylation of the compound of formula (I).
86. The method of any preceding items, comprising expressing a glycosyl transferase in E. coli and performing in vitro glycosylation of the compound of formula (I).
87. The method of items 85-86, wherein the glycosyl transferase has at least 70% sequence identity to any one of the polypeptide sequences comprised in sequence of: Pt73Y (SEQ ID: NO 64); (SEQ ID NO: 66); Bs109_1 (SEQ ID NO: 68); Bs109A1 (SEQ ID NO: 70); Cp73B (SEQ ID NO: 72); Cs73Y (yeast c/o) (SEQ ID NO: 92); Ha88B_2 (yeast c/o) (SEQ ID NO: 94); and/or Pt73Y (yeast c/o) (SEQ ID NO: 96).
88. The method of items 85-86, wherein the glycosyl transferase has at least 70% sequence identity to the polypeptide sequence comprised in sequence of Pt73Y according to SEQ ID NO: 66, such as at least 75%, such as at least 80%, such as at least 85%, such as at least 90%, such as at least 95%, such as 100%.
89. A compound of formula (I):

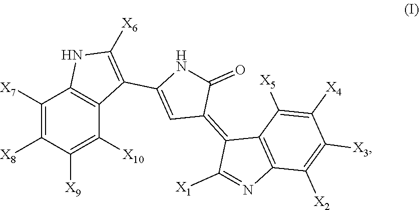

or a tautomer thereof, wherein any one of X₁, X₂, X₃, X₄, X₅, X₆, X₇, X₈, X₉, and X₁₀ are independently of each other selected from the group consisting of: H, R₁, R₂, O, OH, OR₁, NH, NO₂, NH₂, NHR₁, NHR₂, SR₁, F, Cl, Br, I, and SH; wherein R₁ and R₂ are independently of each other selected from the group consisting of a C₁₋₈ alkyl, C₁₋₈ alkenyl, C₁₋₈ alkoyl, C₁₋₈ aryl, and C₁₋₈ aroyl, and R₁ and R₂ are optionally covalently linked to form a ring.
90. The compound according to item 89, wherein the compound is selected from the group consisting of:

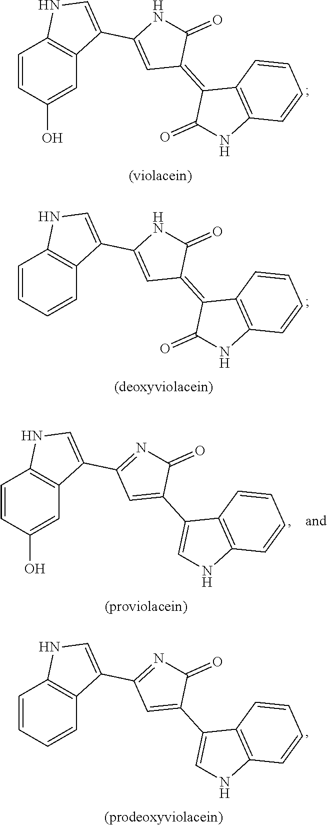 [SMILES: O=C1NC(c2c[nH]c3ccc(O)cc23)=C/C1=C1\C(=O)Nc2ccccc21 ; O=C1NC(c2c[nH]c3ccccc23)=C/C1=C1\C(=O)Nc2ccccc21 ; O=C1N=C(c2c[nH]c3ccc(O)cc23)C=C1c1c[nH]c2ccccc12 ; O=C1N=C(c2c[nH]c3ccccc23)C=C1c1c[nH]c2ccccc12]

and tautomers thereof.
91. The compound according to any one of items 89-90, further covalently linked to a saccharide, preferably by a glycosidic linkage.
92. The compound according to any one of items 89-91, wherein the compound is in enol form covalently linked to a saccharide via an enol oxygen, preferably by a glycosidic linkage.
93. The compound according to item 91, wherein the compound is of formula (III) or formula (IV):

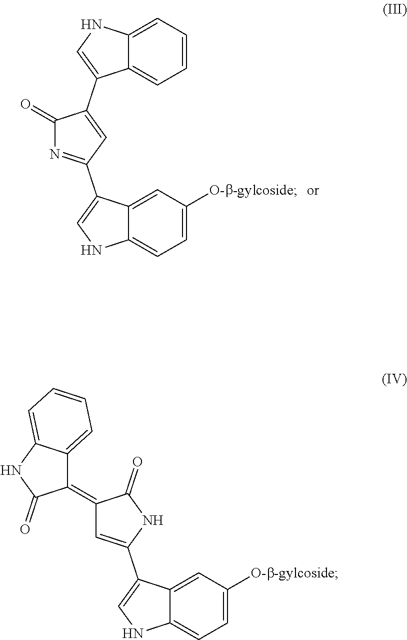

wherein “β-glycoside” is a saccharide linked by a β-glycosidic bond to the remainder of the molecule.
94. The compound according to any of items 91-93, wherein the saccharide is a monosaccharide, a disaccharide, a trisaccharide, or a tetrasaccharide.
95. The compound according to item 94, wherein the monosaccharide is selected from the group consisting of: glucose, fructose, galactose, mannose, arabinose, xylose, ribulose, xylulose, ribose, desoxyribose, desoxygalactose, fucose, and rhamnose, preferably wherein the monosaccharide is glucose, such as D-glucose.
96. A microbial host cell genetically modified to perform any of the method of items 1 to 88 and produce a compound of formula (I),

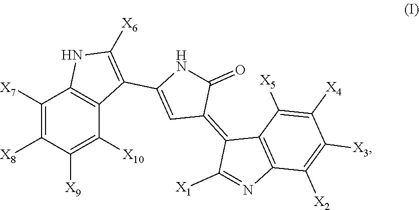

or a tautomer thereof, wherein any one of X₁, X₂, X₃, X₄, X₅, X₆, X₇, X₈, X₉, and X₁₀ are independently of each other selected from the group consisting of: H, R₁, R₂, O, OH, OR₁, NH, NO₂, NH₂, NHR₁, NHR₂, SR₁, F, Cl, Br, I, and SH; wherein R₁ and R₂ are independently of each other selected from the group consisting of a C₁₋₈ alkyl, C₁₋₈ alkenyl, C₁₋₈ alkoyl, C₁₋₈ aryl, and C₁₋₈ aroyl, and R₁ and R₂ are optionally covalently linked to form a ring;
wherein the host cell expresses one or more heterologous genes encoding the one or more enzymes.
97. The host cell of item 96, further comprising an operative biosynthetic pathway for producing violacein, wherein the host cell expresses one or more pathway genes encoding polypeptides selected from:
   a) one or more enzymes capable of converting glucose to fructose-6-phosphate;
   b) one or more enzymes capable of converting glucose to D-ribulose-5-phosphate;
   c) a transketolase capable of converting xylulose-5-phosphate and ribose-5-phosphate to glyceraldehyde-3-phosphate and sedoheptulose-7-phosphate, such as the transketolase TKL1;
   d) a transaldolase capable of converting glyceraldehyde 3-phosphate and sedoheptulose 7-phosphate to erythrose 4-phosphate and fructose 6-phosphate, such as the transaldolase TAL1;
   e) a fructose-6-phosphate phosphoketolase capable of converting fructose-6-phosphate to Erythrose-4-phosphate and acetyl phosphate, such as the phosphoketolase BfXfpk;
   f) a Phosphotransacetylase capable of converting Acetyl phosphate to Acetyl-CoA, such as the phosphotransacetylase CkPTa;
   g) one or more enzymes capable of converting Fructose-6-phosphate to Phosphoenolpyruvate;
   h) a 3-deoxy-D-arabino-heptulosonate 7-phosphate synthase (DAHP synthase) capable of converting Phosphoenolpyruvate and Erythrose-4-phosphate to 3-deoxy-D-arabino-heptulosonate-7-phosphate (DAHP), such as the synthase ARO4(K229L);
   i) a 3-dehydroquinate synthase capable of converting 3-deoxy-D-arabino-heptulosonate 7-phosphate to 3-dehydroquinate, such as the synthase ARO1;
   j) a 3-dehydroquinate dehydratase capable of converting 3-dehydroquinate to 3-dehydroshikimate, such as the dehydratase ARO1;
   k) a Shikimate dehydrogenase capable of converting 3-dehydroshikimate to Shikimate, such as the dehydrogenase ARO1;
   l) a Shikimate kinase capable of converting Shikimate to Shikimate-3-phosphate, such as the kinase ARO1 and/or EcAroL;
   m) a 3-phosphoshikimate 1-carboxyvinyltransferase capable of converting Shikimate-3-phosphate and Phosphoenolpyruvate to 5-enolpyruvoyl-shikimate 3-phosphate, such as the transferase ARO1;
   n) a Chorismate synthase capable of converting 5-enolpyruvoyl-shikimate 3-phosphate to Chorismate, such as the synthase ARO2;
   o) an Anthranilate synthase capable of converting Chorismate to Anthranilate, such as the synthase TRP2(S65R, S76L);
   p) a Ribose-phosphate pyrophosphokinase capable of converting Ribose-5-phosphate to Phospho-alpha-D-ribosyl-1-pyrophosphate, such as the pyrophosphokinase BsPrs;
   q) an Anthranilate phosphoribosyl transferase capable of converting Anthranilate and Phospho-alpha-D-ribosyl-1-pyrophosphate to N-(5-phosphoribosyl)-anthranilate, such as the transferase TRP4;
   r) a N-(5′-phosphoribosyl)-anthranilate isomerase capable of converting N-(5-phosphoribosyl)-anthranilate to 1-(o-carboxyphenylamino)-1′-deoxyribulose 5′-phosphate, such as the isomerase TRP1;
   s) a Indole-3-glycerol phosphate synthase capable of converting 1-(o-carboxyphenylamino)-1′-deoxyribulose 5′-phosphate to (1S,2R)-1-C-(indol-3-yl)glycerol 3-phosphate, such as the synthase TRP3;
   t) a Tryptophan synthase capable of converting (1S,2R)-1-C-(indol-3-yl)glycerol 3-phosphate and Serine to L-Tryptophan, such as the synthase TRP5;
   u) a tryptophan synthase capable of converting Indole and Serine to L-Tryptophan, such as the synthase TRP5;
   v) a Flavin-dependent L-tryptophan oxidase capable of converting L-Tryptophan to IPA imine, such as CvVioA;
   w) a tryptophan oxidase, such as SsStaO, NlInkO, or AmAtmO;
   x) a 2-imino-3-(indol-3-yl)propanoate dimerase capable of converting IPA imine to IPA imine dimer, such as the dimerase CvVioB;
   y) an IPA imine dimer synthase, such as LaRebD, SsStaD, NlInkD, and/or AmAtmD;
   z) a Prodeoxyviolacein synthase capable of converting IPA imine dimer to Protodeoxyviolaceinic acid, such as the synthase CvVioE;
   aa) a Protodeoxyviolaceinate monooxygenase synthase capable of converting Protodeoxyviolaceinic acid to Protoviolaceinic acid, such as the synthase CvVioD; and
   bb) a Violacein synthase capable of converting Protoviolaceinic acid to Violaceinic acid and capable of converting Protodeoxyviolaceinic acid to Protoviolaceinic acid, such as CvVioC.
98. The host cell of items 96-97, further comprising an operative biosynthetic pathway for heme biosynthesis, wherein the host cell expresses one or more pathway genes encoding polypeptides selected from:
   a) one or more enzymes capable of converting glucose to glycine;
   b) one or more enzymes capable of converting glycine to porphobilinogen;
   c) a Porphobilinogen deaminase capable of converting Porphobilinogen to Hydroxymethylbilane, such as the deaminase HEM3; and
   d) one or more enzymes capable of converting Hydroxymethylbilane to Ferroheme b.
99. The host cell of items 96-90, further comprising an operative biosynthetic pathway for flavin biosynthesis, wherein the host cell expresses one or more pathway genes encoding polypeptides selected from:
   a) a GTP cyclohydrolase II capable of converting GTP to 2,5-diamino-6-ribosylamino-4(3H)-pyrimidinone 5′-phosphate, such as the cyclohydrolase RIB1; and
   b) one or more enzymes capable of converting 2,5-diamino-6-ribosylamino-4(3H)-pyrimidinone 5′-phosphate to FAD.
100. The host cell of items 88-99, wherein the host cell further expresses one or more genes encoding catalytic or non-catalytic polypeptides selected from:
   a) a NADH kinase capable of converting NADH and ATP to NADPH and ADP, such as the kinase POS5; and
   b) a Mitochondrial flavin adenine dinucleotide transporter, such as FLX1.
101. The host cell of items 96-100, wherein one or more genes has been attenuated, disrupted and/or deleted, said one or more genes encoding catalytic or non-catalytic polypeptides selected from:
   a) a Heme oxygenase capable of converting Ferroheme b to Biliverdin, such as the oxygenase HMX1;
   b) a Heme-responsive transcription factor, such as HAP1;
   c) a mRNA-binding ubiquitin-specific protease, such as UBP3;
   d) a Cis-Golgi network transporter protein, such as RIC1; and
   e) a Heme-dependent repressor of hypoxic genes, such as ROX1,
102. The host cell of items 96-101, wherein the corresponding:
   a) transketolase capable of converting xylulose-5-phosphate and ribose-5-phosphate to glyceraldehyde-3-phosphate and sedoheptulose-7-phosphate has at least 70% identity to the sequence comprised in SEQ ID NO: 40;
   b) transaldolase capable of converting glyceraldehyde 3-phosphate and sedoheptulose 7-phosphate to erythrose 4-phosphate and fructose 6-phosphate has at least 70% identity to the sequence comprised in SEQ ID NO: 38;
   c) fructose-6-phosphate phosphoketolase capable of converting fructose-6-phosphate to Erythrose-4-phosphate and acetyl phosphate has at least 70% identity to the sequence comprised in SEQ ID NO: 2;
   d) Phosphotransacetylase capable of converting Acetyl phosphate to Acetyl-CoA has at least 70% identity to the sequence comprised in SEQ ID NO: 4;
   e) 3-deoxy-D-arabino-heptulosonate 7-phosphate synthase (DAHP synthase) capable of converting Phosphoenolpyruvate and Erythrose-4-phosphate to 3-deoxy-D-arabino-heptulosonate-7-phosphate (DAHP) has at least 70% identity to the sequence comprised in SEQ ID NO: 6;
   f) 3-dehydroquinate synthase capable of converting 3-deoxy-D-arabino-heptulosonate 7-phosphate to 3-dehydroquinate has at least 70% identity to the sequence comprised in SEQ ID NO: 8;
   g) 3-dehydroquinate dehydratase capable of converting 3-dehydroquinate to 3-dehydroshikimate has at least 70% identity to the sequence comprised in SEQ ID NO: 8; h) Shikimate dehydrogenase capable of converting 3-dehydroshikimate to Shikimate has at least 70% identity to the sequence comprised in SEQ ID NO: 8;
   i) Shikimate kinase capable of converting Shikimate to Shikimate-3-phosphate has at least 70% identity to the sequence comprised in SEQ ID NO: 8; and/or at least 70% identity to the sequence comprised in SEQ ID NO: 10;
   j) 3-phosphoshikimate 1-carboxyvinyltransferase capable of converting Shikimate-3-phosphate and Phosphoenolpyruvate to 5-enolpyruvoyl-shikimate 3-phosphate has at least 70% identity to the sequence comprised in SEQ ID NO: 8;
   k) Chorismate synthase capable of converting 5-enolpyruvoyl-shikimate 3-phosphate to Chorismate has at least 70% identity to the sequence comprised in SEQ ID NO: 12;
   l) Anthranilate synthase capable of converting Chorismate to Anthranilate has at least 70% identity to the sequence comprised in SEQ ID NO: 14;
   m) Ribose-phosphate pyrophosphokinase capable of converting Ribose-5-phosphate to Phospho-alpha-D-ribosyl-1-pyrophosphate has at least 70% identity to the sequence comprised in SEQ ID NO: 16;
   n) Anthranilate phosphoribosyl transferase capable of converting Anthranilate and Phospho-alpha-D-ribosyl-1-pyrophosphate to N-(5-phosphoribosyl)-anthranilate has at least 70% identity to the sequence comprised in SEQ ID NO: 18;
   o) N-(5′-phosphoribosyl)-anthranilate isomerase capable of converting N-(5-phosphoribosyl)-anthranilate to 1-(o-carboxyphenylamino)-1′-deoxyribulose 5′-phosphate has at least 70% identity to the sequence comprised in SEQ ID NO: 20;
   p) Indole-3-glycerol phosphate synthase capable of converting 1-(o-carboxyphenylamino)-1′-deoxyribulose 5′-phosphate to (1S,2R)-1-C-(indol-3-yl)glycerol 3-phosphate has at least 70% identity to the sequence comprised in SEQ ID NO: 22;
   q) Tryptophan synthase capable of converting (1S,2R)-1-C-(indol-3-yl)glycerol 3-phosphate and Serine to L-Tryptophan has at least 70% identity to the sequence comprised in SEQ ID NO: 24;
   r) Flavin-dependent L-tryptophan oxidase capable of converting L-Tryptophan to IPA imine has at least 70% identity to the sequence comprised in SEQ ID NO: 26;
   s) tryptophan oxidase has at least 70% identity to the sequence comprised in SsStaO (SEQ ID NO: 80), NlInkO (SEQ ID NO: 84), and/or AmAtmO (SEQ ID NO: 88);
   t) 2-imino-3-(indol-3-yl)propanoate dimerase capable of converting IPA imine to IPA imine dimer has at least 70% identity to the sequence comprised in SEQ ID NO: 28;
   u) IPA imine dimer synthase has at least 70% identity to the sequence comprised in LaRebD (SEQ ID NO: 78), SsStaD (SEQ ID NO: 82), NlInkD (SEQ ID NO: 86), and/or AmAtmD (SEQ ID NO: 90);
   v) Prodeoxyviolacein synthase capable of converting IPA imine dimer to Protodeoxyviolaceinic acid has at least 70% identity to the sequence comprised in SEQ ID NO: 30;
   w) Protodeoxyviolaceinate monooxygenase synthase capable of converting Protodeoxyviolaceinic acid to Protoviolaceinic acid has at least 70% identity to the sequence comprised in SEQ ID NO: 32; and/or
   x) Violacein synthase capable of converting Protoviolaceinic acid to Violaceinic acid and capable of converting Protodeoxyviolaceinic acid to Protoviolaceinic acid has at least 70% identity to the sequence comprised in SEQ ID NO: 34.
103. The host cell of items 96-102, wherein the one or more expressed genes are selected from:
   a) genes encoding a transketolase capable of converting xylulose-5-phosphate and ribose-5-phosphate to glyceraldehyde-3-phosphate and sedoheptulose-7-phosphate, said genes being at least 70% identical to the polynucleotide sequence comprised in SEQ ID NO: 39 or genomic DNA thereof;
   b) genes encoding a transaldolase capable of converting glyceraldehyde 3-phosphate and sedoheptulose 7-phosphate to erythrose 4-phosphate and fructose 6-phosphate, said genes being at least 70% identical to the polynucleotide sequence comprised in SEQ ID NO: 37 or genomic DNA thereof;
   c) genes encoding a fructose-6-phosphate phosphoketolase capable of converting fructose-6-phosphate to Erythrose-4-phosphate and acetyl phosphate, said genes being at least 70% identical to the polynucleotide sequence comprised in SEQ ID NO: 1 or genomic DNA thereof;
   d) genes encoding a Glycerol-1-phosphatase capable of converting Acetyl phosphate to Acetate, said genes being at least 70% identical to the polynucleotide sequence comprised in SEQ ID NO: 59 or genomic DNA thereof;
   e) genes encoding a Phosphotransacetylase capable of converting Acetyl phosphate to Acetyl-CoA, said genes being at least 70% identical to the polynucleotide sequence comprised in SEQ ID NO: 3 or genomic DNA thereof;
   f) genes encoding a 3-deoxy-D-arabino-heptulosonate 7-phosphate synthase (DAHP synthase) capable of converting Phosphoenolpyruvate and Erythrose-4-phosphate to 3-deoxy-D-arabino-heptulosonate-7-phosphate (DAHP), said genes being at least 70% identical to the polynucleotide sequence comprised in SEQ ID NO: 5 or genomic DNA thereof;
   g) genes encoding a 3-dehydroquinate synthase capable of converting 3-deoxy-D-arabino-heptulosonate 7-phosphate to 3-dehydroquinate, said genes being at least 70% identical to the polynucleotide sequence comprised in SEQ ID NO: 7 or genomic DNA thereof;
   h) genes encoding a 3-dehydroquinate dehydratase capable of converting 3-dehydroquinate to 3-dehydroshikimate, said genes being at least 70% identical to the polynucleotide sequence comprised in SEQ ID NO: 7 or genomic DNA thereof;
   i) genes encoding a Shikimate dehydrogenase capable of converting 3-dehydroshikimate to Shikimate, said genes being at least 70% identical to the polynucleotide sequence comprised in SEQ ID NO: 7 or genomic DNA thereof;
   j) genes encoding a Shikimate kinase capable of converting Shikimate to Shikimate-3-phosphate, said genes being at least 70% identical to the polynucleotide sequence comprised in SEQ ID NO: 7 or genomic DNA thereof and/or at least 70% identical to the polynucleotide sequence comprised in SEQ ID NO: 9 or genomic DNA thereof;
   k) genes encoding a 3-phosphoshikimate 1-carboxyvinyltransferase capable of converting Shikimate-3-phosphate and Phosphoenolpyruvate to 5-enolpyruvoyl-shikimate 3-phosphate, said genes being at least 70% identical to the polynucleotide sequence comprised in SEQ ID NO: 7 or genomic DNA thereof;
   l) genes encoding a Chorismate synthase capable of converting 5-enolpyruvoyl-shikimate 3-phosphate to Chorismate, said genes being at least 70% identical to the polynucleotide sequence comprised in SEQ ID NO: 11 or genomic DNA thereof;
   m) genes encoding an Anthranilate synthase capable of converting Chorismate to Anthranilate, said genes being at least 70% identical to the polynucleotide sequence comprised in SEQ ID NO: 13 or genomic DNA thereof;
   n) genes encoding a Ribose-phosphate pyrophosphokinase capable of converting Ribose-5-phosphate to Phospho-alpha-D-ribosyl-1-pyrophosphate, said genes being at least 70% identical to the polynucleotide sequence comprised in SEQ ID NO: 15 or genomic DNA thereof;
   o) genes encoding an Anthranilate phosphoribosyl transferase capable of converting Anthranilate and Phospho-alpha-D-ribosyl-1-pyrophosphate to N-(5-phosphoribosyl)-anthranilate, said genes being at least 70% identical to the polynucleotide sequence comprised in SEQ ID NO: 17 or genomic DNA thereof;
   p) genes encoding a N-(5′-phosphoribosyl)-anthranilate isomerase capable of converting N-(5-phosphoribosyl)-anthranilate to 1-(o-carboxyphenylamino)-1′-deoxyribulose 5′-phosphate, said genes being at least 70% identical to the polynucleotide sequence comprised in SEQ ID NO: 19 or genomic DNA thereof;
   q) genes encoding a Indole-3-glycerol phosphate synthase capable of converting 1-(o-carboxyphenylamino)-1′-deoxyribulose 5′-phosphate to (1S,2R)-1-C-(indol-3-yl)glycerol 3-phosphate, said genes being at least 70% identical to the polynucleotide sequence comprised in SEQ ID NO: 21 or genomic DNA thereof;
   r) genes encoding a Tryptophan synthase capable of converting (1S,2R)-1-C-(indol-3-yl)glycerol 3-phosphate and Serine to L-Tryptophan, said genes being at least 70% identical to the polynucleotide sequence comprised in SEQ ID NO: 23 or genomic DNA thereof;
   s) genes encoding a Flavin-dependent L-tryptophan oxidase capable of converting L-Tryptophan to IPA imine, said genes being at least 70% identical to the polynucleotide sequence comprised in SEQ ID NO: 25 or genomic DNA thereof;
   t) genes encoding a tryptophan oxidase, said genes being at least 70% identical to the polynucleotide sequence comprised in any one of SEQ ID NO: 79, SEQ ID NO: 83, SEQ ID NO: 87, or genomic DNA thereof;
   u) genes encoding a 2-imino-3-(indol-3-yl)propanoate dimerase capable of converting IPA imine to IPA imine dimer, said genes being at least 70% identical to the polynucleotide sequence comprised in SEQ ID NO: 27 or genomic DNA thereof;
   v) genes encoding a IPA imine dimer synthase, said genes being at least 70% identical to the polynucleotide sequence comprised in any one of SEQ ID NO: 77, SEQ ID NO: 81, SEQ ID NO: 85, SEQ ID NO: 89, or genomic DNA thereof;
   w) genes encoding a Prodeoxyviolacein synthase capable of converting IPA imine dimer to Protodeoxyviolaceinic acid, said genes being at least 70% identical to the polynucleotide sequence comprised in SEQ ID NO: 29 or genomic DNA thereof;
   x) genes encoding a Protodeoxyviolaceinate monooxygenase synthase capable of converting Protodeoxyviolaceinic acid to Protoviolaceinic acid, said genes being at least 70% identical to the polynucleotide sequence comprised in SEQ ID NO: 31 or genomic DNA thereof;
   y) genes encoding a Violacein synthase capable of converting Protoviolaceinic acid to Violaceinic acid, said genes being at least 70% identical to the polynucleotide sequence comprised in SEQ ID NO: 33 or genomic DNA thereof.
104. The host cell of items 96-103, wherein the corresponding:
   a) Porphobilinogen deaminase capable of converting Porphobilinogen to Hydroxymethylbilane has at least 70% identity to the sequence comprised in SEQ ID NO: 47.
105. The host cell of items 96-104, wherein the one or more expressed genes are selected from:
   a) genes encoding Porphobilinogen deaminase capable of converting Porphobilinogen to Hydroxymethylbilane, said genes being at least 70% identical to the polynucleotide sequence comprised in SEQ ID NO: 46 or genomic DNA thereof.
106. The host cell of items 96-105, wherein the corresponding:
   a) GTP cyclohydrolase II capable of converting GTP to 2,5-diamino-6-ribosylamino-4(3H)-pyrimidinone 5′-phosphate has at least 70% identity to the sequence comprised in SEQ ID NO: 42.
107. The host cell of items 96-106, wherein the one or more expressed genes are selected from:
   a) genes encoding GTP cyclohydrolase II capable of converting GTP to 2,5-diamino-6-ribosylamino-4(3H)-pyrimidinone 5′-phosphate, said genes being at least 70% identical to the polynucleotide sequence comprised in SEQ ID NO: 41 or genomic DNA thereof.
108. The host cell of items 100-107, wherein the corresponding:
   a) NADH kinase capable of converting NADH and ATP to NADPH and ADP has at least 70% identity to the sequence comprised in SEQ ID NO: 51;
   b) Mitochondrial flavin adenine dinucleotide transporter has at least 70% identity to the sequence comprised in SEQ ID NO: 44;
   c) Tryptophan synthase has at least 70% identity to the sequence comprised in SEQ ID NO: 63; and/or
   d) Glycosyltransferase has at least 70% identity to the sequence comprised in SEQ ID NO: 66, SEQ ID NO: 68, SEQ ID NO: 70, SEQ ID NO: 72, SEQ ID NO: 92, SEQ ID NO: 94, and/or SEQ ID NO: 96.
109. The host cell of items 96-108, wherein the one or more expressed genes are selected from:
   a) genes encoding a NADH kinase capable of converting NADH and ATP to NADPH and ADP, said genes being at least 70% identical to the polynucleotide sequence comprised in SEQ ID NO: 50 or genomic DNA thereof;
   b) genes encoding a Mitochondrial flavin adenine dinucleotide transporter, said genes being at least 70% identical to the polynucleotide sequence comprised in SEQ ID NO: 43 or genomic DNA thereof;
   c) genes encoding a Tryptophan synthase, said genes being at least 70% identical to the polynucleotide sequence comprised in SEQ ID NO: 62 or genomic DNA thereof; and
   d) genes encoding a Glycosyltransferase, said genes being at least 70% identical to the polynucleotide sequence comprised in SEQ ID NO: 64, SEQ ID NO: 65, SEQ ID NO: 67, SEQ ID NO: 69, SEQ ID NO: 71, SEQ ID NO: 91, SEQ ID NO: 93, and/or SEQ ID NO: 95, or genomic DNA thereof.
110. The host cell of items 96-109, wherein the sequence identity is least 90%, such as at least 91%, such as at least 92%, such as at least 93%, such as at least 94%, such as at least 95%, such as at least 96%, such as at least 97%, such as at least 98%, such as at least 99%, such as 100%,
111. The host cell of item 110, wherein the sequence identity is at least 99%, such as 100%,
112. The host cell of items 96-111, comprising at least two copies of one or more of the heterologous genes encoding the one or more enzymes of the pathway genes.
113. The host cell of item 112, wherein one or more of the heterologous genes encoding the one or more enzymes are overexpressed.
114. The host cell of items 96-113, further genetically modified to provide an increased amount of a substrate for at least one polypeptide of the violacein pathway.
115. The host cell of items 96-114, further genetically modified to exhibit increased tolerance towards one or more substrates, intermediates, or product molecules from the indole acceptor pathway.
116. The host cell of items 96-115, wherein the host cell is an eukaryotic, prokaryotic or archaic host cell.
117. The host cell of item 116, wherein the host cell is an eukaryote cell selected from the group consisting of a mammalian, insect, plant, or fungal host cell.
118. The host cell of item 117, wherein the host cell is a fungal host cell selected from phylas consisting of Ascomycota, Basidiomycota, Neocallimastigomycota, Glomeromycota, Blastocladiomycota, Chytridiomycota, Zygomycota, Oomycota and Microsporidia.
119. The host cell of item 117, wherein the fungal host cell is a yeast host cell selected from the group consisting of ascosporogenous yeast (Endomycetales), basidiosporogenous yeast, and Fungi Imperfecti yeast (Blastomycetes).
120. The host cell of item 119, wherein the yeast host cell is selected from the genera consisting of Saccharomyces, Kluveromyces, Candida, Pichia, Debaromyces, Hansenula, Yarrowia, Zygosaccharomyces, and Schizosaccharomyces.
121. The host cell of item 119, wherein the yeast host cell is selected from the species consisting of Kluyveromyces lactis, Saccharomyces carlsbergensis, Saccharomyces cerevisiae, Saccharomyces diastaticus, Saccharomyces douglasii, Saccharomyces kluyveri, Saccharomyces norbensis, Saccharomyces oviformis, Saccharomyces boulardii, Pichia pastoris and Yarrowia lipolytica.
122. The host cell of item 117, wherein the fungal host cell is filamentous fungus host cell.
123. The host cell of item 122, wherein the filamentous fungal host cell is selected from the phylas consisting of Ascomycota, Eumycota and Oomycota.
124. The host cell of item 122, wherein the filamentous fungal host cell is selected from the genera consisting of Acremonium, Aspergillus, Aureobasidium, Bjerkandera, Ceriporiopsis, Chrysosporium, Coprinus, Corio/us, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, and Trichoderma.
125. The host cell of item 122, wherein the filamentous fungal host cell is selected from the species consisting of Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporiuminops, Chrysosporiumkeratinophilum, Chrysosporium lucknowense, Chrysosporium merdarium, Chrysosporium pannicola, Chrysosporium queenslandicum, Chrysosporium tropicum, Chrysosporium zonatum, Coprinus cinereus, Coriolus hirsutus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Thielavia terrestris, Trametes villosa, Trametes versicolor, Trichoderma harzianum, Trichoderma koningii, Trichoderma longibrachiatum, Trichoderma reesei, and Trichoderma viride.
126. The host cell of item 116, wherein the host cell is a prokaryotic cell.
127. The host cell of item 126, wherein the prokaryotic cell is E. coli.
128. The host cell of item 116, wherein the host cell is an archaic cell.
129. The host cell of item 128, wherein the archaic cell is an algae.
130. The host cell of items 96-129, wherein one or more native genes are attenuated, disrupted and/or deleted.
131. The host cell of items 96-125, wherein the host cell is a yeast strain modified by attenuating, disrupting and/or deleting one or more native genes selected from:
   a) The ARO10 gene comprised in anyone of SEQ ID NO: 49 or any of its paralogs or orthologs having at least 70% identity to SEQ ID NO: 49;
   b) The PDC5 gene comprised in anyone of SEQ ID NO: 48 or any of its paralogs or orthologs having at least 70% identity to SEQ ID NO: 48;
   c) The UBP3 gene comprised in anyone of SEQ ID NO: 57 or any of its paralogs or orthologs having at least 70% identity to SEQ ID NO: 57;
   d) The RIC1 gene comprised in anyone of SEQ ID NO: 58 or any of its paralogs or orthologs having at least 70% identity to SEQ ID NO: 58;
   e) The GPP1 gene comprised in anyone of SEQ ID NO: 59 or any of its paralogs or orthologs having at least 70% identity to SEQ ID NO: 59;
   f) The ROX1 gene comprised in anyone of SEQ ID NO: 59 or any of its paralogs or orthologs having at least 70% identity to SEQ ID NO: 60;
   g) The HMX1 gene comprised in anyone of SEQ ID NO: 59 or any of its paralogs or orthologs having at least 70% identity to SEQ ID NO: 45; and
   h) The HAP1 gene comprised in anyone of SEQ ID NO: 59 or any of its paralogs or orthologs having at least 70% identity to SEQ ID NO: 61.
132. The host cell of items 96-131, wherein the host cell is a yeast strain modified by overexpressing one or more genes selected from:
   a) The ARO1 gene comprised in SEQ ID NO: 7 or any of its paralogs or orthologs having at least 70% identity to SEQ ID NO: 7;
   b) The ARO2 gene comprised in SEQ ID NO: 11 or any of its paralogs or orthologs having at least 70% identity to SEQ ID NO:11;
   c) The TRP4 gene comprised in SEQ ID NO: 17 or any of its paralogs or orthologs having at least 70% identity to SEQ ID NO: 17;
   d) The TRP1 gene comprised in SEQ ID NO: 19 or any of its paralogs or orthologs having at least 70% identity to SEQ ID NO: 19;
   e) The TRP3 gene comprised in SEQ ID NO: 21 or any of its paralogs or orthologs having at least 70% identity to SEQ ID NO: 21;
   f) The TRP5 gene comprised in SEQ ID NO: 23 or any of its paralogs or orthologs having at least 70% identity to SEQ ID NO: 23;
   g) The TAL1 gene comprised in SEQ ID NO: 37 or any of its paralogs or orthologs having at least 70% identity to SEQ ID NO: 37;
   h) The TKL1 gene comprised in SEQ ID NO: 39 or any of its paralogs or orthologs having at least 70% identity to SEQ ID NO: 39;
   i) The RIB1 gene comprised in SEQ ID NO: 41 or any of its paralogs or orthologs having at least 70% identity to SEQ ID NO: 41;
   j) The FLX1 gene comprised in SEQ ID NO: 43 or any of its paralogs or orthologs having at least 70% identity to SEQ ID NO: 43;
   k) The POS5 gene comprised in SEQ ID NO: 50 or any of its paralogs or orthologs having at least 70% identity to SEQ ID NO: 50; and
   l) The HEM3 gene comprised in SEQ ID NO: 46 or any of its paralogs or orthologs having at least 70% identity to SEQ ID NO: 46.
133. The host cell of any items 96-132, wherein the host cell is a yeast strain modified by overexpressing one or more genes selected from:
   a) The K229L modified ARO4 gene, ARO4(K229L) comprised in SEQ ID NO: 5 or any of its paralogs or orthologs having at least 70% identity to SEQ ID NO: 5; and
   b) The (S65R, S76L) modified TRP2 gene, TRP2(S65R, S76L) comprised in SEQ ID NO: 13 or any of its paralogs or orthologs having at least 70% identity to SEQ ID NO: 13.
134. The host cell of items 96-133, wherein the host cell is a yeast strain modified by heterologous gene overexpressing of one or more genes selected from:
   a) CvVioA encoding comprised in SEQ ID NO: 25 or any of its paralogs or orthologs having at least 70% identity to SEQ ID NO: 25;
   b) CvVioB comprised in SEQ ID NO: 27 or any of its paralogs or orthologs having at least 70% identity to SEQ ID NO: 27;
   c) CvVioC comprised in SEQ ID NO: 33 or any of its paralogs or orthologs having at least 70% identity to SEQ ID NO: 33;
   d) CvVioD comprised in SEQ ID NO: 31 or any of its paralogs or orthologs having at least 70% identity to SEQ ID NO: 31;
   e) CvVioE comprised in SEQ ID NO: 29 or any of its paralogs or orthologs having at least 70% identity to SEQ ID NO: 29;
   f) BfXfpk comprised in SEQ ID NO: 1 or any of its paralogs or orthologs having at least 70% identity to SEQ ID NO: 1;
   g) CkPta comprised in SEQ ID NO: 3 or any of its paralogs or orthologs having at least 70% identity to SEQ ID NO: 3;
   h) EcAroL comprised in SEQ ID NO: 9 or any of its paralogs or orthologs having at least 70% identity to SEQ ID NO: 9; and
   i) BsPrs comprised in SEQ ID NO: 15 or any of its paralogs or orthologs having at least 70% identity to SEQ ID NO: 15.
135. The host cell of items 96-133, wherein the host cell is a yeast strain modified by heterologous gene overexpressing of one or more genes selected from:
   a) CvVioA encoding comprised in SEQ ID NO: 25 or any of its paralogs or orthologs having at least 70% identity to SEQ ID NO: 25;
   b) CvVioB comprised in SEQ ID NO: 27 or any of its paralogs or orthologs having at least 70% identity to SEQ ID NO: 27;
   c) CvVioC comprised in SEQ ID NO: 33 or any of its paralogs or orthologs having at least 70% identity to SEQ ID NO: 33;
   d) CvVioD comprised in SEQ ID NO: 31 or any of its paralogs or orthologs having at least 70% identity to SEQ ID NO: 31;
   e) CvVioE comprised in SEQ ID NO: 29 or any of its paralogs or orthologs having at least 70% identity to SEQ ID NO: 29;
   f) CvVioB-E fusion GGGGS3 linker comprised in SEQ ID NO:73 or any of its paralogs or orthologs having at least 70% identity to SEQ ID NO: 73;
   g) CvVioB-E fusion EAAAK3 linker comprised in SEQ ID NO: 75 or any of its paralogs or orthologs having at least 70% identity to SEQ ID NO: 75;
   h) BfXfpk comprised in SEQ ID NO: 1 or any of its paralogs or orthologs having at least 70% identity to SEQ ID NO: 1;
   i) CkPta comprised in SEQ ID NO: 3 or any of its paralogs or orthologs having at least 70% identity to SEQ ID NO: 3;
   j) EcAroL comprised in SEQ ID NO: 9 or any of its paralogs or orthologs having at least 70% identity to SEQ ID NO: 9; and
   k) BsPrs comprised in SEQ ID NO: 15 or any of its paralogs or orthologs having at least 70% identity to SEQ ID NO: 15.
136. The host cell according to any one of items 96-135, wherein the host cell is genetically engineered to produce one or more glycosyl transferases, such as one or more UDP-glucuronosyltransferases (UGT's).
137. The host cell according to item 136, wherein the one or more glycosyl transferases are configured for or capable of glycosylating the compound of formula (I).
138. The host cell according to any one of items 136-137, wherein the one or more glycosyl transferases have at least 70% sequence identity to the polypeptide sequence comprised in the sequence of Pt73Y according to SEQ ID NO: 66, such as at least 75%, such as at least 80%, such as at least 85%, such as at least 90%, such as at least 95%, such as 100%.
139. The host cell according to any one of items 136-138, wherein the one or more glycosyl transferases have at least 70% sequence identity to any one of the polypeptide sequences comprised in the sequence of: Pt73Y (SEQ ID: NO 64); (SEQ ID NO: 66); Bs109_1 (SEQ ID NO: 68); Bs109A1 (SEQ ID NO: 70); Cp73B (SEQ ID NO: 72); Cs73Y (yeast c/o) (SEQ ID NO: 92); Ha88B_2 (yeast c/o) (SEQ ID NO: 94); and/or Pt73Y (yeast c/o) (SEQ ID NO: 96).
140. The host cell according to any one of items 136-139, wherein the one or more glycosyl transferases produced by the host cell have at least 70% sequence identity to any one of the polypeptide sequences comprised in sequence of: Pt73Y (SEQ ID: NO 64); Cs73Y (yeast c/o) (SEQ ID NO: 92); and/or Pt73Y (yeast c/o) (SEQ ID NO: 96), such as at least 75%, such as at least 80%, such as at least 85%, such as at least 90%, such as at least 95%, such as 100%.
141. The host cell according to any one of items 136-140, wherein said host cell is a yeast, such as S. cerevisiae, and expresses the one or more glycosyl transferases.
142. The host cell according to any one of items 136-140, wherein said host cell is an E. coli, and expresses the one or more glycosyl transferases.
143. A cell culture, comprising a host cell as defined in any of items 96-142 and a growth medium.
144. The method of any items 1 to 95 further comprising:
   a) culturing the cell culture of item 143 at conditions allowing the host cell to produce the compound of formula (I); and
   b) optionally recovering and/or isolating the compound of formula (I).
145. The method of item 144, further comprising one or more elements selected from:
   a) culturing the cell culture in a nutrient growth medium;
   b) culturing the cell culture under aerobic or anaerobic conditions
   c) culturing the cell culture under agitation;
   d) culturing the cell culture at a temperature of between 25 to 50° C.;
   e) culturing the cell culture at a pH of between 3-9;
   f) culturing the cell culture for between 10 hours to 30 days; and
   g) culturing the cell culture under fed-batch, repeated fed-batch, continuous, or semi-continuous conditions.
146. The method of items 144 to 145, further comprising feeding one or more exogenous indoles of formula (II) to the cell culture.
147. The method of items 144 to 146, wherein the recovering and/or isolation step comprises separating a liquid phase of host cell or cell culture from a solid phase of host cell or cell culture to obtain a supernatant comprising the compound of formula (I) by one or more steps selected from:
   a) disrupting the host cell to release intracellular the compound of formula (I) into the supernatant;
   b) separating the supernatant from the solid phase of the host cell, such as by filtration or gravity separation;
   c) contacting the supernatant with one or more adsorbent resins in order to obtain at least a portion of the produced compound of formula (I);
   d) contacting the supernatant with one or more ion exchange or reversed-phase chromatography columns in order to obtain at least a portion of the compound of formula (I);
   e) extracting the compound of formula (I); and
   f) precipitating the compound of formula (I) by crystallization or evaporating the solvent of the liquid phase; and optionally isolating the compound of formula (I) by filtration or gravity separation;
   thereby recovering and/or isolating the compound of formula (I).
148. A fermentation liquid comprising the compound of formula (I) comprised in the cell culture of item 143.
149. The fermentation liquid of item 148, wherein at least 50%, such as at least 75%, such as at least 95%, such as at least 99% of the host cells are disrupted.
150. The fermentation liquid of item 148 to 149, wherein at least 50%, such as at least 75%, such as at least 95%, such as at least 99% of solid cellular material has separated from the liquid.
151. The fermentation liquid of item 148 to 150, further comprising one or more compounds selected from:
   a) precursors or products of the operative biosynthetic pathway producing the compound of formula (I);
   b) supplemental nutrients comprising trace metals, vitamins, salts, yeast nitrogen base, YNB, and/or amino acids; and
   wherein the concentration of the compound of formula (I) is at least 1 mg/I liquid.
152. A composition comprising the fermentation liquid of items 148 to 151 and/or the compound of formula (I) of items 89 to 95 and one or more agents, additives and/or excipients.
153. The composition of item 152, wherein the fermentation liquid and/or the compound of formula (I) have been processed into in a dry solid form, optionally in form of a powder.
154. The composition of item 152, wherein the composition is in a liquid form, optionally in a stabilized liquid form.
155. A method for modification of a microbial host cell producing the compound formula (I) as defined in item 1, comprising:
   a) Providing a microbial host cell, such as a yeast host cell, such as S. cerevisiae;
   b) Engineering the microbial host cell by inserting one or more genes encoding one or more of the enzymes as defined in items 1-71.
156. The method according to item 155, wherein the microbial host cell is as defined in any one of items 96-134.
157. A method for in-situ extraction of the compound of formula (I) or the glycosylated compound of formula (I), comprising:
   a) Providing a host cell as defined in any one of items 96-134 or the cell culture as defined in item 143 comprising the compound of formula (I) or the glycosylated compound of formula (I) in an aqueous phase;
   b) Subjecting the aqueous phase to extraction with an extractant, optionally wherein the extractant is a non-ionic surfactant, preferably wherein the extraction is performed during cultivation of the host cell.
158. The method according to item 157, wherein the method further comprises producing the compound of formula (I) using the method as defined in any one of items 1-88.
159. The method according to any one of items 157-158, wherein the extractant is a surfactant or a lipophilic extractant.
160. The method according to item 159, wherein the surfactant is a non-ionic or ionic surfactant.
161. The method according to item 159, wherein the extractant is a lipophilic extractant, preferably a non-toxic lipophilic extractant.
162. The method according to item 161, wherein the extractant is a lipophilic non-volatile extractant.
163. The method according to any one of items 161-162, wherein the lipophilic extractant is selected from the group consisting of: an ester, such as a C₂-C₂₀ ester, an alcohol, such as a C₂-C₂₀ alcohol, and a vegetable oil, such as grapeseed oil, olive oil, sunflower oil, or canola oil.
164. The method according to any one of items 157-160, wherein the extractant is subjected to the aqueous phase to form a liquid media with the aqueous phase such that the concentration of the extractant with respect to the liquid media is at least at the cloud-point of the extractant.
165. The method according to any one of items 157-160, wherein the extractant is subjected to the aqueous phase to form a liquid media with the aqueous phase such that the concentration of the extractant with respect to the liquid media is at least at the cloud-point of the extractant and below the toxicity level for the host cell, such as the LD50.
166. The method according to any one of items 157-165, wherein the extractant is (1,1,3,3-Tetramethylbutyl)phenyl-polyethylene glycol, Polyethylene glycol tert-octylphenyl ether (Triton X-114).
167. The method according to any one of items 157-165, wherein the extractant is selected from the group consisting of Antifoam-A, Triton-X 114, isopropyl myristate, isopropyl palmitate, polysorbate 20, ethyl laurate, castor oil, oleyl alcohol, butyl caprilate, grapeseed oil, 2-butyl-1-octanol, and oleic acid, or any combination thereof.
168. The method according to any one of items 157-165, wherein the extractant is polydimethylsiloxane (such as Antifoam A).
169. The method according to any one of items 157-165, wherein the extractant is isopropyl myristate.
170. The method according to any one of items 157-169, wherein the extractant is added such as to provide a concentration of the extractant of at least 1%, such as from 1-20%, such as from 1-2%, such as from 2-3%, such as from 3-4%, such as from 4-5%, such as from 5-6%, such as from 6-7%, such as from 7-8%, such as from 8-9%, such as from 9-10%, such as from 10-11%, such as from 11-12%, such as from 12-13%, such as from 13-14%, such as from 14-15%, such as from 15-16%, such as from 16-17%, such as from 17-18%, such as from 18-19%, such as from 19-20%.
171. The method according to any one of items 157-170, wherein the compound of formula (I) is isolated subsequent to extraction.
172. The method according to any one of items 157-171, wherein the method comprises one or more steps of:
   a. removing biomass by filtration or centrifugation from the aqueous phase or the extractant;
   b. separating and recovering the extractant comprising the compound of formula (I) or the glycosylated compound of formula (I) from the aqueous phase;
   c. separating and recovering the compound of formula (I) or the glycosylated compound of formula (I) from the extractant by precipitation;
   d. recovering the extractant.
173. The method according to item 172, wherein the step b of separating and recovering the extractant involves one or more steps of i) increasing the temperature, ii) adding one or more salts to the mixture of the aqueous phase and extractant, and/or iii) centrifuging the mixture.
174. The method according to item 172, wherein the step b of separating and recovering the extractant involves one or more steps of i) increasing the temperature, ii) adding one or more salts to the mixture of the aqueous phase and extractant, and/or iii) centrifuging the mixture, such that one or more of these steps moves the mixture above its cloud point.
175. The method according to any one of items 172-174, wherein the step c of separating and recovering the compound of formula (I) or the glycosylated compound of formula (I) involves one or more steps of i) lowering the temperature, ii) adding an alcohol to the extractant, such as ethanol, and/or iii) centrifuging the extractant.
176. The method according to any one of items 172-175, wherein the step d of recovering the extractant involves one or more steps of i) increasing the temperature, ii) evaporating the alcohol, such as ethanol, iii) adding one or more salts to the extractant, and/or iv) centrifuging.
177. The method according to any one of items 172-176, wherein the removing of biomass by centrifugation in step a is performed at room temperature.
178. The method according to any one of items 172-177, wherein the extractant is a non-ionic surfactant.
179. The method according to item 178, wherein the non-ionic surfactant is selected from the group consisting of: antifoam-A, Triton and polysorbate 20.
180. The method according to item 173 or 174, wherein the salt added in step ii is a sulfate salt, such as Na₂SO₄.
181. The method according to item 175, wherein the alcohol added in step ii is ethanol at a final concentration of from 15% to 30%, such as 25%.
182. The method according to any one of items 172-176, wherein the precipitation of the compound of formula (I) or the glycosylated compound of formula (I) in step c is accelerated by centrifuging at room temperature.
183. The method according to any one of items 172-176, wherein after the step c, the precipitated compound of formula (I) or the glycosylated compound of formula (I) is resuspended in ethanol and subjected to evaporation to remove ethanol.
184. The method according to item 183, wherein the remaining solution after evaporation is further subjected to freeze drying to obtain a dried form of the compound of formula (I) or the glycosylated compound of formula (I).
185. The method according to any one of items 172-176, wherein the step d of recovering the extractant comprises evaporating the ethanol using a vacuum centrifuge.
186. The method according to any one of items 172-176, wherein after the step d, the method involves using the recovered extractant in subsequent extractions.
187. The method according to any one of items 172-186, wherein the method further comprises cultivating the host cell in a growth medium.
188. The method according to item 187, further comprising the steps of i) separating the cultivation into distinct phases comprising a biomass phase, an aqueous phase, and an extractant phase; and subsequently ii) collecting the extractant phase comprising the compound of formula (I) or the glycosylated compound of formula (I).
189. The method according to any one of items 187-188, wherein the extractant is added to a final concentration of from 6 to 14%, such as from 8 to 12%, for example 10%.
190. The method according to any one of items 187-189, wherein the host cell is cultivated for one or more days, such as from 2 to 7 days, for example 3 to 6 days, such as 4 days, wherein the host cell is cultivated at from 25 to 40° C., such as from 25 to 38° C., such as from 26 to 36° C., such as from 28 to 34° C., for example 30° C.
191. The method according to any one of items 187-190, wherein the extractant is at least one of isopropyl myristate, isopropyl palmitate, antifoam-A, polysorbate, ethyl laurate, and castor oil.
192. The method according to any one of items 187-190, wherein the compound of formula (I) is violacein or deoxyviolacein and the extractant is at least one of Antifoam-A, isopropyl myristate, isopropyl palmitate, ethyl laurate, grapeseed oil, 2-butyl-1-octanol, and oleic acid.
193. The method according to any one of items 157-192, wherein the extractant comprising the compound of formula (I) or the glycosylated compound of formula (I) is loaded onto dry silica to provide an extractant bound to silica.
194. The method according to item 193, wherein the dry silica has a pore size ranging from 50 A to 70 A, preferably 60 A, and a particle size ranging from 0.4 mm to 1.2 mm, more preferably 0.5-1 mm.
195. The method according to item 193 or 194, wherein the binding to silica is done at a weight-to-weight ratio ranging from 0.8:1 to 1.2:1, with a preferable ratio being 1:1 (w/w).
196. The method according to any one of items 193-195, wherein the extractant bound to silica is washed with a volatile solvent one or more times to remove the extractant.
197. The method according to item 196, wherein the volatile solvent is selected from the group consisting of dichloromethane, hexane, and ethyl acetate.
198. The method according to any one of items 193-197, wherein the method further comprises a step of eluting the compound of formula (I) or the glycosylated compound of formula (I) from the silica using a polar protic solvent, such as an alcohol, for example ethanol.
199. The method according to item 198, wherein the method further comprises a step of evaporating the polar protic solvent used in elution to obtain the compound of formula (I) or the glycosylated compound of formula (I) in solid form.
200. The method according to any one of items 157-192, wherein purification of the compound of formula (I) or the glycosylated compound of formula (I) is done using column chromatography.
201. The method according to item 200, wherein the column chromatography is gravity or peristaltic pump driven.
202. The method according to any one of items 200-201, wherein the elution is done using a mixture comprising ethyl acetate in hexane.
203. The method according to any one of items 157-202, wherein the recovered compound of formula (I) or the glycosylated compound of formula (I) has a residual solvent concentration below 0.1%.
204. The method according to any one of items 157-203, wherein the compound of formula (I) is violacein, deoxyviolacein, proviolacein, or prodeoxyviolacein, for example deoxyviolacein.
205. The method according to any one of items 157-204, further comprising the steps of:
   a. collecting the extractant comprising the compound of formula (I) or the glycosylated compound of formula (I),
   b. subsequently diluting the extractant with an alcohol, such as ethanol to a predefined concentration of the extractant with respect to the alcohol to provide a mixture of extractant and alcohol, and
   c. cooling the mixture of extractant and alcohol to a preset temperature, optionally under stirring, to solidify the extractant thereby increasing the concentration of the compound of formula (I) or the glycosylated compound of formula (I) in the alcohol.
206. The method according to item 205, further comprising a step of filtration, such that solidified extractant is removed, optionally at the preset temperature.
207. The method according to item 206, further comprising a step of evaporating the alcohol to provide the compound of formula (I) or the glycosylated compound of formula (I) in concentrated form relative to the concentration of the compound of formula (I) or the glycosylated compound of formula (I) in the extractant collected in step a of item 205, optionally wherein the concentrated form is a paste.
208. The method according to any one of items 205-207, wherein the predefined concentration of the extractant with respect to the alcohol is from 20 to 40% extractant, such as from 20 to 21%, such as from 21 to 22%, such as from 22 to 23%, such as from 23 to 24%, such as from 24 to 25%, such as from 25 to 26%, such as from 26 to 27%, such as from 27 to 28%, such as from 28 to 29%, such as from 29 to 30%, such as from 30 to 31%, such as from 31 to 32%, such as from 32 to 33%, such as from 33 to 34%, such as from 34 to 35%, such as from 35 to 36%, such as from 36 to 37%, such as from 37 to 38%, such as from 38 to 39%, such as from 39 to 40%, for example 33%.
209. The method according to any one of items 205-208, wherein the preset temperature is at the solidification temperature (melting point) of the extractant or less.
210. The method according to any one of items 205-208, wherein the preset temperature is 20° C. or less, such as 19° C. or less, such as 18° C. or less, such as 17° C. or less, such as 16° C. or less, such as 15° C. or less, such as 14° C. or less, such as 13° C. or less, such as 12° C. or less, such as 11° C. or less, such as 10° C. or less, such as 9° C. or less, such as 8° C. or less, such as 7° C. or less, such as 6° C. or less, such as 5° C. or less, such as 4° C. or less, such as 3° C. or less, such as 2° C. or less, such as 1° C. or less, such as 0° C. or less, such as −1° C. or less, such as −2° C. or less, such as −3° C. or less, such as −4° C. or less, such as −5° C. or less, such as −6° C. or less, such as −7° C. or less, such as −8° C. or less, such as −9° C. or less, such as −10° C. or less.
211. The method according to any one of items 205-208, wherein the preset temperature is from 20° C. to −5° C., such as from 19° C. to −5° C., such as from 18° C. to −5° C., such as from 17° C. to −5° C., such as from 16° C. to −5° C., such as from 15° C. to −5° C., such as from 14° C. to −5° C., such as from 13° C. to −5° C., such as from 12° C. to −5° C., such as from 11° C. to −5° C., such as from 10° C. to −5° C., such as from 9° C. to −5° C., such as from 8° C. to −5° C., such as from 7° C. to −5° C., such as from 6° C. to −5° C., such as from 5° C. to −5° C.
212. A method for dyeing a textile material, comprising:
   a. providing an optionally dried composition of one or more compounds as defined in any one of items 89-95, for example violacein, proviolacein, prodeoxyviolacein, and/or deoxyviolacein; and subsequently preparing a dye solution by suspending said composition in a liquid, such as an alcohol, for example ethanol; or
   b. providing a colored fermentation extract comprising an extractant and one or more compounds as defined in any one of items 89-95, for example violacein, proviolacein, prodeoxyviolacein, and/or deoxyviolacein
   c. contacting a textile material with said dye solution or said colored fermentation extract, optionally for a predetermined duration, thereby dyeing the textile material.
213. The method according to item 212, further comprising a step d) of removing the textile material from said dye solution or colored fermentation extract and washing with water to remove any excess dye.
214. The method according to item 213, further comprising a step e) of drying the dyed textile material without the use of pre-treatments, mordants, or other chemical processing steps, and wherein the textile material retains a color change indicative of dyeing.
215. The method according to any one of items 212-214, wherein the liquid is at least 90% ethanol, such as 100% ethanol.
216. The method according to any one of items 212-215, wherein the textile material is selected from the group consisting of nylon 6,6, diacetate, polyester, cotton, such as bleached cotton, wool, hemp rayon, denim, viscose, and silk.
217. The method according to any one of items 212-216, wherein the predetermined duration is from 10 minutes to 2 hours, such as 30 minutes.
218. The method according to any one of items 212-217, wherein the dyeing imparts both color and bioactivity to the textile material.
219. The method according to any one of items 212-218, wherein the composition is derived from the host cell as defined in any one of items 96-135.
220. The method according to any one of items 212-218, wherein the composition is in the form of a purified fermentation extract.
221. The method according to any one of items 212-218, wherein the colored fermentation extract is obtainable by the method as defined in any one of items 157-211.
222. The method according to any one of items 212-220, further comprising providing a colored fermentation extract using the method as defined in any one of items 157-211, wherein the method further comprises a step of diluting the colored fermentation extract in a liquid to provide a dye bath.
223. The method according to item 222, wherein the method comprises diluting the colored fermentation extract to an extractant concentration of from 2% to 30%, such as from 2 to 4%, such as from 4 to 6%, such as from 6 to 8%, such as from 8 to 10%, such as from 10 to 12%, such as from 12 to 14%, such as from 14 to 16%, such as from 16 to 18%, such as from 18 to 20%, such as from 20 to 22%, such as from 22 to 24%, such as from 24 to 26%, such as from 26 to 28%, such as from 28 to 30%, for example to a concentration of 10% extractant in 90% of the liquid.
224. The method according to any one of items 222-223, wherein the liquid is a polar protic solvent, such as an alcohol or water, for example ethanol.
225. The method according to any one of items 222-223, wherein the extractant is a lipophilic non-volatile solvent.
226. The method according to any one of items 222-223, wherein the extractant is selected from the group consisting of: Antifoam-A, Triton-X 114, isopropyl myristate, isopropyl palmitate, polysorbate, ethyl laurate, castor oil, oleyl alcohol, butyl caprilate, grapeseed oil, 2-butyl-1-octanol, and oleic acid, for example isopropyl myristate.
227. The method according to any one of items 212-226, wherein the method comprises diluting the colored fermentation extract with water, optionally at room temperature, until a single phase is produced between the colored fermentation extract and the water.
228. The method according to item 227, wherein the method comprises dilution until the concentration of the extractant is below its cloud point at room temperature.
229. The method according to any one of items 227-228, wherein the method provides a colored aqueous suspension.
230. The method according to item 229, wherein the method comprises dyeing textile material by contacting the textile material with the aqueous suspension and incubating at room temperature.
231. The method according to any one of items 227-230, wherein the extractant is a non-ionic surfactant, such as Antifoam-A.
232. The method according to any one of items 227-231, wherein the textile material is selected from the group consisting of: nylon 6,6, diacetate, polyester, cotton, such as bleached cotton, wool, hemp rayon, denim, viscose, and silk, for example nylon 6,6.
233. The method according to any one of items 212-232, wherein the liquid is water and the one or more compounds are glycosides as defined in any one of items 91-95.
234. The method according to item 233, wherein the compound is a glycoside of violacein or proviolacein.
235. The method according to item 234, further comprising the steps of:
   a. incubating the textile material in a dye bath comprising the one or more compounds in any one of items 91-95 and water; and
   b. adding a glucosidase, such as a beta-glucosidase to the dye bath to de-glycosylate the one or more compounds thereby providing a dyed textile material.
236. The method according to item 235, wherein the method comprises incubating for from 15 minutes to 24 hours, such as from 15 minutes to 30 minutes, such as from 30 minutes to 45 minutes, such as from 45 minutes to 1 hour, such as from 1 hour to 2 hours, such as from 2 hours to 3 hours, such as from 3 hours to 4 hours, such as from 4 hours to 5 hours, such as from 5 hours to 6 hours, such as from 6 hours to 7 hours, such as from 7 hours to 8 hours, such as from 8 hours to 9 hours, such as from 9 hours to 10 hours, such as from 10 hours to 11 hours, such as from 11 hours to 12 hours, such as from 12 hours to 13 hours, such as from 13 hours to 14 hours, such as from 14 hours to 15 hours, such as from 15 hours to 16 hours, such as from 16 hours to 17 hours, such as from 17 hours to 18 hours, such as from 18 hours to 19 hours, such as from 19 hours to 20 hours, such as from 20 hours to 21 hours, such as from 21 hours to 22 hours, such as from 22 hours to 23 hours, such as from 23 hours to 24 hours, preferably at room temperature.
237. The method according to any one of items 235-236, wherein the method further comprises washing the dyed textile material after step b.
238. A method for dyeing textile material in a growth medium, comprising:
   a. Cultivating a microbial host cell as defined in any one of items 96-135 in a growth medium;
   b. Adding textile material to the growth medium to provide a dyed textile material comprising a compound of formula (I), optionally for a predefined duration, optionally during the cultivation process.
239. The method according to item 238, wherein the microbial host cell is cultivated at from 25 to 35° C., such as 30° C. for a number of days, such as for from 2 to 8 days, such as 4 days.
240. The method according to item 238, wherein the method further comprises a step of sterilizing the textile material prior to step b, such as by adding the textile material into an alcohol or a solution of alcohol in water, for example ethanol, such as 75% ethanol in water.
241. The method according to any one of items 238-240, wherein the method further comprises a step
   c) of recovering the dyed textile material from the growth medium.
242. The method according to any one of items 144-241, wherein the host cell is of a strain selected from the group consisting of SC-139, SC-141, SC-144, and SC-145.
243. The method according to any one of items 238-242, wherein the textile material is dyed without requiring additional downstream processing, pre-treatments, mordants, and/or other chemicals.
244. The method according to any one of items 238-243, comprising a step of extracting the compound of formula (I) from the growth medium by recovering the dyed textile material from the growth medium.
245. The method according to any one of items 238-244, wherein the textile material is selected from the group consisting of diacetate, bleached cotton, nylon 6,6, polyester, acrylic, and wool.
246. The method according to any one of items 238-245, further comprising a step of washing the dyed textile material with water post-cultivation.
247. A dyed textile material comprising the compound as defined in any one of items 89-95.
248. The dyed textile material according to item 247, wherein the textile material is selected from the group consisting of: Nylon 6,6, Diacetate, Bleached cotton, Polyester, Wool, and Acrylic.
249. The dyed textile material according to any one of items 247-248, wherein the compound is selected from the group consisting of: deoxyviolacein, violacein, prodeoxyviolacein, proviolacein, or a combination thereof.
250. The dyed textile material according to any one of items 247-249 obtainable by the method of any one of items 212-246.
251. The dyed textile material according to any one of items 247-250, wherein the dyed textile material is antimicrobial, i.e. has antimicrobial activity.
252. A method of colouring a beverage, comprising:
   a. providing an optionally dried composition of one or more compounds as defined in any one of items 91-95, for example glycosylated violacein, glycosylated proviolacein, glycosylated prodeoxyviolacein, and/or glycosylated deoxyviolacein; and optionally subsequently preparing a dye solution by suspending said composition in a liquid, such as an alcohol or water; and
   b. contacting a beverage with said dye solution or said composition, optionally for a predetermined duration, thereby colouring the beverage.
253. A method for enhancing the antimicrobial properties of a textile material, such as clothing or a wound dressing, or a beverage, comprising dyeing the textile material or colouring the beverage with the compound as defined in any one of items 89-95, or with an extractant comprising the compound thereby enhancing the antimicrobial properties of the textile material or beverage.
254. A method for enhancing the antioxidant properties of a textile material, such as clothing, or a beverage comprising dyeing the textile material or colouring the beverage with the compound as defined in any one of items 89-95, or with an extractant comprising the compound thereby enhancing the antioxidant properties of the textile material or beverage.
255. A method for enhancing the UV resistance of a textile material, such as clothing, or of a beverage comprising dyeing the textile material or colouring the beverage with the compound as defined in any one of items 89-95, or with an extractant comprising the compound thereby enhancing the UV resistance of the textile material or beverage.
256. A beverage comprising the comprising the compound as defined in any one of items 91-95.
257. A nanocellulose comprising a compound as defined in any one of items 89-95.
258. The nanocellulose of item 257, wherein the nanocellulose is selected from the group consisting of bacterial nanocellulose (BNC), nanofabricated cellulose (NFC), cellulose nanocrystals (CNC), cellulose nanofibrils (CNF), and electrospun cellulose nanofibers.
259. The nanocellulose of item 258, wherein the nanocellulose is bacterial nanocellulose (BNC) or nanofabricated cellulose (NFC).
260. The nanocellulose according to any one of items 257-259, further comprising a non-ionic surfactant, such as Triton-X 100, Tween 20, sodium dodecyl sulfate (SDS), or polyvinyl alcohol (PVA).
261. The nanocellulose according to any one of items 257-260, wherein the nanocellulose is derived from a microbial culture.
262. The nanocellulose according to item 261, wherein the microbial culture comprises one or more of Acetobacter xylinum, Gluconacetobacter hansenii, and Komagataeibacter medellinensis.
263. The nanocellulose according to any one of items 257-262, wherein the nanocellulose is derived from a Kombucha starter culture.
264. The nanocellulose according to any one of items 257-262, wherein the nanocellulose is derived from a Kombucha starter culture comprising green tea and sucrose.
265. The nanocellulose of any one of items 257-263, wherein the nanocellulose further comprises an additive selected from the group consisting of starch, lignin, and chitosan.
266. The nanocellulose of any one of items 257-265, wherein the compound is selected from the group consisting of: violacein, proviolacein, deoxyviolacein, and prodeoxyviolacein.
267. The nanocellulose of any one of items 257-265, wherein the compound is deoxyviolacein and the nanocellulose is NFC.
268. The nanocellulose of any one of items 257-267, wherein the nanocellulose is in a form selected from the group consisting of a hydrogel, an aerogel, and a film.
269. A method for dyeing nanocellulose, comprising
   a. providing a compound as defined in any one of items 89-95, optionally in a dye bath comprising an alcohol and optionally a surfactant;
   b. providing nanocellulose, such as bacterial nanocellulose (BNC) or nanofabricated cellulose (NFC);
   c. incubating the cellulose with the compound, optionally in the dye bath, at a predefined temperature until the nanocellulose takes on the color of the compound, thereby providing dyed nanocellulose.
270. The method of item 269, wherein the predefined temperature is from 20 to 50° C.
271. The method of item 269, wherein the predefined temperature is room temperature.
272. The method of any one of items 269-270, wherein the dye bath comprises from 70 to 95% alcohol in non-ionic surfactant, such as 90%.
273. The method of any one of items 269-272, wherein the alcohol is ethanol.
274. The method of any one of items 269-273, wherein the extractant is selected from the group consisting of isopropyl myristate, Triton-X 100, Tween-20, and Tween-80.
275. The method of any one of items 269-274, wherein the nanocellulose is selected from the group consisting of bacterial nanocellulose (BNC), nanofabricated cellulose (NFC), cellulose nanocrystals (CNC), cellulose nanofibrils (CNF), and electrospun cellulose nanofibers.
276. The method of any one of items 269-275, wherein the nanocellulose is selected from the group consisting of bacterial nanocellulose (BNC), and nanofabricated cellulose (NFC).
277. The method of any one of items 269-275, further comprising a step of: d. drying the dyed nanocellulose at room temperature.
278. The method of any one of items 269-277, wherein the compound is provided in a dye bath, and wherein the dye bath further comprises a non-ionic surfactant at a concentration of approximately 0.01%, for example Triton-X 100.
279. A dyed product comprising the nanocellulose of any one of items 257-268.
280. The dyed product according to item 279, wherein the product is selected from the group consisting of: a wound healing product, such as a wound dressing, a food packaging, a cosmetic product, a textile fiber, a bio-based paint, a paper, and a textile dye.
281. A method for dyeing a product, comprising:
   a. Providing a nanocellulose as defined in any one of items 257-268;
   b. Providing a product;
   c. Contacting the nanocellulose with the product, optionally incubating the product with the nanocellulose for a duration.
282. The method according to item 281, wherein the product is selected from the group consisting of: a wound healing product, such as a wound dressing, a food packaging, a cosmetic product, a textile fiber, a bio-based paint, a paper, and a textile dye.
283. The method according to item 281, wherein the product is paper and the nanocellulose comprises NFC.
284. A method of producing a dye bath, the method comprising the steps of:
   a. cultivating a host cell as defined in any one of items 96-142 in growth medium to produce the compound as defined in any one of items 89-95, such as engineered S. cerevisiae production strains producing at least one of violacein, deoxyviolacein, prodeoxyviolacein, and proviolacien;
   b. adding an extractant to the growth medium thereby providing a compound enriched extractant;
   c. optionally collecting the compound enriched extractant and adding further extractant to the growth medium;
   d. optionally repeating step c a number of times to provide a collection of compound enriched extractants,
   e. diluting the compound enriched extractant or the collection of compound enriched extractants with a liquid, such as an organic solvent, thereby providing a dye bath.
285. The method according to item 284, wherein the extractant is selected from the group consisting of: isopropyl myristate, Antifoam-A, Triton-X 114, isopropyl palmitate, polysorbate, ethyl laurate, castor oil, oleyl alcohol, butyl caprilate, grapeseed oil, 2-butyl-1-octanol, and oleic acid, or any combination thereof.
286. The method according to item 285, wherein the extractant is isopropyl myristate.
287. The method according to any one of items 284-286, wherein the liquid is ethanol.
288. A dye bath obtainable using the method of any one of items 284-287.
289. A method for dyeing a product, comprising the steps of:
   a. adding a product to a dye bath comprising a compound of formula (I) as defined in any one of items 89-95, and a liquid and optionally an extractant;
   b. optionally pre/post-treating the product to modify its pH;
   c. optionally dyeing the product at a predetermined temperature for a predetermined time to obtain a dyed product, optionally in a dyeing machine;
   d. washing the dyed product with water; and
   e. optionally drying the product.
290. The method of item 289, wherein the product is selected from the group consisting of: a fabric, a fiber, a yarn, a textile, a filament, a weave, a non-woven material, a twill, a felt, a lace, a mesh, a cord, a tapestry, a tuft, and a batting; for example a fabric, a fiber, or a yarn.
291. The method according to any one of items 289-290, wherein the product comprises a material selected from the group consisting of nylon 6,6, diacetate, polyester, cotton, such as bleached cotton, wool, hemp rayon, denim, viscose, and silk.
292. The method of any one of items 289-291, wherein the predetermined temperature is from 15 to 50° C., such as from 20 to 35° C., for example about 23° C.
293. The method of any one of items 289-292, wherein the predetermined temperature is from 80 to 180° C., such as from 85 to 170° C., such as from 90 to 160° C., such as from 95 to 155° C., such as from 100 to 150° C., such as from 105 to 145° C., such as from 110 to 140° C., for example 130° C.
294. The method of any one of items 289-293, wherein the predetermined temperature is from 80 to 180° C., such as from 85 to 170° C., such as from 90 to 160° C., such as from 95 to 155° C., such as from 100 to 150° C., such as from 105 to 145° C., such as from 110 to 140° C., for example 130° C.; and wherein the product comprises polyester.
295. The method of any one of items 289-294, wherein the predetermined time is from 5 minutes to 360 minutes, such as for 10 minutes to 60 minutes, for example 15 minutes.
296. The method of any one of items 289-295, wherein the final concentration of the extractant in the dye bath is less than 70%, such as less than 69%, such as less than 68%, such as less than 67%, such as less than 66%, such as less than 65%, such as less than 64%, such as less than 63%, such as less than 62%, such as less than 61%, such as less than 60%, such as less than 59%, such as less than 58%, such as less than 57%, such as less than 56%, such as less than 55%, such as less than 54%, such as less than 53%, such as less than 52%, such as less than 51%, such as less than 50%.
297. The method of any one of items 289-296, wherein the final concentration of the extractant in the dye bath is from 20 to 70%, such as from 30 to 69%, such as from 35 to 68%, such as from 40 to 67%, for example from 45 to 66%.
298. The method of any one of items 212-297, further comprising a step of adding a dispersing agent, such as a soap, for example dish soap.
299. The method of item 298, wherein the dispersing agent is selected from the group consisting of: an anionic surfactant, such as sodium dodecyl sulfate or alkylbenzene sulfonate; a cationic surfactant, such as a quaternary ammonium compound; a non-ionic surfactant, such as an ethoxylated alcohol, an alkylphenol, or a polysorbate; a zwitterionic surfactant, such as cocamidopropyl betaine; a polysaccharide, a cellulose derivative, such as carboxymethylcellulose, or hydroxyethylcellulose; a protein, such as casein, a gum, such as xanthan gum, guar gum, or acacia gum, and lecithin.
300. The method of any one of items 298-299, wherein the dispersing agent is added to provide a final concentration of from 0.05 to 3 g/L, for example from 1 to 2 g/L.
301. A method of recycling a used dye bath, comprising the steps of:
   a. subjecting a used dye bath comprising i) a liquid, ii) an extractant, and iii) a compound of formula (I) as defined in any one of items 89-95 to evaporation, optionally in vacuo to remove the liquid, wherein the dye bath has been used for dyeing a product;
   b. passing the remaining extractant and compound from step a through silica to obtain a recycled dye bath.

### EXAMPLES

### Materials and methods

### Materials

Chemicals used in the examples herein e.g. for buffers and substrates are commercial products of at least reagent grade.

### Background Strains

BY4741 is a common strain of S. cerevisiae derived from S288C and available e.g. from American Type Culture Collection (ATCC #200885). DH5a and XJb (DE3) are common strains of E. coli available from E.g. Zymo Research.

### Example 1—Construction of Genetically Modified S. cerevisiae Strains for De Novo Production of Violacein and Violacein Derivatives

S. cerevisiae was first genetically engineered to produce violacein and related derivatives in 3 broad steps. In step 1 wild-type S. cerevisiae was engineered to produce a related product tryptamine. While Tryptophan is the native precursor to violacein, engineering its (over-)production in S. cerevisiae is complicated by the fact that i) tryptophan is minimally exported from the cell and ii) S. cerevisiae has efficient metabolic mechanisms to prevent tryptophan accumulation by converting tryptophan to other degradation products. To overcome this issue tryptamine was used as a proxy molecule since it i) is efficiently transported across the cell membrane, ii) is not further metabolized by S. cerevisiae to unwanted degradation products, and iii) the enzyme converting tryptophan to tryptamine (tryptophan decarboxylase CrTdc (SEQ ID NO: 35, 36) is known to work very efficiently in S. cerevisiae. A variety of metabolic engineering strategies (outlined in Table 1 and 2) were employed to increase tryptamine production in S. cerevisiae leading to the generation of tryptamine producing strain SC-106.

In step 2 the biosynthetic pathway to violacein (and its natural derivatives) was introduced into SC-106. The integration cassettes were designed so that integration of the violacein biosynthetic genes took place at the site where tryptophan decarboxylase CrTdc was integrated into the genome, thereby facilitating the simultaneous removal of CrTdc and integration of the target biosynthetic genes. Different combinations of genes from the violacein biosynthetic pathway were integrated into the genome to produce the different violacein derivatives.

In step 3 improved production of violacein and its derivatives was realized through a series of genetic modifications aimed at improving flux specifically through the violacein branch of the pathway and improving the production of co-factors required for violacein biosynthesis.

TABLE 1
Overview of genetic strategies employed to produce violacein
and natural violacein derivatives in S. cerevisiae

| Modification | Gene | | |
|---|---|---|---|
| type | name | SEQ ID NO's | Purpose |
| Native gene | ARO1 | SEQ ID NO: 7, 8 | Boost flux through native tryptophan pathway |
| overexpression | ARO2 | SEQ ID NO: 11, 12 | Boost flux through native tryptophan pathway |
| | TRP4 | SEQ ID NO: 17, 18 | Boost flux through native tryptophan pathway |
| | TRP1 | SEQ ID NO: 19, 20 | Boost flux through native tryptophan pathway |
| | TRP3 | SEQ ID NO: 21, 22 | Boost flux through native tryptophan pathway |
| | TRP5 | SEQ ID NO: 23, 24 | Boost flux through native tryptophan pathway |
| | TAL1 | SEQ ID NO: 37, 38 | Pull flux through non-oxidative pentose |
| | | | phosphate pathway |
| | TKL1 | SEQ ID NO: 39, 40 | Pull flux through non-oxidative pentose |
| | | | phosphate pathway |
| | RIB1 | SEQ ID NO: 41, 42 | Overcome rate-limiting step in FAD |
| | | | biosynthesis pathway |
| | FLX1 | SEQ ID NO: 43, 44 | Improve transport of FAD from mitochondria |
| | | | to cytosol |
| | POS5 | SEQ ID NO: 50, 51 | Convert NADH to NADPH, improve NADPH |
| | | | supply |
| | HEM3 | SEQ ID NO: 46, 47 | Overcome rate-limiting step in heme |
| | | | biosynthesis pathway |
| Modified native | ARO4(K229L) | SEQ ID NO: 5, 6 | Boost flux through native tryptophan pathway |
| gene | TRP2(S65R, | SEQ ID NO: 13, 14 | Boost flux through native tryptophan pathway |
| overexpression | S76L) | | |
| Heterologous | CvVioA | SEQ ID NO: 25, 26 | Violacein biosynthesis pathway |
| gene | CvVioB | SEQ ID NO: 27, 28 | Violacein biosynthesis pathway |
| overexpression | CvVioC | SEQ ID NO: 33, 34 | Violacein biosynthesis pathway |
| | CvVioD | SEQ ID NO: 31, 32 | Violacein biosynthesis pathway |
| | CvVioE | SEQ ID NO: 29, 30 | Violacein biosynthesis pathway |
| | CrTdc | SEQ ID NO: 35, 36 | Divert tryptophan flux to temporary proxy |
| | | | product |
| | BfXfpk | SEQ ID NO: 1, 2 | Increase E4P supply |
| | CkPta | SEQ ID NO: 3, 4 | Increase E4P supply |
| | EcAroL | SEQ ID NO: 9, 10 | Overcome rate limiting step in shikimate |
| | | | pathway |
| | BsPrs | SEQ ID NO: 15, 16 | Improve PRPP supply |
| Native gene | ARO10 | SEQ ID NO: 49 | Remove tryptophan degradation pathway |
| deletions | PDC5 | SEQ ID NO: 48 | Remove tryptophan degradation pathway |
| | UBP3 | SEQ ID NO: 57 | Decrease regulation to increase pathway flux |
| | RIC1 | SEQ ID NO: 58 | Decrease regulation to increase pathway flux |
| | GPP1 | SEQ ID NO: 59 | Reduce toxic acetate formation |
| | ROX1 | SEQ ID NO: 60 | Increase heme biosynthesis |
| | HMX1 | SEQ ID NO: 45 | Decrease heme degradation |
| | HAP1 | SEQ ID NO: 61 | Increase heme biosynthesis |

TABLE 2
Overview of additional genetic strategies employed to produce violacein
and natural violacein derivatives in S. cerevisiae

| Modification | | SEQ ID | |
|---|---|---|---|
| type | Gene name | NO's | Purpose |
| Overexpression | CvVioB-E fusion | 75, 76 | Fusing biosynthetic |
| | EAAAK3 linker | | enzymes together |
| | CvVioB-E fusion | 73, 74 | to reduce by-product |
| | GGGGS3 linker | | formation |

Genes for the biosynthetic pathway were integrated into pre-defined genomic “landing pads” using custom-made overexpression plasmids similar to the system described by (Mikkelsen et al., 2012). Linear integration fragments are produced by NotI digestion of custom designed plasmids containing strong constitutive S. cerevisiae promoters and terminators and are flanked by upstream and downstream homology regions to facilitate assembly by homologous recombination. To facilitate assembly of multiple integration plasmids at a single genomic locus, upstream and downstream homology arms are designed so that after NotI digestion (New England Bio Labs Inc.), linear integration fragments can recombine into a single linear integration fragment and integrate in the target genomic loci. To select for transformants that have successfully integrated the fragments of interest, an endonuclease such as MAD7 can be used as described above or alternatively a selection marker such as LEU2 or URA3 can be incorporated into the linear integration fragments and transformed into S. cerevisiae strains that are auxotrophic for Leucine as is known in the art. To reduce the occurrence of false positives the selection marker can be split across 2 linear integration fragments such that a functional LEU2 or URA3 selection marker can only be generated upon successful homologous recombination. Integration cassettes consisting of a gene of interest flanked by strong constitutive promoters and terminators and finally flanked by homologous recombination arms were constructed in a manner similar to the MoClo golden gate assembly system described by (Michael E. Lee, 2015). Genes were codon-optimized for expression in S. cerevisiae and synthesized and cloned into custom integration plasmids by Twist Biosciences (Table 3). After linearization by restriction digestion with NotI (New England Bio Labs Inc.) plasmids are transformed into S. cerevisiae according to (Gietz & Woods, 2002). Transformants are plated on selective media. In some cases, genes were overexpressed in S. cerevisiae by introduction of a self-replicating plasmid with a selectable marker. In these cases genes were synthesized by Twist and cloned directly into the expression plasmid (e.g. p416-TEF). Plasmids were directly transformed into S. cerevisiae and selected on media lacking the appropriate amino acid.

TABLE 3
Integration plasmids used to construct violacein and violacein
derivatives producing S. cerevisiae strains.

| Plasmid name | SEQ ID NO. |
|---|---|
| PL-478(Rec2-URA: BbXfpk) | SEQ ID NO: 1, 2 |
| PL-479(Rec1-XII-1-URA: CkPta) | SEQ ID NO: 3, 4 |
| PL-480(Rec5-XII-1: BsPrs) | SEQ ID NO: 15, 16 |
| PL-481(Rec4: ARO1) | SEQ ID NO: 7, 8 |
| PL-482(Rec5-XI-5: ARO2) | SEQ ID NO: 11, 12 |
| PL-483(Rec1-XI-5-LEU2: CrTdc) | SEQ ID NO: 35, 36 |
| PL-484(Rec3: EcAroL) | SEQ ID NO: 9, 10 |
| PL-486(Rec2-LEU: ARO4 (K229L)) | SEQ ID NO: 5, 6 |
| PL-487(Rec3: TRP2 (S65R, S76L)) | SEQ ID NO: 13, 14 |
| PL-606(Single Int-XII-2-SpHIS5: TRP4) | SEQ ID NO: 17, 18 |
| PL-646(MoClo: RIC1 Up-TRP2 (S65R, S76L)-Split KlUra3 + DR L) | SEQ ID NO: 13, 14, 58 |
| PL-647(MoClo: Split KlUra3 + DR R-ARO4 (K229L)-RIC1 Down) | SEQ ID NO: 5, 6, 58 |
| PL-660(MoClo: X-3-TRP1-Split KlUra + DR L) | SEQ ID NO: 19, 20 |
| PL-661(MoClo: Split KlUra3 + DR R-TRP3-X-3) | SEQ ID NO: 21, 22 |
| PL-908(MoClo: XI-5 int-CvVioA-split KlUra3 + DR L) | SEQ ID NO: 25, 26 |
| PL-909(MoClo: split KlUra3 + DR R-CvVioB-B hom) | SEQ ID NO: 27, 28 |
| PL-910(MoClo: B hom-VioE-C hom) | SEQ ID NO: 29, 30 |
| PL-911(MoClo: B hom-VioE-Rec1 Leu2 hom) | SEQ ID NO: 29, 30 |
| PL-912(MoClo: D hom-VioD-Rec1 Leu2 hom) | SEQ ID NO: 31, 32 |
| PL-915(MoClo: C hom-VioC-D hom) | SEQ ID NO: 33, 34 |
| PL-946(MoClo: XII-5 int-VioC-KlUra3-XII-5) | SEQ ID NO: 33, 34 |
| PL-950(MoClo: XII-5 int-VioD-KlUra3-XII-5) | SEQ ID NO: 31, 32 |
| PL-1354(MoClo: X-4 int-TRP5-A hom) | SEQ ID NO: 23, 24 |
| PL-1355(MoClo: A hom-TAL1-B hom) | SEQ ID NO: 37, 38 |
| PL-1356(MoClo: B hom-TKL1-C hom) | SEQ ID NO: 39, 40 |
| PL-1357(MoClo: C hom-RIB1-D hom) | SEQ ID NO: 41, 42 |
| PL-1358(MoClo: D-hom-FLX1-E hom) | SEQ ID NO: 43, 44 |
| PL-1359(MoClo: E hom-POS5-F hom) | SEQ ID NO: 50, 51 |
| PL-1360(MoClo: F hom-HEM3-X-4 int) | SEQ ID NO: 46, 47 |

TABLE 4
Additional integration plasmids used to construct violacein and
violacein derivatives producing S. cerevisiae strains.

| | SEQ ID |
|---|---|
| Plasmid name | NO's |
| PL-1026(MoClo: X-4 int-POS5-X-4 int) | 50, 51 |
| PL-1048(MoClo: XI-5 int-pGAL−>CvVioA-split KlUra3 + | 25, 26 |
| DR L) | |
| PL-1093(MoClo: split KlUra3 + DR R-pGAL1−>VioB-B hom) | 27, 28 |
| PL-3210(MoClo: XI-5 int-NlInkO-split KlUra3 + DR L) | 83, 84 |
| PL-3211(MoClo: XI-5 int-AmAtmO-split KlUra3 + DR L) | 87, 88 |
| PL-3212(MoClo: XI-5 int-SsStaO-split KlUra3 + DR L) | 79, 80 |
| PL-3213(MoClo: split KlUra3 + DR R-LaRebD-B hom) | 77, 78 |
| PL-3214(MoClo: split KlUra3 + DR R-SsStaD-B hom) | 81, 82 |
| PL-3215(MoClo: split KlUra3 + DR R-NlInkD-B hom) | 85, 86 |
| PL-3216(MoClo: split KlUra3 + DR R-AmAtmD-B hom) | 89, 90 |
| PL-908(MoClo: XI-5 int-CvVioA-split KlUra3 + DR L) | 25, 26 |
| PL-1043(MoClo: split KlUra3 + DR R-CvVioB-Leu2 hom) | 27, 28 |
| PL-1027(MoClo: X-4 int-HEM3-X-4 int) | 46, 47 |
| PL-1024(MoClo: X-4 int-RIB1-X-4 int) | 41, 42 |
| PL-1025(MoClo: X-4 int-FLX1-X-4 int) | 43, 44 |
| PL-1115(MoClo: XII-5 up-VioC-XII-5 down) | 33, 34 |
| PL-1008(MoClo P3: VioB-EAAAK3-VioE) | 75, 76 |
| PL-1009(MoClo P3: VioB-GGGGS3-VioE) | 73, 74 |
| PL-1093(MoClo: split KlUra3 + DR R-pGAL1−>VioB-B hom) | 27, 28 |
| PL-4845(split KlUra3 + DR R-VioB-EAAAK3 | 75, 76 |
| PL-4847(EAAAK3-VioE-Rec1 Leu2 down hom) | 75, 76 |
| PL-4846(split KlUra3 + DR R-VioB-GGGGS3) | 73, 74 |
| PL-4848(GGGGS3-VioE-Rec1 Leu2 down hom) | 73, 74 |
| PL-1118(MoClo: X-2 up-pGAL1-VioA-A hom) | 25, 26 |
| PL-1119(MoClo: A hom-VioB-B hom) | 27, 28 |
| PL-1120(MoClo: B hom-VioC- C hom) | 33, 34 |
| PL-1121(MoClo:C hom-VioE-X-2-down) | 29, 30 |
| PL-998(MoClo: X-4 int-Cs73Y-X-4 int) | 91, 92 |
| PL-999(MoClo: X-4 int-Pt73Y-X-4 int) | 95, 96 |
| PL-1000(MoClo: X-4 int-Ha88B_2-X-4 int) | 93, 94 |

Native S. cerevisiae genes were deleted by marker replacement in S. cerevisiae strains which were auxotrophic for essential amino acids e.g., BY4741. Deletion cassettes were prepared by PCR amplification of auxotrophic marker cassettes with primers which included upstream and downstream homology to the coding region of the gene of interest to be deleted. Subsequent transformation of the integration cassette into S. cerevisiae strains and selection on media lacking the required amino acid resulted in integration of the marker cassette at the gene of interest. In some cases, additional direct repeats were introduced flanking the marker cassette to subsequently counter select the marker for repeated use. E.g. the Ura3 cassette was flanked with direct repeats so that the marker could be looped out upon incubation of the S. cerevisiae strain on media containing 5-Fluoroorotic acid (5-FOA) (Table 3 and 4). In some cases, integration cassettes to overexpress gene(s) of interest were flanked with homology arms upstream and downstream of targets for gene deletion, thereby facilitating the simultaneous integration and deletion of genes of interest (Table 5). Finally, in some cases an endonuclease such as MAD7 was used to induce a DNA break at a gene of interest and the gene removed by replacement with a DNA cassette consisting of DNA flanking the gene of interest.

TABLE 5
Gene deletion cassettes generated by PCR amplification of
auxotrophic markers from prototrophic S. cerevisiae.

| Cassette name | SEQ ID NO. |
|---|---|
| BB-501(MET17 GPP1 hom) | SEQ ID NO: 59 |
| BB-509(URA3 ARO10 hom) | SEQ ID NO: 49 |
| BB-1230(KlUra3 + DR PDC5 hom) | SEQ ID NO: 48 |
| BB-1231(KlUra3 + DR UBP3 hom) | SEQ ID NO: 57 |
| BB-1232(KlUra3 + DR ROX1 hom) | SEQ ID NO: 60 |
| BB-1233(KlUra3 + DR HMX1 hom) | SEQ ID NO: 45 |
| BB-1234(KlUra3 + DR HAP1 hom) | SEQ ID NO: 61 |

A list of all S. cerevisiae strains constructed by Example 1 is shown in Table 6 and 7 along with the corresponding genotypes.

TABLE 6
S. cerevisiae strains constructed in Example 1.

| Strain | | |
|---|---|---|
| name | Phenotype | Genotype |
| SC-106 | Tryptamine producer | MATa his341 leu2Δ0 met15Δ0 ura3Δ0 XII-1::(pTDH3−>CkPta, |
| | parental strain | pTEF1−>BbXfpk, EcAroL<−pTEF2, BsPrs<−pPGK1), |
| | | gpp1Δ::MET17, XII-2::(pTDH3−>TRP4, SpHis5), aro10Δ, |
| | | ric1Δ::(pTDH3−>TRP2(S65R, S76L), pPGK1−>ARO4 |
| | | (K229L)), X-3::(pTEF2−>TRP1, pCCW12−>TRP3), XI- |
| | | 5::(KiLeu2<−pKiLeu2, pTEF1−>ARO4(K229L), TRP2 (S65R, |
| | | S76L)<−pTEF2, pFBA1−>ARO1, ARO2<−pPGK1, pTDH3−>CrTdc) |
| SC-139 | Prodeoxyviolacein | MATa his3Δ1 leu2Δ0 met15Δ0 ura3Δ0 XII-1::(pTDH3−>CkPta, |
| | producer | pTEF1−>BbXfpk, EcAroL<−pTEF2, BsPrs<−pPGK1), |
| | | gpp1Δ::MET17, XII-2::(pTDH3−>TRP4, SpHis5), aro10Δ, |
| | | ric1Δ::(pTDH3−>TRP2(S65R, S76L), pPGK1−>ARO4 |
| | | (K229L)), X-3::(pTEF2−>TRP1, pCCW12−>TRP3), XI- |
| | | 5::(KiLeu2<−pKiLeu2, pTEF1−>ARO4(K229L), TRP2 (S65R, |
| | | S76L)<−pTEF2, pFBA1−>ARO1, ARO2<−pPGK1, pTDH3−>CvVioA, |
| | | pPGK1−>CvVioB, pCCW1−>VioE, pKlUra3−>KlUra3) |
| SC-141 | Violacein producer | MATa his3Δ1 leu2Δ0 met15Δ0 ura3Δ0 XII-1::(pTDH3−>CkPta, |
| | | pTEF1−>BbXfpk, EcAroL<−pTEF2, BsPrs<−pPGK1), |
| | | gpp1Δ::MET17, XII-2::(pTDH3−>TRP4, SpHis5), aro10Δ, |
| | | ric1Δ::(pTDH3−>TRP2(S65R, S76L), pPGK1−>ARO4 |
| | | (K229L)), X-3::(pTEF2−>TRP1, pCCW12−>TRP3), XI- |
| | | 5::(KiLeu2<−pKiLeu2, pTEF1−>ARO4(K229L), TRP2 (S65R, |
| | | S76L)<−pTEF2, pFBA1−>ARO1, ARO2<−pPGK1, pTDH3−>CvVioA, |
| | | pPGK1−>CvVioB, pCCW1−>VioE, pTEF1−>CvVioC, pHHF2−>CvVioD, |
| | | pKlUra3−>KlUra3) |
| SC-144 | Deoxyviolacein | MATa his3Δ1 leu2Δ0 met15Δ0 ura3Δ0 XII-1::(pTDH3−>CkPta, |
| | producer | pTEF1−>BbXfpk, EcAroL<−pTEF2, BsPrs<−pPGK1), |
| | | gpp1Δ::MET17, XII-2::(pTDH3−>TRP4, SpHis5), aro10Δ, |
| | | ric1Δ::(pTDH3−>TRP2(S65R, S76L), pPGK1−>ARO4 |
| | | (K229L)), X-3::(pTEF2−>TRP1, pCCW12−>TRP3), XI- |
| | | 5::(KiLeu2<−pKiLeu2, pTEF1−>ARO4(K229L), TRP2 (S65R, |
| | | S76L)<−pTEF2, pFBA1−>ARO1, ARO2<−pPGK1, pTDH3−>CvVioA, |
| | | pPGK1−>CvVioB, pCCW1−>VioE), XII-5::(pTEF1−>CvVioC, |
| | | pKlUra3−>KlUra3) |
| SC-145 | Proviolacein | MATa his3Δ1 leu2Δ0 met15Δ0 ura3Δ0 XII-1::(pTDH3−>CkPta, |
| | producer | pTEF1−>BbXfpk, EcAroL<−pTEF2, BsPrs<−pPGK1), |
| | | gpp1Δ::MET17, XII-2::(pTDH3−>TRP4, SpHis5), aro10Δ, |
| | | ric1Δ::(pTDH3−>TRP2(S65R, S76L), pPGK1−>ARO4 (K229L)), |
| | | X-3::(pTEF2−>TRP1, pCCW12−>TRP3), XI-5::(KiLeu2<−pKiLeu2, |
| | | pTEF1−>ARO4(K229L), TRP2 (S65R, S76L)<−pTEF2, |
| | | pFBA1−>ARO1, ARO2<−pPGK1, pTDH3−>CvVioA, pPGK1−>CvVioB, |
| | | pCCW1−>VioE), XII-5::(pHHF2−>CvVioD, pKlUra3−>KlUra3) |

TABLE 7
Additional S. cerevisiae strains constructed in Example 1.

| Strain | | |
|---|---|---|
| name | Phenotype | Genotype |
| SC-4567 | Prodeoxyviolacein | MATa his3Δ1 leu2Δ0 met15Δ0 ura3Δ0 XII-1::(pTDH3−>CkPta, |
| | producer: NlInkO | pTEF1−>BbXfpk, EcAroL<−pTEF2, BsPrs<−pPGK1), gpp1Δ::MET17, |
| | | XII-2::(pTDH3−>TRP4, SpHis5), aro10Δ, ric1Δ::(pTDH3−>TRP2(S65R, |
| | | S76L), pPGK1−>ARO4 (K229L)), X-3::(pTEF2−>TRP1, pCCW12−>TRP3), |
| | | XI-5::(KiLeu2<−pKiLeu2, pTEF1−>ARO4(K229L), TRP2 (S65R, |
| | | S76L)<−pTEF2, pFBA1−>ARO1, ARO2<−pPGK1, pTDH3−>NlInkO, |
| | | pPGK1−>CvVioB, pCCW1−>VioE, pKlUra3−>KlUra3) |
| SC-4568 | Prodeoxyviolacein | MATa his3Δ1 leu2Δ0 met15Δ0 ura3Δ0 XII-1::(pTDH3−>CkPta, |
| | producer: AmAtmO | pTEF1−>BbXfpk, EcAroL<−pTEF2, BsPrs<−pPGK1), gpp1Δ::MET17, |
| | | XII-2::(pTDH3−>TRP4, SpHis5), aro10Δ, ric1Δ::(pTDH3−>TRP2(S65R, |
| | | S76L), pPGK1−>ARO4 (K229L)), X-3::(pTEF2−>TRP1, pCCW12−>TRP3), |
| | | XI-5::(KiLeu2<−pKiLeu2, pTEF1−>ARO4(K229L), TRP2 (S65R, |
| | | S76L)<−pTEF2, pFBA1−>ARO1, ARO2<−pPGK1, pTDH3−>AmAtmO, |
| | | pPGK1−>CvVioB, pCCW1−>VioE, pKlUra3−>KlUra3) |
| SC-4569 | Prodeoxyviolacein | MATa his3Δ1 leu2Δ0 met15Δ0 ura3Δ0 XII-1::(pTDH3−>CkPta, |
| | producer: SsStaO | pTEF1−>BbXfpk, EcAroL<−pTEF2, BsPrs<−pPGK1), gpp1Δ::MET17, |
| | | XII-2::(pTDH3−>TRP4, SpHis5), aro10Δ, ric1Δ::(pTDH3−>TRP2(S65R, |
| | | S76L), pPGK1−>ARO4 (K229L)), X-3::(pTEF2−>TRP1, pCCW12−>TRP3), |
| | | XI-5::(KiLeu2<−pKiLeu2, pTEF1−>ARO4(K229L), TRP2 (S65R, |
| | | S76L)<−pTEF2, pFBA1−>ARO1, ARO2<−pPGK1, pTDH3−>SsStaO, |
| | | pPGK1−>CvVioB, pCCW1−>VioE, pKlUra3−>KlUra3) |
| SC-4570 | Prodeoxyviolacein | MATa his3Δ1 leu2Δ0 met15Δ0 ura3Δ0 XII-1::(pTDH3−>CkPta, |
| | producer: LaRebD | pTEF1−>BbXfpk, EcAroL<−pTEF2, BsPrs<−pPGK1), gpp1Δ::MET17, |
| | | XII-2::(pTDH3−>TRP4, SpHis5), aro10Δ, ric1Δ::(pTDH3−>TRP2(S65R, |
| | | S76L), pPGK1−>ARO4 (K229L)), X-3::(pTEF2−>TRP1, pCCW12−>TRP3), |
| | | XI-5::(KiLeu2<−pKiLeu2, pTEF1−>ARO4(K229L), TRP2 (S65R, |
| | | S76L)<−pTEF2, pFBA1−>ARO1, ARO2<−pPGK1, pTDH3−>CvVioA, |
| | | pPGK1−>LaRebD, pCCW1−>VioE, pKlUra3−>KlUra3) |
| SC-4571 | Prodeoxyviolacein | MATa his3Δ1 leu2Δ0 met15Δ0 ura3Δ0 XII-1::(pTDH3−>CkPta, |
| | producer: SsStaD | pTEF1−>BbXfpk, EcAroL<−pTEF2, BsPrs<−pPGK1), gpp1Δ::MET17, |
| | | XII-2::(pTDH3−>TRP4, SpHis5), aro10Δ, ric1Δ::(pTDH3−>TRP2(S65R, |
| | | S76L), pPGK1−>ARO4 (K229L)), X-3::(pTEF2−>TRP1, pCCW12−>TRP3), |
| | | XI-5::(KiLeu2<−pKiLeu2, pTEF1−>ARO4(K229L), TRP2 (S65R, |
| | | S76L)<−pTEF2, pFBA1−>ARO1, ARO2<−pPGK1, pTDH3−>CvVioA, |
| | | pPGK1−>SsStaD, pCCW1−>VioE, pKlUra3−>KlUra3) |
| SC-4572 | Prodeoxyviolacein | MATa his3Δ1 leu2Δ0 met15Δ0 ura3Δ0 XII-1::(pTDH3−>CkPta, |
| | producer: NlInkD | pTEF1−>BbXfpk, EcAroL<−pTEF2, BsPrs<−pPGK1), gpp1Δ::MET17, |
| | | XII-2::(pTDH3−>TRP4, SpHis5), aro10Δ, ric1Δ::(pTDH3−>TRP2(S65R, |
| | | S76L), pPGK1−>ARO4 (K229L)), X-3::(pTEF2−>TRP1, pCCW12−>TRP3), |
| | | XI-5::(KiLeu2<−pKiLeu2, pTEF1−>ARO4(K229L), TRP2 (S65R, |
| | | S76L)<−pTEF2, pFBA1−>ARO1, ARO2<−pPGK1, pTDH3−>CvVioA, |
| | | pPGK1−>NlInkD, pCCW1−>VioE, pKlUra3−>KlUra3) |
| SC-4573 | Prodeoxyviolacein | MATa his3Δ1 leu2Δ0 met15Δ0 ura3Δ0 XII-1::(pTDH3−>CkPta, |
| | producer: AmAtmD | pTEF1−>BbXfpk, EcAroL<−pTEF2, BsPrs<−pPGK1), gpp1Δ::MET17, |
| | | XII-2::(pTDH3−>TRP4, SpHis5), aro10Δ, ric1Δ::(pTDH3−>TRP2(S65R, |
| | | S76L), pPGK1−>ARO4 (K229L)), X-3::(pTEF2−>TRP1, pCCW12−>TRP3), |
| | | XI-5::(KiLeu2<−pKiLeu2, pTEF1−>ARO4(K229L), TRP2 (S65R, |
| | | S76L)<−pTEF2, pFBA1−>ARO1, ARO2<−pPGK1, pTDH3−>CvVioA, |
| | | pPGK1−>AmAtmD, pCCW1−>VioE, pKlUra3−>KlUra3) |
| SC-183 | Prodeoxyviolacein | MATa his3Δ1 leu2Δ0 met15Δ0 ura3Δ0 XI-5::(pGAL1−>VioA, |
| | producer CvVioA | pKlUra3−>KlUra3, pPGK1−>VioB, pCCW12−>VioE, pKiLeu2−>KiLeu2, |
| | Gal inducible | pTEF1−>ARO4(K229L), pTEF2−>TRP2 (S65R, S76L), pFBA1−>ARO1, |
| | | pPGK1−>ARO2), XII-1::(pTDH3−>CkPta, pTEF1−>BbXfpk, pTEF2−>EcAroL, |
| | | pPGK1−>BsPrs), gpp1Δ::MET17, XII-2::(pTDH3−>TRP4, pAgTEF−>SpHis5), |
| | | aro10Δ, ric1Δ::(pTDH3−>TRP2(S65R, S76L), pPGK1−>ARO4 (K229L)), |
| | | X-3::(pTEF2−>TRP1, pCCW12−>TRP3) |
| SC-186 | Violacein | MATa his3Δ1 leu2Δ0 met15Δ0 ura3Δ0 XI-5::(pGAL1−>VioA, |
| | producer CvVioA | pKlUra3−>KlUra3, pPGK1−>VioB, pCCW12−>VioE, pTEF1−>VioC, |
| | Gal inducible | pHHF2−>VioD, pKiLeu2−>KiLeu2, pTEF1−>ARO4(K229L), pTEF2−>TRP2 |
| | | (S65R, S76L), pFBA1−>ARO1, pPGK1−>ARO2), XII- |
| | | 1::(pTDH3−>CkPta, pTEF1−>BbXfpk, pTEF2−>EcAroL, pPGK1−>BsPrs), |
| | | gpp1Δ::MET17, XII-2::(pTDH3−>TRP4, pAgTEF−>SpHis5), aro10Δ, |
| | | ric1Δ::(pTDH3−>TRP2(S65R, S76L), pPGK1−>ARO4 (K229L)), |
| | | X-3::(pTEF2−>TRP1, pCCW12−>TRP3) |
| SC-184 | Deoxyviolacein | MATa his3Δ1 leu2Δ0 met15Δ0 ura3Δ0 XI-5::(pGAL1−>VioA, |
| | producer CvVioA | pKlUra3−>KlUra3, pPGK1−>VioB, pCCW12−>VioE, pTEF1−>VioC, |
| | Gal inducible | pKiLeu2−>KiLeu2, pTEF1−>ARO4(K229L), pTEF2−>TRP2 (S65R, |
| | | S76L), pFBA1−>ARO1, pPGK1−>ARO2), XII-1::(pTDH3−>CkPta, |
| | | pTEF1−>BbXfpk, pTEF2−>EcAroL, pPGK1−>BsPrs), gpp1Δ::MET17, |
| | | XII-2::(pTDH3−>TRP4, pAgTEF−>SpHis5), aro10Δ, ric1Δ::(pTDH3−>TRP2(S65R, |
| | | S76L), pPGK1−>ARO4 (K229L)), X-3::(pTEF2−>TRP1, pCCW12−>TRP3) |
| SC-185 | Proviolacein | MATa his3Δ1 leu2Δ0 met15Δ0 ura3Δ0 XI-5::(pGAL1−>VioA, |
| | producer CvVioA | pKlUra3−>KlUra3, pPGK1−>VioB, pCCW12−>VioE, pHHF2−>VioD, |
| | Gal inducible | pKiLeu2−>KiLeu2, pTEF1−>ARO4(K229L), pTEF2−>TRP2 (S65R, |
| | | S76L), pFBA1−>ARO1, pPGK1−>ARO2), XII-1::(pTDH3−>CkPta, |
| | | pTEF1−>BbXfpk, pTEF2−>EcAroL, pPGK1−>BsPrs), gpp1Δ::MET17, |
| | | XII-2::(pTDH3−>TRP4, pAgTEF−>SpHis5), aro10Δ, ric1Δ::(pTDH3−>TRP2(S65R, |
| | | S76L), pPGK1−>ARO4 (K229L)), X-3::(pTEF2−>TRP1, pCCW12−>TRP3) |
| SC-193 | Deoxyviolacein | MATa his3Δ1 leu2Δ0 met15Δ0 ura3Δ0 XI-5::(pGAL1−>VioA, |
| | producer POS5 | pKlUra3−>KlUra3, pPGK1−>VioB, pCCW12−>VioE, pTEF1−>VioC, |
| | overexpression | pKiLeu2−>KiLeu2, pTEF1−>ARO4(K229L), pTEF2−>TRP2 (S65R, |
| | | S76L), pFBA1−>ARO1, pPGK1−>ARO2), XII-1::(pTDH3−>CkPta, |
| | | pTEF1−>BbXfpk, pTEF2−>EcAroL, pPGK1−>BsPrs), gpp1Δ::MET17, |
| | | XII-2::(pTDH3−>TRP4, pAgTEF−>SpHis5), aro10Δ, ric1Δ::(pTDH3−>TRP2(S65R, |
| | | S76L), pPGK1−>ARO4 (K229L)), X-3::(pTEF2−>TRP1, pCCW12−>TRP3), |
| | | X-4::(pTDH3−>POS5) |
| SC-199 | Prodeoxyviolacein | MATa his3Δ1 leu2Δ0 met15Δ0 ura3Δ0 XI-5::(pTDH3−>VioA, |
| | producer CvVioB | pKlUra3−>KlUra3, pGAL1−>VioB, pCCW12−>VioE, pKiLeu2−>KiLeu2, |
| | Gal inducible | pTEF1−>ARO4(K229L), pTEF2−>TRP2 (S65R, S76L), pFBA1−>ARO1, |
| | | pPGK1−>ARO2), XII-1::(pTDH3−>CkPta, pTEF1−>BbXfpk, pTEF2−>EcAroL, |
| | | pPGK1−>BsPrs), gpp1Δ::MET17, XII-2::(pTDH3−>TRP4, pAgTEF−>SpHis5), |
| | | aro10Δ, ric1Δ::(pTDH3−>TRP2(S65R, S76L), pPGK1−>ARO4 (K229L)), |
| | | X-3::(pTEF2−>TRP1, pCCW12−>TRP3) |
| SC-200 | Deoxyviolacein | MATa his3Δ1 leu2Δ0 met15Δ0 ura3Δ0 XI-5::(pTDH3−>VioA, |
| | producer CvVioB | pKlUra3−>KlUra3, pGAL1−>VioB, pCCW12−>VioE, pTEF1−>VioC, |
| | Gal inducible | pKiLeu2−>KiLeu2, pTEF1−>ARO4(K229L), pTEF2−>TRP2 (S65R, |
| | | S76L), pFBA1−>ARO1, pPGK1−>ARO2), XII-1::(pTDH3−>CkPta, |
| | | pTEF1−>BbXfpk, pTEF2−>EcAroL, pPGK1−>BsPrs), gpp1Δ::MET17, |
| | | XII-2::(pTDH3−>TRP4, pAgTEF−>SpHis5), aro10Δ, ric1Δ::(pTDH3−>TRP2(S65R, |
| | | S76L), pPGK1−>ARO4 (K229L)), X-3::(pTEF2−>TRP1, pCCW12−>TRP3) |
| SC-201 | Proviolacein | MATa his3Δ1 leu2Δ0 met15Δ0 ura3Δ0 XI-5::(pTDH3−>VioA, |
| | producer CvVioB | pKlUra3−>KlUra3, pGAL1−>VioB, pCCW12−>VioE, pHHF2−>VioD, |
| | Gal inducible | pKiLeu2−>KiLeu2, pTEF1−>ARO4(K229L), pTEF2−>TRP2 (S65R, |
| | | S76L), pFBA1−>ARO1, pPGK1−>ARO2), XII-1::(pTDH3−>CkPta, |
| | | pTEF1−>BbXfpk, pTEF2−>EcAroL, pPGK1−>BsPrs), gpp1Δ::MET17, |
| | | XII-2::(pTDH3−>TRP4, pAgTEF−>SpHis5), aro10Δ, ric1Δ::(pTDH3−>TRP2(S65R, |
| | | S76L), pPGK1−>ARO4 (K229L)), X-3::(pTEF2−>TRP1, pCCW12−>TRP3) |
| SC-243 | Violacein | MATa his3Δ1 leu2Δ0 met15Δ0 ura3Δ0 XI-5::(pTDH3−>VioA, |
| | producer CvVioB | pKlUra3−>KlUra3, pGAL1−>VioB, pCCW12−>VioE, pTEF1−>VioC, |
| | Gal inducible | pHHF2−>VioD, pKiLeu2−>KiLeu2, pTEF1−>ARO4(K229L), pTEF2−>TRP2 |
| | | (S65R, S76L), pFBA1−>ARO1, pPGK1−>ARO2), XII- |
| | | 1::(pTDH3−>CkPta, pTEF1−>BbXfpk, pTEF2−>EcAroL, pPGK1−>BsPrs), |
| | | gpp1Δ::MET17, XII-2::(pTDH3−>TRP4, pAgTEF−>SpHis5), aro10Δ, |
| | | ric1Δ::(pTDH3−>TRP2(S65R, S76L), pPGK1−>ARO4 (K229L)), |
| | | X-3::(pTEF2−>TRP1, pCCW12−>TRP3) |
| SC-236 | Chromopyrrolic | MATa his3Δ1 leu2Δ0 met15Δ0 ura3Δ0 XII-1::(pTDH3−>CkPta, |
| | acid producer | pTEF1−>BbXfpk, EcAroL<−pTEF2, BsPrs<−pPGK1), gpp1Δ::MET17, |
| | | XII-2::(pTDH3−>TRP4, SpHis5), aro10Δ, ric1Δ::(pTDH3−>TRP2(S65R, |
| | | S76L), pPGK1−>ARO4 (K229L)), X-3::(pTEF2−>TRP1, pCCW12−>TRP3), |
| | | XI-5::(KiLeu2<−pKiLeu2, pTEF1−>ARO4(K229L), TRP2 (S65R, |
| | | S76L)<−pTEF2, pFBA1−>ARO1, ARO2<−pPGK1, pTDH3−>CvVioA, |
| | | pPGK1−>CvVioB, pKlUra3−>KlUra3) |
| SC-244 | Deoxyviolacein | MATa his3Δ1 leu2Δ0 met15Δ0 ura3Δ0 XI-5::(pGAL1−>VioA, |
| | producer CvVioB- | pPGK1−>VioB-EAAAK3-VioE, pKlUra3−>KlUra3, pPGK1−>VioB, |
| | E fusion EAAAK3 | pCCW12−>VioE, pKiLeu2−>KiLeu2, pTEF1−>ARO4(K229L), pTEF2−>TRP2 |
| | linker | (S65R, S76L), pFBA1−>ARO1, pPGK1−>ARO2), XII-1::(pTDH3−>CkPta, |
| | | pTEF1−>BbXfpk, pTEF2−>EcAroL, pPGK1−>BsPrs), gpp1Δ::MET17, |
| | | XII-2::(pTDH3−>TRP4, pAgTEF−>SpHis5), aro10Δ, |
| | | ric1Δ::(pTDH3−>TRP2(S65R, S76L), pPGK1−>ARO4 (K229L)), |
| | | X-3::(pTEF2−>TRP1, pCCW12−>TRP3), XII-5::(pTEF1−>CvVioC) |
| SC-245 | Deoxyviolacein | MATa his3Δ1 leu2Δ0 met15Δ0 ura3Δ0 XI-5::(pGAL1−>VioA, |
| | producer CvVioB- | pPGK1−>VioB-GGGGS3-VioE, pKlUra3−>KlUra3, pPGK1−>VioB, |
| | E fusion GGGGS3 | pCCW12−>VioE, pKiLeu2−>KiLeu2, pTEF1−>ARO4(K229L), pTEF2−>TRP2 |
| | linker | (S65R, S76L), pFBA1−>ARO1, pPGK1−>ARO2), XII-1::(pTDH3−>CkPta, |
| | | pTEF1−>BbXfpk, pTEF2−>EcAroL, pPGK1−>BsPrs), gpp1Δ::MET17, |
| | | XII-2::(pTDH3−>TRP4, pAgTEF−>SpHis5), aro10Δ, |
| | | ric1Δ::(pTDH3−>TRP2(S65R, S76L), pPGK1−>ARO4 (K229L)), |
| | | X-3::(pTEF2−>TRP1, pCCW12−>TRP3), XII-5::(pTEF1−>CvVioC) |
| SC-191 | Deoxyviolacein | MATa his3Δ1 leu2Δ0 met15Δ0 ura3Δ0 XI-5::(pGAL1−>VioA, |
| | producer with | pKlUra3−>KlUra3, pPGK1−>VioB, pCCW12−>VioE, pTEF1−>VioC, |
| | overexpression | pKiLeu2−>KiLeu2, pTEF1−>ARO4(K229L), pTEF2−>TRP2 (S65R, |
| | of RIB1 | S76L), pFBA1−>ARO1, pPGK1−>ARO2), XII-1::(pTDH3−>CkPta, |
| | | pTEF1−>BbXfpk, pTEF2−>EcAroL, pPGK1−>BsPrs), gpp1Δ::MET17, |
| | | XII-2::(pTDH3−>TRP4, pAgTEF−>SpHis5), aro10Δ, ric1Δ::(pTDH3−>TRP2(S65R, |
| | | S76L), pPGK1−>ARO4 (K229L)), X-3::(pTEF2−>TRP1, pCCW12−>TRP3), |
| | | XII-5::(pTEF2−>RIB1) |
| SC-192 | Deoxyviolacein | MATa his3Δ1 leu2Δ0 met15Δ0 ura3Δ0 XI-5::(pGAL1−>VioA, |
| | producer with | pKlUra3−>KlUra3, pPGK1−>VioB, pCCW12−>VioE, pTEF1−>VioC, |
| | overexpression | pKiLeu2−>KiLeu2, pTEF1−>ARO4(K229L), pTEF2−>TRP2 (S65R, |
| | of FLX1 | S76L), pFBA1−>ARO1, pPGK1−>ARO2), XII-1::(pTDH3−>CkPta, |
| | | pTEF1−>BbXfpk, pTEF2−>EcAroL, pPGK1−>BsPrs), gpp1Δ::MET17, |
| | | XII-2::(pTDH3−>TRP4, pAgTEF−>SpHis5), aro10Δ, ric1Δ::(pTDH3−>TRP2(S65R, |
| | | S76L), pPGK1−>ARO4 (K229L)), X-3::(pTEF2−>TRP1, pCCW12−>TRP3), |
| | | XII-5::(pTEF2−>FLX1) |
| SC-226 | Deoxyviolacein | MATa his3Δ1 leu2Δ0 met15Δ0 ura3Δ0 XI-5::(pGAL1−>VioA, |
| | producer with | pKlUra3−>KlUra3, pPGK1−>VioB, pCCW12−>VioE, pTEF1−>VioC, |
| | two pathway | pKiLeu2−>KiLeu2, pTEF1−>ARO4(K229L), pTEF2−>TRP2 (S65R, |
| | copies | S76L), pFBA1−>ARO1, pPGK1−>ARO2), XII-1::(pTDH3−>CkPta, |
| | | pTEF1−>BbXfpk, pTEF2−>EcAroL, pPGK1−>BsPrs), gpp1Δ::MET17, |
| | | XII-2::(pTDH3−>TRP4, pAgTEF−>SpHis5), aro10Δ, ric1Δ::(pTDH3−>TRP2(S65R, |
| | | S76L), pPGK1−>ARO4 (K229L)), X-3::(pTEF2−>TRP1, pCCW12−>TRP3), |
| | | X-2::(pGAL1−>VioA, pTDH3−>VioB, pHHF2−>VioC, pTEF1−>VioE) |
| SC-253 | Deoxyviolacein | MATa his3Δ1 leu2Δ0 met15Δ0 ura3Δ0 XI-5::(pGAL1−>VioA, |
| | producer with | pKlUra3−>KlUra3, pPGK1−>VioB, pCCW12−>VioE, pTEF1−>VioC, |
| | HEM3 | pKiLeu2−>KiLeu2, pTEF1−>ARO4(K229L), pTEF2−>TRP2 (S65R, |
| | overexpression | S76L), pFBA1−>ARO1, pPGK1−>ARO2), XII-1::(pTDH3−>CkPta, |
| | | pTEF1−>BbXfpk, pTEF2−>EcAroL, pPGK1−>BsPrs), gpp1Δ::MET17, |
| | | XII-2::(pTDH3−>TRP4, pAgTEF−>SpHis5), aro10Δ, ric1Δ::(pTDH3−>TRP2(S65R, |
| | | S76L), pPGK1−>ARO4 (K229L)), X-3::(pTEF2−>TRP1, pCCW12−>TRP3), |
| | | X-2::(pGAL1−>VioA, pTDH3−>VioB, pHHF2−>VioC, pTEF1−>VioE), |
| | | X-4::(pTEF1−>HEM3) |
| SC-256 | Deoxyviolacein | MATa his3Δ1 leu2Δ0 met15Δ0 ura3Δ0 XI-5::(pGAL1−>VioA, |
| | producer with | pKlUra3−>KlUra3, pPGK1−>VioB, pCCW12−>VioE, pTEF1−>VioC, |
| | HMX1 deletion | pKiLeu2−>KiLeu2, pTEF1−>ARO4(K229L), pTEF2−>TRP2 (S65R, |
| | | S76L), pFBA1−>ARO1, pPGK1−>ARO2), XII-1::(pTDH3−>CkPta, |
| | | pTEF1−>BbXfpk, pTEF2−>EcAroL, pPGK1−>BsPrs), gpp1Δ::MET17, |
| | | XII-2::(pTDH3−>TRP4, pAgTEF−>SpHis5), aro10Δ, ric1Δ::(pTDH3−>TRP2(S65R, |
| | | S76L), pPGK1−>ARO4 (K229L)), X-3::(pTEF2−>TRP1, pCCW12−>TRP3), |
| | | X-2::(pGAL1−>VioA, pTDH3−>VioB, pHHF2−>VioC, pTEF1−>VioE), hmx1::AmdS |
| SC-257 | Deoxyviolacein | MATa his3Δ1 leu2Δ0 met15Δ0 ura3Δ0 XI-5::(pGAL1−>VioA, |
| | producer with | pKlUra3−>KlUra3, pPGK1−>VioB, pCCW12−>VioE, pTEF1−>VioC, |
| | HAP1 deletion | pKiLeu2−>KiLeu2, pTEF1−>ARO4(K229L), pTEF2−>TRP2 (S65R, |
| | | S76L), pFBA1−>ARO1, pPGK1−>ARO2), XII-1::(pTDH3−>CkPta, |
| | | pTEF1−>BbXfpk, pTEF2−>EcAroL, pPGK1−>BsPrs), gpp1Δ::MET17, |
| | | XII-2::(pTDH3−>TRP4, pAgTEF−>SpHis5), aro10Δ, ric1Δ::(pTDH3−>TRP2(S65R, |
| | | S76L), pPGK1−>ARO4 (K229L)), X-3::(pTEF2−>TRP1, pCCW12−>TRP3), |
| | | X-2::(pGAL1−>VioA, pTDH3−>VioB, pHHF2−>VioC, pTEF1−>VioE), hap1::AmdS |
| SC-259 | Deoxyviolacein | MATa his3Δ1 leu2Δ0 met15Δ0 ura3Δ0 XI-5::(pGAL1−>VioA, |
| | producer with | pKlUra3−>KlUra3, pPGK1−>VioB, pCCW12−>VioE, pTEF1−>VioC, |
| | ROX1 deletion | pKiLeu2−>KiLeu2, pTEF1−>ARO4(K229L), pTEF2−>TRP2 (S65R, |
| | | S76L), pFBA1−>ARO1, pPGK1−>ARO2), XII-1::(pTDH3−>CkPta, |
| | | pTEF1−>BbXfpk, pTEF2−>EcAroL, pPGK1−>BsPrs), gpp1Δ::MET17, |
| | | XII-2::(pTDH3−>TRP4, pAgTEF−>SpHis5), aro10Δ, ric1Δ::(pTDH3−>TRP2(S65R, |
| | | S76L), pPGK1−>ARO4 (K229L)), X-3::(pTEF2−>TRP1, pCCW12−>TRP3), |
| | | X-2::(pGAL1−>VioA, pTDH3−>VioB, pHHF2−>VioC, pTEF1−>VioE), rox1::AmdS |
| SC-157 | Deoxyviolacein | MATa his3Δ1 leu2Δ0 met15Δ0 ura3Δ0 XII-1::(pTDH3−>CkPta, |
| | producer with | pTEF1−>BbXfpk, EcAroL<−pTEF2, BsPrs<−pPGK1), gpp1Δ::MET17, |
| | UGT Cs73Y | XII-2::(pTDH3−>TRP4, SpHis5), aro10Δ, ric1Δ::(pTDH3−>TRP2(S65R, |
| | | S76L), pPGK1−>ARO4 (K229L)), X-3::(pTEF2−>TRP1, pCCW12−>TRP3), |
| | | XI-5::(KiLeu2<−pKiLeu2, pTEF1−>ARO4(K229L), TRP2 (S65R, |
| | | S76L)<−pTEF2, pFBA1−>ARO1, ARO2<−pPGK1, pTDH3−>CvVioA, |
| | | pPGK1−>CvVioB, pCCW1−>VioE), XII-5::(pTEF1−>CvVioC, |
| | | pKlUra3−>KlUra3), X-4::pTEF1−>Cs73Y |
| SC-158 | Deoxyviolacein | MATa his3Δ1 leu2Δ0 met15Δ0 ura3Δ0 XII-1::(pTDH3−>CkPta, |
| | producer with | pTEF1−>BbXfpk, EcAroL<−pTEF2, BsPrs<−pPGK1), gpp1Δ::MET17, |
| | UGT Pt73Y | XII-2::(pTDH3−>TRP4, SpHis5), aro10Δ, ric1Δ::(pTDH3−>TRP2(S65R, |
| | | S76L), pPGK1−>ARO4 (K229L)), X-3::(pTEF2−>TRP1, |
| | | pCCW12−>TRP3), XI-5::(KiLeu2<−pKiLeu2, pTEF1−>ARO4(K229L), |
| | | TRP2 (S65R, S76L)<−pTEF2, pFBA1−>ARO1, ARO2<−pPGK1, pTDH3−>CvVioA, |
| | | pPGK1−>CvVioB, pCCW1−>VioE), XII-5::(pTEF1−>CvVioC, pKlUra3−>KlUra3), |
| | | X-4::pTEF1−>Pt73Y |
| SC-160 | Deoxyviolacein | MATa his3Δ1 leu2Δ0 met15Δ0 ura3Δ0 XII-1::(pTDH3−>CkPta, |
| | producer with | pTEF1−>BbXfpk, EcAroL<−pTEF2, BsPrs<−pPGK1), gpp1Δ::MET17, |
| | UGT Ha88B_2 | XII-2::(pTDH3−>TRP4, SpHis5), aro10Δ, ric1Δ::(pTDH3−>TRP2(S65R, |
| | | S76L), pPGK1−>ARO4 (K229L)), X-3::(pTEF2−>TRP1, |
| | | pCCW12−>TRP3), XI-5::(KiLeu2<−pKiLeu2, pTEF1−>ARO4(K229L), |
| | | TRP2 (S65R, S76L)<−pTEF2, pFBA1−>ARO1, ARO2<−pPGK1, pTDH3−>CvVioA, |
| | | pPGK1−>CvVioB, pCCW1−>VioE), XII-5::(pTEF1−>CvVioC, pKlUra3−>KlUra3), |
| | | X-4::pTEF1−>Ha88B_2 |
| SC-161 | Proviolacein | MATa his3Δ1 leu2Δ0 met15Δ0 ura3Δ0 XII-1::(pTDH3−>CkPta, |
| | producer with | pTEF1−>BbXfpk, EcAroL<−pTEF2, BsPrs<−pPGK1), gpp1Δ::MET17, |
| | UGT Cs73Y | XII-2::(pTDH3−>TRP4, SpHis5), aro10Δ, ric1Δ::(pTDH3−>TRP2(S65R, |
| | | S76L), pPGK1−>ARO4 (K229L)), X-3::(pTEF2−>TRP1, pCCW12−>TRP3), |
| | | XI-5::(KiLeu2<−pKiLeu2, pTEF1−>ARO4(K229L), TRP2 (S65R, |
| | | S76L)<−pTEF2, pFBA1−>ARO1, ARO2<−pPGK1, pTDH3−>CvVioA, pPGK1−>CvVioB, |
| | | pCCW1−>VioE), XII-5::(pHHF2−>CvVioD, pKlUra3−>KlUra3), X-4::pTEF1−>Cs73Y |
| SC-162 | Proviolacein | MATa his3Δ1 leu2Δ0 met15Δ0 ura3Δ0 XII-1::(pTDH3−>CkPta, |
| | producer with | pTEF1−>BbXfpk, EcAroL<−pTEF2, BsPrs<−pPGK1), gpp1Δ::MET17, |
| | UGT Pt73Y | XII-2::(pTDH3−>TRP4, SpHis5), aro10Δ, ric1Δ::(pTDH3−>TRP2(S65R, |
| | | S76L), pPGK1−>ARO4 (K229L)), X-3::(pTEF2−>TRP1, pCCW12−>TRP3), |
| | | XI-5::(KiLeu2<−pKiLeu2, pTEF1−>ARO4(K229L), TRP2 (S65R, |
| | | S76L)<−pTEF2, pFBA1−>ARO1, ARO2<−pPGK1, pTDH3−>CvVioA, pPGK1−>CvVioB, |
| | | pCCW1−>VioE), XII-5::(pHHF2−>CvVioD, pKlUra3−>KlUra3), X-4::pTEF1−>Pt73Y |
| SC-164 | Proviolacein | MATa his3Δ1 leu2Δ0 met15Δ0 ura3Δ0 XII-1::(pTDH3−>CkPta, |
| | producer with | pTEF1−>BbXfpk, EcAroL<−pTEF2, BsPrs<−pPGK1), gpp1Δ::MET17, |
| | UGT Ha88B_2 | XII-2::(pTDH3−>TRP4, SpHis5), aro10Δ, ric1Δ::(pTDH3−>TRP2(S65R, |
| | | S76L), pPGK1−>ARO4 (K229L)), X-3::(pTEF2−>TRP1, pCCW12−>TRP3), |
| | | XI-5::(KiLeu2<−pKiLeu2, pTEF1−>ARO4(K229L), TRP2 (S65R, |
| | | S76L)<−pTEF2, pFBA1−>ARO1, ARO2<−pPGK1, pTDH3−>CvVioA, pPGK1−>CvVioB, |
| | | pCCW1−>VioE), XII-5::(pHHF2−>CvVioD, pKlUra3−>KlUra3), X-4::pTEF1−>Ha88B_2 |

### Example 2—Construction of E. coli Strains for Production of Violacein, Violacein Derivatives, and Substituted Violacein Derivatives by Feeding Substituted Indole Acceptors and Serine or Feeding Violacein and Violacein Derivatives Themselves Either In Vitro or In Vivo

E. coli strains expressing enzymes to convert indole or substituted indole and serine into violacein, violacein derivatives and substituted violacein derivatives, or violacein and violacein derivatives into substituted violacein and violacein derivatives were constructed as follows. Genes were codon optimized for E. coli, synthesized by Twist Biosciences and cloned into pET28a(+). Fully assembled plasmids were transformed into E. coli DH5a strains or E. coli XJb (DE3) autolysis strains (Zymo Research). Plasmids are shown in Table 8 and 9. In some cases, genes were expressed on their own to facilitate E. coli in vivo feeding experiments as well as to facilitate in vitro biocatalytic experiments where the E. coli strains were used to produce and purify enzymes for in vitro reactions (Enzyme purification procedure outlined below).

TABLE 8
Expression plasmids to construct violacein and
derivatives production strains in E. coli

| Plasmid name | SEQ ID NO. |
|---|---|
| PL-543(PcTrpB) | SEQ ID NO: 62, 63 |
| PL-1092(CvVioA) | SEQ ID NO: 52, 26 |
| PL-1093(CvVioB) | SEQ ID NO: 53, 27 |
| PL-1094(CvVioC) | SEQ ID NO: 54, 34 |
| PL-1095(CvVioD) | SEQ ID NO: 55, 32 |
| PL-1096(CvVioE) | SEQ ID NO: 56, 30 |
| PL-226(Pt73Y_GA) | SEQ ID NO: 64, 66 |

TABLE 9
Additional list of expression plasmids to
produce substituted violacein derivatives

| Plasmid name | SEQ ID NO |
|---|---|
| PL-342(Cp73B_GA) | 71, 72 |
| PL-1086(Bs109A1) | 69, 70 |
| PL-1101(Bs109_1) | 67, 68 |

### Example 3—Cultivation of Genetically Modified S. cerevisiae Strains for the De Novo Production of Violacein and Violacein Derivatives

Genetically engineered yeast strains were pre-cultured in 500 μL or 5 mL of liquid Delft minimal media (Table 10) with 20 g/L glucose and relevant amino acid supplements for 48 h at 30° C. and 280 rpm in 2 mL microtiter plates with air-permeable sealing or 12 mL cultivation tubes. Subsequently, 10 μL or 1 mL of yeast preculture was transferred to 490 μL or 49 mL of Delft minimal media with 20 g/L glucose and relevant amino acid supplements and cultivated for 72 h at 30° C. and 280 rpm in either a 2 mL microtiter plate with air-permeable sealing, or a 200 mL shakeflask. After cultivation, extracellular metabolites were extracted by mixing whole cell broth 1:1 with 100% methanol, vortexing thoroughly and centrifuging at 4000×g for 5 min. The supernatant was subsequently diluted in MilliQ water to obtain a final methanol concentration of 12.5% in the samples, which were then analyzed using UHPLC or LC-MS/MS as described in Example 5. Intracellular metabolites were extracted by mixing whole cell broth 1:3 with 100% methanol in 2 mL screw cap tubes containing glass beads (Ø 1 mm) and lysing the cells by bead-bashing on a FastPrep® FP120 Cell Disrupter (Thermo Savant) at 6.5 m/s for 45 sec followed by centrifugation at 4000×g for 5 min. The supernatants with final methanol concentration of 75% were then analyzed using UHPLC or LC-MS/MS as described in Example 5.

In some cases, where a non-ionic surfactant was added to the cultivation media, extraction of extracellular metabolites was done by centrifuging the entire culture broth at 4000×g for 5 min to separate the culture into 3 phases, a biomass “pellet” a dilute surfactant phase, and a violacein or violacein derivative concentrated surfactant phase. Isolation of the violacein or violacein derivative rich phase was done by pipetting the phase into a new container. Where possible, authentic analytical standards were used for quantification. In some cases, where a non-volatile solvent was added to the cultivation media extraction of metabolites in the extracellular space and ISPR solvent phase were performed by adding 500 uL 100% acetonitrile directly to the culture broths followed by brief vortexing thereby disrupting the phase separation between the media and ISPR solvent phases. Subsequently, 200 μL of this suspension was transferred to new tubes and mixed 1:1 with 100% acetonitrile resulting in final concentrations of 75% acetonitrile and 2.5% ISPR solvent. Yeast cells were pelleted by centrifugation at 11.000×G for 5 min., and the supernatant was recovered and analyzed by UHPLC or LC-MS/MS.

In some cases, violacein biosynthetic genes were placed under the control of galactose inducible promoters. In such cases the yeast strains were pre-cultured in 500 μL liquid Delft media (Table 10) with 20 g/L glucose and relevant amino acid supplements for 48 h at 30° C. and 280 rpm in 2 mL microtiter plates with air-permeable sealing. Subsequently, 10 μL of yeast preculture was transferred to 490 μL Delft minimal media with 20 g/L glucose and relevant amino acid supplements and cultivated for 48 h at 30° C. and 280 rpm in a 2 mL microtiter plate with air-permeable sealing. The culture plate was thereafter centrifuged at 3000×g for 5 min, the supernatant removed and 500 μL of Delft media with 20 g/L of galactose added to each well. The plate was then cultured for 72 h at 280 rpm. In some cases, the media contained 10-20% of an in situ extractant during the final cultivation with galactose induced production of violacein and violacein derivatives.

TABLE 10
Composition of Delft minimal media

| | | |
|---|---|---|
| Succinate | 10 | g/L |
| Glucose | 20 | g/L |
| NaOH | 6 | g/L |
| (NH₄)₂SO₄ | 5 | g/L |
| H₂KO₄P | 3 | g/L |
| MgSO₄ * 7 H₂O | 500 | mg/L |
| Na₂-EDTA | 150 | mg/L |
| ZnSO₄ * 7H₂O | 45 | mg/L |
| MnCl₂ * 4H₂O | 10.27 | mg/L |
| CuSO₄ * 5H₂O | 3 | mg/L |
| CaCl₂ *2H₂O | 22.65 | mg/L |
| FeSO₄ * 7H₂O | 30 | mg/L |
| H₃BO₃ | 10 | mg/L |
| Na₂MoO₄ * 2H₂O | 4 | mg/L |
| CoCl₂ * 6H₂O | 3 | mg/L |
| Kl | 1 | mg/L |
| d-biotin | 0.6 | mg/L |
| Ca-pantothenate | 12 | mg/L |
| Nicotinic acid | 12 | mg/L |
| Thiamine-HCl | 12 | mg/L |
| Pyridoxine-HCl | 12 | mg/L |
| 4-aminobenzoic acid | 2.4 | mg/L |
| Myo-inositol | 300 | mg/L |

### Example 4—Producing and Purifying Enzymes for In Vitro Enzyme Reactions

In some instances, production of violacein derivatives can be carried out in vitro using purified enzymes with addition of required co-factors and substrates. Preparation of the purified enzymes for the biocatalytic reactions were performed as follows: [0387]5 mL of 2× concentrated LB medium+kanamycin (50 μg/mL) was inoculated with E. coli XJb (DE3) strains expressing a gene of interest and incubated overnight at 30° C. with shaking. The following day, 0.5 mL cell cultures were inoculated in 50 mL AIM TB medium+kanamycin (50 μg/mL) (supplemented with 1 mM 6-aminolevulinic acid and 40 μM ammonium iron sulfate for VioB) at 30° C. with shaking. After 3-3.5 hr of incubation, 3 mM arabinose was added to the cell cultures and incubated for 24 h at 16° C. with shaking. The following day, the cells were collected by centrifugation at 6500×g for 10 mins at 4° C. Cells were resuspended in 5 mL ice-cold GT buffer (50 mM Tris-HCl pH7.4 15+1 mM phenylmethanesulfonyl fluoride+1 cOmplete™, mini, EDTA-free Protease Inhibitor Cocktail tablet (Roche)). The resuspended material was transferred to a 50 mL falcon tube and kept at −80° C. for at least 15 mins. Falcon tubes were then thawed at room temperature, as the tubes were thawing the following reagents were added; 2.6 mM MgCl₂, 1 mM CaCl₂, 250 μL of a 1.4 mg/ml DNase solution (Sigma) dissolved in MilliQ water. Tubes were gently inverted to mix then were incubated for 5 mins at 37° C. 4× Binding buffer was then added to the tubes (to final conc of 50 mM Tris-HCl pH7.4, 10 mM imidazole, 500 mM NaCl and the pH adjusted to 7.4 with HCl). The mix was centrifuged at 10000×g for 30 mins at 4° C., the supernatant transferred to a fresh 50 mL falcon tubes and centrifuged again to remove any remaining cellular debris at 10000×g for 30 minutes at 4° C. Collected supernatant from the centrifuged enzyme preparations were reconstituted with co-factor FAD (VioA, VioC, and VioD) and metal ion (Cu²⁺ for VioD) for 1 hr before transferring it to tube containing HIS-Select resin. While the enzyme prep was incubating, 3 mL of HIS-Select (available from Sigma P6611) column material was added to a fresh 50 mL tube and washed by adding MilliQ water up to 50 mL, centrifuging at 2000×g for 2 mins and discarding the supernatant. This washing step was repeated. Finally, MilliQ water was added to the HIS-Select material to an approximate 50% volume. Reconstituted enzyme preparation was transferred to the tube containing the HIS-Select material through a Miracloth (available from Merck Millipore), and then incubated at 4° C. with gently shaking by inversion for 2 h. After 2 h the mix was centrifuged at 2000×g for 4 minutes at 4° C. and the supernatant discarded. The remaining HIS-Select material was washed twice with 1× binding buffer (50 mM Tris-HCl, 0.5M NaCl, 10 mM Imidazole, pH 7.4) with centrifugation at 2000×g for 4 minutes at 4° C. The HIS-Select material was resuspended in 5 mL 1× binding buffer and transferred to a Poly-Prep® Chromatography Column (available from BioRad, 7311550). The HIS-Select material was kept at 4° C. and washed twice with 1× binding buffer by filling up the column and allowing it to drip through. Finally, purified enzymes were eluted from the HIS-Select material by adding 7.5 mL of elution buffer (50 mM Tris-HCl, 250 mM Imidazole, pH7.4) and collecting the flow through. VioE was purified in presence of 2 mM MgCl₂ and 1 mM DTT. Enzymes were used immediately in in vitro enzyme assays or stored at −20° C. in 50% glycerol until needed.

Glycosyltransferase (UGT) enzymes were produced and purified as follows. 5 mL of 2× concentrated LB medium+appropriate antibiotic (50 μg/mL) was inoculated with E. coli XJb (DE3) strains expressing a plasmid of interest and incubated overnight at 30° C. with shaking. The following day, 50 ml TB medium+antibiotic in 250 ml baffled flasks was inoculated with 0.5 mL overnight cell culture and incubated for 3.5 hrs at 30° C. The cell cultures were then induced with 3 mM arabinose, 0.1 mM IPTG and Incubated for 24 h at 25° C., with shaking. The following day, the cells were collected by centrifugation at 6500×g for 10 mins at 4° C. Cells were resuspended in 5 mL ice-cold protein extraction buffer (50 mM Tris-HCl pH7.4+1 mM phenylmethanesulfonyl fluoride+1 cOmplete™, mini, EDTA-free Protease Inhibitor Cocktail tablet (Roche). The resuspended material was transferred to five 1.5 mL eppendorf tubes and kept at −80° C. for at least 15 mins. The tubes were then thawed at room temperature, as the tubes were thawing the following reagents were added; 2.6 mM MgCl₂, 1 mM CaCl₂, 300 U/mL DNase solution (DENARASE®, C-lecta) and 0.2 mg/mL lysozyme (Sigma) dissolved in MilliQwater. Tubes were gently inverted to mix then were incubated for 10-15 mins at 37° C. 3 volumes of 4× Binding buffer was then added to the tubes (to final concentration of 50 mM Tris-HCl pH7.4, 10 mM imidazole, 500 mM NaCl and the pH adjusted to 7.4 with HCl). The mix was centrifuged at 10000×g for 30 mins at 4° C., the supernatant transferred to a fresh eppendorf tubes and centrifuged again to remove any remaining cellular debris at 10000×g for 30 minutes at 4° C. While the enzyme prep was centrifuging, HisPur 0.2 ml spin column (Thermo Scientific) are prepared as instructed by manufacturer. HisPur column was washed with MilliQ water and equilibrated with two resin-bed volumes of 1×his-binding buffer two times. HisPur column was centrifuged at 700×g for 2 minutes to remove buffer. 600 μL of collected supernatant from the centrifuged enzyme preparation was transferred to the HisPur column and then incubated at 4° C. with gentle shaking by inversion for 30 minutes. After 30 minutes, the unbound protein was removed by centrifugation 700×g for 2 min. The remaining 600 μL of collected supernatant from the centrifuged enzyme preparation was loaded to the HisPur column and incubated again at 4° C. with shaking. Unspecific binding of other proteins was removed by washing twice with 1× binding buffer (50 mM Tris-HCl, 0.5M NaCl, 10 mM Imidazole, pH 7.4). Enzyme was resuspended in 200 μl elution buffer for 2 minutes before elution by centrifugation (700×g for 2 minutes at 4° C.). Enzymes were used immediately in in vitro enzyme assays or stored at −20° C. in 50% glycerol until needed.

### Example 5—Detection and Quantification Methods for Violacein and Violacein Derivatives

LC-MS/QTOF analysis for violacein and other substituted derivatives was performed on a Dionex UltiMate 3000 Quaternary Rapid Separation UHPLC′ focused system (Thermo Fisher Scientific, Germering, Germany) coupled to a Compact micrOTOF-Q mass spectrometer (Bruker, Bremen, Germany) equipped with an electrospray ion source (ESI) operated in positive ion mode. Separation was achieved on a Kinetex XB-C18 column (150×2.1 mm, 1.7 μm, 100 A, Phenomenex). For eluting 0.05% (v/v) formic acid in H₂O and 0.05% (v/v) formic acid in acetonitrile were employed as mobile phases A and B, respectively. Gradient conditions were as follows: 0.0-1.0 min 2% B; 1.0-24.0 min 2-75% B, 24.0-25.0 min 75-100% B, 25.0-27.5 min 100% B, 27.5-28.0 min 100-2% B, and 28.0-30.0 min 2% B. The flow rate of the mobile phase was 300 μL/min and the injection volume was 10 μL. The column oven temperature was maintained at 30° C. UV spectra for each sample were acquired at 220, 230, 240, and 280 nm. The ion spray voltage was maintained at +4500 V. Dry temperature was set to 250° C., and the dry gas flow was set to 8 L/min. Nitrogen was used as the dry gas, nebulizing gas, and collision gas. The nebulizing gas was set to 2.5 bar and collision energy to 10 eV. MS spectra were acquired in an m/z range from 50 to 1000 amu and MS/MS spectra in a range from 100-800 amu. Sampling rate was 2 Hz. Sodium formate clusters were used for mass calibration. All files were automatically calibrated by postprocessing. The data was processed using Bruker Compass DataAnalysis 4.3.

UHPLC analysis of violacein, and other substituted violacein derivatives was performed on an Agilent 1290 Infinity II LC system (Agilent Technologies, Böblingen, Germany). Separation was achieved on a Waters Acquity UPLC CSH Fluoro-Phenyl column (100×2.1 mm, 1.7 μm, 130 A, Waters). For elution, 10 mM ammonium acetate in H₂O and 10 mM ammonium acetate in 90% methanol were used as mobile phases A and B, respectively. Gradient conditions were as follows: 0-5 min 5-95% B; 5-8 min 95% B; 8-8.1 min 95-5% B, and 8.1-9 min 5% B. The column oven temperature was maintained at 35° C. and the injection volume was 2 μL. UV/Vis spectra for each sample were acquired at 230 and 575 nm. The data was processed using Agilent Openlab CDS Chemstation Rev. C.01.10.

In some cases, the concentration of violacein and violacein derivatives was determined by a simple plate-based spectrophotometric assay. Since violacein and violacein derivatives all give a different visible colour, a simple colour based quantification can be performed. Here, supernatant from S. cerevisiae cultivation, or reaction mix from in vitro enzyme assays were diluted in water or organic solvent to get within the linear range of the spectrophotometric plate-reader. Diluted samples were added to a 96-well plate and the full absorbance spectrum of each sample was measured in a spectrophotometric plate reader (e.g. a BMG Labtech FLUO star Omega). Where possible a dilution curve was prepared from authentic analytical standards to accurately quantify the amount of a compound in solution. Where an authentic analytical standard was not available, relative absorbance was used to compare the amount of a compound in solution with an internal benchmark.

Alternatively, the chromatographic separation was obtained on a F5 LC column Kinetex 1.7 μm 100A 100×2.1 mm, with SecutityGuard ULTRA (Phenomenex). For elution, 0.05% formic acid in water and 0.05% formic acid in acetonitrile were used as mobile phases A and B, respectively. The elution flow was maintained at 0.4 ml/min. Gradient conditions were as follows: 0-3 min 3-20% B, 3-16 min 20-70% B, 16-17.5 min at 70% B, 17.5-18 min 70-3% B, 18-19 min 3% B. The column oven temperature was maintained at 35° C. and the injection volume was 5 μL. The 1290 Infinity II Diode Array Detector FS mediated the collection of absorbance curves at 230 nm, 575 nm and 620 nm, as well as absorption spectra between 190 and 640 nm. The software Agilent Openlab CDS Chemstation Rev. C.01.10 was used for data analysis and processing. The quantification of violacein and deoxyviolacein was based on external standard calibration at 575 nm using authentic analytical standards.

### Example 6—Test of Chemical Stability of Violacein and Violacein Derivatives

Chemical stability of violacein and violacein derivatives was determined under alkaline, acidic, oxidative and heat stress as follows. 25 mM stock solutions of the target molecules were prepared in 100% methanol. 15 μL is mixed with 5 μL of 400 mM HCl solution (final pH=1.1), 400 mM NaOH solution (Final pH=12.5), 12% H₂O₂ solution (final concentration 3%), or H₂O pH 7.0. Acidic, alkaline and oxidative samples were incubated at 30° C. for 24 h while samples in water were incubated at 80° C. for 24 h. A control under ambient conditions was also prepared where 15 μL of the molecule was added to 5 μL H₂O pH 7.0 and incubated at 30° C. After 24 h samples were placed on ice and 60 μL of ice-cold 100% methanol is added to each sample. Samples were centrifuged and transferred to HPLC vials for analysis. The remaining concentration was quantified by comparing to authentic analytical standards. Determining the presence of degradation products were determined by comparing with authentic analytical standards.

### Example 7—High Level De Novo Production of Violacein and Violacein Derivatives by Engineered S. cerevisiae

### Part I

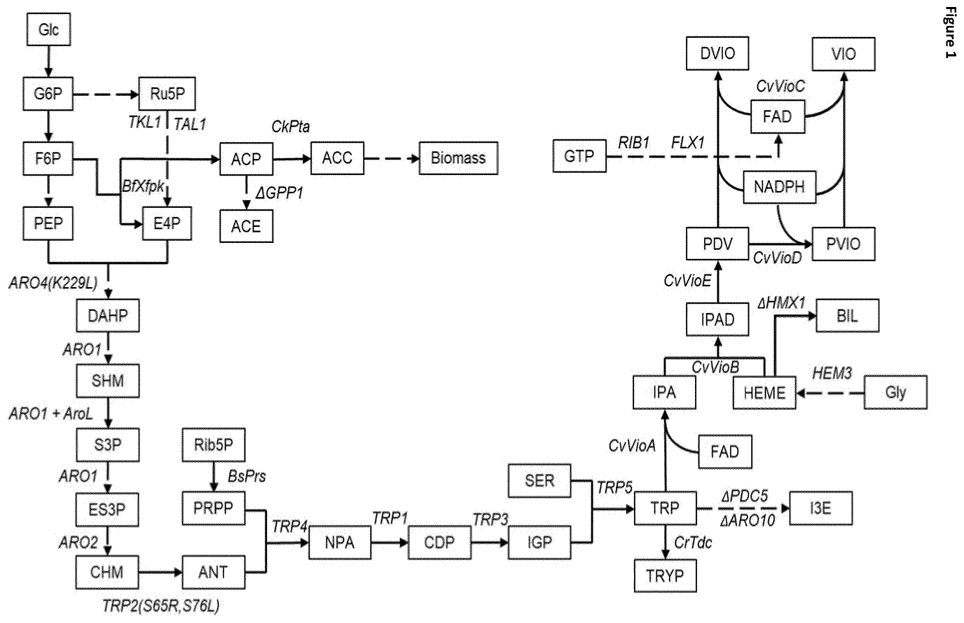

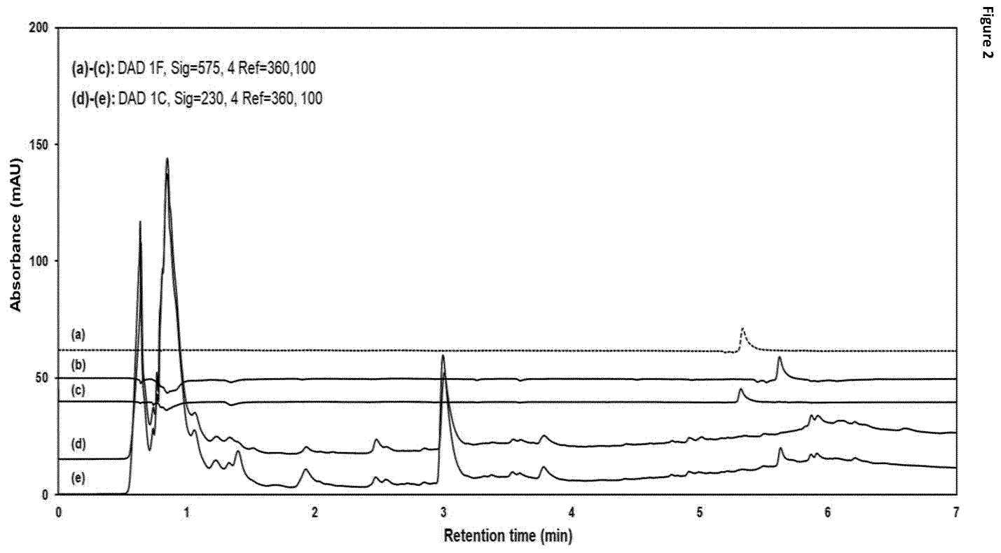

To efficiently produce violacein and related natural derivatives, S. cerevisiae strains were constructed as described in Example 1, testing of the resulting strains and the impact of the genetic modification on violacein was performed as described in Example 3, and quantified as described in Example 5. First, a high tryptamine producer was constructed by combining a series of genetic modifications in wild-type S. cerevisiae strain S288C leading to SC-106. To produce violacein, introduction of all 5 violacein biosynthetic genes is required (CvVioA-E) (SC-141), however, to produce other natural violacein derivatives, different combinations of the biosynthetic pathway must be introduced (as outlined in FIG. 1). Introduction of CvVioA, CvVioB and CvVioE led to the production of prodeoxyviolacein (SC-139), introduction of CvVioA, CvVioB, CvVioE and CvVioC led to the production of deoxyviolacein (SC-144), and introduction of CvVioA, CvVioB, CvVioE and CvVioD led to the production of proviolacein (SC-145). Integration of the violacein biosynthetic genes occurred at the genomic integration site containing tryptophan decarboxylase gene CrTdc thereby facilitating simultaneous introduction of the biosynthetic genes and removal of CrTdc which would otherwise compete with the biosynthetic genes for tryptophan flux. Successful production of all 4 coloured compounds was initially verified by observing an intense colour in the culture, and subsequently by observing a peak at the expected absorbance and retention time on HPLC. Finally, where possible, authentic analytical standards were used to conclusively demonstrate the production of the target compound. In a follow-up experiment, the parental tryptamine production strain as well as the 4 producer strains were cultivated in liquid media in order to assess and quantify production. After centrifuging the cultures, it was observed that the vast majority of violacein or violacein derivative was contained in the cell pellet, suggesting the compounds were not exported from the cell. After performing an intracellular extraction (as described in Example 3), the resulting samples were analyzed by HPLC. As shown in FIG. 2 each strain produces a new and unique peak on the HPLC between 5 and 6 minutes. By comparing to an authentic analytical standard it was confirmed that the peak produced by SC-141 was indeed violacein and the peak produced by SC-144 was indeed deoxyviolacein. LC-MS/QTOF was used to confirm that the other observed peaks corresponded to the expected compound based on the genotype of each strain. Based on the mass and fragmentation pattern it was confirmed that SC-141 produced violacein, SC-144 produced deoxyviolacein, SC-139 produced prodeoxyviolacein and SC-145 produced proviolacein. Based on the peak areas, and by comparing to a violacein standard curve, the amount of each compound produced in this cultivation experiment was quantified. The results presented in Table 11 demonstrate how the genetic modifications introduced into each strain results in efficient production of each target compound with notable examples like deoxyviolacein in SC-144 reaching 173.2 mg/L. Also of note was the absence of by-products and intermediates in some strains. Prior art consistently demonstrates for example the high accumulation of deoxyviolacein in violacein producing strains due to the promiscuity of CvVioC which readily accepts prodeoxyviolaceinic acid as substrate as well as the native substrate proviolaceinic acid. In this experiment, no deoxyviolacein was detected in the violacein producing strains SC-141 indicating efficient production and presumably efficient enzymatic activity from CvVioD.

TABLE 11
S. cerevisiae strains engineered for the production of violacein
and violacein derivatives. Titer of each metabolite produced after
cultivation for 72 h at 30° c. and intracellular extraction.

| Strain | Genotype | Produced metabolite (mg/L) | | | | |
|---|---|---|---|---|---|---|
| name | description | TRYP | PDV | DV | PV | VIO |
| SC-106 | Tryptamine producer | 449.1 | ND | ND | ND | ND |
| SC-139 | Prodeoxyviolacein | ND | 147.4 | ND | ND | ND |
| | producer | | | | | |
| SC-141 | Violacein producer | ND | ND | ND | ND | 80.2 |
| SC-144 | Deoxyviolacein | ND | 25.7 | 173.2 | ND | ND |
| | producer | | | | | |
| SC-145 | Proviolacein | ND | 23.5 | ND | 90.7 | ND |
| | producer | | | | | |

TRYP: Tryptamine, PDV: Prodeoxyviolacein, DV: Deoxyviolacein, PV: Proviolacein, VIO: Violacein, ND: Not Detected.

Conclusions: Overall this experiment shows that S. cerevisiae is a viable host for the production of violacein and other natural violacein derivatives, and that by introducing various genetic modifications into the host strain, efficient production of these compounds can be realized.

### Part II

To further improve S. cerevisiae's ability to produce violacein and violacein derivatives a series of additional genetic modifications was performed. During phenotypic evaluation of the first set of production strains (SC-139, SC-141, SC-144, SC-145) it was found that the accumulation of each target product was highly toxic to the yeast production host with a noticeable decrease in growth rate and final OD600. During the various pre-culture steps it was also frequently observed that production strains lost the ability to produce each target compound (observed by a loss of colour of the biomass, either in liquid or solid media), suggesting strong selection pressure against the introduced biosynthetic pathway. This observation is not surprising given the strict intracellular accumulation observed and the fact that these compounds are known to be toxic to many fungal species. As an initial step to overcome this product toxicity, the gene encoding the first step of the biosynthetic pathway, conversion of tryptophan to IPA imine catalyzed by CvVioA was placed under the control of the galactose inducible promoter pGAL10. In this way, production of the toxic products is suppressed when cells are grown with glucose as the carbon source, and only produced when cells are grown with galactose as the carbon source. This enables a production strategy whereby production strains are first grown on glucose to build up biomass without accumulation of the toxic product, then switched to growth on galactose to produce the compound of interest, thereby delaying the toxicity effect.

In an initial test, similar production strains to those described in Part I were generated but with the first step of the biosynthetic pathway CvVioA under the control of the pGAL10 promoter, and a production test carried out where strains were first grown on glucose, then switched to galactose. The results are presented in Table 12 and show that strains with an inducible biosynthetic pathway reach a significantly higher final OD600 than constitutive strains while producing comparable titers.

TABLE 12
S. cerevisiae strains engineered for the production of violacein and violacein derivatives
with the first step CvVioA under control of the galactose inducible promoter pGAL10.
Titer of each metabolite produced after cultivation for 72 h at 30° c.

| Strain | Genotype | Galactose | Produced metabolite (mg/L) | | | | | Final |
|---|---|---|---|---|---|---|---|---|
| name | description | inducible | TRYP | PDV | DV | PV | VIO | OD600 |
| SC-106 | Tryptamine | No | 450.2 | ND | ND | ND | ND | 12.56 |
| | producer | | | | | | | |
| SC-139 | Prodeoxyviolacein | No | ND | 12.6 | ND | ND | ND | 5.45 |
| SC-183 | producer | Yes | ND | 30.12 | ND | ND | ND | 9.56 |
| SC-141 | Violacein producer | No | ND | ND | ND | 2.5 | 14.4 | 2.54 |
| SC-186 | | Yes | ND | ND | ND | 7.26 | 48.4 | 7.54 |
| SC-144 | Deoxyviolacein | No | ND | 3.54 | 10.3 | ND | ND | 3.56 |
| SC-184 | producer | Yes | ND | 4.93 | 14.8 | ND | ND | 8.99 |
| SC-145 | Proviolacein | No | ND | 1.23 | ND | 5.4 | ND | 5.67 |
| SC-185 | producer | Yes | ND | 6.86 | ND | 30.45 | ND | 9.65 |

TRYP: Tryptamine, PDV: Prodeoxyviolacein, DV: Deoxyviolacein, PV: Proviolacein, VIO: Violacein, ND: Not Detected. Also shown is the final OD600 at the end of the cultivation.

In a related follow up study, the effect of placing CvVioB under control of the galactose inducible promoter pGAL10 instead of CvVioA was tested. The parental strains used in these studies have all been engineering to significantly over produce tryptophan where all gene overexpressions are under constitutive control. Thus, when CvVioA is placed under control of a galactose inducible promoter and strains grown on glucose, while the production of violacein or a violacein derivative is repressed, the overproduction of tryptophan is not. This is potentially problematic given the fact that S. cerevisiae contains many endogenous pathways to further metabolize tryptophan, notably by conversion to dead end products such as tryptophol. A potential solution to this is to overexpress CvVioB under a galactose inducible promoter and have CvVioA under constitutive control. This would lead to biomass production and accumulation of IPA imine when strains are cultivated and glucose, then conversion of the accumulated substrate to violacein or violacein derivatives when grown on galactose. pGAL10->CvVioB strains producing violacein, proviolacein, prodeoxyviolacein and deoxyviolacein were constructed and tested for improved production compared to strains with CvVioA under the pGAL10 promoter in a cultivation experiment. During the growth phase on glucose the strains grew at approximately the same growth rate and reached the same final OD600 as pGAL10->CvVioA strains, and extraction of metabolites from both the intracellular and extracellular space prior to induction with galactose showed no IPA imine in the extracellular space and high concentration in the intracellular space. These combined results suggest that IPA imine is not exported from the cell, but also is not toxic to S. cerevisiae (in contrast to the other downstream biosynthetic products which are highly toxic). This is particularly useful since, in contrast to Tryptophan, there are unlikely to be any endogenous pathways in S. cerevisiae that are able to further metabolize IPA imine. Samples were taking and analyzed after the production phase where cells were cultivated in galactose media and in the presence of 10% isopropyl myristate as the in situ extractant. The results shown in Table 13 show strains where CvVioB is under the control of the inducible promoter produce more of the target product than comparable strains with CvVioA under the inducible promoter (Table 12).

TABLE 13
S. cerevisiae strains engineered for the production of violacein
and violacein derivatives with the second step CvVioB under control
of the galactose inducible promoter pGAL10. Titer of each metabolite
produced after cultivation for 72 h at 30° c.

| Strain | | Produced metabolite (mg/L) | | | | |
|---|---|---|---|---|---|---|
| name | Genotype description | TRYP | PDV | DV | PV | VIO |
| SC-199 | Prodeoxyviolacein | ND | 40.12 | ND | ND | ND |
| | producer | | | | | |
| SC-243 | Violacein producer | ND | ND | ND | 12.45 | 69.87 |
| SC-200 | Deoxyviolacein producer | ND | 8.12 | 35.12 | ND | ND |
| SC-201 | Proviolacein producer | ND | 14.12 | ND | 50.12 | ND |

TRYP: Tryptamine, PDV: Prodeoxyviolacein, DV: Deoxyviolacein, PV: Proviolacein, VIO: Violacein, ND: Not Detected. Also shown in the final OD600 at the end of the cultivation.

Conclusions: Overall these experiments demonstrate that placing one or more enzymes of the violacein biosynthetic pathway under inducible control is an effective strategy to alleviate product toxicity and that a particularly effective strategy would be to place the second step CvVioB under inducible control to allow the intracellular accumulation of the non-toxic and non-convertible substrate IPA imine.

Given the success of the galactose inducible strains, they were used as parental strains to test additional genetic modifications to improve the production of each coloured product.

The effect of boosting NADPH production by overexpression of native yeast gene POS5 was tested by introducing a POS5 overexpression cassette into deoxyviolacein producing parental strain SC-184 resulting in strain SC-193. Many of the enzymes of the violacein biosynthetic pathway require NADPH as a co-factor, so boosting NADPH is a good strategy to further increase production. Results from a cultivation (Table 14) show a significant improvement in titer compared to the parental strain indicating that overexpression of POS5 (and by extension improving NADPH availability) improves production.

TABLE 14
S. cerevisiae strains engineered for the production of deoxyviolacein
with overexpression of native yeast gene POS5. Titer of each metabolite
produced after cultivation for 72 h at 30° c.

| Strain | | Produced metabolite (mg/L) | | | | |
|---|---|---|---|---|---|---|
| name | Genotype description | TRYP | PDV | DV | PV | VIO |
| SC-184 | Parental strain | ND | 13.4 | 15.9 | ND | ND |
| SC-193 | POS5 overexpression | ND | 19.4 | 25.3 | ND | ND |

TRYP: Tryptamine, PDV: Prodeoxyviolacein, DV: Deoxyviolacein, PV: Proviolacein, VIO: Violacein, ND: Not Detected.

Conclusions: This experiment shows that overexpression of POS5 leads to improved production of violacein and violacein derivatives.

The effect of boosting heme production was evaluated through a series of native gene deletions and overexpressions hypothesized to increase heme production. Heme is an essential co-factor for CvVioB so it is reasoned that under high CvVioB activity, additional heme may be required.

The gene deletions and expressions were evaluated by introducing them into a deoxyviolacein producing parental strain that contained 2 copies of all 4 biosynthetic genes (CvVioA, CvVioB, CvVioE, CvVioC) and produced significantly more deoxyviolacein than strains with only 1 copy of each biosynthetic gene. Table 15 shows the results of a cultivation experiment comparing the parental strain with strains containing a gene deletion or overexpression related to heme production. The table shows that under these conditions, deletion of ROX1 and overexpression of HEM3 both lead to a significant improvement in deoxyviolacein production. Deletion of HAP1 and HMX1 under these conditions did not lead to any significant change in titer, however, as these are both involved in heme biosynthesis, their deletion may prove beneficial in strains with even higher titers.

TABLE 15
Production of deoxyviolacein in S. cerevisiae strains with
genetic strategies to boost heme production. Titer of each
metabolite produced after cultivation for 72 h at 30°
c., TRYP; Tryptophan, PDV: Prodeoxyviolacein, DV: Deoxyviolacein,
PV: Proviolacein, VIO: Violacein, ND: Not detected.

| Strain | | Produced metabolite (mg/L) | | | | |
|---|---|---|---|---|---|---|
| name | Genotype description | TRYP | PDV | DV | PV | VIO |
| SC-226 | Parental strain | ND | 27.7 | 36.6 | ND | ND |
| SC-253 | HEM3 overexpression | ND | 25.7 | 59.3 | ND | ND |
| SC-256 | HMX1 deletion | ND | 11.8 | 36.9 | ND | ND |
| SC-257 | HAP1 deletion | ND | 20.3 | 37.8 | ND | ND |
| SC-259 | ROX1 deletion | ND | 35.7 | 56.8 | ND | ND |

Conclusions: This experiment demonstrates how boosting heme production though deletion of HAP1 and overexpression of HMX1 leads to improvement in production of violacein and violacein derivatives.

The effect of boosting flavin adenine dinucleotide (FAD) was evaluated through a series of native gene overexpressions. FAD is used as a co-factor for the reactions catalyzed by CvVioA and CvVioC so it is reasoned that at high production titers, extra FAD would be required to ensure full activity. To test the effect of boosting flavin, RIB1 and FLX1 were both overexpressed in a strain containing 2 copies of the deoxyviolacein biosynthetic pathway. The results, shown in Table 16 show that under these conditions a slight increase in titer is observed, however in this background strain both CvVioA and CvVioC do not appear to be rate limiting as indicated by the low residual concentration of each substrate in the parental strain. This suggests that while FAD may not be limiting in this background strain, in strains with higher flux through the pathway, boosting FAD through strategies such as overexpression of RIB1 and FLX1 would be required.

TABLE 16
Production of deoxyviolacein in S. cerevisiae strains with
genetic strategies to boost FAD production. Titer of each
metabolite produced after cultivation for 72 h at 30°
c., TRYP; Tryptophan, PDV: Prodeoxyviolacein, DV: Deoxyviolacein,
PV: Proviolacein, VIO: Violacein, ND: Not detected.

| Strain | Genotype | Produced metabolite (mg/L) | | | | |
|---|---|---|---|---|---|---|
| name | description | TRYP | PDV | DV | PV | VIO |
| SC-184 | Parental strain | ND | 13.4 | 15.9 | ND | ND |
| SC-191 | RIB1 overexpression | ND | 12.8 | 20.1 | ND | ND |
| SC-192 | FLX1 overexpression | ND | 11.5 | 19.6 | ND | ND |

Literature reports on heterologous production of violacein frequently report that CvVioE is a rate-limiting step in the biosynthetic pathway. CvVioE can be thought of as a sort of chaperone protein which functions to bind its substrate (IPA imine dimer) and stabilize it in a favorable conformation to enable the specific 1,2 shift reaction forming protodeoxyviolaceinic acid through classic acid-base or oxidative chemistry. In the absence of CvVioE IPA imine dimer rapidly and spontaneously rearranges to form the dead-end by-product chromopyrrolic acid, with literature reports frequently reporting the occurrence of high concentrations of chromopyrrolic acid in engineered violacein strains. Given the instability of IPA imine dimer and its propensity to rapidly and spontaneously form chromopyrrolic acid, CvVioE must bind and stabilize its substrate as soon as it's produced by CvVioB before this spontaneous reaction can occur. We hypothesized that this could be more easily achieved through a fusion protein where CvVioB and CvVioE are fused together to form a single catalytic unit. Putting these two enzymes in close proximity to each other allows CvVioE to bind to the product of CvVioB (IPA imine dimer) before it can spontaneously convert to chromopyrollic acid. To test this hypothesis S. cerevisiae deoxyviolacein producing strains were constructed where CvVioB and CvVioE were fused together (CvVioB-E) with both a flexible (GGGGS3) and rigid (EAAAK3) linker. Strains were cultivated and analyzed as described above and compared to a deoxyviolacein producing strains where CvVioB and CvVioE are expressed individually and not fused together, as well as a control strain only producing chromopyrrolic acid (by overexpression of only CvVioA and CvVioB). The results shown in Table 17 demonstrate how fusion of CvVioB and CvVioE with either a flexible or rigid linker is an effective strategy to reduce chromopyrrolic acid accumulation with only trace amounts observed in these strains and a corresponding increase in deoxyviolacein and prodeoxyviolacein production.

TABLE 17
S. cerevisiae strains engineered for the production
of deoxyviolacein with fusion of CvVioB and CvVioE with
a flexible (GGGGS3) and rigid (EAAAK3) linker. Titer of
each metabolite produced after cultivation for 72 h at 30° c.

| Strain | | Produced metabolite (mg/L) | | |
|---|---|---|---|---|
| name | Genotype description | CPA | PDV | DV |
| SC-236 | Chromopyrrolic acid producer | 50.43 | ND | ND |
| SC-184 | Deoxyviolacein producer | 10.21 | 12.34 | 40.54 |
| | No CvVioB-E fusion | | | |
| SC-244 | Deoxyviolacein producer | 1.12 | 16.16 | 46.98 |
| | CvVioB-E fusion EAAAK3 linker | | | |
| SC-245 | Deoxyviolacein producer | 1.01 | 14.48 | 49.64 |
| | CvVioB-E fusion GGGGS3 linker | | | |

CPA: Chromopyrrolic acid, PDV: Prodeoxyviolacein, DV: Deoxyviolacein, ND: Not Detected.

Conclusions: This experiment shows that fusion of CvVioB and CvVioE is an effective strategy to reduce chromopyrrolic acid accumulation and increase violacein and violacein derivative production.

In these experiments, violacein biosynthetic pathway genes were sourced from native violacein producer Chromobacterium violaceum, one of many bacterial species known to producer violacein, with enzymes from these different violacein producers sharing relatively high sequence homology/similarity (typically above 90%). Other more diverse bacterial species are however known to producer other bis-indole compounds through closely related biosynthetic pathways, most notably staurosporine, rebeccamycin, K252a, AT2433-A1, borregomycin A and BE-54017. While producers of these secondary metabolites all share the same first two biosynthetic steps with violacein producers (conversion of tryptophan to IPA imine by CvVioA and conversion of IPA imine to IPA imine dimer by CvVioB), interestingly, these enzymes share relatively low sequence homology/identity (around 30% identity). These enzymes from diverse bacterial species represent alternative enzyme variants for the first two steps of the violacein biosynthetic pathway. To test whether alternative enzymes from different biosynthetic pathways could replace the native violacein biosynthetic enzymes successfully expressed previously in yeast, strains were constructed where CvVioA or CvVioB was replaced with an alternative from another species. These strains also expressed a copy of CvVioE, meaning that functional expression of an alternative gene would lead to the production and accumulation of prodeoxyviolacein. In this way functional expression could be verified qualitatively by observing a green colour in the engineered cells. The results (shown in Table 18) show that all strains constructed turned a dark green colour indicating successful production of prodeoxyviolacein and functional expression of each gene.

TABLE 18
S. cerevisiae strains engineered for the production of prodeoxyviolacein
with alternative enzymes for the first and second steps of the
biosynthetic pathway. Table indicates colour of engineered cells
after restreaking on solid media after transformation.

| | Relevant | SEQ | Alternative | Colony |
|---|---|---|---|---|
| Strain | gene | ID NO | for | colour |
| SC-139 | CvVioA | 25, 26, | Control | Dark green |
| | CvVioB | 27, 28 | | |
| SC-4567 | NlInkO | 83, 84 | CvVioA | Dark green |
| SC-4568 | AmAtmO | 87, 88 | CvVioA | Dark green |
| SC-4569 | SsStaO | 79, 80 | CvVioA | Dark green |
| SC-4570 | LaRebD | 77, 78 | CvVioB | Dark green |
| SC-4571 | SsStaD | 81, 82 | CvVioB | Dark green |
| SC-4572 | NlInkD | 85, 86 | CvVioB | Dark green |
| SC-4573 | AmAtmD | 89, 90 | CvVioB | Dark green |

Conclusions: This experiment shows how a range of enzymes with relatively low sequence homology can functionally complement the first two steps of the native violacein biosynthetic pathway catalyzed by CvVioA and CvVioB.

### Example 8—In Vitro Glycosylation of Violacein and Violacein Derivatives Part 1

In Example 7, S. cerevisiae was engineered to produce violacein and 3 other violacein derivatives (deoxyviolacein, proviolacein, and prodeoxyviolacein). However, during cultivation experiments a number of issues were observed. Firstly, engineered cells grew significantly slower and had a significantly lower final OD600 than a parental strain, and on agar plate produced noticeably smaller colonies. On agar plate we also observed a number of colonies losing their colour, indicating rapid selection against the introduced biosynthetic pathway. Overall, these findings suggested that these compounds were toxic to the engineered host strain which is not surprising given the fact that these molecules are described as being potent anti-fungals. We additionally observed that all 4 produced compounds accumulated almost entirely intracellularly indicating that the compounds could not be transported from the cell. Finally, we observed that after cell lysis and release of the compounds into the surrounding media, the compounds rapidly precipitated, indicating a very low solubility which was further confirmed by the difficulties we had preparing stock solutions of violacein where only 5 mM was soluble in 100% methanol. Issues with toxicity, solubility, and product export have significant negative effects on production of these compounds and limit the commercial viability. To overcome these issues, we investigated whether glycosylation of the compounds could provide a positive impact and improve production. Because violacein and violacein derivatives are not naturally glycosylated, we first sought to identify heterologous glycosyltransferase enzymes displaying promiscuous activity towards these compounds by running a series of in vitro glycosyltransferase enzyme assays using violacein and violacein derivatives as substrate

For in vitro studies of glycosyl transferase performance in glycosylating violacein and violacein derivatives, purified glycosyl transferases were prepared as described in Example 4 and in vitro enzyme assays run as described below in Table 19.

TABLE 19
Assay set up for producing substituted
tryptamine glycosides in vitro

| Reagent | Volume (μL) |
|---|---|
| Purified glycosyl transferase enzyme | 5 |
| 5 mM substrate | 4 |
| 1M Tris-HCl pH 7.4 | 2 |
| Milli-Q water | 8.18 |
| Alkaline phosphatase (1 U/μL) | 0.02 |
| 100 mM UDP-sugar | 0.8 |
| TOTAL | 20 |

The reaction mixture was scaled up or down as required. The reaction mixture was incubated without shaking at 30° C. for 24 hours. Extraction and analysis were performed as described in Examples 3 and 5. To confirm the identity of the produced glycosides LC-MS/QTOF was used as described above to confirm the expected mass and fragmentation pattern of each detected molecule. Quantification of violacein glycoside or other violacein derivative glycoside production was done by comparing the peak area of the substrate and the produced glycoside with authentic analytical standards (where available), where a standard was unavailable, quantification was achieved by comparing with an authentic analytical standard of the substrate. % Conversion of a substrate to a glycoside by specific Glycosyl transferases was calculated by measuring the decrease in substrate and increase in product after 24 h incubation.

In an initial proof-of-concept, glycosyltransferase enzyme Pt73Y (SEQ ID NO. 64, 66) was tested for its ability to glycosylate violacein in vitro. After 24 h incubation at 30° C. the reaction was extracted and initially analyzed by HPLC. Here a significant decrease in violacein concentration was observed relative to a no-enzyme control (75% reduction in concentration compared to the no-enzyme control). In addition to the decrease in violacein concentration, a new peak was observed at an earlier retention time (4.5 minutes compared to 5.3 minutes for violacein at 575 nm) indicative of a violacein glucoside. Further comparison of the change in retention time between the violacein peak and this new peak compared well with the change in logP of violacein (1.574) and violacein glucoside (−0.523), overall suggesting the formation of violacein glucoside. Further analysis by LC-MS/QTOF confirmed the formation of violacein glucoside with a peak at a comparable retention time observed with a mass and fragmentation pattern corresponding to that of violacein glucoside.

Conclusions: Overall this experiment demonstrates the surprising observation that glycosyltransferases from plant species can catalyze the glycosylation of violacein leading to the production of new-to-nature derivatives.

### Part II

In a follow up experiment, additional UGT enzymes were tested for their ability to glycosylate violacein and the violacein derivative proviolacein in vitro. Violacein and proviolacein glycosylation activity was tested using four different UGTs and in an in vitro enzymatic reaction containing 10% of an In situ extractant containing 3.5 mM of each substrate (350 μM final concentration), generated from culivations of SC-144 (deoxyviolacein producer) and SC-145 (proviolacein producer) with 10% isopropyl myristate as the in situ extractant. An assay mixture containing 25 mM UDP-glucose, 10 L of purified UGT enzyme and alkaline phosphatase in 100 mM Tris-HCl buffer (pH 7.4) was incubated at 30° C., 700 rpm for 66 h. The reaction was stopped by addition of 100% methanol, extracted, and analyzed as described above.

It was found that a range of UGT enzymes could catalyze the conversion of violacein and proviolacein to their respective glycosides. It was also found that different enzymes produced different glycosylation patterns. Cp73B for examples produced only a mono-glucoside while Bs109_1 and Bs109A1 predominantly produced tri and tetra-glucosides. By optimizing the reaction conditions and using a biphasic production system it was also found the Pt73Y was capable of producing higher glycosides also as compared to part I where only a mono-glucoside was detected. It was also found that all enzymes were better able to glycosylate violacein than proviolacein. Table 20 shows the % conversion of violacein to its respective glycoside for each enzyme tested, while Table 21 shows the % conversion of proviolacein.

TABLE 20
Conversion of violacein to its respective glycosides by purified
UGT enzymes. Shown is % conversion of 350 μM of substrate
to product calculated by measuring the decrease in substrate
and increase in product after 24 h incubation at 30° c.

| Product % | Enzyme | | | |
|---|---|---|---|---|
| production | Bs109_1 | Bs109A1 | Cp73B | Pt73Y |
| Mono-glucoside | 9.02 | 11.05 | 25.65 | 8.69 |
| Di-glucoside | ND | 10.57 | ND | 8.95 |
| Tri-glucoside | 10.80 | 15.29 | ND | 15.47 |
| Tetra-glucoside | 11.12 | 14.10 | ND | 10.31 |
| Penta-glucoside | ND | 11.46 | ND | 10.98 |
| Total conversion | 30.93 | 62.47 | 25.65 | 54.41 |

TABLE 21
Conversion of proviolacein to its respective glycosides by purified
UGT enzymes. Shown is % conversion of 350 μM of substrate
to product calculated by measuring the decrease in substrate
and increase in product after 24 h incubation at 30° c.

| Product % | Enzyme | | | |
|---|---|---|---|---|
| production | Bs109_1 | Bs109A1 | Cp73B | Pt73Y |
| Mono-glucoside | 2.12 | 2.45 | 8.12 | 0.12 |
| Di-glucoside | ND | 1.45 | ND | 0.15 |
| Tri-glucoside | 1.23 | 2.12 | ND | 1.13 |
| Tetra-glucoside | 3.34 | 1.98 | ND | 1.22 |
| Penta-glucoside | ND | 2.11 | ND | 0.78 |
| Total conversion | 6.69 | 10.11 | 8.12 | 3.4 |

Conclusions: Part II of this experiment demonstrates that several UGT enzymes are able to catalyze the glycosylation of violacein and violacein derivatives in vitro, and that different enzymes can be selectively used to produce different glycosides with different numbers of sugar groups attached.

### Example 9—In Vitro Production of Novel Violacein Derivatives by Feeding Serine and Substituted Indoles

While previous examples demonstrated the de novo production of violacein and 3 other natural violacein derivatives, in vitro production of these compounds from simple starting substrates using purified enzymes can offer several advantages compared to in vivo production. Furthermore, it allows for the production of novel new-to-nature derivatives by feeding different substituted substrates. Indole is a relatively cheap and abundant starting substrate which in combination with serine can be converted to tryptophan in vitro by tryptophan synthase. A broad range of substituted indoles are also commercially available which can further serve as substrates for a sufficiently promiscuous tryptophan synthases leading to new substituted tryptophan derivatives. Violacein biosynthetic genes can then incorporate these substituted tryptophan derivatives into the biosynthetic cascade, leading to new substituted violacein derivatives. These new substituted violacein derivatives not only offer an expanded colour palette but also potentially provide new bioactive function.

To demonstrate this idea, tryptophan synthase (PcTrpB, SEQ ID NO. 62, 63) and violacein biosynthetic genes (CvVioA-E SEQ ID NO's 52-56, 26, 28, 30, 32, 34) were purified from E. coli (as described in Example 4) and used in a one-pot in vitro biocatalytic reaction using serine and indole/substituted indoles as substrate, with the addition of all required co-factors. The reaction was set up according to Table 22

TABLE 22
Assay set up for producing substituted violacein derivatives in vitro

| Reagents | Volume (μL) |
|---|---|
| Purified enzymes | 30 |
| Catalase enzyme | 50 (units) |
| 50 mM indole substrate | 3 |
| 50 mM L-serine | 3 |
| 1 mM Pyridoxal phosphate (PLP) | 0.05 |
| 1M Tris-HCl pH 9.0 | 2.5 |
| 50 mM FAD | 3 |
| 50 mM NAD(P)H | 3 |
| Milli-Q water | 5.45 |
| TOTAL | 50 |

Furthermore, 50 units of catalase was added to the above reaction to improve product formation. The reaction mixture was scaled up or down as required. The reaction mixture was incubated at 30° C. for 24 hours. Reactions were stopped by freezing. Samples were extracted and analyzed as described in Examples 3 and 5. Quantification of reaction intermediates and products was done by comparing the peak area of analytical standards, where a standard was unavailable, quantification was achieved by measuring the decrease in substrate. Conversion (%) of substrates to substituted violacein derivatives by enzymatic biocatalysis was calculated by measuring the decrease in substrate and increase in product after 24 h incubation.

### Example 10—In Situ Extraction of Violacein and Violacein Derivatives by Cultivation in the Presence of Non-Ionic Surfactants at Concentrations Above their Cloud Point

As previously observed, violacein and violacein derivatives accumulated intracellularly in engineered S. cerevisiae production strains limiting the overall production of these compounds and causing cell toxicity due to the fact that these compounds have well documented anti-fungal activity. To facilitate product export and decrease cellular toxicity, engineered production strains can be cultivated in the presence of a non-ionic surfactant at a concentration above its cloud point. A cloud point is defined as the temperature at which an aqueous solution of a non-ionic surfactant transforms from uniform solution or emulsion to liquid-liquid phase separation. By cultivating a violacein or violacein derivative production strain in cultivation media with a non-ionic surfactant above its cloud point a system where with adequate mixing the cells in the aqueous phase can interact with the surfactant phase facilitating transfer of the hydrophobic compound into the surfactant phase and sequestration from the cell.

Prior art teaches that following cultivation of microorganisms engineered to produce violacein, and intracellular accumulation of violacein, prolonged incubation in the presence of high concentrations of a non-ionic surfactant leads to release of violacein from the intracellular space into the surfactant phase, facilitating downstream purification. While this method results in extraction of violacein from inside the cell, the high non-ionic surfactant concentration can lead to cell death, and since the extraction occurs after cultivation, it can't deal with the toxicity effect of intracellularly accumulating violacein. An improved method using a non-ionic surfactant at a concentration above its cloud point but below a concentration which leads to toxicity effect facilitates the in situ extraction of violacein and violacein derivatives produced during cultivation, thereby enabling the extraction of the intracellularly accumulation compound, and sequestration into a non-aqueous phase and reducing the toxicity effect of the compound.

In an initial study, the non-ionic surfactant Triton X-114 was tested for its ability to form a cloud point in liquid cultures and sequester intracellularly produced deoxyviolacein. Previous testing had shown that at 30° C. (the temperature at which S. cerevisiae strains are typically cultivated), Triton X-114 forms a cloud point in yeast liquid media at concentrations above 1%. Deoxyviolacein producer SC-144 was cultivated in 50 mL of Delft media with 0%, 2.5%, 5% and 10% Triton X-114 added for 3 days at 30° c. After cultivation cultures were transferred to 50 mL falcon tubes and centrifuged 4000×g for 5 min. After centrifugation, cultures containing Triton X-114 had separated into 3 distinct layers; a cell pellet at the bottom, an intensely dark pink/purple middle layer containing concentrated deoxyviolacein, and a clear top layer containing the majority of the non-ionic surfactant. For the control culture without Triton X-114 added only 2 layers were observed, a cell pellet at the bottom and a pale pink aqueous layer. The aqueous layers were separated from the cell pellets to observe the colour of the cells. While cells cultured without Triton X-114 we're an intense dark purple colour (indicating intracellular accumulation of deoxyviolacein), cells cultured at all 3 concentrations of Triton X-114 were completely pale white (indicating that no deoxyviolacein had accumulated intracellularly). To quantify the amount of deoxyviolacein produced under each condition, the deoxyviolacein rich aqueous phase from Triton X-114 cultures and the supernatant phase from the control culture (without Triton X-114) were measured spectrophotometrically. Samples were diluted in water to a final volume of 100 μL until they were within the linear range of the spectrophotometric plate reader. Each sample was measured at 575 nm (experimentally determined to be the emission maxima of deoxyviolacein). As shown in Table 23, samples from the Triton X-114 cultivations had significantly higher concentrations of deoxyviolacein than a sample from the control cultivation without Triton X-114. A linear correlation of deoxyviolacein concentration and total non-ionic surfactant concentration was observed with lower concentrations of surfactant giving higher concentrations of deoxyviolacein. The final OD600 of each cultivation was also measured by first washing the cell pellet with water (to remove as much deoxyviolacein from the media as possible) and finally resuspending in 50 mL water. The results shown in Table 23 show a significant increase in final OD600 for strains cultivated with the non-ionic surfactant indicating that not only is Triton X-114 non-toxic to S. cerevisiae but in situ extraction of deoxyviolacein reduces product toxicity.

Overall, this experiment demonstrates that addition of non-ionic surfactants at concentrations above their cloud point is a viable strategy to extract deoxyviolacein and other violacein derivatives from production strains.

TABLE 23
Concentration of deoxyviolacein and final OD600 from SC-144 cultivated
in the presence of Triton X-114. Qualitative concentration represented
as a relative fold change in absorbance (measured in A.U at 575
nm) relative to the control with 0% Triton X-114.

| Triton X- | Relative fold change in | Final |
|---|---|---|
| 114 (%) | Abs (A.U at 575 nm) | OD600 |
| 0 | 0 | 7.47 |
| 2.5 | 52.6 | 17.8 |
| 5 | 28.1 | 16.4 |
| 10 | 17.3 | 15.8 |

### Conclusions

The use of a non-ionic surfactant added to the cultivation at a concentration above its cloud point but below its toxicity limit is a viable strategy to facilitate in situ extraction of violacein and violacein derivatives from inside the cell thereby removing intracellular accumulation and toxicity effects from the produced compounds.

### Example 11—In Situ Extraction of Violacein and Violacein Derivatives by Cultivation in the Presence of Isopropyl Myristate

In Example 10 the problem of intracellular accumulation of violacein and violacein derivatives was solved by cultivating producer strains with non-ionic surfactants at concentrations above their cloud point. In this experiment this in situ extraction concept was expanded using isopropyl myristate as the in situ extractant. Isopropyl myristate is an attractive extractive solvent since it is relatively cheap, can be produced sustainably, is non-volatile, non-flammable, non-toxic, and generally safe for human use. To test the ability of this solvent at the in situ extraction of violacein and other violacein derivatives, deoxyviolacein producer SC-144 was cultivated in media containing 10% isopropyl myristate. After 72 h of cultivation at 30° c., cultures were centrifuged, and the supernatant collected. In the absence of isopropyl myristate the culture supernatant was faint pink colour while the cell pellet was an intense dark pink/purple colour as seen in previous experiments indicating minimal extracellular accumulation. In the presence of 10% isopropyl myristate, the culture supernatant rapidly separated into a clear aqueous layer and a dark pink/purple solvent layer. Furthermore the cell pellet was clear, overall indicating that deoxyviolacein was efficiently extracted into the isopropyl myristate which subsequently has clear phase separation from the aqueous cultivation media. To compare the amount of deoxyviolacein produced in both conditions, semi-quantification was done using a spectrophotometric plate reader as described in Example 5. The results shown in Table 24 clearly show the effectiveness of in situ extraction with isopropyl myristate. The final OD600 of each cultivation was also measured by first washing the cell pellet with water (to remove as much deoxyviolacein from the media as possible) and finally resuspending in 50 mL water. The results shown in Table 24 show a significant increase in final OD600 for strains cultivated with the solvent indicating that not only is isopropyl myristate non-toxic to S. cerevisiae but in situ extraction of deoxyviolacein reduces product toxicity.

TABLE 24
Concentration of deoxyviolacein and final OD600 from SC-144 cultivated
in the presence of Isopropyl myristate. Qualitative concentration
represented as a relative fold change in absorbance (measured in
A.U at 575 nm) relative to the control with 0% isopropyl myristate.

| Isopropyl | Relative fold change in | Final |
|---|---|---|
| myristate (%) | Abs (A.U at 575 nm) | OD600 |
| 0 | 0 | 7.37 |
| 10 | 22.3 | 17.45 |

### Conclusions

The use of isopropyl myristate added during cultivation is a viable strategy to facilitate in situ extraction of violacein and violacein derivatives from inside the cell thereby removing intracellular accumulation and toxicity effects from the produced compounds.

### Example 12—In Situ Extraction of Violacein and Violacein Derivatives by Cultivation in the Presence of Non-Ionic Surfactants Like Antifoam-A at Concentrations Above its Cloud Point

In Example 10 it was demonstrated how non-ionic surfactants such as Triton X-114 could be used for the in situ extraction of violacein and violacein derivatives such as deoxyviolacein when the non-ionic surfactant is added at a concentration above its cloud point. In this example this concept was expanded to the common antifoaming agent Antifoam-A. Antifoam-A is a silicon polymer consisting of an aqueous emulsion of a polydimethylsiloxane typically used at concentrations<0.1% to effectively suppress foaming during fermentation. At concentrations above approximately 1% a cloud point forms at 30° c. similar to other non-ionic surfactants, because Antifoam-A is commonly used in yeast fermentations and is generally non-toxic to yeast, we hypothesized that it could also be used as an in situ extractant. To test this, deoxyviolacein producing strain SC-144 was cultivated in 50 mL delft medium with 0%, 2.5% and 5% Antifoam-A added. After 72 h cultivation at 30° c. the cultures were spun down and the supernatant separated from the cell pellet. As with other experiments it was observed that the resulting cell pellet from cultivations in 2.5% and 5% Antifoam-A was a pale white indicating no intracellular accumulation of deoxyviolacein, the supernatant had split into 2 distinct layers, a clear aqueous layer and an intense dark purple surfactant layer containing the extracted deoxyviolacein. For the cultivation with no Antifoam-A added the supernatant was a very pale pink colour while the cell pellet was a dark purple colour, indicating almost exclusive intracellular accumulation. To compare the amount of deoxyviolacein produced in both conditions, semi-quantification was done using a spectrophotometric plate reader as described in Example 5. The results shown in Table 25 clearly show the effectiveness of in situ extraction with Antifoam-A. The final OD600 of each cultivation was also measured by first washing the cell pellet with water (to remove as much deoxyviolacein from the media as possible) and finally resuspending in 50 mL water. The results shown in Table 25 show a significant increase in final OD600 for strains cultivated with Antifoam-A indicating that not only is it non-toxic to S. cerevisiae but in situ extraction of deoxyviolacein reduces product toxicity.

TABLE 25
Concentration of deoxyviolacein and final OD600 from SC-144 cultivated
in the presence of Antifoam-A. Qualitative concentration represented
as a relative fold change in absorbance (measured in A.U at 575
nm) relative to the control with 0% Antifoam-A.

| Antifoam- | Relative fold change in | Final |
|---|---|---|
| A (%) | Abs (A.U at 575 nm) | OD600 |
| 0 | 0 | 7.89 |
| 2.5 | 52.6 | 18.98 |
| 5 | 28.1 | 16.8 |

### Conclusions

The use of Antifoam-A added to the cultivation at a concentration above its cloud point but below its toxicity limit is a viable strategy to facilitate in situ extraction of violacein and violacein derivatives from inside the cell thereby removing intracellular accumulation and toxicity effects from the produced compounds.

### Example 13—Simple Downstream Purification of Violacein and Violacein Derivatives from In Situ Extraction

In Example 10-12 it was shown how violacein and violacein derivatives can be efficiently extracted from inside the cell using an in situ extractant, either with non-volatile solvents like isopropyl myristate, or with non-ionic surfactants/detergents at concentrations above their cloud point. In this example a simple DSP procedure is shown to efficiently and easily purify the resulting compound from the in situ extractant. In a first example using deoxyviolacein producing strain SC-144 cultivated in the presence of 5% Antifoam-A as the in situ extractant in a total volume of 100 mL of media with 8% glucose, the following DSP procedure was performed to obtain pure compound. In the first step, the biomass was separated from the supernatant either by centrifugation or simple filtration. The resulting cell free liquid had clear phase separation with a clear bottom aqueous layer (containing cultivation media and other contaminants) and a dark purple upper layer (containing deoxyviolacein in Antifoam-A). Due to the very clear phase separation the aqueous layer was removed using a separating funnel (or similar). The deoxyviolacein rich Antifoam-A mixture was transferred to a new vessel and diluted until the antifoam layer was broken and the deoxyviolacein precipitated from solution. To isolate the precipitated deoxyviolacein the mixture was separated from the liquid by centrifugation or filtration. The resulting wet deoxyviolacein solid was resuspended in 100% ethanol to redissolve the compound. Finally, the ethanol was precipitated using a rotary evaporator or freeze drier leaving a dry dark purple powder of pure deoxyviolacein. The powder was collected and weighed to determine the final yield. From an initial 100 mL culture, 37.8 mg of deoxyviolacein was isolated. HPLC and LC-MS/QTOF analysis of the resulting powder showed a main peak corresponding to deoxyviolacein with only trace contaminating peaks observed indicating a relatively high purity.

### Conclusions

These results show that a simple DSP procedure can be implemented to efficiently purify violacein and violacein derivatives from culture broth.

### Example 14—Improved Method for the Isolation and Purification of Violacein and Violacein Derivatives from Cultivations Performed with Non-Ionic Surfactants as the In Situ Extractant

In previous examples it was demonstrated how non-ionic surfactants such as antifoam-A and Triton X-114 added to cultivations and fermentations of violacein and violacein derivative producing strains acted as efficient in situ extractants at concentrations above their cloud point. And in Example 13 it was demonstrated how the unique properties of a cloud point system could be exploited to selectively separate and extract violacein and violacein derivatives by transitioning a solution containing the compound of interest, a non-ionic surfactant such as antifoam-A or Triton X-144, and an aqueous solution such as water or cultivation media either above or below the cloud point of the system. In this example, the basic principles of the cloud point extraction presented in Example 13 were improved and refined to make it amenable to industrial scale production.

The method presented here is divided into the following broad steps with practical experimental details given to demonstrate the method in practice.
(1) Perform a fermentation or cultivation with violacein or violacein derivative producing yeast strains where a non-ionic surfactant is added at a sufficiently high concentration so that a cloud point forms between the surfactant and the aqueous media at 30° c. (the temperature that yeast is typically cultivated at). At this concentration the surfactant acts as an in situ extractant efficiently sequestering all of the product into the organic surfactant phase. At the end of the fermentation the entire fermentation broth including the product rich surfactant phase is collected for further processing.

Details on the various suitable cultivation conditions are given in other examples, in this example deoxyviolacein producer SC-144 was cultivated in the presence of 10% Antifoam-A. When performed in shakeflask the entire broth was collected at the end of the cultivation, when performed in fed-batch fermentation the product rich Antifoam-A upper phase was periodically collected and replenished with fresh Antifoam-A. The collected product rich Antifoam-A was added to later DSP steps.
(1) Separate the biomass from the liquid phase. This can be performed by methods such as centrifugation or filtration. The final cell free liquid is collected for the next phase.

In this example 100 mL of fermentation broth was centrifuged at room temperature for 5 mins in 2×50 mL falcon tubes.
(1) To remove salts and other impurities coming from the fermentation broth to achieve a higher final product purity an optional step is to separate the product rich surfactant phase from the aqueous fermentation broth phase by moving the solution further above its cloud point to achieve complete phase separation. While at the beginning of the cultivation/fermentation a clear phase separation is observed, due to continuous mixing of the phases and the accumulation of the product in the surfactant phase, by the end of the cultivation the phase separation is significantly less pronounced, with the appearance of product containing micelles in the aqueous phase and a concentration gradient in the surfactant phase. To move the solution far above its cloud point to re-establish complete phase separation various combinations of increased temperature, increase salt concentration (known as “salting out”) and enhanced separation methods like centrifugation and gravity separation can be used.

In this example, the 2×50 mL of cell free solutions were saturated with Na₂SO₄ (i.e., salt was added until the solubility limit was reached) then centrifuged at room temperature for 5 mins. The product rich upper layer was collected by pipette for the next step.
(1) In this step the product is separated from the surfactant phase by moving the solution far below its cloud point causing it to coalesce into a single aqueous phase, which causes the product to precipitate due to its low aqueous solubility. First, the product rich surfactant phase which was collected in the previous step is diluted again in distilled (salt free) water, however if a final product free of salt and other impurities from the cultivation broth is not required, then the cell free liquid from step 2 can be used directly for the next step. Next, the solution is brought far below its cloud point by various combinations of decreased temperature, the addition of chemicals known to lower the cloud point such as ethanol, and enhanced separation methods like centrifugation and gravity separation causing the product to precipitate from solution. To recover the precipitate, after removing the single surfactant-aqueous phase, it can be re-dissolved in a volatile organic solvent such as ethanol and evaporated to produce a dry powder by methods such as rotary evaporation, freezer drying etc.

In this example the product rich surfactant was diluted back to the original volume with distilled water (2×50 mL). 2×50 mL of cell free liquid from step 2 was also included to demonstrate purification directly from this step. Ethanol was added to all solutions at a final concentration of 25% causing deoxyviolacein to begin to precipitate. To speed up this process the solutions were centrifuged at room temperature for 5 mins which caused complete precipitation leaving a clear surfactant-aqueous solution and a solid deoxyviolacein precipitate. After removing the liquid phase by decanting the precipitated deoxyviolacein was resuspended in 5 mL of ethanol, pooled together and transferred to a rotary evaporator to remove most of the ethanol. Finally, to obtain a dry powder the remaining solution was transferred to a freezer dryer and run until completely dry. From an initial 200 mL fermentation sample containing deoxyviolacein at 100 mg/L, approx. 15 mg of deoxyviolacein powder was obtained, indicating a final yield of approx. 75%.
(1) In a final step the product free non-ionic surfactant is recycled to be reused for in situ extraction by again moving the remaining product free solution far above its cloud point resulting in phase separation of the surfactant in the upper phase, allowing it to be separated from the aqueous phase consisting of water or culture media. To move the solution far above its cloud point to re-establish complete phase separation various combinations of increased temperature, increase salt concentration (known as “salting out”) and enhanced separation methods like centrifugation and gravity separation can be used. Optionally, the ethanol can be removed by rotary evaporation or vacuum centrifugation to further help increase the cloud point.

In this example the decanted ethanol—Antifoam-A—solution was collected and the ethanol evaporated by vacuum centrifuge. The ethanol free solution was then saturated with Na₂SO₄ (i.e., salt was added until the solubility limit was reached) then centrifuged at room temperature for 5 mins. The upper Antifoam-A layer was collected by pipette to be reused.

Conclusions: This experiment demonstrates a simple and effective DSP method to isolate and purify violacein and violacein derivatives from cultivations with non-ionic surfactants as the in situ extractant by utilizing a cloud point system. The method comprises a first step to remove biomass by filtration or centrifugation, a second step to separate and recover the product rich non-ionic surfactant phase from the aqueous cultivation media phase by moving the solution far above its cloud point by a combination of elevated temperatures, high salt concentrations and/or centrifugation, a third step to separate and recover the product from the non-ionic surfactant by precipitation, whereby the collected non-ionic surfactant containing the product is diluted in water then moved far below its cloud point by a combination of lowered temperatures, ethanol and/or centrifugation, and finally a forth step where the non-ionic surfactant is recovered and reused by moving the product free solution once again far above its cloud point by a combination of elevated temperatures, evaporation of ethanol, high salt concentrations and/or centrifugation.

### Example 15—In Situ Extraction of Violacein and Violacein Derivatives by Cultivation in the Presence of a Range of In Situ Extractants

Given the success of extracting violacein and violacein derivatives directly from fermentation broth by adding extractants such as Antifoam-A, Triton-X 114 and isopropyl myristate directly to the fermentation, a range of additional extractants from several different chemical classes were tested to assess their ability to extract intracellularly accumulating pigments produced during the fermentation and sequester them into an organic phase, thereby simplifying DSP but also alleviating concerns around product toxicity. Given the success of Antifoam-A and isopropyl myristate in Example 11 and 12, both were also included in this experiment to quantify the amount of compound sequestered into each phase. To test different extractants, the following experiment was performed. Violacein and violacein derivative producing strains were cultivated in 50 mL of media each with a different in situ extractant added to a final concentration of 10%. Strains were cultivated for 4 days at 30° C. after which centrifugation was used to separate the cultivation into a (bottom) biomass layer, a (middle) aqueous media layer, and a (top) extractant layer. Samples were collected from each phase and produced metabolites extracted and quantified. The results (presented in Table 26) show that a surprisingly large number of extractants across several different compound classes could effectively sequester the intracellularly produced pigment into the organic extractant phase sitting at the top of the cultivation, compared to control strains with no in situ extractant where the pigment accumulated almost exclusively intracellularly. In most cases it was observed that close to 100% of the total product was found in the extractant with minimal or negligible amounts found in the aqueous and/or intracellular phase. Also of note was the difference in concentration of pigments in different organic extractant phases, with some in situ extractants containing significantly higher concentrations than others (e.g. isopropyl myristate, isopropyl palmitate, antifoam-A, polysorbate, ethyl laurate, castor oil all contained significantly higher amounts of the target pigment than other extractants). This difference is presumably a result of lower toxicity of the in situ extractant as well as higher solubility of the compound in the organic phase. Finally, it was noted that some extractants were unselective, and able to extract all pigments relatively equally (e.g. polysorbate, oleyl alcohol, castor oil, butyl caprilate), while surprisingly, some extractants displayed a clear preference for violacein and deoxyviolacein while being relatively unselective for proviolacein and prodeoxyviolacein (e.g. antifoam-a, isopropyl myristate, isopropyl palmitate, ethyl laurate, grapeseed oil, 2-butyl-1-octanol, oleic acid). Table 26 shows the efficiency of each solvent for the ability to extract intracellularly accumulating compound from yeast production strains. Shown is the concentration of each compound in the in situ extractant as well as the concentration in the extracellular and intracellular space.

TABLE 26
Violacein and violacein derivative production in the presence of 10% of various in situ extractants.
Shown is the concentration detected in each phase (in mg/L) after cultivation for 4 days at 30° c.

| | | Concentration in each phase (mg/L) | | | | | | | | | | | |
|---|---|---|---|---|---|---|---|---|---|---|---|---|---|
| | In situ solvent | Solvent | | | | Aqueous | | | | Intracellular | | | |
| Strain | (10%) | PDV | DV | PV | V | PDV | DV | PV | V | PDV | DV | PV | V |
| SC-144 | Control | N/A | N/A | N/A | N/A | 1.3 | 16.1 | N/A | N/A | 2.2 | 17.9 | N/A | N/A |
| (Deoxyviolacein | Antifoam-A | 56.7 | 483.4 | | | ND | 2.5 | | | ND | ND | | |
| producer) | N-dodecane | ND | 87.0 | | | ND | ND | | | 20.8 | ND | | |
| | Isopropyl myristate | 89.8 | 867.9 | | | 10.2 | 10.8 | | | 100.4 | ND | | |
| | Oleic acid | 27.6 | 254.5 | | | ND | 1.2 | | | 12.1 | ND | | |
| | Oleyl alcohol | 91.5 | 101.7 | | | ND | ND | | | 12.1 | ND | | |
| | Ethyl oleate | ND | 20.3 | | | 0.8 | 9.1 | | | 2.6 | ND | | |
| | 2-Butyl-1-octanol | 19.2 | 154.9 | | | ND | 1.7 | | | 14.6 | ND | | |
| | Sunflower oil | ND | 37.5 | | | ND | ND | | | 0.3 | ND | | |
| | Isopropyl palmitate | 125.6 | 1209.8 | | | 10.5 | 12.2 | | | 131.3 | 0.4 | | |
| | Ethyl laureate | 146.7 | 546.5 | | | 16.6 | 9.2 | | | 90.1 | ND | | |
| | Grapeseed oil | 71.6 | 410.6 | | | 11.7 | 18.6 | | | 48.2 | ND | | |
| | Castor oil | 474.8 | 468.7 | | | 9.5 | ND | | | 10.1 | ND | | |
| | Butyl caprilate | 80.1 | 148.7 | | | 9.5 | 7.9 | | | 15.1 | ND | | |
| | Polysorbate 20 | 193.2 | 157.1 | | | 37.4 | 24.9 | | | 6.2 | 5.2 | | |
| SC-139 | Control | N/A | N/A | N/A | N/A | ND | N/A | N/A | N/A | 123.3 | N/A | N/A | N/A |
| (Prodeoxyviolacein | Antifoam-A | 14.2 | | | | 0.4 | | | | ND | | | |
| producer) | N-dodecane | 11.3 | | | | ND | | | | ND | | | |
| | Isopropyl myristate | 45.8 | | | | 4.6 | | | | 5.1 | | | |
| | Oleic acid | 59.5 | | | | 8.3 | | | | 13.1 | | | |
| | Oleyl alcohol | 454.1 | | | | 0.0 | | | | 8.0 | | | |
| | Ethyl oleate | 15.1 | | | | ND | | | | ND | | | |
| | 2-Butyl-1-octanol | 50.5 | | | | ND | | | | 50.2 | | | |
| | Sunflower oil | 11.1 | | | | ND | | | | ND | | | |
| | Isopropyl palmitate | 121.3 | | | | 10.0 | | | | 154.3 | | | |
| | Ethyl laureate | 210.1 | | | | 11.1 | | | | 242.1 | | | |
| | Grapeseed oil | 110.2 | | | | 8.1 | | | | 115.1 | | | |
| | Castor oil | 781.3 | | | | ND | | | | 10.6 | | | |
| | Butyl caprilate | 554.1 | | | | 8.9 | | | | 14.4 | | | |
| | Polysorbate 20 | 545.1 | | | | 10.8 | | | | ND | | | |
| SC-145 | Control | N/A | N/A | N/A | N/A | ND | N/A | ND | N/A | 34.3 | N/A | 110.5 | N/A |
| (Proviolacein | Antifoam-A | 7.4 | | 17.8 | | ND | | ND | | ND | | ND | |
| producer) | N-dodecane | 3.4 | | 10.1 | | ND | | ND | | 34.3 | | 44.1 | |
| | Isopropyl myristate | 98.1 | | 99.1 | | 9.4 | | 8.7 | | 102.1 | | 129.1 | |
| | Oleic acid | 21.4 | | 67.5 | | 1.2 | | 2.4 | | 45.5 | | 98.1 | |
| | Oleyl alcohol | 123.3 | | 412.6 | | 7.8 | | 11.3 | | 10.1 | | 9.9 | |
| | Ethyl oleate | 9.1 | | 14.4 | | ND | | ND | | 2.3 | | 4.3 | |
| | 2-Butyl-1-octanol | 37.7 | | 61.2 | | ND | | ND | | 25.1 | | 58.5 | |
| | Sunflower oil | 2.1 | | 10.1 | | ND | | ND | | 2.4 | | 6.4 | |
| | Isopropyl palmitate | 45.4 | | 165.3 | | 7.5 | | 10.3 | | 45.6 | | 147.0 | |
| | Ethyl laureate | 34.2 | | 155.4 | | 9.4 | | 8.5 | | 39.4 | | 144.3 | |
| | Grapeseed oil | 35.5 | | 66.5 | | 5.6 | | 7.6 | | 41.2 | | 120.4 | |
| | Castor oil | 232.2 | | 678.7 | | 10.1 | | 12.2 | | 12.3 | | 15.5 | |
| | Butyl caprilate | 111.2 | | 564.4 | | 11.5 | | 12.3 | | 10.2 | | 17.9 | |
| | Polysorbate 20 | 123.1 | | 456.4 | | 7.4 | | 5.6 | | 1.2 | | ND | |
| SC-141 | Control | N/A | N/A | N/A | N/A | ND | N/A | ND | ND | 10.5 | N/A | 11.2 | 134.3 |
| (Violacein | Antifoam-A | 47.8 | | 26.2 | 497.1 | ND | | ND | ND | 9.1 | | 7.9 | ND |
| producer) | N-dodecane | 2.4 | | 1.2 | 78.6 | ND | | ND | ND | 13.3 | | 11.1 | 10.3 |
| | Isopropyl myristate | 78.1 | | 100.7 | 512.1 | 9.1 | | 8.7 | 7.6 | 89.1 | | 78.1 | ND |
| | Oleic acid | 21.2 | | 31.1 | 312.5 | 1.1 | | 1.4 | 9.3 | 22.2 | | 31.1 | ND |
| | Oleyl alcohol | 98.1 | | 89.3 | 231.5 | 1.3 | | 2.1 | 10.1 | 12.2 | | 14.4 | ND |
| | Ethyl oleate | 10.1 | | 9.3 | 31.1 | ND | | ND | ND | 1.1 | | 1.3 | ND |
| | 2-Butyl-1-octanol | 14.4 | | 24.4 | 145.3 | 1.1 | | 2.1 | 1.4 | 13.1 | | 20.1 | ND |
| | Sunflower oil | ND | | ND | 44.5 | ND | | ND | ND | 1.1 | | 2.3 | ND |
| | Isopropyl palmitate | 123.3 | | 254.3 | 1343.5 | 10.1 | | 12.4 | 21.4 | 11.5 | | 20.2 | 1.2 |
| | Ethyl laureate | 110.2 | | 187.2 | 555.3 | 8.3 | | 9.3 | 9.4 | 90.1 | | 110.2 | ND |
| | Grapeseed oil | 90.4 | | 123.5 | 498.3 | 5.5 | | 9.1 | 12.5 | 83.1 | | 130.3 | ND |
| | Castor oil | 156.7 | | 345.4 | 512.3 | 10.6 | | 12.4 | ND | 10.5 | | 9.3 | ND |
| | Butyl caprilate | 79.6 | | 110.4 | 210.2 | 5.6 | | 6.7 | 9.4 | 45.4 | | 34.5 | ND |
| | Polysorbate 20 | 98.1 | | 54.4 | 210.3 | 24.1 | | 10.1 | 34.1 | 1.2 | | 2.6 | 4.5 |

PDV: Prodeoxyviolacein, DV: Deoxyviolacein, PV: Proviolacein, V: Violacein, ND: Not detected, N/A: Not applicable.

Conclusions: This experiment demonstrates how a wide range of chemicals make effective in situ extractants, able to efficiently sequester violacein and violacein derivatives completely into the organic extractant phase, thereby alleviating product toxicity, solubility and intracellularly accumulation concerns, but also facilitating a much simpler DSP approach to isolating, purifying and using the produced pigment in downstream products.

### Example 16—Method for the Purification and Isolation of Violacein and Violacein Derivatives from Cultivations Performed with Lipophilic Non-Volatile Solvents as the In Situ Extractant

### Part I

In order to purify violacein and violacein derivatives from in situ extractants that do not form a cloud point (e.g. isopropyl myristate and other vegetable oils and esters thereof) the following DSP procedure can be performed using violacein and violacein derivative producers cultivated in the presence of in situ extractants.

In the first step, the pigment rich extractant phase is collected free from cell biomass and aqueous cultivation media by either filtration, centrifugation or gravity separation whereby the pigment rich extract phase forms a phase separation at the top of the cultivation and can easily be separated.

In the second step, the pigment extract is loaded 1:1 (w/w) to dry silica (60 Å pores size, 0.5-1 mm particle size) and incubated at room temperature for 1 h to allow the pigment to bind to the silica. The silica loaded with the extract is then agitated with 5 load volumes of a volatile solvent or solvent mix (for example dichloromethane, hexane, ethyl acetate) for 10 s to remove the in situ extractant. The resulting supernatant is then removed by filtration. This step can be repeated multiple times to fully remove all in situ extractant. Finally, the retentate is left to dry.

In the third step the bound pigments are eluted from the silica with 10 load volumes of ethanol. The ethanol in the collected elution fraction is then evaporated to obtain the pigments in a solid form.

This process was tested in an experiment with deoxyviolacein producer SC-144 cultivated with 20% isopropyl myristate. After loading 1 g of extract containing deoxyviolacein onto 1 g of silica and binding for 1 h, the deoxyviolacein bound silica was washed twice with 5 mL of dichloromethane. After removing the solvent by simple filtration, the deoxyviolacein was eluted by adding 10 mL of 100% ethanol. Finally, the ethanol fraction containing deoxyviolacein was transferred to a vacuum centrifuge to evaporate the ethanol, leaving a dry deoxyvviolacein powder. Subsequent quantification revealed an overall deoxyviolacein recovery of 70% and a residual solvent concentration below 0.1%.

### Part II

Alternatively, instead of bind/elute, a classic gravity or peristaltic pump driven column chromatography process can be used following the same general procedure as above (bind to silica, wash with solvent, elute with ethanol). This was demonstrated in the following experiment using deoxyviolacein producer SC-144 cultivated with 10% ethyl laurate. A glass column was packed with 10 g of silica gel in 100% hexane. 100 mL of liquid ethyl laurate extract was loaded onto the column allowing the deoxyviolacein to bind to the silica, and unbound ethyl laurate to flow through. It was determined that approximately 10% of the loaded ethyl laurate was retained in the column, while 90% flowed through. The flowthrough did not contain any deoxyviolacein. The column was subsequently washed with 150 mL hexane to remove bound ethyl laureate, subsequent analysis revealed that washing with hexane removed 95% of bound ethyl laureate while removing none of the bound deoxyviolacein. Finally, the deoxyviolacein was eluted in 100 mL of 50% ethyl acetate in hexane and the elution fraction was evaporated first in a rotary evaporator, then to dryness in a vacuum centrifuge. Subsequent analysis revealed that dry deoxyviolacein was purified with a yield of 65% and a residual solvent concentration below 0.1%.

### Example 17—Method for the Up-Concentration of Violacein and Violacein Derivatives from Cultivations Performed with Lipophilic Non-Volatile Solvents by Bringing Extractant Below its Solidification Point

For some applications, a highly pure pigment powder will be required. However for some applications e.g. in dye bath preparation, a small amount of the in situ extractant present in the final product can be tolerated. In such a case, it would be advantageous instead of investing resources in purifying the product back to a dry powder, to instead up-concentrate the product to remove as much of the in situ extractant as possible. Many of the successful in situ solvents identified in Example 15 are (or are derived from) vegetable oils, and many have solidification points at temperatures above 0° c. (e.g. isopropyl palmitate has a solidification point of approx. 16° c.). This property can be exploited to remove the in situ extractant as a solid, and further, by adding a volatile organic solvent like ethanol, the pigment can enter the organic phase as it precipitates from the solidifying oil phase. After recovery of the organic phase containing the pigment and evaporation of the volatile organic solvent. The resulting product is a product rich paste containing only a fraction of the original extractant content. Finally, the process can be repeated with the collected extractant to further increase the yield. This process was demonstrated in the following experiment. Deoxyviolacein producer SC-144 was cultivated in the presence of 10% isopropyl palmitate, at the end of the cultivation, the resulting deoxyviolacein rich extractant was collected and diluted with ethanol to a final concentration of 33% extractant (66% ethanol). The mix was slowly cooled to 4° c. with gentle stirring, causing the isopropyl palmitate to solidify and drawing the deoxyviolacein into the ethanol phase. The liquid deoxyviolacein in ethanol phase was separated from the solid isopropyl palmitate by simple filtration at 4° c. after which the ethanol was evaporated in a vacuum centrifuge resulting in a dark purple paste (containing the remaining isopropyl palmitate that had solubilized in the ethanol). After solubilization and subsequent analysis it was revealed that this procedure resulted in a 10 fold increase in deoxyviolacein concentration with a corresponding 10 fold decrease in the amount of isopropyl palmitate present.

Conclusions: This experiment shows how a significant amount of in situ extractant can be removed from the final DSP product by solidifying the extractant and removing by filtration.

### Example 18—Direct Dyeing of Textile Fabrics with Violacein and Violacein Derivatives from Purified Extracts

Given their bright colours and potent bioactivities, a useful application of violacein and violacein derivatives is to dye textiles, imparting both colour and potent bioactivities to the fabric. Most commercially available dyes don't bind directly to textiles but instead require mordanting whereby a mediator chemical (the mordant) acts to facilitate binding of the dye to the textile. This additional step adds significant cost and environmental impact to the dyeing process so it would be advantageous to use a dye which binds directly to the textile without any further additions. In an initial test to assess whether violacein and violacein derivatives could dye directly onto fabrics without any pre-treatment steps or mordants or other chemicals, purified extracts of violacein, proviolacien, prodeoxyviolacein and deoxyviolacein were prepared from yeast production strains as described above. The dried powder from each extract was resuspended in 100% ethanol and added to 5 mL glass vials containing 2×2 cm strip of various undyed fabrics (nylon 6,6, diacetate, polyester, bleached cotton, wool, hemp rayon, denim). The fabrics were incubated with each ethanol extract at room temperature (approx. 21° c.) for 30 minutes after which the fabric was removed and washed thoroughly with water to remove any excess dye and dried overnight at 30° c. The color coordinates of the resulting dyed fabric were determined according to the CIE L*a*b* System, established by the “Commission Internationale de l'Eclairage-CIE” according to ASTM D 2244-68 using a colorimeter (DataColor ColorReader Spectro), where “L” indicates the brightness, “a” describes the red-green content and “b” the yellow-blue content. The color change (ΔE) is calculated by the following equation:

$$Δ E = \sqrt{{( L 2 - L 1 )}^{2} + {( a 2 - a 1 )}^{2} + {( b 2 - b 1 )}^{2}}$$

Where L2, a2 and b2 are the colour coordinates of an undyed fabric sample and L1, a1 and b1 are the colour coordinates of the dyed fabric sample. The results of the dyeing experiment are presented in Table 27. It was found that all 4 dyes successfully imparted good colour change directly to each fabric with all fabrics retaining at least some of the colour of the dye solution. By looking at AE values it can be seen that nylon 6,6 is the best fabric for dyeing followed by diacetate, denim and bleached cotton. In general polyester is poorly dyed and wool only slightly dyed. It can also be seen that dyeing with violacein and deoxyviolacein gives stronger colour intensity than prodeoxyviolacein or proviolacein, however this could be due to the relative concentration of each dye bath (the violacein and deoxyviolacein dye baths had a much higher colour absorbance), or the fact that the colours of violacein and deoxyviolacein (purple to dark blue) are much stronger than the colours of prodeoxyviolacein and proviolacein (green and light blue). Also surprising was the observation that both synthetic (nylon 6,6) and (semi-)natural (cotton, diacetate, denim, silk, viscose) fabrics were dyed. However, what is most remarkable is the observation that a wide range of fabrics were effectively dyed under ambient dyeing conditions without any mordanting or chemical treatment steps.

TABLE 27
Direct dyeing of fabrics with pigments dissolved in 100% ethanol.
Fabrics were incubated in a dye bath at room temperature for
30 minutes. ΔE was calculated from measured colour coordinates
of fabric after dyeing and undyed fabric controls.

| Colour compound | Fabric dyed | L | a | b | ΔE |
|---|---|---|---|---|---|
| Violacein | Nylon 6,6 | 42.55 | 5.15 | −15.59 | 48.45 |
| | Diacetate | 46 | 9.04 | −9.93 | 42.18 |
| | Bleached cotton | 63.39 | 4.95 | −5.28 | 26.95 |
| | Denim | 60.57 | 0.77 | −10.83 | 33.65 |
| | Polyester | 68.51 | 2.89 | −0.37 | 20.67 |
| | Wool | 64.53 | 0.76 | 1.21 | 19.76 |
| | Viscose | 64.89 | −0.95 | −2.06 | 23.50 |
| | Silk | 72.14 | 3.83 | −5.27 | 19.22 |
| | Hemp | 70.83 | 2.66 | −6.04 | 20.87 |
| Deoxyviolacein | Nylon 6,6 | 44.17 | 2.55 | −14.56 | 46.42 |
| | Diacetate | 46.46 | 10.78 | −13.47 | 43.10 |
| | Bleached cotton | 64.7 | 2.55 | −7.44 | 25.98 |
| | Denim | 55.38 | 2.59 | −8.78 | 38.02 |
| | Polyester | 67.73 | 0.17 | 0.03 | 21.29 |
| | Wool | 62.08 | −1.13 | 1.71 | 22.14 |
| | Viscose | 62.42 | 2.98 | −1.89 | 25.86 |
| | Silk | 72.02 | 2.4 | −2.37 | 18.38 |
| | Hemp | 75.81 | 1.7 | −5.6 | 15.97 |
| Prodeoxyviolacein | Nylon 6,6 | 61.99 | 1.4 | 6 | 27.44 |
| | Diacetate | 67.15 | 1.39 | 9.16 | 21.05 |
| | Bleached cotton | 74.15 | 2.01 | 4.43 | 15.86 |
| | Denim | 73.29 | 3.33 | 5.2 | 19.44 |
| | Polyester | 71.81 | −0.85 | 3.08 | 17.57 |
| | Wool | 65.86 | −1.83 | 6.18 | 18.19 |
| | Viscose | 70.58 | 1.7 | 4.57 | 17.25 |
| | Silk | 74.63 | −0.01 | 5.09 | 15.89 |
| | Hemp | 80.4 | 0.87 | 4.4 | 11.01 |
| Proviolacein | Nylon 6,6 | 64.73 | 3.29 | 2.07 | 24.14 |
| | Diacetate | 70.57 | 2.52 | 5.61 | 16.60 |
| | Bleached cotton | 77.07 | 3.28 | 2.46 | 12.77 |
| | Denim | 73.29 | 3.33 | 5.2 | 19.44 |
| | Polyester | 74.88 | −0.22 | 1.66 | 14.28 |
| | Wool | 70.77 | −0.51 | 5.17 | 13.14 |
| | Viscose | 78.01 | 0.84 | 7.88 | 10.91 |
| | Silk | 82.02 | 1.68 | 3.73 | 8.50 |
| | Hemp | 84.21 | 2.59 | 3.19 | 7.26 |
| Undyed control | Nylon 6,6 | 88.51 | 0.5 | −0.97 | N/A |
| | Diacetate | 86.15 | 0.87 | 0.11 | N/A |
| | Bleached cotton | 89.44 | 0.94 | 0.34 | N/A |
| | Denim | 92.1 | 0.9 | 0.93 | N/A |
| | Polyester | 89.02 | 0.29 | −0.27 | N/A |
| | Wool | 83.87 | 0.55 | 5.28 | N/A |
| | Viscose | 87.75 | 1.14 | 2.97 | N/A |
| | Silk | 89.98 | 0.44 | 1.02 | N/A |
| | Hemp | 90.62 | 0.76 | 0.31 | N/A |

Conclusions: This experiment shows that violacein and violacein derivatives can dye directly onto a range of synthetic and natural fibers under ambient conditions with good colour strength and without the need for any pre-treatments, mordants or other chemical processing steps.

### Example 19—Direct Dyeing of Textile Fabrics with Violacein and Violacein Derivatives from Lipophilic In Situ Extractants Diluted in Organic Solvents

### Part I

In Example 18 it was shown that purified pigments dissolved in ethanol were able to directly dye various fabrics with good colour strength, however the requirement to first purify each pigment to a powder before resuspending in ethanol is an obvious drawback, adding additional cost and environmental impact to the process. In previous experiments it was shown that in situ extraction was a viable and successful technique to overcome the solubility, toxicity and transport issues of violacein and violacein derivatives, with 100% of the produced pigment being sequestered into the extractant phase allowing simple product recovery from the fermentation broth. To avoid the additional purification process, it would be advantageous to use the in situ extractant containing violacein or a violacein derivative to dye fabrics directly. In an initial test, deoxyviolacein producer SC-144 was cultivated in the presence of 20% isopropyl myristate to effectively sequester deoxyviolacein into the extractant phase. The deoxyviolacein rich isopropyl myristate fraction was collected and used directly as a dye bath to dye Nylon 6,6. A 2×2 cm piece of undyed Nylon 6,6 was incubated in the dye bath at room temperature for 30 mins with gentle shaking, after 30 mins the fabric was washed with water and dried overnight at 30° c. Colour coordinates and colour change were determined as described in Example 18. It was shown that this procedure was effective to dye Nylon 6,6 with the piece of fabric turning a deep mauve colour. It was however noted that the nylon 6,6 fabric was not evenly dyed which was assumed to be due to the high viscosity of the dye bath and its difficulty penetrating fully through the fabric. Since it was noted (in this experiment and in previous experiments) that violacein and violacein derivatives have excellent colour strength, it was reasoned that the viscous isopropyl myristate extract could be diluted to improve the ability of the dye to penetrate the fabric, without affecting the colour strength of the fabric.

To confirm this, a follow-up experiment was performed whereby all 4 colour production strains were grown in media containing 20% isopropyl myristate and the colour rich extractant phase collected after cultivation. The colour rich isopropyl myristate extractant was then diluted 10× in 100% ethanol (10% isopropyl myristate final concentration) and used as dye baths to dye various fabrics (according to Example 18). The results of the experiment are shown in Table 28. The results demonstrate effective dyeing of a range of fabrics with results broadly similar to, and in some cases exceeding dyeing with purified compounds in 100% ethanol.

TABLE 28
Direct dyeing of fabrics with pigments dissolved in 90% ethanol 10%
isopropyl myristate. Fabrics were incubated in a dye bath at room
temperature for 30 minutes. ΔE was calculated from measured colour
coordinates of fabric after dyeing and undyed fabric controls.

| Colour compound | Fabric dyed | L | a | b | ΔE |
|---|---|---|---|---|---|
| Violacein | Nylon 6,6 | 18.84 | 4.8 | −13.54 | 70.93 |
| | Diacetate | 34.09 | 6.1 | −16.13 | 54.78 |
| | Bleached cotton | 56.27 | 1.09 | −11.8 | 35.32 |
| | Denim | 60.57 | 0.77 | −10.83 | 33.65 |
| | Polyester | 69.61 | 1.87 | −2.7 | 19.63 |
| | Wool | 59.82 | 1.09 | −10 | 28.50 |
| | Viscose | 55.54 | 1.75 | −5.49 | 33.31 |
| | Silk | 65.74 | 2.69 | −6.89 | 25.60 |
| | Hemp | 63.82 | 3.08 | −7.55 | 28.03 |
| Deoxyviolacein | Nylon 6,6 | 26.71 | 7.86 | −18.25 | 64.59 |
| | Diacetate | 40.75 | 10.56 | −26 | 53.26 |
| | Bleached cotton | 59.86 | 2.98 | −6.24 | 30.37 |
| | Denim | 55.38 | 2.59 | −8.78 | 38.02 |
| | Polyester | 62.13 | 2.37 | −1.86 | 27.02 |
| | Wool | 58.95 | 1.83 | −3.21 | 26.36 |
| | Viscose | 63.87 | 10.08 | −8.76 | 28.07 |
| | Silk | 70.65 | 2.7 | −8.92 | 21.85 |
| | Hemp | 78.38 | 2.65 | −5.77 | 13.80 |
| Prodeoxyviolacein | Nylon 6,6 | 51.94 | 2.82 | −4.13 | 36.78 |
| | Diacetate | 65.8 | 8.71 | 9.17 | 23.62 |
| | Bleached cotton | 73.52 | 5.18 | 2.53 | 16.62 |
| | Denim | 73.29 | 3.33 | 5.2 | 19.44 |
| | Polyester | 72.21 | 0.23 | 1.95 | 16.96 |
| | Wool | 72.7 | 0.13 | 6.6 | 11.26 |
| | Viscose | 81.33 | 3.09 | 5.66 | 7.23 |
| | Silk | 84.35 | 1.48 | 3.06 | 6.08 |
| | Hemp | 86.5 | 0.98 | 1.87 | 4.41 |
| Proviolacein | Nylon 6,6 | 53.02 | −1.14 | −6.72 | 35.99 |
| | Diacetate | 67.56 | 5.46 | 6.12 | 20.07 |
| | Bleached cotton | 70.58 | 1.62 | 2.48 | 18.99 |
| | Denim | 76.44 | 1.64 | 3.03 | 15.82 |
| | Polyester | 69.14 | −0.11 | 1.58 | 19.97 |
| | Wool | 70.02 | −0.52 | 5.88 | 13.90 |
| | Viscose | 74.97 | 4.49 | 9.62 | 14.79 |
| | Silk | 77.85 | 3.1 | 4.86 | 13.00 |
| | Hemp | 81.04 | 1.25 | 4.12 | 10.32 |
| Undyed control | Nylon 6,6 | 88.51 | 0.5 | −0.97 | N/A |
| | Diacetate | 86.15 | 0.87 | 0.11 | N/A |
| | Bleached cotton | 89.44 | 0.94 | 0.34 | N/A |
| | Denim | 92.1 | 0.9 | 0.93 | N/A |
| | Polyester | 89.02 | 0.29 | −0.27 | N/A |
| | Wool | 83.87 | 0.55 | 5.28 | N/A |
| | Viscose | 87.75 | 1.14 | 2.97 | N/A |
| | Silk | 89.98 | 0.44 | 1.02 | N/A |
| | Hemp | 90.62 | 0.76 | 0.31 | N/A |

Conclusions: This experiment shows that the pigment rich extractant phase harvested directly from fermentation with in situ product recovery can be used “as is” or diluted in another organic solvent like ethanol to effectively dye fabrics, without the need for further downstream purification. In the case of diluting the extractant phase in ethanol, these results show that the impact of the in situ solvent on the dyeing process is negligible with dyeing results broadly similar to 100% ethanol.

### Part II

Given the success of dyeing fabrics directly with violacein and violacein derivatives from isopropyl myristate in situ extractant diluted in ethanol a follow-up experiment was performed to test whether other successful lipophilic solvents identified in Example 15 containing pigment could also be used directly to dye fabrics after diluting in ethanol. The most successful extractants containing deoxyviolacein were diluted with ethanol and used to dye a range of fabrics as described above (incubating at room temperature for 30 mins before washing, drying, and measuring colour coordinates). The results presented in Table 29 show that remarkably, all solvents tested containing deoxyviolacein could effectively dye a wide range of fabrics with high AE values reported across the different solvents and fabrics.

TABLE 29
Direct dyeing of fabrics with deoxyviolacein dissolved
in 66% ethanol 33% in situ extractant produced from in
situ cultivations with 10% extractant added. Fabrics were
incubated in a dye bath at room temperature for 30 minutes.
ΔE was calculated from measured colour coordinates
of fabric after dyeing and undyed fabric controls.

| Extractant | Fabric dyed | L | a | b | ΔE |
|---|---|---|---|---|---|
| Isopropyl palmitate | Diacetate | 30.6 | 10.2 | −16.4 | 58.7 |
| | Bleached cotton | 56.3 | 1.6 | −11.5 | 35.2 |
| | Nylon 6,6 | 31.1 | 8.9 | −22.5 | 61.9 |
| | Polyester | 60.4 | 0.7 | −5.2 | 29.1 |
| | Acrylic | 54.7 | 0.7 | −4.9 | 25.9 |
| | Wool | 58.9 | −0.5 | −5.3 | 27.1 |
| Ethyl laureate | Diacetate | 38.3 | 12.4 | −15.0 | 51.5 |
| | Bleached cotton | 59.3 | 1.0 | −8.0 | 31.3 |
| | Nylon 6,6 | 37.1 | 7.8 | −20.9 | 55.6 |
| | Polyester | 60.6 | 2.6 | −2.0 | 28.5 |
| | Acrylic | 58.1 | 2.2 | −0.5 | 22.1 |
| | Wool | 59.4 | 2.4 | −3.0 | 25.9 |
| Grapeseed oil | Diacetate | 34.9 | 12.4 | −17.5 | 55.4 |
| | Bleached cotton | 62.9 | 2.1 | −10.0 | 28.5 |
| | Nylon 6,6 | 33.6 | 8.8 | −23.5 | 59.9 |
| | Polyester | 64.4 | 0.0 | −2.4 | 24.7 |
| | Acrylic | 62.1 | −0.2 | 0.2 | 18.2 |
| | Wool | 62.9 | −0.6 | −1.4 | 22.0 |
| Castor oil FAME | Diacetate | 46.4 | 13.2 | −5.1 | 41.9 |
| | Bleached cotton | 61.0 | 4.8 | −1.4 | 28.7 |
| | Nylon 6,6 | 42.2 | 8.1 | −14.1 | 48.7 |
| | Polyester | 53.8 | 8.3 | −1.7 | 36.2 |
| | Acrylic | 56.5 | 9.1 | −1.2 | 25.0 |
| | Wool | 58.1 | 7.8 | 1.6 | 27.0 |
| Polysorbate 20 | Diacetate | 67.1 | 8.2 | 2.7 | 20.6 |
| | Bleached cotton | 79.3 | 2.3 | −2.4 | 10.6 |
| | Nylon 6,6 | 57.4 | 3.1 | −9.4 | 32.4 |
| | Polyester | 78.0 | −0.2 | −3.5 | 11.5 |
| | Acrylic | 77.5 | 0.1 | −2.2 | 3.4 |
| | Wool | 74.3 | −2.4 | 1.5 | 10.7 |
| Undyed control | Diacetate | 88.5 | 0.5 | −1.0 | N/A |
| | Bleached cotton | 86.2 | 0.9 | 0.1 | N/A |
| | Nylon 6,6 | 89.4 | 0.9 | 0.3 | N/A |
| | Polyester | 92.1 | 0.9 | 0.9 | N/A |
| | Acrylic | 89.0 | 0.3 | −0.3 | N/A |
| | Wool | 83.9 | 0.6 | 5.3 | N/A |

Conclusions: This experiment demonstrates that a wide range of in situ extractants containing violacein and violacein derivatives can be used for dyeing fabrics directly.

### Example 20—Direct Dyeing of Textile Fabrics with Violacein and Violacein Derivatives from Non-Ionic Surfactant In Situ Extractants Diluted in Water

Almost all organic solvents compatible with in situ product recovery are highly immiscible in water (in fact water immiscibility is typically a pre-requisite for use in in situ product recovery). This means that in order to dilute the pigment rich extract for dyeing, it must be diluted in another organic solvent and not in an aqueous solvent (e.g., water), as is the case with pigments in ispropyl myristate which need to be diluted in ethanol and can't be diluted in water. While using a dye bath consisting of mostly ethanol (or other organic solvents) has some clear advantages (e.g., reducing water use, high wettability and dyeability, better dye economics, ability to recycle the solvent), it also has some disadvantages (e.g., increased cost, more complicated dye process). In some cases, it may therefore be preferable to have a dye bath consisting of mostly water or other aqueous solvents. While the organic solvents used for in situ product recovery cannot be made to be miscible with water, the non-ionic surfactants previously demonstrated to be effective in situ extractants can be made to be miscible in water simply by diluting below their cloud point, and in fact, non-ionic surfactants are commonly used as dispersing agents to prepare aqueous dye baths containing hydrophobic pigments. This suggests that using a non-ionic surfactant as the in situ extractant (above its cloud point) then diluting the pigment rich extractant phase in water (below its cloud point) could create an effective aqueous dye bath, while still alleviating the need to invest in costly DSP operations to generate a pure dry powder.

To test this hypothesis an experiment was set up where deoxyviolacein producer SC-144 was cultivated with 10% of the non-ionic surfactant Antifoam-A. At the end of the cultivation, because Antifoam-A was added above its cloud point at 30° c. a clear phase separation had occurred where the deoxyviolacein rich Antifoam-A layer was sitting at the top of the aqueous cultivation media, allowing for simple extraction. The harvested extract was then cooled to room temperature and diluted in room temperature water until a single phase was produced (i.e. diluted until the Antifoam-A was below its cloud point at room temperature) (approx. 1%). The resulting aqueous suspension was still an intensely purple colour indicating it would make a suitable dye bath. To test this a piece of nylon 6,6 fabric was dyed according to Example 18, with incubation at room temperature overnight followed by measurement of the resulting colour coordinates and calculation of the AE by comparison to undyed fabric control. The following results were obtained L=21.23, a=7.43, b=−16.54, ΔE=32.31 which are broadly similar to the coordinates obtained by diluting an isopropyl myristate extract in ethanol but was a lower ΔE presumably due to the higher dilution of the extract/pigment (1% vs 10%), overall indicating that the aqueous water bath could be used to dye fabrics.

Conclusions: This experiment shows that an aqueous water bath can be created by diluting a non-ionic surfactant extractant phase in water with the resulting dyed fabrics displaying good colour properties.

### Example 21—Direct Dyeing of Textile Fabrics by Cultivating with Violacein and Violacein Derivative Producing Strains

In previous experiments it was found that violacein and violacein derivatives extracted from the fermentation broth, either by in situ extraction during the fermentation or liquid-liquid and/or liquid/solid extraction after the fermentation, could be used directly to dye fabrics without any additional downstream processing. To further simplify the procedure, it may be advantageous to add the fabrics directly to the fermentation so that they can be dyed directly during the fermentation. Given the surprising observation that violacein and violacein derivatives can dye a range of fabrics directly without any pre-treatments, mordants or other chemicals, it is feasible that fabrics added directly to the fermentation will be effectively dyed as the coloured pigment is produced by the engineered yeast strains. To test this hypothesis, yeast strains producing each of the 4 pigments (SC-139: Prodeoxyviolacein, SC-141: Violacein, SC-144: Deoxyviolacein, SC-145: Proviolacein) were cultivated in 5 mL cultivation tubes with 8% glucose with no in situ extractant. 2×2 cm pieces of various undyed fabrics were sterilized by submerging in 75% ethanol, air drying in a laminar air flow hood then were added to each cultivation. Undyed fabric incubated with a wild-type strain of S. cerevisiae was used as a negative control. Strains were cultivated at 30° c. and shaking at 200 RPM for 4 days, after which the fabric was washed thoroughly with water and dried overnight at 30° c. Colour coordinates and colour change were determined as described in Example 18 (by comparing to the negative control). The results are presented in Table 30. Surprisingly, it was found that the fabrics were efficiently dyed when added during the cultivation with colour change equivalent and in some cases even higher than dyeing from recovered extractant. Also surprising was that both the aqueous cultivation media and the resulting cell biomass were completely colourless, indicating that all of the produced pigment was being efficiently extracted from the cell and dyed directly onto the fabric, indicating that the fabrics themselves act as efficient in situ extractants.

TABLE 30
Direct dyeing of fabrics incubated with pigment production strains.
Strains were cultivated for 4 days at 30° c. with each fabric
added directly to the cultivation. ΔE was calculated from measured
colour coordinates of fabric after dyeing and fabric controls
incubated with a wild-type (non-pigment producing) strain.

| | Pigment | Fabric | | | | |
|---|---|---|---|---|---|---|
| Strain | produced | added | L | a | b | ΔE |
| SC-139 | Prodeoxy- | Diacetate | 55.24 | 18.25 | 12.42 | 37.54 |
| | violacein | Bleached cotton | 46.69 | 2.72 | 3 | 42.87 |
| | | Nylon 6,6 | 51.71 | 17.02 | 5.49 | 40.85 |
| | | Polyester | 68.58 | −2.85 | −0.74 | 20.69 |
| | | Acrylic | 67.35 | 1.52 | −2.43 | 12.99 |
| | | Wool | 65.21 | 0.01 | 4.92 | 18.67 |
| SC-141 | Violacein | Diacetate | 19.08 | 3.93 | −3.99 | 67.26 |
| | | Bleached cotton | 33.54 | 0.87 | −3.74 | 56.05 |
| | | Nylon 6,6 | 23.99 | 1.9 | −11.95 | 65.46 |
| | | Polyester | 47.14 | 1.59 | −2.13 | 41.94 |
| | | Acrylic | 43.82 | 2.27 | 0 | 36.37 |
| | | Wool | 40.77 | 1.23 | 3.1 | 43.16 |
| SC-144 | Deoxy- | Diacetate | 18.84 | 5.78 | −8.82 | 68.08 |
| | violacein | Bleached cotton | 31.3 | 0.29 | −8.76 | 58.85 |
| | | Nylon 6,6 | 18.34 | 4.31 | −11.75 | 71.10 |
| | | Polyester | 41.47 | 0.76 | −2.41 | 47.60 |
| | | Acrylic | 46.01 | 3.09 | −2.94 | 34.31 |
| | | Wool | 34.46 | −0.66 | −4.15 | 50.32 |
| SC-145 | Pro- | Diacetate | 77.65 | 5.81 | 4.19 | 10.64 |
| | violacein | Bleached cotton | 80.87 | 2.95 | 1.27 | 8.85 |
| | | Nylon 6,6 | 73.8 | 8.49 | 5.95 | 18.11 |
| | | Polyester | 85.35 | 1.03 | 0.11 | 3.76 |
| | | Acrylic | 81.95 | 1.76 | −0.26 | 1.85 |
| | | Wool | 81.13 | 1.01 | 5.66 | 2.80 |
| Wild- | N/A | Diacetate | 75.62 | 2.23 | 5.97 | 12.13 |
| type | | Bleached cotton | 80.32 | 0.85 | 10.23 | 13.45 |
| | | Nylon 6,6 | 76.59 | 2.64 | 10.12 | 16.42 |
| | | Polyester | 82.32 | 0.93 | 3.89 | 7.91 |
| | | Acrylic | 81.82 | 1.17 | 2.44 | 3.26 |
| | | Wool | 76.91 | 2.43 | 8.09 | 7.74 |

Conclusions: This experiment further shows the versatility of violacein and violacein derivatives to simply and efficiently dye a range of fabrics, in this case demonstrating that fabrics added directly to the fermentation of production strains are efficiently dyed with excellent colour strength.

### Example 22—Improved Properties of Violacein and Violacein Derivative Glucosides

In Example 8 it was shown that specific glycosyltransferase enzymes (UGT's) could surprisingly catalyze the conversion of violacein and proviolacein to their respective glucosides. In this experiment, qualitative assessments of the solubility, stability and colour of glycosylated violacein and proviolacein were made. The first notable observation was that after the scale-up glycosylation reaction the aqueous phase of the reaction mix was purple (for violacein) and blue (for proviolacein). This indicated that not only were glycosides of violacein and proviolacein water soluble, but that the glycosylated derivatives retained the original colour. This observation was confirmed by HPLC of the aqueous phase of the reaction which showed that the produced glycosides were in the purple and blue colour spectrum. While its well known that glycosylation greatly improves the water solubility of molecules, the attachment of a sugar group typically changes the physiochemical properties of the molecule. In the case of coloured compounds, this typically eliminates their colour. The observation here that glycosylation results in a derivative that retains the original colour is both surprising and commercially useful for use in aqueous products e.g. beverages. To test the stability of violacein and proviolacein glycosides in aqueous solution the solutions were exposed to heat (incubated at 80° c. for 6 h) and UV (incubated in a UV hood for 6 h). HPLC analysis before after each test was used to measure the amount that had degraded during exposure. It was found that exposure to each of these conditions results in negligible changes in the absorbance indicating high stability. (approx. 0.1% lost during exposure).

Conclusions: This experiment demonstrates the water solubility and high stability of glycosides of violacein and violacein derivatives and further shows the retention in colour, making these compounds highly suitable for aqueous formulations e.g. beverages.

### Example 23—Direct Dyeing of Textile Fabrics with Violacein Glucoside after Activation by Beta-Glucosidases

While in previous examples it was shown that violacein and violacein derivatives formulated in organic solvents like ethanol could be efficiently dyed onto a range of fabrics, in some cases the use of a volatile solvent like ethanol may not be advantageous. In such a situation, using an aqueous dye solution may be preferred. In Example 8 it was found that glycosides of violacein and proviolacein were soluble in water allowing for a simple dye bath formulation. In an initial experiment it was found that unsurprisingly due to the hydrophilicity of the compound, violacein glycoside did not dye onto fabrics. To get around this a strategy was devised whereby fabric would be added to an inert dye bath containing violacein glycoside in water and incubated with a beta-glucosidase which could cleave off the attached sugar group and convert violacein glycoside back to violacein and thereby facilitate its attachment to the fabric. To test this, an aqueous solution of violacein glucoside was prepared and a 2×2 cm strip of nylon 6,6 fabric was added along with 10 units of a beta-glucosidase cocktail (Viscozyme, Novozymes). This was incubated for 1 h at room temperature then the resulting fabric was washed and evaluated as described above. The results are shown in Table 31 and are compared to a reaction where the beta-glucosidase cocktail was not added and to a control reaction containing unglycosylated violacein in 100% ethanol.

TABLE 31
Colour coordinates of nylon fabric dyed with violacein
glucoside and beta glucosidase. Dyeing for 1 h at room
temperature with 10 units of beta glucosidase as added.

| | Beta-glucosidase | | | | |
|---|---|---|---|---|---|
| Compound | added? | L | a | b | ΔE |
| Violacein glucoside | Yes | 21.37 | 1.9 | −11.3 | 61.54 |
| Violacein glucoside | No | 76.12 | 2.59 | 10.11 | 16.32 |
| Violacein | No | 22.34 | 1.8 | −11.75 | 65.16 |

The results show that the violacein glycoside beta-glucosidase reaction has equivalent dyeing strength to a violacein only solution, indicating that violacein glycoside was effectively converted back to violacein by beta-glucosidase allowing it to dye effectively onto the nylon fabric.

Conclusions: This experiment demonstrates how violacein and violacein derivatives can be formulated in an aqueous dye bath through conversion to their respective glycosides and how this can be used to effectively dye fabrics.

### Example 24—Testing the Colour Fastness of Textile Fabrics Directly Dyed with Violacein and Violacein Derivatives Under a Range of Conditions

In order to assess the stability and colour fastness of violacein and violacein derivatives dyed onto fabrics, dyed fabrics were subjected to a number of conditions. Fabrics were dyed according to Example 18 and colour coordinates and colour change before and after exposure to a given condition were determined as described in Example 18. To measure temperature stability, fabrics were incubated in an oven set to 80° c. for 4 h. To measure stability under acid and alkaline conditions, fabrics were fully submerged in water solutions set to pH 8 with NaOH and pH 5 with HCl and incubated at room temperature for 30 minutes. Fabrics were then incubated at 37° c. for 4 h. To measure stability during laundering, fabrics were washed at 30° c. under the “mix” setting of a Siemens IQ300 washing machine, then dried in a Siemens IQ300 dryer until dry. To measure artificial light stability, fabrics were placed under an LED lamp at room temperature for 16 h. To measure UV light stability, fabrics were placed under a UV lamp at room temperature for 1 h. The results for each stability test are presented in Tables 31-36.

TABLE 31
Stability during laundering of fabrics dyed with violacein and
violacein derivatives. ΔE was calculated from measured colour
coordinates of fabric before and after the stability test.

| Product | Fabric | Measurement | L | a | b | ΔE |
|---|---|---|---|---|---|---|
| Deoxy- | Nylon 6,6 | Before test | 44.46 | 6.19 | −22.02 | 3.61 |
| violacein | | After test | 47.79 | 6.03 | −20.63 | |
| | Diacetate | Before test | 50.89 | 9.08 | −15.27 | 4.25 |
| | | After test | 54.82 | 10.32 | −14.22 | |
| | Bleached | Before test | 61.14 | 0.73 | −8.03 | 8.69 |
| | cotton | After test | 69.57 | 0.39 | −5.93 | |
| | Polyester | Before test | 64.92 | 1.08 | −5.1 | 10.50 |
| | | After test | 74.17 | −0.99 | −0.58 | |
| | Wool | Before test | 63.37 | −0.25 | −1.82 | 10.79 |
| | | After test | 72.9 | −0.9 | 3.2 | |
| | Acrylic | Before test | 60.9 | 0.39 | −2.66 | 9.20 |
| | | After test | 69.42 | −1.13 | 0.45 | |
| Violacein | Nylon 6,6 | Before test | 34.78 | 4.7 | −19.46 | 3.02 |
| | | After test | 36.95 | 5.38 | −21.45 | |
| | Diacetate | Before test | 44.66 | 6.86 | −11.23 | 3.80 |
| | | After test | 48.28 | 7.64 | −12.1 | |
| | Bleached | Before test | 59.44 | 2.08 | −7.15 | 8.00 |
| | cotton | After test | 67.09 | 2.56 | −9.43 | |
| | Polyester | Before test | 69.07 | 1.41 | −0.67 | 9.71 |
| | | After test | 78.67 | 0.26 | 0.27 | |
| | Wool | Before test | 66.17 | 1.43 | 0.12 | 7.91 |
| | | After test | 73.9 | 1.48 | 1.8 | |
| | Acrylic | Before test | 62.97 | 2.52 | 0.79 | 13.65 |
| | | After test | 76.54 | 1.43 | 1.73 | |
| Prodeox- | Nylon 6,6 | Before test | 56.14 | −3.71 | −6.71 | 5.59 |
| violacein | | After test | 61.63 | −3.37 | −7.69 | |
| | Diacetate | Before test | 67.33 | 1.9 | 4.62 | 5.17 |
| | | After test | 72.04 | 3.38 | 3.1 | |
| | Bleached | Before test | 66.63 | −0.36 | 2.52 | 9.90 |
| | cotton | After test | 76.23 | 0.12 | 0.13 | |
| | Polyester | Before test | 67.43 | −1.9 | 1.6 | 7.66 |
| | | After test | 74.95 | −2.68 | 0.37 | |
| | Wool | Before test | 68.36 | −2.09 | 5.81 | 5.75 |
| | | After test | 73.46 | −0.29 | 3.85 | |
| | Acrylic | Before test | 61.29 | −1.67 | 3.19 | 9.68 |
| | | After test | 70.87 | −1.74 | 1.8 | |
| Pro- | Nylon 6,6 | Before test | 50.65 | −1.57 | −2.84 | 6.35 |
| violacein | | After test | 56.92 | −1.51 | −3.83 | |
| | Diacetate | Before test | 62.86 | 4.96 | 5.09 | 5.50 |
| | | After test | 68.23 | 3.76 | 5.07 | |
| | Bleached | Before test | 65.1 | 1.34 | 3.23 | 8.09 |
| | cotton | After test | 73 | 0.55 | 1.68 | |
| | Polyester | Before test | 65.21 | −1.18 | 1.47 | 7.70 |
| | | After test | 72.82 | −0.46 | 0.54 | |
| | Wool | Before test | 64.87 | 0.09 | 5 | 7.78 |
| | | After test | 72.54 | −1.14 | 5.37 | |
| | Acrylic | Before test | 58.81 | 0.01 | 1.72 | 9.14 |
| | | After test | 67.87 | −1.12 | 2.23 | |

TABLE 32
Temperature stability of fabrics dyed with violacein and violacein
derivatives. ΔE was calculated from measured colour coordinates
of fabric before and after the stability test.

| Product | Fabric | Measurement | L | a | b | ΔE |
|---|---|---|---|---|---|---|
| Deoxy- | Nylon 6,6 | Before test | 44.99 | 6.18 | −21.43 | 0.67 |
| violacein | | After test | 45.2 | 6.79 | −21.25 | |
| | Diacetate | Before test | 51.65 | 10.65 | −14.56 | 0.92 |
| | | After test | 51.96 | 10.78 | −13.7 | |
| | Bleached | Before test | 63.16 | 1.56 | −7.66 | 0.92 |
| | cotton | After test | 63.74 | 2.09 | −8.13 | |
| | Polyester | Before test | 64.62 | 1.61 | −2.79 | 0.98 |
| | | After test | 65.03 | 2.08 | −2.03 | |
| | Wool | Before test | 66.67 | −0.09 | −0.62 | 2.10 |
| | | After test | 66.12 | 1.93 | −0.48 | |
| | Acrylic | Before test | 59.1 | 1.55 | −1.29 | 1.77 |
| | | After test | 59.66 | 0.86 | −2.82 | |
| Violacein | Nylon 6,6 | Before test | 34.91 | 5 | −20.42 | 1.41 |
| | | After test | 33.74 | 5.53 | −19.85 | |
| | Diacetate | Before test | 43.1 | 8.4 | −12.5 | 2.79 |
| | | After test | 41.38 | 10.6 | −12.61 | |
| | Bleached | Before test | 60.51 | 2.66 | −6.54 | 2.28 |
| | cotton | After test | 59.33 | 3.72 | −8.18 | |
| | Polyester | Before test | 68.98 | 3.25 | −1.56 | 2.57 |
| | | After test | 67.81 | 4.37 | −3.55 | |
| | Wool | Before test | 67 | 1.5 | 0.36 | 0.73 |
| | | After test | 67.34 | 1.87 | −0.17 | |
| | Acrylic | Before test | 64.83 | 1.28 | 1.7 | 3.52 |
| | | After test | 62.99 | 2.58 | −1.01 | |
| Prodeox- | Nylon 6,6 | Before test | 57.34 | −3.52 | −6.88 | 2.79 |
| violacein | | After test | 59.71 | −3.41 | −8.35 | |
| | Diacetate | Before test | 69.55 | 2.76 | 2.67 | 0.43 |
| | | After test | 69.37 | 2.5 | 2.96 | |
| | Bleached | Before test | 70.58 | −0.09 | 2.17 | 1.87 |
| | cotton | After test | 68.72 | −0.2 | 2.02 | |
| | Polyester | Before test | 67.62 | −1.52 | 1.53 | 2.73 |
| | | After test | 69.63 | −0.95 | −0.22 | |
| | Wool | Before test | 68.78 | −0.45 | 5.2 | 1.73 |
| | | After test | 70.4 | −0.98 | 5.49 | |
| | Acrylic | Before test | 66 | −0.97 | 2.37 | 1.95 |
| | | After test | 65.87 | −0.63 | 0.45 | |
| Pro- | Nylon 6,6 | Before test | 50.83 | −2.09 | −3.92 | 1.05 |
| violacein | | After test | 49.91 | −1.59 | −4 | |
| | Diacetate | Before test | 61.94 | 4.17 | 5.25 | 2.44 |
| | | After test | 59.98 | 5.39 | 6.04 | |
| | Bleached | Before test | 62.46 | 0.6 | 2.51 | 0.79 |
| | cotton | After test | 61.95 | 1.2 | 2.46 | |
| | Polyester | Before test | 64.1 | −1.81 | 1.31 | 1.85 |
| | | After test | 62.77 | −0.52 | 1.37 | |
| | Wool | Before test | 65.67 | −0.22 | 4.25 | 1.65 |
| | | After test | 64.23 | 0.18 | 4.96 | |
| | Acrylic | Before test | 56.45 | −2.01 | 2.1 | 2.34 |
| | | After test | 58.24 | −0.6 | 1.57 | |

TABLE 33
Acidic pH stability of fabrics dyed with violacein and violacein
derivatives. ΔE was calculated from measured colour coordinates
of fabric before and after the stability test.

| Product | Fabric | Measurement | L | a | b | ΔE |
|---|---|---|---|---|---|---|
| Deoxy- | Nylon 6,6 | Before test | 43 | 5.21 | −20.47 | 2.59 |
| violacein | | After test | 44.46 | 6.37 | −22.27 | |
| | Diacetate | Before test | 46.86 | 12.01 | −17.99 | 1.27 |
| | | After test | 46.41 | 12.25 | −16.83 | |
| | Bleached | Before test | 58.08 | 1.02 | −7.23 | 2.64 |
| | cotton | After test | 60.27 | 0.74 | −8.67 | |
| | Polyester | Before test | 62.77 | 2 | −5.04 | 2.24 |
| | | After test | 64.95 | 2.03 | −5.57 | |
| | Wool | Before test | 63.73 | 0.62 | −0.94 | 0.37 |
| | | After test | 64.01 | 0.41 | −0.81 | |
| | Acrylic | Before test | 58.82 | 2.34 | −2.86 | 1.38 |
| | | After test | 59.8 | 2.01 | −3.77 | |
| Violacein | Nylon 6,6 | Before test | 34.82 | 5.47 | −20.22 | 0.16 |
| | | After test | 34.94 | 5.43 | −20.31 | |
| | Diacetate | Before test | 45.97 | 8.41 | −13 | 1.50 |
| | | After test | 45.59 | 9.13 | −11.74 | |
| | Bleached | Before test | 59.65 | 3.71 | −6.68 | 1.43 |
| | cotton | After test | 59.6 | 2.72 | −7.71 | |
| | Polyester | Before test | 66.71 | 3.52 | −2.04 | 2.17 |
| | | After test | 68.04 | 1.96 | −1.34 | |
| | Wool | Before test | 69.04 | 0.77 | 1.2 | 0.79 |
| | | After test | 69.34 | 1.2 | 0.61 | |
| | Acrylic | Before test | 65.53 | 2.41 | 0.93 | 1.10 |
| | | After test | 66.03 | 2.78 | 0.02 | |
| Prodeox- | Nylon 6,6 | Before test | 60.43 | −4.73 | −6.98 | 2.46 |
| violacein | | After test | 62 | −2.95 | −7.62 | |
| | Diacetate | Before test | 71.53 | 3.37 | 3.36 | 1.23 |
| | | After test | 70.6 | 3.41 | 4.17 | |
| | Bleached | Before test | 72.68 | 0.85 | 1.38 | 1.17 |
| | cotton | After test | 73.7 | 0.64 | 1.91 | |
| | Polyester | Before test | 72.61 | −1.65 | 1.3 | 1.40 |
| | | After test | 73.29 | −0.67 | 0.57 | |
| | Wool | Before test | 70.58 | −1.44 | 5.61 | 0.91 |
| | | After test | 70.8 | −1.73 | 6.44 | |
| | Acrylic | Before test | 66.65 | −1.02 | 2.25 | 1.92 |
| | | After test | 68.57 | −1.02 | 2.18 | |
| Pro- | Nylon 6,6 | Before test | 50.84 | 0.04 | −4.68 | 2.79 |
| violacein | | After test | 52.72 | −1.67 | −3.52 | |
| | Diacetate | Before test | 60.94 | 4.79 | 4.9 | 3.29 |
| | | After test | 59.6 | 4.35 | 7.87 | |
| | Bleached | Before test | 62.48 | 1.73 | 2.27 | 3.00 |
| | cotton | After test | 65.48 | 1.87 | 2.2 | |
| | Polyester | Before test | 64.55 | −1.43 | 1.3 | 1.68 |
| | | After test | 64.14 | −1.28 | 2.92 | |
| | Wool | Before test | 65.49 | −1.55 | 5.81 | 1.26 |
| | | After test | 66.56 | −0.93 | 5.56 | |
| | Acrylic | Before test | 59.15 | −0.61 | 1.45 | 2.13 |
| | | After test | 58.2 | −1.53 | 3.12 | |

TABLE 34
Alkaline pH stability of fabrics dyed with violacein and violacein
derivatives. ΔE was calculated from measured colour coordinates
of fabric before and after the stability test.

| Product | Fabric | Measurement | L | a | b | ΔE |
|---|---|---|---|---|---|---|
| Deoxy- | Nylon 6,6 | Before test | 45.47 | 6.49 | −22.12 | 2.79 |
| violacein | | After test | 46 | 4.44 | −20.31 | |
| | Diacetate | Before test | 53.4 | 9.48 | −14.88 | 1.54 |
| | | After test | 53.77 | 9.48 | −13.38 | |
| | Bleached | Before test | 61.33 | 1.92 | −9.6 | 1.93 |
| | cotton | After test | 62.83 | 2.23 | −8.43 | |
| | Polyester | Before test | 66.7 | 1.3 | −4.19 | 0.94 |
| | | After test | 67.22 | 2.02 | −3.88 | |
| | Wool | Before test | 65.33 | 0.19 | 0.13 | 1.82 |
| | | After test | 66.95 | 0.82 | −0.41 | |
| | Acrylic | Before test | 60.91 | 1.48 | −2.6 | 1.48 |
| | | After test | 59.52 | 0.99 | −2.75 | |
| Violacein | Nylon 6,6 | Before test | 34.13 | 6.3 | −20.23 | 1.39 |
| | | After test | 33.91 | 5.25 | −19.35 | |
| | Diacetate | Before test | 43.93 | 8.84 | −14.67 | 3.76 |
| | | After test | 44.97 | 7.85 | −11.19 | |
| | Bleached | Before test | 59.94 | 2.69 | −8.23 | 1.20 |
| | cotton | After test | 60.76 | 3.22 | −7.53 | |
| | Polyester | Before test | 66.44 | 4.15 | −2.11 | 3.98 |
| | | After test | 68.28 | 1.01 | −0.5 | |
| | Wool | Before test | 67.52 | 0.35 | 1.01 | 0.77 |
| | | After test | 66.99 | −0.18 | 1.19 | |
| | Acrylic | Before test | 65.79 | 3.11 | 0.28 | 2.04 |
| | | After test | 63.85 | 2.52 | 0.06 | |
| Prodeox- | Nylon 6,6 | Before test | 58.1 | −3.9 | −7.44 | 2.09 |
| violacein | | After test | 59.99 | −3.03 | −7.58 | |
| | Diacetate | Before test | 66.45 | 3.65 | 3.66 | 1.71 |
| | | After test | 67.98 | 2.94 | 3.91 | |
| | Bleached | Before test | 66.25 | 0.37 | 3.43 | 4.42 |
| | cotton | After test | 70.27 | 0.86 | 1.65 | |
| | Polyester | Before test | 70.69 | −1.45 | 1.11 | 1.87 |
| | | After test | 71.66 | −0.57 | −0.22 | |
| | Wool | Before test | 70.9 | −0.24 | 5.35 | 0.44 |
| | | After test | 71.22 | −0.29 | 5.65 | |
| | Acrylic | Before test | 67.02 | −1.67 | 2.25 | 0.34 |
| | | After test | 66.95 | −1.36 | 2.36 | |
| Pro- | Nylon 6,6 | Before test | 52.98 | −2.5 | −3.65 | 2.29 |
| violacein | | After test | 50.89 | −1.8 | −3.04 | |
| | Diacetate | Before test | 60.55 | 3.12 | 6.29 | 1.11 |
| | | After test | 61.54 | 3.25 | 6.78 | |
| | Bleached | Before test | 63.78 | 2.31 | 1.4 | 0.90 |
| | cotton | After test | 64.37 | 2.22 | 2.08 | |
| | Polyester | Before test | 64.55 | −2.15 | 1.47 | 2.13 |
| | | After test | 64.69 | −1.03 | −0.34 | |
| | Wool | Before test | 65.56 | −2.33 | 7.35 | 1.98 |
| | | After test | 65.28 | −1.97 | 5.42 | |
| | Acrylic | Before test | 57.87 | −0.46 | 1.91 | 2.73 |
| | | After test | 58.76 | −2.16 | 3.85 | |

TABLE 35
Artificial light stability of fabrics dyed with violacein and
violacein derivatives. ΔE was calculated from measured colour
coordinates of fabric before and after the stability test.

| Product | Fabric | Measurement | L | a | b | ΔE |
|---|---|---|---|---|---|---|
| Deoxy- | Nylon 6,6 | Before test | 43.65 | 5.57 | −20.95 | 3.63 |
| violacein | | After test | 46.23 | 5.57 | −18.4 | |
| | Diacetate | Before test | 47.05 | 13.99 | −17.37 | 9.33 |
| | | After test | 49.82 | 9.13 | −9.9 | |
| | Bleached | Before test | 60.49 | 0.97 | −7.49 | 2.33 |
| | cotton | After test | 62.47 | 2 | −6.81 | |
| | Polyester | Before test | 64.39 | 1.76 | −4.83 | 9.23 |
| | | After test | 70.32 | 0.28 | 2.09 | |
| | Wool | Before test | 64.28 | −0.4 | −0.37 | 6.49 |
| | | After test | 69.63 | 0.44 | 3.2 | |
| | Acrylic | Before test | 60.52 | 1.59 | −2.77 | 6.58 |
| | | After test | 64.84 | 0.76 | 2.12 | |
| Violacein | Nylon 6,6 | Before test | 38.2 | 11.52 | −16.9 | 7.65 |
| | | After test | 34.82 | 4.72 | −17.83 | |
| | Diacetate | Before test | 43.88 | 8.03 | 13.02 | 14.73 |
| | | After test | 52.2 | 6.06 | 1.03 | |
| | Bleached | Before test | 60.03 | 2.81 | −7.43 | 3.15 |
| | cotton | After test | 62.61 | 4.18 | −6.26 | |
| | Polyester | Before test | 68.58 | 3.06 | −1.75 | 5.72 |
| | | After test | 73.11 | 2.28 | 1.66 | |
| | Wool | Before test | 67.82 | 1.06 | 0.85 | 4.25 |
| | | After test | 71.32 | 1.18 | 3.25 | |
| | Acrylic | Before test | 65.94 | 3.06 | 1.37 | 4.41 |
| | | After test | 69.82 | 3.01 | 3.46 | |
| Prodeox- | Nylon 6,6 | Before test | 61.29 | −3.52 | −7.38 | 3.54 |
| violacein | | After test | 63.18 | −2.52 | −4.56 | |
| | Diacetate | Before test | 68.08 | 1.74 | 3.88 | 3.07 |
| | | After test | 70.05 | 3.2 | 5.72 | |
| | Bleached | Before test | 69.92 | −1.14 | 3.25 | 4.18 |
| | cotton | After test | 72.08 | 2.42 | 2.87 | |
| | Polyester | Before test | 70.5 | −2.19 | 0.75 | 3.32 |
| | | After test | 72.84 | −0.23 | 2.05 | |
| | Wool | Before test | 69.65 | −1.77 | 5.88 | 4.74 |
| | | After test | 73.57 | 0.8 | 6.59 | |
| | Acrylic | Before test | 65.78 | −1.59 | 3.3 | 3.05 |
| | | After test | 68.69 | −0.68 | 3.2 | |
| Pro- | Nylon 6,6 | Before test | 47.03 | −1.98 | −2.52 | 4.24 |
| violacein | | After test | 51.07 | −0.68 | −2.49 | |
| | Diacetate | Before test | 56.45 | 3.46 | 5.75 | 7.18 |
| | | After test | 63.43 | 4.56 | 7.02 | |
| | Bleached | Before test | 58.83 | −0.52 | 4.04 | 5.27 |
| | cotton | After test | 62.69 | 3.04 | 3.55 | |
| | Polyester | Before test | 59.49 | −1.38 | 2.13 | 5.63 |
| | | After test | 65.01 | −0.35 | 1.74 | |
| | Wool | Before test | 65.25 | −0.55 | 5.71 | 2.41 |
| | | After test | 67.51 | −0.15 | 6.46 | |
| | Acrylic | Before test | 56.17 | −0.68 | 3.21 | 2.43 |
| | | After test | 58.47 | −0.05 | 2.76 | |

TABLE 36
UV stability of fabrics dyed with violacein and violacein
derivatives. ΔE was calculated from measured colour
coordinates of fabric before and after the stability test.

| Product | Fabric | Measurement | L | a | b | ΔE |
|---|---|---|---|---|---|---|
| Deoxy- | Nylon 6,6 | Before test | 45.92 | 4.94 | −21.23 | 1.60 |
| violacein | | After test | 46.19 | 4.4 | −19.75 | |
| | Diacetate | Before test | 52.05 | 9.98 | −14.88 | 4.43 |
| | | After test | 53.68 | 8.75 | −10.95 | |
| | Bleached | Before test | 61.4 | 1.8 | −7.92 | 1.47 |
| | cotton | After test | 62.17 | 1.62 | −6.68 | |
| | Polyester | Before test | 66.55 | 1.03 | −2.87 | 0.99 |
| | | After test | 67.09 | 1.19 | −3.68 | |
| | Wool | Before test | 65.56 | 0.65 | 0.31 | 2.26 |
| | | After test | 67.65 | 0.75 | 1.17 | |
| | Acrylic | Before test | 60.15 | 1.07 | −2.07 | 1.75 |
| | | After test | 61.34 | 2.05 | −2.89 | |
| Violacein | Nylon 6,6 | Before test | 34.58 | 4.58 | −19.61 | 1.75 |
| | | After test | 35.29 | 4.36 | −18.03 | |
| | Diacetate | Before test | 41.1 | 9.04 | −13.41 | 4.43 |
| | | After test | 43.08 | 10.1 | −9.59 | |
| | Bleached | Before test | 58.11 | 3.63 | −8.4 | 2.66 |
| | cotton | After test | 58.38 | 3.17 | −5.79 | |
| | Polyester | Before test | 67.13 | 1.68 | −1.12 | 1.12 |
| | | After test | 68.13 | 1.99 | −0.71 | |
| | Wool | Before test | 68.14 | 0.77 | 1.77 | 1.59 |
| | | After test | 68.91 | 2.13 | 1.49 | |
| | Acrylic | Before test | 59.81 | 2.39 | 0.08 | 2.83 |
| | | After test | 62.36 | 1.52 | 0.96 | |
| Prodeox- | Nylon 6,6 | Before test | 58.89 | −3.07 | −7.48 | 1.44 |
| violacein | | After test | 57.71 | −3.86 | −7.27 | |
| | Diacetate | Before test | 69.07 | 3.64 | 2.57 | 1.20 |
| | | After test | 69.73 | 4.04 | 3.49 | |
| | Bleached | Before test | 69.93 | −0.02 | 2.29 | 0.24 |
| | cotton | After test | 69.81 | −0.11 | 2.48 | |
| | Polyester | Before test | 69.55 | −1.07 | 0.38 | 2.12 |
| | | After test | 68.48 | −2.21 | 1.81 | |
| | Wool | Before test | 69.89 | −2.05 | 5.81 | 0.42 |
| | | After test | 69.92 | −1.63 | 5.81 | |
| | Acrylic | Before test | 65.29 | −1.26 | 2.69 | 1.09 |
| | | After test | 64.75 | −0.69 | 1.94 | |
| Pro- | Nylon 6,6 | Before test | 57.31 | −0.99 | 2.73 | 1.30 |
| violacein | | After test | 57.07 | 0.02 | 1.94 | |
| | Diacetate | Before test | 61.25 | 3.87 | 4.46 | 1.88 |
| | | After test | 61.92 | 3.89 | 6.22 | |
| | Bleached | Before test | 64.65 | 0.4 | 2.43 | 1.25 |
| | cotton | After test | 64.92 | 1.27 | 3.29 | |
| | Polyester | Before test | 64.26 | −2.11 | 1.21 | 1.69 |
| | | After test | 65.53 | −1.02 | 0.99 | |
| | Wool | Before test | 62.43 | −0.15 | 5.23 | 0.51 |
| | | After test | 62.87 | −0.07 | 5.48 | |
| | Acrylic | Before test | 57.31 | −0.99 | 2.73 | 1.30 |
| | | After test | 57.07 | 0.02 | 1.94 | |

The results showed that under all conditions tested, fabrics dyed with violacein and violacein derivatives showed remarkable colour fastness and were generally stable under all conditions tested with minimal change in the colour coordinates or colour strength of the fabrics. In general, under all conditions tested the AE was typically less than 10 which indicates negligible change.

Conclusions: This experiment shows that violacein and violacein derivatives dyed onto fabrics show remarkably good colour fastness, indicating their suitability for textile dyeing applications.

### Example 25—Testing the Antimicrobial Activity of Textile Fabrics Directly Dyed with Violacein and Violacein Derivatives

Violacein is reported as being a potent antibiotic, capable of inhibiting the growth of a wide range of microbial kingdoms including bacteria, yeast, fungi, viruses, protozoa and nematode. Much less is known about the other violacein derivatives, and importantly, whether fabrics dyed with these pigments also possess anti-microbial activity. To test this, overnight cultures of wild-type E. coli DH5a and S. cerevisiae S228c strains were diluted in 5 mL of cultivation media (LB for E. coli, minimal media for S. cerevisiae) to a starting OD600 of 0.1. 1.5×1.5 cm dyed Nylon 6,6 fabric strips were sterilized by submerging in 75% ethanol for 15 mins and air drying in a laminar air flow hood before adding to each culture (with undyed Nylon 6,6 fabric as a negative control). Cultures were incubated overnight at 30° c. and 220 RPM for S. cerevisiae and E. coli after which the final OD600 was recorded. % inhibition was calculated by comparing the difference in OD600 between control cultures (cultures with undyed fabrics) and test cultures (cultures with dyed fabrics). The results are presented in Table 37 and show that fabric dyed with violacein and violacein derivatives have a significant impact on the growth of both fungi like S. cerevisiae and bacteria like E. coli as demonstrated by the significantly lower OD600 compared to undyed fabric samples. Most surprising was that while E. coli was most inhibited by violacein, S. cerevisiae was most inhibited by proviolacein, and while the antimicrobial activity of violacein is well documented, the antimicrobial activity of the other violacein derivatives is unstudied. This experiment provides evidence that all violacein derivatives display antimicrobial properties.

TABLE 37
Final OD600 and % inhibition of E. coli and S. cerevisiae
strains cultured in the presence of Nylon fabic dyed with
violacein and violacein derivatives. NA: Not applicable.

| | Final OD600 | | % Inhibition | |
|---|---|---|---|---|
| Compound | E. Coli | S. cerevisiae | E. Coli | S. cerevisiae |
| Deoxyviolacein | 4.62 | 4.99 | 11.45 | 6.90 |
| Violacein | 4.16 | 4.64 | 20.24 | 13.43 |
| Prodeoxyviolacein | 4.67 | 4.70 | 10.49 | 12.35 |
| Proviolacein | 4.52 | 4.39 | 13.33 | 18.13 |
| Control | 5.21 | 5.36 | NA | NA |

Conclusions: This experiment demonstrates how fabric dyed with violacein and violacein derivatives possess antimicrobial activity demonstrating their application to enhance the antimicrobial properties of clothing and other textiles.

### Example 26—Testing the Antioxidant Activity of Textile Fabrics Directly Dyed with Violacein and Violacein Derivatives

Violacein is reported as being a potent antioxidant able to effectively scavenge free radicals. Much less is known about the other violacein derivatives, and importantly, whether fabrics dyed with these pigments also possess antioxidant activity. To test this, 1.5×1.5 cm dyed Nylon 6,6 fabric strips were incubated in the dark at room temperature for 1 h in 5 mL of a 5×10⁻⁵ M DPPH free radical solution in 100% ethanol. Undyed Nylon 6,6 fabric strips were included as a negative control. After 1 h absorbance was measured at 517 nm and % scavenging ability calculated as above. The results presented in Table 38 show that all compounds display good antioxidant ability.

TABLE 38
Free radical scavenging ability of fabric dyed with violacein
and violacein derivatives. NA: Not applicable.

| Compound | OD517 | Scavenging ability % |
|---|---|---|
| Control | 2.27 | NA |
| Deoxyviolacein | 1.43 | 37.05 |
| Violacein | 1.64 | 27.53 |
| Prodeoxyviolacein | 1.99 | 12.28 |
| Proviolacein | 1.88 | 17.27 |

Conclusions: This experiment shows that fabric dyed with violacein and violacein derivatives display good antioxidant properties with the ability to scavenge free radicals.

### Example 27—Testing the UV Protection Factor (UPF) Potential of Violacein and Violacein Derivatives

Violacein is reported as being a potent UV absorbent able to effectively absorb UV radiation, however, much less is known about the other violacein derivatives. To investigate the potential of using violacein and violacein derivatives as UV inhibitors and to potentially impart UV protection to fabrics, the UV absorption spectra of each compound was analyzed by HPLC and the total absorption capability at each of the most relevant UV wavelengths quantified.

For the quantification of UV absorption capacity of the compound of interest, a chromatographic separation on the F5 column and a gradient of water/acetonitrile containing 0.05% formic acid was run according to Example 5). Absorbance curves at 230, 245, 305, 360 and 575 nm were registered, as well as absorption spectra between 190 and 640 nm. It was found that violacein has absorbance maxima at 220, 270, 375, and 575 nm; deoxyviolacein at 200, 260, 370 and 570 nm; proviolacein at 210, 300, 415 and 600 nm; and prodeoxyviolacein at 210, 290, 420 and 610 nm. The compounds were quantified based on the signal at 230 nm, using calibration curves of authentic analytical standards.

The AUC for the peaks at 360 nm, 305 nm and 245 nm were used for estimating the UV-absorption capacity of the compounds in the UV-A, UV-B and UV-C regions, respectively. The UV absorption capacity is represented in mAU.min/mM. UV-A, UV-B and UV-C represent the most relevant regions where UV protection is required. UV-C is the most harmful type of UV radiation and can be generated by inorganic sources. UV-B radiation predominantly comes from the suns rays and is responsible for sun burn. UV-A radiation is also derived predominantly from the suns rays, and while UV-A radiation is less harmful than UV-B or UV-C but is much more prevalent. The results, presented in Table 39 demonstrate that all 4 compounds display good UV absorption capability, absorbing a significant amount of UV radiation across the UV spectrum. In particular, it was found that both violacein and deoxyviolacein had particularly high UV absorption capability in the UV-C region, while proviolacein and prodeoxyviolacein had particularly high UV absorption capability in the UV-B region. Also of interest was that violacein has UV absorption capability across all 3 important UV regions, suggesting that it could be effective in blocking the most harmful UV radiation when applied to fabrics and other materials.

TABLE 39
UV absorption capability of violacein and violacein derivatives.
Shown is the UV absorbance maxima for each compound and
the total UV absorption per mM of compound at wavelengths
indicative of UV-A, UV-B and UV-C radiation.

| | | Total UV absorption | | |
|---|---|---|---|---|
| | UV | [(mAU · min)/mM] | | |
| | absorbance | UV A | UV B | UV C |
| Compound | maxima | (360 nm) | (305 nm) | (245 nm) |
| Violacein | 200, 275, 375 | 411.4 | 1011.6 | 8876.8 |
| Deoxyviolacein | 200, 260, 370 | 702.8 | 0 | 9251.6 |
| Proviolacein | 210, 300 | 0 | 4097.7 | 3142.9 |
| Prodeoxyviolacein | 210, 290 | 0 | 3778.8 | 3516.1 |

### Example 28—Incorporation of Violacein and Violacein Derivatives into Nanocellulose

For some applications it may not be advantageous to have violacein or violacein derivatives as a dry powder or formulated in organic solvents like ethanol and isopropyl myristate. In order to increase the applicability of these pigments it was tested whether they could be incorporated into different types of nanocellulose including bacterial nanocellulose (BNC) and nanofabricated cellulose (NFC). BNC is a type of nano-structured cellulose produced by microorganisms including multiple species of bacteria and has application in a myriad of applications including wound dressing, food packaging, plastic alternatives, food additives, anti-bacterial barriers, cosmetics, and even textile fibers. NFC is a bio-based additive which displays a broad range of applications including as ingredients for cosmetics and personal care products, resins and adhesives, cleaning products, and paper and packaging products. NFC's are also particularly useful as carriers for pigments, allowing them to be formulated for applications as paints, coatings and textile dyes. For these applications, functional groups on the small cellulose fibrils bind both the pigment and the surface to which it is to be applied (i.e. textile fabric, paper etc) acting as a binding agent. All of the applications listed above would be enhanced and improved by the incorporation of violacein and violacein derivatives into the cellulosic structures prior to their incorporation into other products. Potential improved applications include (but are not limited to); Adding anti-bacterial properties to wound healing products, adding anti-bacterial, anti-oxidant, UV protective and colouration to food packaging products, adding anti-bacterial, anti-oxidant, UV protective and colouration to cosmetics products, adding anti-bacterial, anti-oxidant, UV protective and colouration to textile fibers, and anti-bacterial, anti-oxidant, UV protective and colouration to bio-based paints and textile dyes. This last application would be particularly suited to facilitate the dyeing of violacein and violacein derivatives onto materials where the compounds aren't able to directly dye (or directly dye poorly).

To test whether violacein and violacein derivatives could be incorporated into BNC hydrogels the following experiment was performed. First, a nanocellulose pellicle was produced using a kombucha starter culture (Wellness Drinks, DE) whereby the SCOBY starter culture was used to inoculate a sterile liquid prepared with 8 g of green tea, 100 g of sucrose and 1 L of water. The culture was incubated at approx. 26° c. for 8 days without shaking until a fresh nanocellulose pellicle formed at the top of the liquid. The pellicle was harvested and cleaned by incubation in 100 mM NaOH at 80° c. for 30 mins and washing with water. Finally, the pellicle was dried at room temperature. To incorporate violacein and violacein derivatives into the BNC hydrogels they were simply incubated at room temperature in dye baths containing the coloured compound of interest in 90% ethanol 10% extractant (in this isopropyl myristate) for 1 h. During incubation it was found that the coloured pigment was efficiently transferred to the BNC turning it the corresponding colour of the dye bath. The resulting violacein and violacein derivative incorporated BNC pelicles could then be further processed for different applications. For example they can be pressed into a thin film to act as a protective barrier for food preservation.

To test whether violacein and violacein derivatives could be incorporated into NFC solutions, deoxyviolacein in powder form and suspended in 100% ethanol were added to a 1% NFC solution (Exilva, from Borregaard) and mixed thoroughly for 1 h at 37° c. which allowed for a stable emulsion but also to evaporate the ethanol from solution. To improve dispersion a non-ionic surfactant like Triton-X 100 can be added at 0.01%. After thorough mixing a stable dark blue/purple emulsion was formed which was used for further experiments.

It was found that the deoxyviolacein-NFC emulsion was suitable as a paint, able to effectively colour a range of paper samples. It was also found that the emulsion could be used as a dye bath to dye a range of fabrics. Fabric samples were incubated for 8 h at room temperature in the deoxyviolacein-NFC emulsion, then washed with water and dried, following this the colour coordinates were determined as described above. The results, presented in Table 40 show comparable, and in some cases enhanced colour strength (AE) compared to dye baths in aqueous or organic solvents (presented above). In particular it was found that incorporation into an NFC emulsion was an effective strategy to improve the dye performance on fabrics where deoxyviolacein didn't bind directly as well e.g. polyester. This result demonstrates how the NFC acts as a binding agent, effectively binding both the fabric and the coloured pigment allowing fabrics to be effectively dyed.

TABLE 40
Colour coordinates of fabrics dyed with a deoxyviolacein-NFC
emulsion for 8 h at room temperature. ΔE is calculated
by comparison to the colour coordinates of an undyed control.

Colour coordinates

| Fabric | L | a | b | ΔE |
|---|---|---|---|---|
| Diacetate | 68.73 | 7.04 | 7.76 | 20.00119 |
| Bleached cotton | 69.16 | 10.07 | 4.95 | 22.71315 |
| Polyamide | 58.14 | 7.83 | 2.24 | 31.40653 |
| Polyester | 61.49 | 10.06 | −4.38 | 39.49993 |
| Acrylic | 72.28 | −0.07 | 8.07 | 11.62876 |
| Wool | 63.4 | 2.16 | 7.26 | 20.62846 |

Conclusions: This experiment shows how violacein and violacein derivatives can be effectively incorporated into a range of cellulose based products, and the resulting pigment-cellulose product can be used in a range of value-added applications.

### Example 29—In Vivo Production of Violacein and Violacein Derivative Glucosides by Engineered S. Cerevisiae Strains

In Example 8 it was shown that certain UGT enzymes were able to catalyze the glycosylation of violacein and proviolacein in an in vitro enzyme reaction. In this experiment, UGT's were tested for their ability to glycosylate violacein and proviolacein directly in vivo by overexpressing them in de novo violacein and proviolacein producing strains. Overexpression cassettes for UGT enzymes Pt73Y (identified in the previous in vitro screen), Ha88B_2 and Cs73Y (identified in subsequent assays) were introduced into violacein and proviolacein producing strains according to Example 1, cultivated according to Example 3, and produced metabolites were detected and quantified according to Example 5. HPLC and QTOF analysis showed that violacein and proviolacein mono-glucoside was produced de novo in all engineered strains. Furthermore, analysis of accumulation in the intracellular and extracellular space revealed that in contrast to violacein and proviolacein, which accumulate exclusively intracellularly, violacein and proviolacein mono-glucoside accumulated almost exclusively in the extracellular space. Overall this indicates that violacein and proviolacein glycosylation can be performed in vivo in de novo production strains, but also that glycosylation is another effective strategy to overcome the solubility and intracellular accumulation issue of violacein and proviolacein.

Conclusion: This experiment demonstrates that glycosides of violacein and violacein derivatives can be produced de novo by engineered cells expression violacein (and derivatives) biosynthetic pathways and UGT enzymes and that this is an effective strategy to overcome the solubility and intracellular accumulation issue of violacein and proviolacein.

### Example 30—Integrated Process for the Production and DSP of Violacein and Violacein Derivatives and their Dyeing onto Fabrics, Fibers, and Yarns

By combining several examples presented above its possible to design and implement an integrated process to produce violacein or one of several violacein derivatives by fermentation of engineered strains of S. cerevisiae in the presence of an in situ extractant, harvest the organic extractant phase from the fermentation, process it with no further modification into a dye bath, and use the resulting dye bath to dye various fabrics, fibers, and yarns. Such a process enables significant improvements in the environmental footprint of dye manufacturing and textile dyeing but also enables significant cost reductions due to the simple DSP process/dye bath formulation and dyeing process. To demonstrate an exemplified use of this process, the following experiment was performed with viable alternatives presented where relevant.

Engineered S. cerevisiae production strains producing each of the known coloured derivatives; violacein (SC-141), deoxyviolacein (SC-144), prodeoxyviolacein (SC-139), proviolacien (SC-145), were cultivated in a fed-batch fermentation process optimized to maximize production of the compound of interest, with the addition of an in situ extractant such as isopropyl myristate at a concentration up to 20% of the total fermentation volume. A range of other in situ extractants can also be used at this step, with the choice of extractant dependent on downstream application. As the fed-batch fermentation progressed the in situ extractant containing the coloured compound of interest was removed and replaced with fresh extractant (semi-)continuously to maximize extraction efficiency. At the end of the fermentation the collected in situ extractant enriched with the coloured compound of interest was harvested and used directly as the main input for a dye bath by diluting it with an organic solvent such as ethanol so that the final concentration of the extractant in the dye bath was no more than 66%. This mix was then used directly with no further modifications as the dye bath. Alternatives for this step include different dye bath compositions; for example, using a non-ionic surfactant like Antifoam-A as the in situ extractant and preparing an aqueous dye bath by diluting in water below the cloud point of the system. Other alternatives include adding a DSP step to obtain dry powder allowing for more flexibility in dye bath formulation. Or a crude DSP step to partially purify or up-concentrate the product. One particularly useful strategy is to incorporate a crude DSP step in-line with the fermentation to enable the partial recycling of the in situ extractant. In such an alternative the in situ extractant containing the pigment of interested collected (semi-)continuously from the fermentation is passed through a column containing gel silica allowing the pigment to bind to the silica and clean in situ extractant to pass through and be recycled back into the fermentation.

A simple exhaustive dye strategy was set up where fabric, fiber, or yarn to be dyed was added to the dye bath at a liquor ratio of at least 1:5 (i.e., 1 Kg of material to 5 L of dye bath). In some cases, the material to be dyed was pre/post-treated to increase or decrease the pH to improve the dyeing efficiency or to otherwise increase the colour fastness. To increase the pH the material (e.g. cotton) was incubated for 30 mins in a 40 g/L solution of soda ash. The dyeing process was carried out in a laboratory dyeing machine (DataColour AHIBA) at 23° c. (approximating room temperature) and atmospheric pressure for 15 minutes, after which the material was washed with water to remove unbound dye and organic solvent. The resulting material was dried and analyzed for colour strength and colour fastness. While a key advantage of this method is the simplicity of the dye bath formulation, combined with the rapid dyeing process under ambient conditions, additions can be made to further enhance the dyeing process for example by increasing temperature, adjusting pH, or adding other chemicals expedients such as mordants. As with the examples presented above, this simple dyeing method under ambient conditions and short dyeing times still produces materials with excellent colour strength and colour fastness. A range of dye bath compositions can be used in this step, with all containing the pigment of interest at various stages of purification.

Once the dye bath was exhausted, the remaining spent constituents (deoxyviolacein, ethanol and in situ extractant) were recycled for subsequent use as follows. The entire spent dye bath was transferred to a rotary evaporator to collect the ethanol so that it could be recycled for subsequent use. In a similar fashion, the remaining in situ extractant and left over deoxyviolacein from the spent dye bath was recycled by gel silica chromatography as described in Example 16 where both components can be recycled after elution by evaporating the volatile elution solvent using a rotary evaporator or similar. In cases where a non-ionic surfactant containing deoxyviolacein is used in the dye bath, the components can be recycled using cloud point extraction as described in Example 14.

### Example 31—Fabric Dyeing with Violacein and Violacein Derivatives in an Aqueous Dye Bath Formulation

In previous experiments it was shown that in situ extractant containing violacein and violacein derivatives could be formulated into a solvent dye bath (in the case of lipophilic extractants), or an aqueous dye bath (in the case of non-ionic surfactants). In this experiment it was demonstrated how pigments in lipophilic extracts could be formulated into an aqueous dye bath by addition of simple dispersing agents. Furthermore, this experiment demonstrates an improved method for dyeing hydrophobic fibers like polyester by dyeing at increased temperatures. Deoxyviolacein producer SC-144 was cultivated in the presence of 10% ethyl laurate. At the end of the cultivation 10 mL of deoxyviolacein rich extractant phase was collected and diluted with 90 mL of water. Due to its immiscibility in water, ethyl laurate containing deoxyviolacein formed insoluble droplets in water. To counteract this, surfactants can be used as dispersing agents. In this experiment approx. 2 mL of common dish soap was added as a dispersing agent enabling a stable emulsion and even dispersing of deoxyviolacein in an aqueous suspension. This suspension was used directly as a dye bath to dye polyester, a 30×30 cm piece of polyester fabric was added to the liquid and the entire mixture was incubated in an autoclave at 130° c. for 60 mins followed by washing with water to remove any unbound pigment. After incubation the polyester fabric was efficiently dyed with deoxyviolacein forming a dark purple colour and a colour change AE of 82.4 indicating exceptional colour strength.

Conclusions: This experiment demonstrates how dispersing agents such as soap enable stable suspensions of violacein and violacein derivatives in in situ extractants, enabling efficient dyeing in aqueous dye baths. This experiment also demonstrates how high temperatures enable dyeing of polyester with violacein and violacein derivatives.

1. A method for producing a compound of formula (I) selected from the group consisting of: 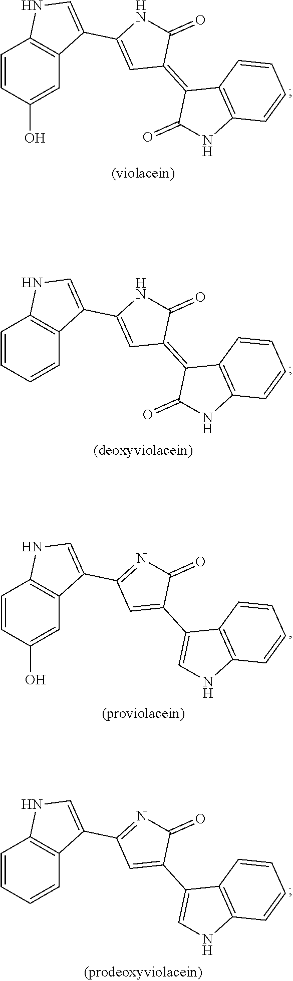 [SMILES: O=C1NC(c2c[nH]c3ccc(O)cc23)=C/C1=C1\C(=O)Nc2ccccc21 ; O=C1NC(c2c[nH]c3ccccc23)=C/C1=C1\C(=O)Nc2ccccc21 ; O=C1N=C(c2c[nH]c3ccc(O)cc23)C=C1c1c[nH]c2ccccc12 ; O=C1N=C(c2c[nH]c3ccccc23)C=C1c1c[nH]c2ccccc12]
and a tautomer thereof; wherein the method comprises providing an indole of formula (II): 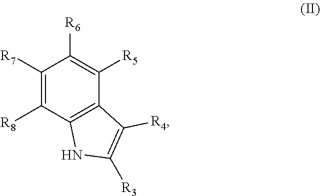
wherein
R₃, R₅, R₆, R₇, and R₈ are independently of each other selected from the group consisting of: H, O, or OH; and wherein R₄ is selected from the group consisting of: H, glycerol-3-phosphate; 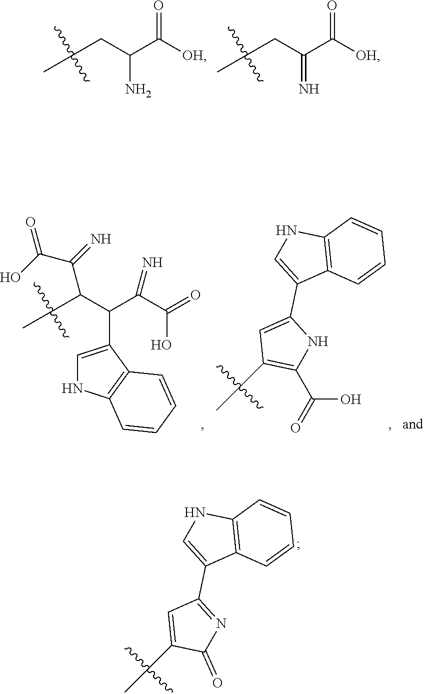 [SMILES: *CC(N)C(=O)O ; *CC(=N)C(=O)O ; *C(C(=N)C(=O)O)C(C(=N)C(=O)O)c1c[nH]c2ccccc12 ; *c1cc(-c2c[nH]c3ccccc23)[nH]c1C(=O)O ; *C1=CC(c2c[nH]c3ccccc23)=NC1=O]
and further comprising contacting the indole of formula (II) with one or more enzymes from an operative biosynthetic pathway for producing violacein comprising, wherein the method involves at least:
a) a tryptophan oxidase having at least 70% identity to the sequence comprised in SsStaO (SEQ ID NO: 80), NlInkO (SEQ ID NO: 84), and/or AmAtmO (SEQ ID NO: 88); and/or
b) an IPA imine dimer synthase has at least 70% identity to the sequence comprised in LaRebD (SEQ ID NO: 78), SsStaD (SEQ ID NO: 82), NlInkD (SEQ ID NO: 86), and/or AmAtmD (SEQ ID NO: 90).

2. The method according to claim 1, wherein the method comprises contacting the compound of formula (II) with an amino acid, such as a proteinogenic amino acid, for example serine, in the presence of the one or more enzymes.

3. The method according to any of the preceding claims, wherein the method comprises contacting the compound of formula (II) with one or more pathway molecules selected from: FAD, HEME, and NADPH.

4. The method according to any of the preceding claims, wherein R₄ is H, and the compound of formula (II) has been prepared in vitro or in vivo.

5. The method according to any of the preceding claims, wherein R₄ is glycerol-3-phosphate and the compound of formula (II) has been prepared in vivo.

6. The method according to any one of the preceding claims, wherein the compound of formula (I) is prepared from glucose.

7. The method according to any of the preceding claims, wherein the indole of formula (II) is selected from indole, and indole-3-glycerol phosphate.

8. The method according to any preceding claims, wherein the compound of formula (I) is contacted with a glycosyl donor comprising a glycosyl group.

9. The method according to claim 8, wherein the method comprises a glycosylation step of the compound of formula (I) to provide a glycosylated compound of formula (I), wherein the glycosylated compound of formula (I) comprises the compound of formula (I) covalently attached to the glycosyl group.

10. The method according to any of claims 8-9, wherein the method comprises a de-glycosylation step such that the glycosylated compound of formula (I) is de-glycosylated to provide the compound of formula (I).

11. The method according to claim 10, wherein the de-glycosylation step is facilitated by a glycosidase, such as a β-glycosidase.

12. The method according to claim 10, wherein the de-glycosylation step is facilitated by a glucosidase, such as a β-glucosidase.

13. The method according to any of claims 8-10, wherein the glycosyl group of the glycosyl donor comprises one or more of glucose, galactose, xylose, mannose, galactofuranose, arabinose, rhamnose, apiose, fucose, glucosamine, galactosamine, N-acetylglucosamine, N-acetylgalactosamine, xylosamine, mannosamine, arabinosamine, rhamnosamine, apiosamine, fucosamine, glucuronate, galacturonate, mannuronate, arabinate, apionate or a combination thereof.

14. The method according to any of claims 8-13, wherein the glycosylation step comprises an O-glycosylation, such as a β-O-glycosylation.

15. The method according to any of claims 8-14, wherein the glycosyl donor is a nucleotide glycoside.

16. The method of claim 15, wherein the nucleotide glycoside is NTP-glycoside, NDP-glycoside or NMP-glycoside.

17. The method of claim 16, wherein the nucleoside of the nucleotide glycoside is selected from Uridine, Adenosin, Guanosin, Cytidin and deoxythymidine.

18. The method of claim 17, wherein the nucleotide glycoside is selected from UDP-glycosides, ADP-glycosides, CDP-glycosides, CMP-glycosides, dTDP-glycosides and GDP-glycosides.

19. The method of claim 18, wherein the nucleotide glycoside is selected from UDP-D-glucose (UDP-Glc); UDP-galactose (UDP-Gal); UDP-D-xylose (UDP-Xyl); UDP-N-acetyl-D-glucosamine (UDP-GlcNAc); UDP-N-acetyl-D-galactosamine (UDP-GalNAc); UDP-D-glucuronic acid (UDP-GlcA); UDP -D-galactofuranose (UDP-Galf); UDP-arabinose; UDP-rhamnose, UDP-apiose; UDP-2-acetamido-2-deoxy-α-D-mannuronate; UDP-N-acetyl-D-galactosamine 4-sulfate; UDP-N-acetyl-D-mannosamine; UDP-2,3-bis(3-hydroxytetradecanoyl)-glucosamine; UDP-4-deoxy-4-formamido-β-L-arabinopyranose; UDP-2,4-bis(acetamido)-2,4,6-trideoxy-α-D-glucopyranose; UDP-galacturonate; UDP-3-amino-3-deoxy-α-D-glucose; guanosine diphospho-D-mannose (GDP-Man); guanosine diphospho-L-fucose (GDP-Fuc); guanosine diphospho-L-rhamnose (GDP-Rha); cytidine monophospho-N-acetylneuraminic acid (CMP-Neu5Ac); cytidine monophospho-2-keto-3-deoxy-D-mannooctanoic acid (CMP-Kdo); and ADP-glucose.

20. The method of any preceding claim, wherein the one or more enzymes are selected from glycosyltransferases, synthases, kinases, transketolases, transaldolase, phosphoketolases, phosphotransketolases, dehydratases, dehydrogenases, carboxyvinyltransferases, phosphoribosyl transferases, isomerases, oxidases, dimerases, and monooxygenases.

21. The method of claim 20, wherein the glycosyltransferase is derived from a plant, a fungus, or a bacterium.

22. The method of claim 21, wherein the plant is selected from Oryza sativa, Crocus sativus, Nicotiana tabacum, Stevia rebaudiana, Nicotiana benthatamiana, Arabidopsis thaliana, Helianthus annuus, and Populus trichocarpa.

23. The method of claim 21, wherein the bacterium is Bacillus subtilis.

24. The method of claim 20 to 22, wherein the glycosyl transferase is an O-glycoside transferase and/or a C-glycoside transferase.

25. The method of claim 24, wherein the glycosyl transferase is an aglycone O-glycosyltransferase.

26. The method of claim 24, wherein the glycosyl transferase is a glycoside O-glycosyltransferase.

27. The method of claim 24, wherein the glycosyl transferase is an aglycone O-glucosyltransferase.

28. The method of claim 24, wherein the glycosyl transferase is an aglycone O-rhamnosyltransferase.

29. The method of claim 24, wherein the glycosyl transferase is an aglycone O-xylosyltransferase.

30. The method of claim 24, wherein the glycosyl transferase is an aglycone O-arabinosyltransferase.

31. The method of claim 24, wherein the glycosyl transferase is an aglycone O—N-acetylgalactosaminyltransferase.

32. The method of claim 24, wherein the glycosyl transferase is an aglycone O—N-acetylglucosaminyltransferase.

33. The method of claim 24, wherein the glycosyl transferase is a hydroxytryptophan glycosyltransferase.

34. The method of claim 24, wherein the glycosyl transferase comprises the sequence of Pt73Y (SEQ ID: NO 64); (SEQ ID NO: 66); Bs109_1 (SEQ ID NO: 68); Bs109A1 (SEQ ID NO: 70); Cp73B (SEQ ID NO: 72); Cs73Y (yeast c/o) (SEQ ID NO: 92); Ha88B_2 (yeast c/o) (SEQ ID NO: 94); and/or Pt73Y (yeast c/o) (SEQ ID NO: 96).

35. The method of claims 20-34, wherein the glycosyl transferase has at least 70%, such as at least 75%, such as at least 80%, such as at least 90%, such as at least 95%, such as at least 99%, such as 100% identity to the glycosyl transferase comprised in anyone of Pt73Y (SEQ ID: NO 64); (SEQ ID NO: 66); Bs109_1 (SEQ ID NO: 68); Bs109A1 (SEQ ID NO: 70); Cp73B (SEQ ID NO: 72); Cs73Y (yeast c/o) (SEQ ID NO: 92); Ha88B_2 (yeast c/o) (SEQ ID NO: 94); and/or Pt73Y (yeast c/o) (SEQ ID NO: 96).

36. The method of claim 20, wherein the synthase is selected from the group consisting of: a Chorismate synthase, an Anthranilate synthase, an Indole-3-glycerol phosphate synthase, a Tryptophan synthase, a Prodeoxyviolacein synthase, and a Violacein Synthase.

37. The method of claim 36, wherein the Chorismate synthase has at least 70%, such as at least 75%, such as at least 80%, such as at least 90%, such as at least 95%, such as at least 99%, such as 100% identity to the synthase comprised in SEQ ID NO: 12.

38. The method of claim 36, wherein the Anthranilate synthase has at least 70%, such as at least 75%, such as at least 80%, such as at least 90%, such as at least 95%, such as at least 99%, such as 100% identity to the synthase comprised in SEQ ID NO: 14.

39. The method of claim 36, wherein the Indole-3-glycerol phosphate synthase has at least 70%, such as at least 75%, such as at least 80%, such as at least 90%, such as at least 95%, such as at least 99%, such as 100% identity to the synthase comprised in SEQ ID NO: 22.

40. The method of claim 36, wherein the Tryptophan synthase has at least 70%, such as at least 75%, such as at least 80%, such as at least 90%, such as at least 95%, such as at least 99%, such as 100% identity to the synthase comprised in SEQ ID NO: 24 (TRP5) and/or 63 (PcTrpB).

41. The method according to claim 40, wherein the synthase has at least 70% identity to the synthase comprised in SEQ ID NO: 24 (TRP5) and the synthase is contacted with the compound of formula (II) in vivo.

42. The method according to claim 41, wherein the compound of formula (II) is indole-3-glycerol phosphate or indole.

43. The method according to claim 40, wherein the synthase has at least 70% identity to the synthase comprised in SEQ ID NO: 63 (TRP5) and the synthase is contacted with the compound of formula (II) in vitro.

44. The method of claim 36, wherein the Prodeoxyviolacein synthase has at least 70%, such as at least 75%, such as at least 80%, such as at least 90%, such as at least 95%, such as at least 99%, such as 100% identity to the synthase comprised in SEQ ID NO: 30.

45. The method of claim 36, wherein the Violacein Synthase has at least 70%, such as at least 75%, such as at least 80%, such as at least 90%, such as at least 95%, such as at least 99%, such as 100% identity to the synthase comprised in SEQ ID NO: 34.

46. The method of claim 20, wherein the kinase is a Shikimate kinase, a Ribose-phosphate pyrophosphokinase, and/or a NADH kinase.

47. The method of claim 46, wherein the Shikimate kinase has at least 70%, such as at least 75%, such as at least 80%, such as at least 90%, such as at least 95%, such as at least 99%, such as 100% identity to the sequence comprised in SEQ ID NO: 10.

48. The method of claim 46, wherein the Ribose-phosphate pyrophosphokinase has at least 70%, such as at least 75%, such as at least 80%, such as at least 90%, such as at least 95%, such as at least 99%, such as 100% identity to the sequence comprised in SEQ ID NO: 16.

49. The method of claim 46, wherein the NADH kinase has at least 70%, such as at least 75%, such as at least 80%, such as at least 90%, such as at least 95%, such as at least 99%, such as 100% identity to the sequence comprised in SEQ ID NO: 51.

50. The method of any preceding claims, wherein the one or more enzymes is an Anthranilate phosphoribosyl transferase having at least 70%, such as at least 75%, such as at least 80%, such as at least 90%, such as at least 95%, such as at least 99%, such as 100% identity to the sequence comprised in SEQ ID NO: 18.

51. The method of any preceding claims, wherein the one or more enzymes is a Flavin-dependent L-tryptophan oxidase having at least 70%, such as at least 75%, such as at least 80%, such as at least 90%, such as at least 95%, such as at least 99%, such as 100% identity to the sequence comprised in SEQ ID NO: 26.

52. The method of any preceding claims, wherein the one or more enzymes is a tryptophan oxidase having at least 70%, such as at least 75%, such as at least 80%, such as at least 90%, such as at least 95%, such as at least 99%, such as 100% identity to the sequence comprised in: SEQ ID NO: 80, SEQ ID NO: 84, and/or SEQ ID NO: 88.

53. The method of any preceding claims, wherein the one or more enzymes is a 2-imino-3-(indol-3-yl)propanoate dimerase having at least 70%, such as at least 75%, such as at least 80%, such as at least 90%, such as at least 95%, such as at least 99%, such as 100% identity to the sequence comprised in SEQ ID NO: 28.

54. The method of any preceding claims, wherein the one or more enzymes is a 2-imino-3-(indol-3-yl)propanoate dimerase Prodeoxyviolacein synthase fusion protein having at least 70%, such as at least 75%, such as at least 80%, such as at least 90%, such as at least 95%, such as at least 99%, such as 100% identity to the sequence comprised in SEQ ID NO: 74 or SEQ ID NO: 76.

55. The method of any one of the preceding claims, wherein the one or more enzymes is an IPA imine dimer synthase having: a) at least 70% identity, such as at least 75%, such as at least 80%, such as at least 90%, such as at least 95%, such as at least 99%, such as 100% identity to LaRebD (SEQ ID NO: 78); b) at least 70% identity, such as at least 75%, such as at least 80%, such as at least 90%, such as at least 95%, such as at least 99%, such as 100% identity to SsStaD (SEQ ID NO: 82); c) at least 70% identity, such as at least 75%, such as at least 80%, such as at least 90%, such as at least 95%, such as at least 99%, such as 100% identity to NlInkD (SEQ ID NO: 86); or d) at least 70% identity, such as at least 75%, such as at least 80%, such as at least 90%, such as at least 95%, such as at least 99%, such as 100% identity to AmAtmD (SEQ ID NO: 90).

56. The method of any preceding claims, wherein the one or more enzymes is a Protodeoxyviolaceinate monooxygenase having at least 70%, such as at least 75%, such as at least 80%, such as at least 90%, such as at least 95%, such as at least 99%, such as 100% identity to the sequence comprised in SEQ ID NO: 32.

57. The method of any preceding claims, wherein the one or more enzymes is a transaldolase having at least 70%, such as at least 75%, such as at least 80%, such as at least 90%, such as at least 95%, such as at least 99%, such as 100% identity to the sequence comprised in SEQ ID NO: 38.

58. The method of any preceding claims, wherein the one or more enzymes is a Transketolase having at least 70%, such as at least 75%, such as at least 80%, such as at least 90%, such as at least 95%, such as at least 99%, such as 100% identity to the sequence comprised in SEQ ID NO: 40.

59. The method of any preceding claims, wherein the one or more enzymes is a GTP cyclohydrolase II having at least 70%, such as at least 75%, such as at least 80%, such as at least 90%, such as at least 95%, such as at least 99%, such as 100% identity to the sequence comprised in SEQ ID NO: 42.

60. The method of any preceding claims, wherein the one or more enzymes is Mitochondrial flavin adenine dinucleotide transporter having at least 70%, such as at least 75%, such as at least 80%, such as at least 90%, such as at least 95%, such as at least 99%, such as 100% identity to the sequence comprised in SEQ ID NO: 44.

61. The method of any preceding claims, wherein the one or more enzymes is Porphobilinogen deaminase having at least 70%, such as at least 75%, such as at least 80%, such as at least 90%, such as at least 95%, such as at least 99%, such as 100% identity to the sequence comprised in SEQ ID NO: 47.

62. The method of claims 37 to 61, wherein the sequence identity is at least 90%, such as at least 91%, such as at least 92%, such as at least 93%, such as at least 94%, such as at least 95%, such as at least 96%, such as at least 97%, such as at least 98%, such as at least 99%, such as 100%.

63. The method of any preceding claim, further comprising one or more steps selected from:
a) converting an indole or indole derivative into tryptophan or a tryptophan derivative;
b) adding an isolated indole of formula (II) to a microbial host cell;
c) converting an indole of formula (II) into tryptophan or a tryptophan derivative;
d) converting tryptophan or tryptophan derivative into the compound of formula (I);
e) converting the compound of formula (I) into a glycosylated compound thereof which is the glycosylated compound of formula (I), optionally in vivo;
f) extraction of the compound of formula (I) or the glycosylated compound of formula (I) using an extractant, such as a surfactant, optionally at a concentration above the extractant's cloud point; and
g) recovering the compound of formula (I) from an extractant phase.

64. The method of claim 63, wherein the method comprises extraction of the glycosylated compound of formula (I).

65. The method of claim 64, wherein the method further comprises de-glycosylation of the glycosylated compound of formula (I) by a β-glycosidase to provide the compound of formula (I), and optionally further isolating the compound of formula (I).

66. The method of claims 63-65, wherein the extractant is a surfactant, such as a non-ionic surfactant; or a lipophilic extractant.

67. The method of claims 63-65, wherein the extractant is non-miscible with water.

68. The method of claim 63, wherein the extractant is isopropyl myristate, (1,1,3,3-Tetramethylbutyl)phenyl-polyethylene glycol, Polyethylene glycol tert-octylphenyl ether (Triton X-114), or polydimethylsiloxane (such as Antifoam A).

69. The method of any of the preceding claims, wherein the steps are performed in vitro or in vivo.

70. The method of claims 63-69, wherein the conversion of the indole into the tryptophan or tryptophan derivative comprises contacting the indole with a tryptophan synthase enzyme, optionally a tryptophan synthase which has at least 70%, such as at least 75%, such as at least 80%, such as at least 90%, such as at least 95%, such as at least 99%, such as 100% identity to the tryptophan synthase comprised in SEQ ID NO: 24 and/or 63.

71. The method of any preceding claim comprising in vitro enzymatic reaction steps and/or optionally in vivo enzymatic reaction steps.

72. The method of any preceding claims, comprising expressing a glycosyl transferase in yeast, such as in S. cerevisiae and performing in vivo glycosylation of the compound of formula (I).

73. The method of claim 72, wherein the glycosyl transferase has at least 70% sequence identity to the polypeptide sequence comprised in sequence of Pt73Y according to SEQ ID NO: 66, such as at least 75%, such as at least 80%, such as at least 85%, such as at least 90%, such as at least 95%, such as 100%.

74. The method of claim 72, wherein the glycosyl transferase has at least 70% sequence identity to any one of the polypeptide sequences comprised in sequence of: Pt73Y (SEQ ID: NO 64); (SEQ ID NO: 66); Bs109_1 (SEQ ID NO: 68); Bs109A1 (SEQ ID NO: 70); Cp73B (SEQ ID NO: 72); Cs73Y (yeast c/o) (SEQ ID NO: 92); Ha88B_2 (yeast c/o) (SEQ ID NO: 94); and/or Pt73Y (yeast c/o) (SEQ ID NO: 96).

75. The method of claim 74, wherein the glycosyl transferase has at least 70% sequence identity to any one of the polypeptide sequences comprised in sequence of: Pt73Y (SEQ ID: NO 64); Cs73Y (yeast c/o) (SEQ ID NO: 92); and/or Pt73Y (yeast c/o) (SEQ ID NO: 96), such as at least 75%, such as at least 80%, such as at least 85%, such as at least 90%, such as at least 95%, such as 100%.

76. The method of claim 71, comprising expressing a glycosyl transferase in yeast, such as in S. cerevisiae or Pichia pastoris and performing in vitro glycosylation of the compound of formula (I).

77. The method of any preceding claims, comprising expressing a glycosyl transferase in E. coli and performing in vitro glycosylation of the compound of formula (I).

78. The method of claims 76-77, wherein the glycosyl transferase has at least 70% sequence identity to any one of the polypeptide sequences comprised in sequence of: Pt73Y (SEQ ID: NO 64); (SEQ ID NO: 66); Bs109_1 (SEQ ID NO: 68); Bs109A1 (SEQ ID NO: 70); Cp73B (SEQ ID NO: 72); Cs73Y (yeast c/o) (SEQ ID NO: 92); Ha88B_2 (yeast c/o) (SEQ ID NO: 94); and/or Pt73Y (yeast c/o) (SEQ ID NO: 96).

79. The method of claims 76-77, wherein the glycosyl transferase has at least 70% sequence identity to the polypeptide sequence comprised in sequence of Pt73Y according to SEQ ID NO: 66, such as at least 75%, such as at least 80%, such as at least 85%, such as at least 90%, such as at least 95%, such as 100%.

80. A microbial host cell genetically modified to perform any of the method of claims 1 to 79 and produce a compound of formula (I) selected from the group consisting of: 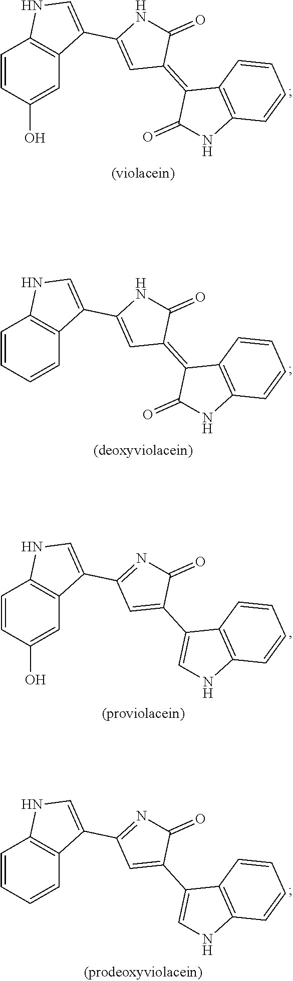 [SMILES: O=C1NC(c2c[nH]c3ccc(O)cc23)=C/C1=C1\C(=O)Nc2ccccc21 ; O=C1NC(c2c[nH]c3ccccc23)=C/C1=C1\C(=O)Nc2ccccc21 ; O=C1N=C(c2c[nH]c3ccc(O)cc23)C=C1c1c[nH]c2ccccc12 ; O=C1N=C(c2c[nH]c3ccccc23)C=C1c1c[nH]c2ccccc12]
and a tautomer thereof; wherein the host cell expresses one or more heterologous genes encoding the one or more enzymes, and wherein the microbial host cell comprises at least:
a) a tryptophan oxidase having at least 70% identity to the sequence comprised in SsStaO (SEQ ID NO: 80), NlInkO (SEQ ID NO: 84), and/or AmAtmO (SEQ ID NO: 88); and/or
b) an IPA imine dimer synthase has at least 70% identity to the sequence comprised in LaRebD (SEQ ID NO: 78), SsStaD (SEQ ID NO: 82), NlInkD (SEQ ID NO: 86), and/or AmAtmD (SEQ ID NO: 90); and wherein the microbial host cell is Saccharomyces cerevisiae.

81. The host cell of claim 80, further comprising an operative biosynthetic pathway for producing violacein, wherein the host cell expresses one or more pathway genes encoding polypeptides selected from:
a) one or more enzymes capable of converting glucose to fructose-6-phosphate;
b) one or more enzymes capable of converting glucose to D-ribulose-5-phosphate;
c) a transketolase capable of converting xylulose-5-phosphate and ribose-5-phosphate to glyceraldehyde-3-phosphate and sedoheptulose-7-phosphate, such as the transketolase TKL1;
d) a transaldolase capable of converting glyceraldehyde 3-phosphate and sedoheptulose 7-phosphate to erythrose 4-phosphate and fructose 6-phosphate, such as the transaldolase TAL1;
e) a fructose-6-phosphate phosphoketolase capable of converting fructose-6-phosphate to Erythrose-4-phosphate and acetyl phosphate, such as the phosphoketolase BfXfpk;
f) a Phosphotransacetylase capable of converting Acetyl phosphate to Acetyl-CoA, such as the phosphotransacetylase CkPTa;
g) one or more enzymes capable of converting Fructose-6-phosphate to Phosphoenolpyruvate;
h) a 3-deoxy-D-arabino-heptulosonate 7-phosphate synthase (DAHP synthase) capable of converting Phosphoenolpyruvate and Erythrose-4-phosphate to 3-deoxy-D-arabino-heptulosonate-7-phosphate (DAHP), such as the synthase ARO4(K229L);
i) a 3-dehydroquinate synthase capable of converting 3-deoxy-D-arabino-heptulosonate 7-phosphate to 3-dehydroquinate, such as the synthase ARO1;
j) a 3-dehydroquinate dehydratase capable of converting 3-dehydroquinate to 3-dehydroshikimate, such as the dehydratase ARO1;
k) a Shikimate dehydrogenase capable of converting 3-dehydroshikimate to Shikimate, such as the dehydrogenase ARO1;
l) a Shikimate kinase capable of converting Shikimate to Shikimate-3-phosphate, such as the kinase ARO1 and/or EcAroL;
m) a 3-phosphoshikimate 1-carboxyvinyltransferase capable of converting Shikimate-3-phosphate and Phosphoenolpyruvate to 5-enolpyruvoyl-shikimate 3-phosphate, such as the transferase ARO1;
n) a Chorismate synthase capable of converting 5-enolpyruvoyl-shikimate 3-phosphate to Chorismate, such as the synthase ARO2;
o) an Anthranilate synthase capable of converting Chorismate to Anthranilate, such as the synthase TRP2(S65R, S76L);
p) a Ribose-phosphate pyrophosphokinase capable of converting Ribose-5-phosphate to Phospho-alpha-D-ribosyl-1-pyrophosphate, such as the pyrophosphokinase BsPrs;
q) an Anthranilate phosphoribosyl transferase capable of converting Anthranilate and Phospho-alpha-D-ribosyl-1-pyrophosphate to N-(5-phosphoribosyl)-anthranilate, such as the transferase TRP4;
r) a N-(5′-phosphoribosyl)-anthranilate isomerase capable of converting N-(5-phosphoribosyl)-anthranilate to 1-(o-carboxyphenylamino)-1′-deoxyribulose 5′-phosphate, such as the isomerase TRP1;
s) a Indole-3-glycerol phosphate synthase capable of converting 1-(o-carboxyphenylamino)-1′-deoxyribulose 5′-phosphate to (1S,2R)-1-C-(indol-3-yl)glycerol 3-phosphate, such as the synthase TRP3
t) a Tryptophan synthase capable of converting (1S,2R)-1-C-(indol-3-yl)glycerol 3-phosphate and Serine to L-Tryptophan, such as the synthase TRP5;
u) a tryptophan synthase capable of converting Indole and Serine to L-Tryptophan, such as the synthase TRP5;
v) a Flavin-dependent L-tryptophan oxidase capable of converting L-Tryptophan to IPA imine, such as CvVioA;
w) a tryptophan oxidase, such as SsStaO, NlInkO, or AmAtmO;
x) a 2-imino-3-(indol-3-yl)propanoate dimerase capable of converting IPA imine to IPA imine dimer, such as the dimerase CvVioB;
y) an IPA imine dimer synthase, such as LaRebD, SsStaD, NlInkD, and/or AmAtmD;
z) a Prodeoxyviolacein synthase capable of converting IPA imine dimer to Protodeoxyviolaceinic acid, such as the synthase CvVioE;
aa) a Protodeoxyviolaceinate monooxygenase synthase capable of converting Protodeoxyviolaceinic acid to Protoviolaceinic acid, such as the synthase CvVioD; and
bb) a Violacein synthase capable of converting Protoviolaceinic acid to Violaceinic acid and capable of converting Protodeoxyviolaceinic acid to Protoviolaceinic acid, such as CvVioC.

82. The host cell of claims 80-81, further comprising an operative biosynthetic pathway for heme biosynthesis, wherein the host cell expresses one or more pathway genes encoding polypeptides selected from:
a) one or more enzymes capable of converting glucose to glycine;
b) one or more enzymes capable of converting glycine to porphobilinogen;
c) a Porphobilinogen deaminase capable of converting Porphobilinogen to Hydroxymethylbilane, such as the deaminase HEM3; and
d) one or more enzymes capable of converting Hydroxymethylbilane to Ferroheme b.

83. The host cell of claims 80-82, further comprising an operative biosynthetic pathway for flavin biosynthesis, wherein the host cell expresses one or more pathway genes encoding polypeptides selected from:
a) a GTP cyclohydrolase II capable of converting GTP to 2,5-diamino-6-ribosylamino-4(3H)-pyrimidinone 5′-phosphate, such as the cyclohydrolase RIB1; and
b) one or more enzymes capable of converting 2,5-diamino-6-ribosylamino-4(3H)-pyrimidinone 5′-phosphate to FAD.

84. The host cell of claims 88-83, wherein the host cell further expresses one or more genes encoding catalytic or non-catalytic polypeptides selected from:
a) a NADH kinase capable of converting NADH and ATP to NADPH and ADP, such as the kinase POS5; and
b) a Mitochondrial flavin adenine dinucleotide transporter, such as FLX1.

85. The host cell of claims 80-84, wherein one or more genes has been attenuated, disrupted and/or deleted, said one or more genes encoding catalytic or non-catalytic polypeptides selected from:
a) a Heme oxygenase capable of converting Ferroheme b to Biliverdin, such as the oxygenase HMX1;
b) a Heme-responsive transcription factor, such as HAP1;
c) a mRNA-binding ubiquitin-specific protease, such as UBP3;
d) a Cis-Golgi network transporter protein, such as RIC1; and
e) a Heme-dependent repressor of hypoxic genes, such as ROX1.

86. The host cell of claims 80-85, comprising at least two copies of one or more of the heterologous genes encoding the one or more enzymes of the pathway genes.

87. The host cell of claim 86, wherein one or more of the heterologous genes encoding the one or more enzymes are overexpressed.

88. The host cell of claims 80-87, further genetically modified to provide an increased amount of a substrate for at least one polypeptide of the violacein pathway.

89. The host cell of claims 80-88, further genetically modified to exhibit increased tolerance towards one or more substrates, intermediates, or product molecules from the indole acceptor pathway.

90. The host cell of claims 80-89, wherein one or more native genes are attenuated, disrupted and/or deleted.

91. The host cell of claims 80-90, wherein the host cell is a yeast strain modified by attenuating, disrupting and/or deleting one or more native genes selected from:
a) The ARO10 gene comprised in anyone of SEQ ID NO: 49 or any of its paralogs or orthologs having at least 70% identity to SEQ ID NO: 49;
b) The PDC5 gene comprised in anyone of SEQ ID NO: 48 or any of its paralogs or orthologs having at least 70% identity to SEQ ID NO: 48;
c) The UBP3 gene comprised in anyone of SEQ ID NO: 57 or any of its paralogs or orthologs having at least 70% identity to SEQ ID NO: 57;
d) The RIC1 gene comprised in anyone of SEQ ID NO: 58 or any of its paralogs or orthologs having at least 70% identity to SEQ ID NO: 58;
e) The GPP1 gene comprised in anyone of SEQ ID NO: 59 or any of its paralogs or orthologs having at least 70% identity to SEQ ID NO: 59;
f) The ROX1 gene comprised in anyone of SEQ ID NO: 59 or any of its paralogs or orthologs having at least 70% identity to SEQ ID NO: 60;
g) The HMX1 gene comprised in anyone of SEQ ID NO: 59 or any of its paralogs or orthologs having at least 70% identity to SEQ ID NO: 45; and
h) The HAP1 gene comprised in anyone of SEQ ID NO: 59 or any of its paralogs or orthologs having at least 70% identity to SEQ ID NO: 61.

92. The host cell of claims 80-91, wherein the host cell is a yeast strain modified by overexpressing one or more genes selected from:
a) The ARO1 gene comprised in SEQ ID NO: 7 or any of its paralogs or orthologs having at least 70% identity to SEQ ID NO: 7;
b) The ARO2 gene comprised in SEQ ID NO: 11 or any of its paralogs or orthologs having at least 70% identity to SEQ ID NO:11;
c) The TRP4 gene comprised in SEQ ID NO: 17 or any of its paralogs or orthologs having at least 70% identity to SEQ ID NO: 17;
d) The TRP1 gene comprised in SEQ ID NO: 19 or any of its paralogs or orthologs having at least 70% identity to SEQ ID NO: 19;
e) The TRP3 gene comprised in SEQ ID NO: 21 or any of its paralogs or orthologs having at least 70% identity to SEQ ID NO: 21;
f) The TRP5 gene comprised in SEQ ID NO: 23 or any of its paralogs or orthologs having at least 70% identity to SEQ ID NO: 23;
g) The TAL1 gene comprised in SEQ ID NO: 37 or any of its paralogs or orthologs having at least 70% identity to SEQ ID NO: 37;
h) The TKL1 gene comprised in SEQ ID NO: 39 or any of its paralogs or orthologs having at least 70% identity to SEQ ID NO: 39;
i) The RIB1 gene comprised in SEQ ID NO: 41 or any of its paralogs or orthologs having at least 70% identity to SEQ ID NO: 41;
j) The FLX1 gene comprised in SEQ ID NO: 43 or any of its paralogs or orthologs having at least 70% identity to SEQ ID NO: 43;
k) The POS5 gene comprised in SEQ ID NO: 50 or any of its paralogs or orthologs having at least 70% identity to SEQ ID NO: 50; and
l) The HEM3 gene comprised in SEQ ID NO: 46 or any of its paralogs or orthologs having at least 70% identity to SEQ ID NO: 46.

93. The host cell of any claims 80-92, wherein the host cell is a yeast strain modified by overexpressing one or more genes selected from:
a) The K229L modified ARO4 gene, ARO4(K229L) comprised in SEQ ID NO: 5 or any of its paralogs or orthologs having at least 70% identity to SEQ ID NO: 5; and
b) The (S65R, S76L) modified TRP2 gene, TRP2(S65R, S76L) comprised in SEQ ID NO: 13 or any of its paralogs or orthologs having at least 70% identity to SEQ ID NO: 13.

94. The host cell of claims 80-93, wherein the host cell is a yeast strain modified by heterologous gene overexpressing of one or more genes selected from:
a) CvVioA encoding comprised in SEQ ID NO: 25 or any of its paralogs or orthologs having at least 70% identity to SEQ ID NO: 25;
b) CvVioB comprised in SEQ ID NO: 27 or any of its paralogs or orthologs having at least 70% identity to SEQ ID NO: 27;
c) CvVioC comprised in SEQ ID NO: 33 or any of its paralogs or orthologs having at least 70% identity to SEQ ID NO: 33;
d) CvVioD comprised in SEQ ID NO: 31 or any of its paralogs or orthologs having at least 70% identity to SEQ ID NO: 31;
e) CvVioE comprised in SEQ ID NO: 29 or any of its paralogs or orthologs having at least 70% identity to SEQ ID NO: 29;
f) BfXfpk comprised in SEQ ID NO: 1 or any of its paralogs or orthologs having at least 70% identity to SEQ ID NO: 1;
g) CkPta comprised in SEQ ID NO: 3 or any of its paralogs or orthologs having at least 70% identity to SEQ ID NO: 3;
h) EcAroL comprised in SEQ ID NO: 9 or any of its paralogs or orthologs having at least 70% identity to SEQ ID NO: 9; and
i) BsPrs comprised in SEQ ID NO: 15 or any of its paralogs or orthologs having at least 70% identity to SEQ ID NO: 15.

95. The host cell of claims 80-93, wherein the host cell is a yeast strain modified by heterologous gene overexpressing of one or more genes selected from:
a) CvVioA encoding comprised in SEQ ID NO: 25 or any of its paralogs or orthologs having at least 70% identity to SEQ ID NO: 25;
b) CvVioB comprised in SEQ ID NO: 27 or any of its paralogs or orthologs having at least 70% identity to SEQ ID NO: 27;
c) CvVioC comprised in SEQ ID NO: 33 or any of its paralogs or orthologs having at least 70% identity to SEQ ID NO: 33;
d) CvVioD comprised in SEQ ID NO: 31 or any of its paralogs or orthologs having at least 70% identity to SEQ ID NO: 31;
e) CvVioE comprised in SEQ ID NO: 29 or any of its paralogs or orthologs having at least 70% identity to SEQ ID NO: 29;
f) CvVioB-E fusion GGGGS3 linker comprised in SEQ ID NO:73 or any of its paralogs or orthologs having at least 70% identity to SEQ ID NO: 73;
g) CvVioB-E fusion EAAAK3 linker comprised in SEQ ID NO: 75 or any of its paralogs or orthologs having at least 70% identity to SEQ ID NO: 75;
h) BfXfpk comprised in SEQ ID NO: 1 or any of its paralogs or orthologs having at least 70% identity to SEQ ID NO: 1;
i) CkPta comprised in SEQ ID NO: 3 or any of its paralogs or orthologs having at least 70% identity to SEQ ID NO: 3;
j) EcAroL comprised in SEQ ID NO: 9 or any of its paralogs or orthologs having at least 70% identity to SEQ ID NO: 9; and
k) BsPrs comprised in SEQ ID NO: 15 or any of its paralogs or orthologs having at least 70% identity to SEQ ID NO: 15.

96. The host cell according to any one of claims 80-95, wherein the host cell is genetically engineered to produce one or more glycosyl transferases, such as one or more UDP-glucuronosyltransferases (UGT's).

97. The host cell according to claim 96, wherein the one or more glycosyl transferases are configured for or capable of glycosylating the compound of formula (I).

98. The host cell according to any one of claims 96-97, wherein the one or more glycosyl transferases have at least 70% sequence identity to the polypeptide sequence comprised in the sequence of Pt73Y according to SEQ ID NO: 66, such as at least 75%, such as at least 80%, such as at least 85%, such as at least 90%, such as at least 95%, such as 100%.

99. The host cell according to any one of claims 96-98, wherein the one or more glycosyl transferases have at least 70% sequence identity to any one of the polypeptide sequences comprised in the sequence of: Pt73Y (SEQ ID: NO 64); (SEQ ID NO: 66); Bs109_1 (SEQ ID NO: 68); Bs109A1 (SEQ ID NO: 70); Cp73B (SEQ ID NO: 72); Cs73Y (yeast c/o) (SEQ ID NO: 92); Ha88B_2 (yeast c/o) (SEQ ID NO: 94); and/or Pt73Y (yeast c/o) (SEQ ID NO: 96).

100. The host cell according to any one of claims 96-99, wherein the one or more glycosyl transferases produced by the host cell have at least 70% sequence identity to any one of the polypeptide sequences comprised in sequence of: Pt73Y (SEQ ID: NO 64); Cs73Y (yeast c/o) (SEQ ID NO: 92); and/or Pt73Y (yeast c/o) (SEQ ID NO: 96), such as at least 75%, such as at least 80%, such as at least 85%, such as at least 90%, such as at least 95%, such as 100%.

101. The host cell according to any one of claims 96-100, wherein said host cell expresses the one or more glycosyl transferases.

102. A compound of formula (I): 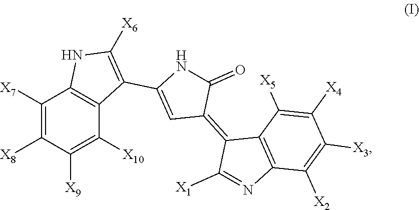
or a tautomer thereof, wherein any one of X₁, X₂, X₃, X₄, X₅, X₆, X₇, X₈, X₉, and X₁₀ are independently of each other selected from the group consisting of: H, R₁, R₂, O, OH, OR₁, NH, NO₂, NH₂, NHR₁, NHR₂, SR₁, F, Cl, Br, I, and SH; wherein R₁ and R₂ are independently of each other selected from the group consisting of a C₁₋₈ alkyl, C₁₋₈ alkenyl, C₁₋₈ alkoyl, C₁₋₈ aryl, and C₁₋₈ aroyl, and R₁ and R₂ are optionally covalently linked to form a ring.

103. The compound according to claim 102, wherein the compound is selected from the group consisting of: 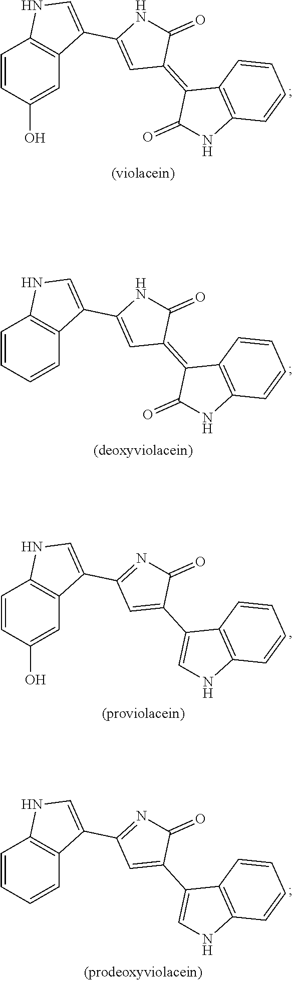 [SMILES: O=C1NC(c2c[nH]c3ccc(O)cc23)=C/C1=C1\C(=O)Nc2ccccc21 ; O=C1NC(c2c[nH]c3ccccc23)=C/C1=C1\C(=O)Nc2ccccc21 ; O=C1N=C(c2c[nH]c3ccc(O)cc23)C=C1c1c[nH]c2ccccc12 ; O=C1N=C(c2c[nH]c3ccccc23)C=C1c1c[nH]c2ccccc12]
and tautomers thereof.

104. The compound according to any one of claims 102-103, further covalently linked to a saccharide, preferably by a glycosidic linkage.

105. The compound according to claim 104, wherein the compound is of formula (III) or formula (IV): 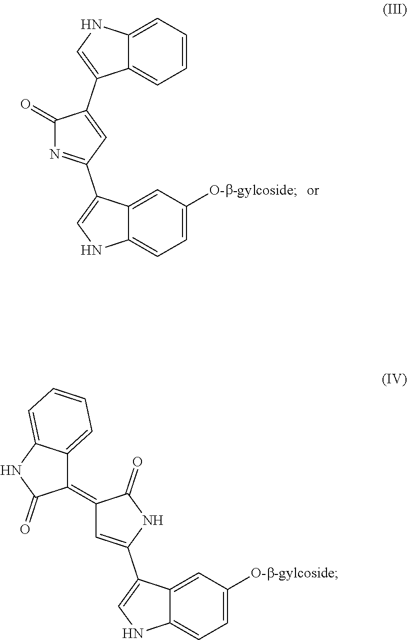
wherein “1-glycoside” is a saccharide linked by a β-glycosidic bond to the remainder of the molecule.

106. The compound according to any of claims 104-105, wherein the saccharide is a monosaccharide, a disaccharide, a trisaccharide, or a tetrasaccharide.

107. The compound according to claim 106, wherein the monosaccharide is selected from the group consisting of: glucose, fructose, galactose, mannose, arabinose, xylose, ribulose, xylulose, ribose, desoxyribose, desoxygalactose, fucose, and rhamnose, preferably wherein the monosaccharide is glucose, such as D-glucose.

108. A cell culture, comprising a host cell as defined in any of claims 80-101 and a growth medium.

109. The method of any claims 1 to 79 further comprising:
a) culturing the cell culture of claim 108 at conditions allowing the host cell to produce the compound of formula (I); and
b) optionally recovering and/or isolating the compound of formula (I).

110. The method of claim 109, further comprising feeding one or more exogenous indoles of formula (II) to the cell culture.

111. The method of claims 109 to 110, wherein the recovering and/or isolation step comprises separating a liquid phase of host cell or cell culture from a solid phase of host cell or cell culture to obtain a supernatant comprising the compound of formula (I) by one or more steps selected from:
a) disrupting the host cell to release intracellular the compound of formula (I) into the supernatant;
b) separating the supernatant from the solid phase of the host cell, such as by filtration or gravity separation;
c) contacting the supernatant with one or more adsorbent resins in order to obtain at least a portion of the produced compound of formula (I);
d) contacting the supernatant with one or more ion exchange or reversed-phase chromatography columns in order to obtain at least a portion of the compound of formula (I);
e) extracting the compound of formula (I); and
f) precipitating the compound of formula (I) by crystallization or evaporating the solvent of the liquid phase; and optionally isolating the compound of formula (I) by filtration or gravity separation;
thereby recovering and/or isolating the compound of formula (I).

112. A fermentation liquid comprising the compound of formula (I) comprised in the cell culture of claim 108.

113. The fermentation liquid of claim 112, wherein at least 50%, such as at least 75%, such as at least 95%, such as at least 99% of the host cells are disrupted.

114. The fermentation liquid of claim 112 to 113, wherein at least 50%, such as at least 75%, such as at least 95%, such as at least 99% of solid cellular material has separated from the liquid.

115. The fermentation liquid of claim 112 to 114, further comprising one or more compounds selected from:
a) precursors or products of the operative biosynthetic pathway producing the compound of formula (I);
b) supplemental nutrients comprising trace metals, vitamins, salts, yeast nitrogen base, YNB, and/or amino acids; and
wherein the concentration of the compound of formula (I) is at least 1 mg/I liquid.

116. A composition comprising the fermentation liquid of claims 112 to 115 and/or the compound of formula (I) of claims 102 to 107 and one or more agents, additives and/or excipients.

117. A method for modification of a microbial host cell producing the compound formula (I) as defined in claim 1, comprising:
a) Providing a microbial host cell which is S. cerevisiae;
b) Engineering the microbial host cell by inserting one or more genes encoding one or more of the enzymes as defined in claims 1-62.

118. The method according to claim 117, wherein the microbial host cell is as defined in any one of claims 80-94.

119. A method for in-situ extraction of the compound of formula (I) or the glycosylated compound of formula (I), comprising:
a) Providing a host cell as defined in any one of claims 80-94 or the cell culture as defined in claim 108 comprising the compound of formula (I) or the glycosylated compound of formula (I) in an aqueous phase;
b) Subjecting the aqueous phase to extraction with an extractant, optionally wherein the extractant is a non-ionic surfactant, preferably wherein the extraction is performed during cultivation of the host cell.

120. The method according to claim 119, wherein the method further comprises producing the compound of formula (I) using the method as defined in any one of claims 1-79.

121. The method according to any one of claims 119-120, wherein the extractant is a surfactant or a lipophilic extractant.

122. The method according to claim 121, wherein the surfactant is a non-ionic or ionic surfactant.

123. The method according to claim 121, wherein the extractant is a lipophilic extractant, preferably a non-toxic lipophilic extractant.

124. The method according to claim 123, wherein the extractant is a lipophilic non-volatile extractant.

125. The method according to any one of claims 123-124, wherein the lipophilic extractant is selected from the group consisting of: an ester, such as a C₂-C₂₀ ester, an alcohol, such as a C₂-C₂₀ alcohol, and a vegetable oil, such as grapeseed oil, olive oil, sunflower oil, or canola oil.

126. The method according to any one of claims 119-122, wherein the extractant is subjected to the aqueous phase to form a liquid media with the aqueous phase such that the concentration of the extractant with respect to the liquid media is at least at the cloud-point of the extractant.

127. The method according to any one of claims 119-122, wherein the extractant is subjected to the aqueous phase to form a liquid media with the aqueous phase such that the concentration of the extractant with respect to the liquid media is at least at the cloud-point of the extractant and below the toxicity level for the host cell, such as the LD50.

128. The method according to any one of claims 119-127, wherein the extractant is (1,1,3,3-Tetramethylbutyl)phenyl-polyethylene glycol, Polyethylene glycol tert-octylphenyl ether (Triton X-114).

129. The method according to any one of claims 119-127, wherein the extractant is selected from the group consisting of Antifoam-A, Triton-X 114, isopropyl myristate, isopropyl palmitate, polysorbate 20, ethyl laurate, castor oil, oleyl alcohol, butyl caprilate, grapeseed oil, 2-butyl-1-octanol, and oleic acid, or any combination thereof.

130. The method according to any one of claims 119-127, wherein the extractant is polydimethylsiloxane (such as Antifoam A) or isopropyl myristate.

131. The method according to any one of claims 119-130, wherein the extractant is added such as to provide a concentration of the extractant of at least 1%, such as from 1-20%, such as from 1-2%, such as from 2-3%, such as from 3-4%, such as from 4-5%, such as from 5-6%, such as from 6-7%, such as from 7-8%, such as from 8-9%, such as from 9-10%, such as from 10-11%, such as from 11-12%, such as from 12-13%, such as from 13-14%, such as from 14-15%, such as from 15-16%, such as from 16-17%, such as from 17-18%, such as from 18-19%, such as from 19-20%.

132. The method according to any one of claims 119-131, wherein the method comprises one or more steps of:
a. removing biomass by filtration or centrifugation from the aqueous phase or the extractant;
b. separating and recovering the extractant comprising the compound of formula (I) or the glycosylated compound of formula (I) from the aqueous phase;
c. separating and recovering the compound of formula (I) or the glycosylated compound of formula (I) from the extractant by precipitation;
d. recovering the extractant.

133. The method according to claim 132, wherein the step b of separating and recovering the extractant involves one or more steps of i) increasing the temperature, ii) adding one or more salts to the mixture of the aqueous phase and extractant, and/or iii) centrifuging the mixture.

134. The method according to claim 132, wherein the step b of separating and recovering the extractant involves one or more steps of i) increasing the temperature, ii) adding one or more salts to the mixture of the aqueous phase and extractant, and/or iii) centrifuging the mixture, such that one or more of these steps moves the mixture above its cloud point.

135. The method according to any one of claims 132-134, wherein the step c of separating and recovering the compound of formula (I) or the glycosylated compound of formula (I) involves one or more steps of i) lowering the temperature, ii) adding an alcohol to the extractant, such as ethanol, and/or iii) centrifuging the extractant.

136. The method according to any one of claims 132-135, wherein the step d of recovering the extractant involves one or more steps of i) increasing the temperature, ii) evaporating the alcohol, such as ethanol, iii) adding one or more salts to the extractant, and/or iv) centrifuging.

137. The method according to any one of claims 132-136, wherein the removing of biomass by centrifugation in step a is performed at room temperature.

138. The method according to any one of claims 132-137, wherein the extractant is a non-ionic surfactant.

139. The method according to claim 138, wherein the non-ionic surfactant is selected from the group consisting of: antifoam-A, Triton and polysorbate 20.

140. The method according to any one of claims 132-139, wherein after the step c, the precipitated compound of formula (I) or the glycosylated compound of formula (I) is resuspended in ethanol and subjected to evaporation to remove ethanol.

141. The method according to claim 140, wherein the remaining solution after evaporation is further subjected to freeze drying to obtain a dried form of the compound of formula (I) or the glycosylated compound of formula (I).

142. The method according to any one of claims 132-136, wherein the step d of recovering the extractant comprises evaporating the ethanol using a vacuum centrifuge.

143. The method according to any one of claims 132-136, wherein after the step d, the method involves using the recovered extractant in subsequent extractions.

144. The method according to any one of claims 132-143, wherein the method further comprises cultivating the host cell in a growth medium.

145. The method according to claim 144, further comprising the steps of i) separating the cultivation into distinct phases comprising a biomass phase, an aqueous phase, and an extractant phase; and subsequently ii) collecting the extractant phase comprising the compound of formula (I) or the glycosylated compound of formula (I).

146. The method according to any one of claims 144-145, wherein the extractant is added to a final concentration of from 6 to 14%, such as from 8 to 12%, for example 10%.

147. The method according to any one of claims 144-146, wherein the host cell is cultivated for one or more days, such as from 2 to 7 days, for example 3 to 6 days, such as 4 days, wherein the host cell is cultivated at from 25 to 40° C., such as from 25 to 38° C., such as from 26 to 36° C., such as from 28 to 34° C., for example 30° C.

148. The method according to any one of claims 144-147, wherein the extractant is at least one of isopropyl myristate, isopropyl palmitate, antifoam-A, polysorbate, ethyl laurate, and castor oil.

149. The method according to any one of claims 144-147, wherein the compound of formula (I) is violacein or deoxyviolacein and the extractant is at least one of Antifoam-A, isopropyl myristate, isopropyl palmitate, ethyl laurate, grapeseed oil, 2-butyl-1-octanol, and oleic acid.

150. The method according to any one of claims 119-149, wherein the extractant comprising the compound of formula (I) or the glycosylated compound of formula (I) is loaded onto dry silica to provide an extractant bound to silica.

151. The method according to claim 150, wherein the extractant bound to silica is washed with a volatile solvent one or more times to remove the extractant.

152. The method according to claim 151, wherein the volatile solvent is selected from the group consisting of dichloromethane, hexane, and ethyl acetate.

153. The method according to any one of claims 150-152, wherein the method further comprises a step of eluting the compound of formula (I) or the glycosylated compound of formula (I) from the silica using a polar protic solvent, such as an alcohol, for example ethanol.

154. The method according to claim 153, wherein the method further comprises a step of evaporating the polar protic solvent used in elution to obtain the compound of formula (I) or the glycosylated compound of formula (I) in solid form.

155. The method according to any one of claims 119-149, wherein purification of the compound of formula (I) or the glycosylated compound of formula (I) is done using column chromatography.

156. The method according to any one of claims 119-155, wherein the compound of formula (I) is violacein, deoxyviolacein, proviolacein, or prodeoxyviolacein, for example deoxyviolacein.

157. The method according to any one of claims 119-156, further comprising the steps of:
a. collecting the extractant comprising the compound of formula (I) or the glycosylated compound of formula (I),
b. subsequently diluting the extractant with an alcohol, such as ethanol to a predefined concentration of the extractant with respect to the alcohol to provide a mixture of extractant and alcohol, and
c. cooling the mixture of extractant and alcohol to a preset temperature, optionally under stirring, to solidify the extractant thereby increasing the concentration of the compound of formula (I) or the glycosylated compound of formula (I) in the alcohol.

158. The method according to claim 157, further comprising a step of filtration, such that solidified extractant is removed, optionally at the preset temperature.

159. The method according to claim 158, further comprising a step of evaporating the alcohol to provide the compound of formula (I) or the glycosylated compound of formula (I) in concentrated form relative to the concentration of the compound of formula (I) or the glycosylated compound of formula (I) in the extractant collected in step a of claim 157, optionally wherein the concentrated form is a paste.

160. The method according to any one of claims 157-159, wherein the predefined concentration of the extractant with respect to the alcohol is from 20 to 40% extractant, such as from 20 to 21%, such as from 21 to 22%, such as from 22 to 23%, such as from 23 to 24%, such as from 24 to 25%, such as from 25 to 26%, such as from 26 to 27%, such as from 27 to 28%, such as from 28 to 29%, such as from 29 to 30%, such as from 30 to 31%, such as from 31 to 32%, such as from 32 to 33%, such as from 33 to 34%, such as from 34 to 35%, such as from 35 to 36%, such as from 36 to 37%, such as from 37 to 38%, such as from 38 to 39%, such as from 39 to 40%, for example 33%.

161. The method according to any one of claims 157-160, wherein the preset temperature is at the solidification temperature (melting point) of the extractant or less.

162. The method according to any one of claims 157-160, wherein the preset temperature is 20° C. or less, such as 19° C. or less, such as 18° C. or less, such as 17° C. or less, such as 16° C. or less, such as 15° C. or less, such as 14° C. or less, such as 13° C. or less, such as 12° C. or less, such as 11° C. or less, such as 10° C. or less, such as 9° C. or less, such as 8° C. or less, such as 7° C. or less, such as 6° C. or less, such as 5° C. or less, such as 4° C. or less, such as 3° C. or less, such as 2° C. or less, such as 1° C. or less, such as 0° C. or less, such as −1° C. or less, such as −2° C. or less, such as −3° C. or less, such as −4° C. or less, such as −5° C. or less, such as −6° C. or less, such as −7° C. or less, such as −8° C. or less, such as −9° C. or less, such as −10° C. or less.

163. The method according to any one of claims 157-160, wherein the preset temperature is from 20° C. to −5° C., such as from 19° C. to −5° C., such as from 18° C. to −5° C., such as from 17° C. to −5° C., such as from 16° C. to −5° C., such as from 15° C. to −5° C., such as from 14° C. to −5° C., such as from 13° C. to −5° C., such as from 12° C. to −5° C., such as from 11° C. to −5° C., such as from 10° C. to −5° C., such as from 9° C. to −5° C., such as from 8° C. to −5° C., such as from 7° C. to −5° C., such as from 6° C. to −5° C., such as from 5° C. to −5° C.

164. A method for dyeing a textile material, comprising:
a. providing an optionally dried composition of one or more compounds as defined in any one of claims 102-107, for example violacein, proviolacein, prodeoxyviolacein, and/or deoxyviolacein; and subsequently preparing a dye solution by suspending said composition in a liquid, such as an alcohol, for example ethanol; or
b. providing a colored fermentation extract comprising an extractant and one or more compounds as defined in any one of claims 102-107, for example violacein, proviolacein, prodeoxyviolacein, and/or deoxyviolacein
c. contacting a textile material with said dye solution or said colored fermentation extract, optionally for a predetermined duration, thereby dyeing the textile material.

165. The method according to claim 164, further comprising a step d) of removing the textile material from said dye solution or colored fermentation extract and washing with water to remove any excess dye.

166. The method according to claim 165, further comprising a step e) of drying the dyed textile material without the use of pre-treatments, mordants, or other chemical processing steps, and wherein the textile material retains a color change indicative of dyeing.

167. The method according to any one of claims 164-166, wherein the liquid is at least 90% ethanol, such as 100% ethanol.

168. The method according to any one of claims 164-167, wherein the textile material is selected from the group consisting of nylon 6,6, diacetate, polyester, cotton, such as bleached cotton, wool, hemp rayon, denim, viscose, and silk.

169. The method according to any one of claims 164-168, wherein the predetermined duration is from 10 minutes to 2 hours, such as 30 minutes.

170. The method according to any one of claims 164-169, wherein the composition is derived from the host cell as defined in any one of claims 80-95.

171. The method according to any one of claims 164-170, wherein the composition is in the form of a purified fermentation extract.

172. The method according to any one of claims 164-171, wherein the colored fermentation extract is obtainable by the method as defined in any one of claims 119-163.

173. The method according to any one of claims 164-171, further comprising providing a colored fermentation extract using the method as defined in any one of claims 119-163, wherein the method further comprises a step of diluting the colored fermentation extract in a liquid to provide a dye bath.

174. The method according to claim 173, wherein the method comprises diluting the colored fermentation extract to an extractant concentration of from 2% to 30%, such as from 2 to 4%, such as from 4 to 6%, such as from 6 to 8%, such as from 8 to 10%, such as from 10 to 12%, such as from 12 to 14%, such as from 14 to 16%, such as from 16 to 18%, such as from 18 to 20%, such as from 20 to 22%, such as from 22 to 24%, such as from 24 to 26%, such as from 26 to 28%, such as from 28 to 30%, for example to a concentration of 10% extractant in 90% of the liquid.

175. The method according to any one of claims 173-174, wherein the liquid is a polar protic solvent, such as an alcohol or water, for example ethanol.

176. The method according to any one of claims 173-174, wherein the extractant is a lipophilic non-volatile solvent.

177. The method according to any one of claims 173-174, wherein the extractant is selected from the group consisting of: Antifoam-A, Triton-X 114, isopropyl myristate, isopropyl palmitate, polysorbate, ethyl laurate, castor oil, oleyl alcohol, butyl caprilate, grapeseed oil, 2-butyl-1-octanol, and oleic acid, for example isopropyl myristate.

178. The method according to any one of claims 164-177, wherein the method comprises diluting the colored fermentation extract with water, optionally at room temperature, until a single phase is produced between the colored fermentation extract and the water.

179. The method according to claim 178, wherein the method comprises dilution until the concentration of the extractant is below its cloud point at room temperature.

180. The method according to any one of claims 178-179, wherein the method provides a colored aqueous suspension.

181. The method according to claim 180, wherein the method comprises dyeing textile material by contacting the textile material with the aqueous suspension and incubating at room temperature.

182. The method according to any one of claims 178-181, wherein the textile material is selected from the group consisting of: nylon 6,6, diacetate, polyester, cotton, such as bleached cotton, wool, hemp rayon, denim, viscose, and silk, for example nylon 6,6.

183. The method according to any one of claims 164-182, wherein the liquid is water and the one or more compounds are glycosides as defined in any one of claims 104-107, for example wherein the one or more compounds is a glycoside of violacein or proviolacein.

184. The method according to claim 183, further comprising the steps of:
a. incubating the textile material in a dye bath comprising the one or more compounds in any one of claims 104-107 and water; and
b. adding a glucosidase, such as a beta-glucosidase to the dye bath to de-glycosylate the one or more compounds thereby providing a dyed textile material.

185. The method according to claim 184, wherein the method comprises incubating for from 15 minutes to 24 hours, such as from 15 minutes to 30 minutes, such as from 30 minutes to 45 minutes, such as from 45 minutes to 1 hour, such as from 1 hour to 2 hours, such as from 2 hours to 3 hours, such as from 3 hours to 4 hours, such as from 4 hours to 5 hours, such as from 5 hours to 6 hours, such as from 6 hours to 7 hours, such as from 7 hours to 8 hours, such as from 8 hours to 9 hours, such as from 9 hours to 10 hours, such as from 10 hours to 11 hours, such as from 11 hours to 12 hours, such as from 12 hours to 13 hours, such as from 13 hours to 14 hours, such as from 14 hours to 15 hours, such as from 15 hours to 16 hours, such as from 16 hours to 17 hours, such as from 17 hours to 18 hours, such as from 18 hours to 19 hours, such as from 19 hours to 20 hours, such as from 20 hours to 21 hours, such as from 21 hours to 22 hours, such as from 22 hours to 23 hours, such as from 23 hours to 24 hours, preferably at room temperature.

186. The method according to any one of claims 184-185, wherein the method further comprises washing the dyed textile material after step b.

187. A method for dyeing textile material in a growth medium, comprising:
a. Cultivating a microbial host cell as defined in any one of claims 80-95 in a growth medium;
b. Adding textile material to the growth medium to provide a dyed textile material comprising a compound of formula (I), optionally for a predefined duration, optionally during the cultivation process.

188. The method according to claim 187, wherein the microbial host cell is cultivated at from 25 to 35° C., such as 30° C. for a number of days, such as for from 2 to 8 days, such as 4 days.

189. The method according to claim 187, wherein the method further comprises a step of sterilizing the textile material prior to step b, such as by adding the textile material into an alcohol or a solution of alcohol in water, for example ethanol, such as 75% ethanol in water.

190. The method according to any one of claims 187-189, wherein the method further comprises a step c) of recovering the dyed textile material from the growth medium.

191. The method according to any one of claims 187-190, comprising a step of extracting the compound of formula (I) from the growth medium by recovering the dyed textile material from the growth medium.

192. The method according to any one of claims 187-191, wherein the textile material is selected from the group consisting of diacetate, bleached cotton, nylon 6,6, polyester, acrylic, and wool.

193. The method according to any one of claims 187-192, further comprising a step of washing the dyed textile material with water post-cultivation.

194. A dyed textile material comprising the compound as defined in any one of claims 102-107.

195. The dyed textile material according to claim 194, wherein the textile material is selected from the group consisting of: Nylon 6,6, Diacetate, Bleached cotton, Polyester, Wool, and Acrylic.

196. The dyed textile material according to any one of claims 194-195, wherein the compound is selected from the group consisting of: deoxyviolacein, violacein, prodeoxyviolacein, proviolacein, or a combination thereof.

197. The dyed textile material according to any one of claims 194-196 obtainable by the method of any one of claims 164-193.

198. A method of colouring a beverage, comprising:
a. providing an optionally dried composition of one or more compounds as defined in any one of claims 104-107, for example glycosylated violacein, glycosylated proviolacein, glycosylated prodeoxyviolacein, and/or glycosylated deoxyviolacein; and optionally subsequently preparing a dye solution by suspending said composition in a liquid, such as an alcohol or water; and
b. contacting a beverage with said dye solution or said composition, optionally for a predetermined duration, thereby colouring the beverage.

199. A method for enhancing the antimicrobial properties of a textile material, such as clothing or a wound dressing, or a beverage, comprising dyeing the textile material or colouring the beverage with the compound as defined in any one of claims 102-107, or with an extractant comprising the compound thereby enhancing the antimicrobial properties of the textile material or beverage.

200. A method for enhancing the antioxidant properties of a textile material, such as clothing, or a beverage comprising dyeing the textile material or colouring the beverage with the compound as defined in any one of claims 102-107, or with an extractant comprising the compound thereby enhancing the antioxidant properties of the textile material or beverage.

201. A method for enhancing the UV resistance of a textile material, such as clothing, or of a beverage comprising dyeing the textile material or colouring the beverage with the compound as defined in any one of claims 104-107, or with an extractant comprising the compound thereby enhancing the UV resistance of the textile material or beverage.

202. A beverage comprising the comprising the compound as defined in any one of claims 104-107.

203. A nanocellulose comprising a compound as defined in any one of claims 104-107.

204. The nanocellulose of claim 203, wherein the nanocellulose is selected from the group consisting of bacterial nanocellulose (BNC), nanofabricated cellulose (NFC), cellulose nanocrystals (CNC), cellulose nanofibrils (CNF), and electrospun cellulose nanofibers.

205. The nanocellulose of claim 204, wherein the nanocellulose is bacterial nanocellulose (BNC) or nanofabricated cellulose (NFC).

206. The nanocellulose according to any one of claims 203-205, further comprising a non-ionic surfactant, such as Triton-X 100, Tween 20, sodium dodecyl sulfate (SDS), or polyvinyl alcohol (PVA).

207. The nanocellulose according to any one of claims 203-206, wherein the nanocellulose is derived from a microbial culture.

208. The nanocellulose according to claim 207, wherein the microbial culture comprises one or more of Acetobacter xylinum, Gluconacetobacter hansenii, and Komagataeibacter medellinensis.

209. The nanocellulose according to any one of claims 203-208, wherein the nanocellulose is derived from a Kombucha starter culture, optionally comprising green tea and sucrose.

210. The nanocellulose of any one of claims 203-209, wherein the compound is selected from the group consisting of: violacein, proviolacein, deoxyviolacein, and prodeoxyviolacein.

211. The nanocellulose of any one of claims 203-210, wherein the compound is deoxyviolacein and the nanocellulose is NFC.

212. The nanocellulose of any one of claims 203-211, wherein the nanocellulose is in a form selected from the group consisting of a hydrogel, an aerogel, and a film.

213. A method for dyeing nanocellulose, comprising
a. providing a compound as defined in any one of claims 104-107, optionally in a dye bath comprising an alcohol and optionally a surfactant;
b. providing nanocellulose, such as bacterial nanocellulose (BNC) or nanofabricated cellulose (NFC);
c. incubating the cellulose with the compound, optionally in the dye bath, at a predefined temperature until the nanocellulose takes on the color of the compound, thereby providing dyed nanocellulose.

214. The method of claim 213, wherein the predefined temperature is from 20 to 50° C.; or is room temperature.

215. The method of claim 213, wherein the dye bath comprises from 70 to 95% alcohol in non-ionic surfactant, such as 90%, for example wherein the alcohol is ethanol.

216. The method of any one of claims 213-215, wherein the extractant is selected from the group consisting of isopropyl myristate, Triton-X 100, Tween-20, and Tween-80.

217. The method of any one of claims 213-216, wherein the nanocellulose is selected from the group consisting of bacterial nanocellulose (BNC), nanofabricated cellulose (NFC), cellulose nanocrystals (CNC), cellulose nanofibrils (CNF), and electrospun cellulose nanofibers.

218. The method of any one of claims 213-217, wherein the nanocellulose is selected from the group consisting of bacterial nanocellulose (BNC), and nanofabricated cellulose (NFC).

219. The method of any one of claims 213-217, further comprising a step of: d. drying the dyed nanocellulose at room temperature.

220. The method of any one of claims 213-219, wherein the compound is provided in a dye bath, and wherein the dye bath further comprises a non-ionic surfactant at a concentration of approximately 0.01%, for example Triton-X 100.

221. A dyed product comprising the nanocellulose of any one of claims 203-212.

222. The dyed product according to claim 221, wherein the product is selected from the group consisting of: a wound healing product, such as a wound dressing, a food packaging, a cosmetic product, a textile fiber, a bio-based paint, a paper, and a textile dye.

223. A method for dyeing a product, comprising:
a. Providing a nanocellulose as defined in any one of claims 203-212;
b. Providing a product;
c. Contacting the nanocellulose with the product, optionally incubating the product with the nanocellulose for a duration.

224. The method according to claim 223, wherein the product is selected from the group consisting of: a wound healing product, such as a wound dressing, a food packaging, a cosmetic product, a textile fiber, a bio-based paint, a paper, and a textile dye.

225. The method according to claim 223, wherein the product is paper and the nanocellulose comprises NFC.

226. A method of producing a dye bath, the method comprising the steps of:
a. cultivating a host cell as defined in any one of claims 80-101 in a growth medium to produce the compound as defined in any one of claims 104-107;
b. adding an extractant to the growth medium thereby providing a compound enriched extractant;
c. optionally collecting the compound enriched extractant and adding further extractant to the growth medium;
d. optionally repeating step c a number of times to provide a collection of compound enriched extractants,
e. diluting the compound enriched extractant or the collection of compound enriched extractants with a liquid, such as an organic solvent, thereby providing a dye bath.

227. The method according to claim 226, wherein the extractant is selected from the group consisting of: isopropyl myristate, Antifoam-A, Triton-X 114, isopropyl palmitate, polysorbate, ethyl laurate, castor oil, oleyl alcohol, butyl caprilate, grapeseed oil, 2-butyl-1-octanol, and oleic acid, or any combination thereof.

228. The method according to claim 227, wherein the extractant is isopropyl myristate.

229. The method according to any one of claims 226-228, wherein the liquid is ethanol.

230. A dye bath obtainable using the method of any one of claims 226-229.

231. A method for dyeing a product, comprising the steps of:
a. adding a product to a dye bath comprising a compound of formula (I) as defined in any one of claims 104-107, and a liquid and optionally an extractant;
b. optionally pre/post-treating the product to modify its pH;
c. optionally dyeing the product at a predetermined temperature for a predetermined time to obtain a dyed product, optionally in a dyeing machine;
d. washing the dyed product with water; and
e. optionally drying the product.

232. The method of claim 231, wherein the product is selected from the group consisting of: a fabric, a fiber, a yarn, a textile, a filament, a weave, a non-woven material, a twill, a felt, a lace, a mesh, a cord, a tapestry, a tuft, and a batting; for example a fabric, a fiber, or a yarn.

233. The method according to any one of claims 231-232, wherein the product comprises a material selected from the group consisting of nylon 6,6, diacetate, polyester, cotton, such as bleached cotton, wool, hemp rayon, denim, viscose, and silk.

234. The method of any one of claims 231-232, wherein the predetermined temperature is from 15 to 50° C., such as from 20 to 35° C., for example about 23° C.

235. The method of any one of claims 231-232, wherein the predetermined temperature is from 80 to 180° C., such as from 85 to 170° C., such as from 90 to 160° C., such as from 95 to 155° C., such as from 100 to 150° C., such as from 105 to 145° C., such as from 110 to 140° C., for example 130° C.

236. The method of any one of claims 231-232, wherein the predetermined temperature is from 80 to 180° C., such as from 85 to 170° C., such as from 90 to 160° C., such as from 95 to 155° C., such as from 100 to 150° C., such as from 105 to 145° C., such as from 110 to 140° C., for example 130° C.; and wherein the product comprises polyester.

237. The method of any one of claims 231-234, wherein the predetermined time is from 5 minutes to 360 minutes, such as for 10 minutes to 60 minutes, for example 15 minutes.

238. The method of any one of claims 231-237, wherein the final concentration of the extractant in the dye bath is less than 70%, such as less than 69%, such as less than 68%, such as less than 67%, such as less than 66%, such as less than 65%, such as less than 64%, such as less than 63%, such as less than 62%, such as less than 61%, such as less than 60%, such as less than 59%, such as less than 58%, such as less than 57%, such as less than 56%, such as less than 55%, such as less than 54%, such as less than 53%, such as less than 52%, such as less than 51%, such as less than 50%.

239. The method of any one of claims 164-238, further comprising a step of adding a dispersing agent, such as a soap.

240. The method of claim 239, wherein the dispersing agent is selected from the group consisting of: an anionic surfactant, such as sodium dodecyl sulfate or alkylbenzene sulfonate; a cationic surfactant, such as a quaternary ammonium compound; a non-ionic surfactant, such as an ethoxylated alcohol, an alkylphenol, or a polysorbate; a zwitterionic surfactant, such as cocamidopropyl betaine; a polysaccharide, a cellulose derivative, such as carboxymethylcellulose, or hydroxyethylcellulose; a protein, such as casein, a gum, such as xanthan gum, guar gum, or acacia gum, and lecithin.

241. The method of any one of claims 239-240, wherein the dispersing agent is added to provide a final concentration of from 0.05 to 3 g/L, for example from 1 to 2 g/L.

242. A method of recycling a used dye bath, comprising the steps of:
a. subjecting a used dye bath comprising i) a liquid, ii) an extractant, and iii) a compound of formula (I) as defined in any one of claims 104-107 to evaporation, optionally in vacuo to remove the liquid, wherein the dye bath has been used for dyeing a product;
b. passing the remaining extractant and compound from step a through silica to obtain a recycled dye bath.


SEQUENCE LISTING (96 sequences)

SEQ ID NO: 1 - DNA, 2478 bp, Bifidobacterium breve
  source 1..2478: mol_type = unassigned DNA
atgacctctccagttattggtactccatggaaaaaattgaacgccccagtttctgaagaatccttggaaggtgttgataagtattggagagttgccaactacttgtccattggtcaaatctacttgaggtctaacccattgatgaaggctccattcactagagaagatgttaagcacagattggttggtcattggggtactactccaggtttgaattttttgatcggtcacatcaacagattcattgctgatcatggtcaaaacaccgttattatcatgggtccaggtcatggtggtccagctggtacttctcaatcttatttggatggtacttacaccgagactttcccaaagattacaaaagatgaagctggcttgcagaagttcttcagacaattttcttatccaggtggtatcccatctcattttgctccagaaacaccaggttctattcatgaaggtggtgaattgggttatgctttgtctcatgcttatggtgccattatggataacccatctttgtttgttccagccatagttggtgatggtgaagctgaaactggtccattggctactggttggcaatctaacaaattggttaacccaagaaccgatggtatcgttttgccaatcttgcatttgaacggttacaagattgctaacccaaccatcttgtccagaatctctgatgaagaattgcacgaattcttccatggtatgggttatgaaccatacgaatttgttgctggtttcgatgatgaagatcacatgtccattcatagaagattcgctgaattgtgggaaaccatttgggacgaaatttgcgatattaaggctgctgctcaaaccgataatgttcatagaccattttaccccatgctgattttcagaactccaaaaggttggacttgcccaaagtacattgatggtaaaaagactgaaggttcttggagagcacatcaagttccattggcatctgctagagatactgaagctcatttcgaagttttgaagaactggttggaaagctacaaacctgaagagttgtttgatgctaatggtgctgttaaggatgatgttttggcttttatgccaaagggtgaattgagaattggtgctaatccaaatgcaaacggtggtgttattagagatgatctgaagttgccaaacttggaagattacgaagtcaaagaagttgccgaatacggtcatggttggggtcaattggaagctactagaactttgggtgcttacaccagagatatcattagaaacaacccaagagacttcagaattttcggtccagacgaaactgcttctaatagattgcaagcttcttacgaggttaccaacaaacaatgggatgctggttacatttccgatgaagttgatgaacatatgcacgtttctggtcaagttgtcgaacaattgtcagaacatcaaatggaaggttttttggaggcttacttgttgactggtagacatggtatttggtcctcttacgaatctttcgttcacgttatcgactctatgttgaatcaacacgctaaatggttggaagccaccgttagagaaattccttggagaaaacctattgcctccatgaacttgttggtttcttcacatgtttggagacaagatcacaacggtttctctcatcaagatccaggtgttacttctgtcttgttgaacaagtgtttccacaacgatcatgtcatcggtatctactttgctactgatgctaacatgttgttggctattgctgaaaagtgttacaagtccaccaacaagattaacgctattattgctggtaaacaaccagctgctacttggttgactttggatgaagctagagctgaattggctaaaggtgctgctgcttgggattgggcttctactgctaaaaacaatgatgaagccgaagttgttttagctgctgctggtgatgttccaactcaagaaattatggctgcttccgacaagttgaaagaattgggtgttaagttcaaggttgttaacgttgccgatttgttgtcattgcaatccgctaaagaaaacgacgaagctttgtcagacgaagaatttgctgatatcttcactgctgataagccagttttgttcgcttatcattcttacgcccatgatgtcagaggtttgatatacgatagaccaaaccacgataacttcaacgttcatggttatgaagaagaaggttctactactaccccatacgatatggttagagttaacagaatcgacaggtacgaattgactgctgaagctttgagaatgattgatgcagataagtacgccgataagatcgacgaattggaaaagttcagagatgaagcctttcaattcgcagttgataagggttatgatcacccagattataccgattgggtttactctggtgttaataccgataagaaaggtgcagttactgctactgctgcaacagctggtgataatgaatga

SEQ ID NO: 2 - protein, 825 aa, Bifidobacterium breve
MTSPVIGTPWKKLNAPVSEESLEGVDKYWRVANYLSIGQIYLRSNPLMKAPFTREDVKHRLVGHWGTTPGLNFLIGHINRFIADHGQNTVIIMGPGHGGPAGTSQSYLDGTYTETFPKITKDEAGLQKFFRQFSYPGGIPSHFAPETPGSIHEGGELGYALSHAYGAIMDNPSLFVPAIVGDGEAETGPLATGWQSNKLVNPRTDGIVLPILHLNGYKIANPTILSRISDEELHEFFHGMGYEPYEFVAGFDDEDHMSIHRRFAELWETIWDEICDIKAAAQTDNVHRPFYPMLIFRTPKGWTCPKYIDGKKTEGSWRAHQVPLASARDTEAHFEVLKNWLESYKPEELFDANGAVKDDVLAFMPKGELRIGANPNANGGVIRDDLKLPNLEDYEVKEVAEYGHGWGQLEATRTLGAYTRDIIRNNPRDFRIFGPDETASNRLQASYEVTNKQWDAGYISDEVDEHMHVSGQVVEQLSEHQMEGFLEAYLLTGRHGIWSSYESFVHVIDSMLNQHAKWLEATVREIPWRKPIASMNLLVSSHVWRQDHNGFSHQDPGVTSVLLNKCFHNDHVIGIYFATDANMLLAIAEKCYKSTNKINAIIAGKQPAATWLTLDEARAELAKGAAAWDWASTAKNNDEAEVVLAAAGDVPTQEIMAASDKLKELGVKFKVVNVADLLSLQSAKENDEALSDEEFADIFTADKPVLFAYHSYAHDVRGLIYDRPNHDNFNVHGYEEEGSTTTPYDMVRVNRIDRYELTAEALRMIDADKYADKIDELEKFRDEAFQFAVDKGYDHPDYTDWVYSGVNTDKKGAVTATAATAGDNE

SEQ ID NO: 3 - DNA, 1002 bp, Clostridium kluyveri
  source 1..1002: mol_type = unassigned DNA
atgaagctgatggaaaacattttcggtttggctaaggctgacaagaagaaaatcgttttggctgaaggtgaagaagagagaaatattagagcctccgaagagattatcagagatggtattgctgatatcatcttggtcggttctgaatccgttatcaaagaaaatgctgctaagttcggtgttaacttggctggtgttgaaatagttgatccagaaacttcttctaagactgctggttacgctaatgccttctacgaaattagaaagaacaagggtgttaccttggaaaaggctgataagatagttagggatccaatctacttcgctaccatgatggttaagttgggtgatgctgatggtttggtttctggtgctattcatacaaccggtgatttgttaagaccaggcttgcaaattgtcaaaactgttccaggtgcttccgttgtttcttctgtttttttgatgtctgttccagactgcgaatatggtgaagatggttttttgttgttcgctgattgtgctgttaacgtttgtccaactgctgaagaattgtcctctattgctattactactgctgaaaccgctaagaacttgtgcaaaattgaacctagagttgccatgttgtctttctctactatgggttctgcttcccatgaattggttgataaggttactaaggctaccaagttggctaaagaagctagaccagatttggatatcgatggtgaattacaattggatgcctccttggttaagaaggttgctgatttgaaagctccaggttctaaagttgctggtaaggctaatgttttgatcttcccagatattcaagctggtaacatcggttacaagttggttcaaagattcgctaaagctgaagccattggtccaatttgtcaaggttttgctaagccaatcaacgacttgtctagaggttgttctgttgatgatatcgttaaggttgttgccgttactgctgttcaagctcaagcacaaggttaa

SEQ ID NO: 4 - protein, 333 aa, Clostridium kluyveri
MKLMENIFGLAKADKKKIVLAEGEEERNIRASEEIIRDGIADIILVGSESVIKENAAKFGVNLAGVEIVDPETSSKTAGYANAFYEIRKNKGVTLEKADKIVRDPIYFATMMVKLGDADGLVSGAIHTTGDLLRPGLQIVKTVPGASVVSSVFLMSVPDCEYGEDGFLLFADCAVNVCPTAEELSSIAITTAETAKNLCKIEPRVAMLSFSTMGSASHELVDKVTKATKLAKEARPDLDIDGELQLDASLVKKVADLKAPGSKVAGKANVLIFPDIQAGNIGYKLVQRFAKAEAIGPICQGFAKPINDLSRGCSVDDIVKVVAVTAVQAQAQG

SEQ ID NO: 5 - DNA, 1113 bp, Saccharomyces cerevisiae
  source 1..1113: mol_type = other DNA
atgagtgaatctccaatgttcgctgccaacggcatgccaaaggtaaatcaaggtgctgaagaagatgtcagaattttaggttacgacccattagcttctccagctctccttcaagtgcaaatcccagccacaccaacttctttggaaactgccaagagaggtagaagagaagctatagatattattaccggtaaagacgacagagttcttgtcattgtcggtccttgttccatccatgatctagaagccgctcaagaatacgctttgagattaaagaaattgtcagatgaattaaaaggtgatttatccatcattatgagagcatacttggagaagccaagaacaaccgtcggctggaaaggtctaattaatgaccctgatgttaacaacactttcaacatcaacaagggtttgcaatccgctagacaattgtttgtcaacttgacaaatatcggtttgccaattggttctgaaatgcttgataccatttctcctcaatacttggctgatttggtgtccttcggtgccattggtgccagaaccaccgaatctcaactgcacagagaattggcctccggtttgtctttcccagttggtttcaagaacggtaccgatggtaccttaaatgttgctgtggatgcttgtcaagccgctgctcattctcaccatttcatgggtgttactctgcatggtgttgctgctatcaccactactaagggtaacgaacactgcttcgttattctaagaggtggtaaaaagggtaccaactacgacgctaagtccgttgcagaagctaaggctcaattgcctgccggttccaacggtctaatgattgactactctcacggtaactccaataaggatttcagaaaccaaccaaaggtcaatgacgttgtttgtgagcaaatcgctaacggtgaaaacgccattaccggtgtcatgattgaatcaaacatcaacgaaggtaaccaaggcatcccagccgaaggtaaagccggcttgaaatatggtgtttccatcactgatgcttgtataggttgggaaactactgaagacgtcttgaggaaattggctgctgctgtcagacaaagaagagaagttaacaagaaatag

SEQ ID NO: 6 - protein, 370 aa, Saccharomyces cerevisiae
MSESPMFAANGMPKVNQGAEEDVRILGYDPLASPALLQVQIPATPTSLETAKRGRREAIDIITGKDDRVLVIVGPCSIHDLEAAQEYALRLKKLSDELKGDLSIIMRAYLEKPRTTVGWKGLINDPDVNNTFNINKGLQSARQLFVNLTNIGLPIGSEMLDTISPQYLADLVSFGAIGARTTESQLHRELASGLSFPVGFKNGTDGTLNVAVDACQAAAHSHHFMGVTLHGVAAITTTKGNEHCFVILRGGKKGTNYDAKSVAEAKAQLPAGSNGLMIDYSHGNSNKDFRNQPKVNDVVCEQIANGENAITGVMIESNINEGNQGIPAEGKAGLKYGVSITDACIGWETTEDVLRKLAAAVRQRREVNKK

SEQ ID NO: 7 - DNA, 4767 bp, Saccharomyces cerevisiae
  source 1..4767: mol_type = other DNA
atggtgcagttagccaaagtcccaattctaggaaatgatattatccacgttgggtataacattcatgaccatttggttgaaaccataattaaacattgtccttcttcgacatacgttatttgcaatgatacgaacttgagtaaagttccatactaccagcaattagtcctggaattcaaggcttctttgccagaaggctctcgtttacttacttatgttgttaaaccaggtgagacaagtaaaagtagagaaaccaaagcgcagctagaagattatcttttagtggaaggatgtactcgtgatacggttatggtagcgatcggtggtggtgttattggtgacatgattgggttcgttgcatctacatttatgagaggtgttcgtgttgtccaagtaccaacatccttattggcaatggtcgattcctccattggtggtaaaactgctattgacactcctctaggtaaaaactttattggtgcattttggcaaccaaaatttgtccttgtagatattaaatggctagaaacgttagccaagagagagtttatcaatgggatggcagaagttatcaagactgcttgtatttggaacgctgacgaatttactagattagaatcaaacgcttcgttgttcttaaatgttgttaatggggcaaaaaatgtcaaggttaccaatcaattgacaaacgagattgacgagatatcgaatacagatattgaagctatgttggatcatacatataagttagttcttgagagtattaaggtcaaagcggaagttgtctcttcggatgaacgtgaatccagtctaagaaaccttttgaacttcggacattctattggtcatgcttatgaagctatactaaccccacaagcattacatggtgaatgtgtgtccattggtatggttaaagaggcggaattatcccgttatttcggtattctctcccctacccaagttgcacgtctatccaagattttggttgcctacgggttgcctgtttcgcctgatgagaaatggtttaaagagctaaccttacataagaaaacaccattggatatcttattgaagaaaatgagtattgacaagaaaaacgagggttccaaaaagaaggtggtcattttagaaagtattggtaagtgctatggtgactccgctcaatttgttagcgatgaagacctgagatttattctaacagatgaaaccctcgtttaccccttcaaggacatccctgctgatcaacagaaagttgttatcccccctggttctaagtccatctccaatcgtgctttaattcttgctgccctcggtgaaggtcaatgtaaaatcaagaacttattacattctgatgatactaaacatatgttaaccgctgttcatgaattgaaaggtgctacgatatcatgggaagataatggtgaaacggtagtggtggaaggacatggtggttccacattgtcagcttgtgctgaccccttatatctaggtaatgcaggtactgcatctagatttttgacttccttggctgccttggtcaattctacttcaagccaaaagtatatcgttttaactggtaacgcaagaatgcaacaaagaccaattgctcctttggtcgattctttgcgtgctaatggtactaaaattgagtacttgaataatgaaggttccctgccaatcaaagtttatactgattcggtattcaaaggtggtagaattgaattagctgctacagtttcttctcagtacgtatcctctatcttgatgtgtgccccatacgctgaagaacctgtaactttggctcttgttggtggtaagccaatctctaaattgtacgtcgatatgacaataaaaatgatggaaaaattcggtatcaatgttgaaacttctactacagaaccttacacttattatattccaaagggacattatattaacccatcagaatacgtcattgaaagtgatgcctcaagtgctacatacccattggccttcgccgcaatgactggtactaccgtaacggttccaaacattggttttgagtcgttacaaggtgatgccagatttgcaagagatgtcttgaaacctatgggttgtaaaataactcaaacggcaacttcaactactgtttcgggtcctcctgtaggtactttaaagccattaaaacatgttgatatggagccaatgactgatgcgttcttaactgcatgtgttgttgccgctatttcgcacgacagtgatccaaattctgcaaatacaaccaccattgaaggtattgcaaaccagcgtgtcaaagagtgtaacagaattttggccatggctacagagctcgccaaatttggcgtcaaaactacagaattaccagatggtattcaagtccatggtttaaactcgataaaagatttgaaggttccttccgactcttctggacctgtcggtgtatgcacatatgatgatcatcgtgtggccatgagtttctcgcttcttgcaggaatggtaaattctcaaaatgaacgtgacgaagttgctaatcctgtaagaatacttgaaagacattgtactggtaaaacctggcctggctggtgggatgtgttacattccgaactaggtgccaaattagatggtgcagaacctttagagtgcacatccaaaaagaactcaaagaaaagcgttgtcattattggcatgagagcagctggcaaaactactataagtaaatggtgcgcatccgctctgggttacaaattagttgacctagacgagctgtttgagcaacagcataacaatcaaagtgttaaacaatttgttgtggagaacggttgggagaagttccgtgaggaagaaacaagaattttcaaggaagttattcaaaattacggcgatgatggatatgttttctcaacaggtggcggtattgttgaaagcgctgagtctagaaaagccttaaaagattttgcctcatcaggtggatacgttttacacttacatagggatattgaggagacaattgtctttttacaaagtgatccttcaagacctgcctatgtggaagaaattcgtgaagtttggaacagaagggaggggtggtataaagaatgctcaaatttctctttctttgctcctcattgctccgcagaagctgagttccaagctctaagaagatcgtttagtaagtacattgcaaccattacaggtgtcagagaaatagaaattccaagcggaagatctgcctttgtgtgtttaacctttgatgacttaactgaacaaactgagaatttgactccaatctgttatggttgtgaggctgtagaggtcagagtagaccatttggctaattactctgctgatttcgtgagtaaacagttatctatattgcgtaaagccactgacagtattcctatcatttttactgtgcgaaccatgaagcaaggtggcaactttcctgatgaagagttcaaaaccttgagagagctatacgatattgccttgaagaatggtgttgaattccttgacttagaactaactttacctactgatatccaatatgaggttattaacaaaaggggcaacaccaagatcattggttcccatcatgacttccaaggattatactcctgggacgacgctgaatgggaaaacagattcaatcaagcgttaactcttgatgtggatgttgtaaaatttgtgggtacggctgttaatttcgaagataatttgagactggaacactttagggatacacacaagaataagcctttaattgcagttaatatgacttctaaaggtagcatttctcgtgttttgaataatgttttaacacctgtgacatcagatttattgcctaactccgctgcccctggccaattgacagtagcacaaattaacaagatgtatacatctatgggaggtatcgagcctaaggaactgtttgttgttggaaagccaattggccactctagatcgccaattttacataacactggctatgaaattttaggtttacctcacaagttcgataaatttgaaactgaatccgcacaattggtgaaagaaaaacttttggacggaaacaagaactttggcggtgctgcagtcacaattcctctgaaattagatataatgcagtacatggatgaattgactgatgctgctaaagttattggtgctgtaaacacagttataccattgggtaacaagaagtttaagggtgataataccgactggttaggtatccgtaatgccttaattaacaatggcgttcccgaatatgttggtcataccgctggtttggttatcggtgcaggtggcacttctagagccgccctttacgccttgcacagtttaggttgcaaaaagatcttcataatcaacaggacaacttcgaaattgaagccattaatagagtcacttccatctgaattcaacattattggaatagagtccactaaatctatagaagagattaaggaacacgttggcgttgctgtcagctgtgtaccagccgacaaaccattagatgacgaacttttaagtaagctggagagattccttgtgaaaggtgcccatgctgcttttgtaccaaccttattggaagccgcatacaaaccaagcgttactcccgttatgacaatttcacaagacaaatatcaatggcacgttgtccctggatcacaaatgttagtacaccaaggtgtagctcagtttgaaaagtggacaggattcaagggccctttcaaggccatttttgatgccgttacgaaagagtag

SEQ ID NO: 8 - protein, 1588 aa, Saccharomyces cerevisiae
MVQLAKVPILGNDIIHVGYNIHDHLVETIIKHCPSSTYVICNDTNLSKVPYYQQLVLEFKASLPEGSRLLTYVVKPGETSKSRETKAQLEDYLLVEGCTRDTVMVAIGGGVIGDMIGFVASTFMRGVRVVQVPTSLLAMVDSSIGGKTAIDTPLGKNFIGAFWQPKFVLVDIKWLETLAKREFINGMAEVIKTACIWNADEFTRLESNASLFLNVVNGAKNVKVTNQLTNEIDEISNTDIEAMLDHTYKLVLESIKVKAEVVSSDERESSLRNLLNFGHSIGHAYEAILTPQALHGECVSIGMVKEAELSRYFGILSPTQVARLSKILVAYGLPVSPDEKWFKELTLHKKTPLDILLKKMSIDKKNEGSKKKVVILESIGKCYGDSAQFVSDEDLRFILTDETLVYPFKDIPADQQKVVIPPGSKSISNRALILAALGEGQCKIKNLLHSDDTKHMLTAVHELKGATISWEDNGETVVVEGHGGSTLSACADPLYLGNAGTASRFLTSLAALVNSTSSQKYIVLTGNARMQQRPIAPLVDSLRANGTKIEYLNNEGSLPIKVYTDSVFKGGRIELAATVSSQYVSSILMCAPYAEEPVTLALVGGKPISKLYVDMTIKMMEKFGINVETSTTEPYTYYIPKGHYINPSEYVIESDASSATYPLAFAAMTGTTVTVPNIGFESLQGDARFARDVLKPMGCKITQTATSTTVSGPPVGTLKPLKHVDMEPMTDAFLTACVVAAISHDSDPNSANTTTIEGIANQRVKECNRILAMATELAKFGVKTTELPDGIQVHGLNSIKDLKVPSDSSGPVGVCTYDDHRVAMSFSLLAGMVNSQNERDEVANPVRILERHCTGKTWPGWWDVLHSELGAKLDGAEPLECTSKKNSKKSVVIIGMRAAGKTTISKWCASALGYKLVDLDELFEQQHNNQSVKQFVVENGWEKFREEETRIFKEVIQNYGDDGYVFSTGGGIVESAESRKALKDFASSGGYVLHLHRDIEETIVFLQSDPSRPAYVEEIREVWNRREGWYKECSNFSFFAPHCSAEAEFQALRRSFSKYIATITGVREIEIPSGRSAFVCLTFDDLTEQTENLTPICYGCEAVEVRVDHLANYSADFVSKQLSILRKATDSIPIIFTVRTMKQGGNFPDEEFKTLRELYDIALKNGVEFLDLELTLPTDIQYEVINKRGNTKIIGSHHDFQGLYSWDDAEWENRFNQALTLDVDVVKFVGTAVNFEDNLRLEHFRDTHKNKPLIAVNMTSKGSISRVLNNVLTPVTSDLLPNSAAPGQLTVAQINKMYTSMGGIEPKELFVVGKPIGHSRSPILHNTGYEILGLPHKFDKFETESAQLVKEKLLDGNKNFGGAAVTIPLKLDIMQYMDELTDAAKVIGAVNTVIPLGNKKFKGDNTDWLGIRNALINNGVPEYVGHTAGLVIGAGGTSRAALYALHSLGCKKIFIINRTTSKLKPLIESLPSEFNIIGIESTKSIEEIKEHVGVAVSCVPADKPLDDELLSKLERFLVKGAHAAFVPTLLEAAYKPSVTPVMTISQDKYQWHVVPGSQMLVHQGVAQFEKWTGFKGPFKAIFDAVTKE

SEQ ID NO: 9 - DNA, 525 bp, Escherichia coli
  source 1..525: mol_type = other DNA
atgacccaacctttgtttttgattggtccaagaggttgtggtaagactactgttggtatggctttggctgattctttgaacagaagattcgttgataccgaccaatggttgcaatctcaattgaatatgaccgttgccgaaatcgttgaaagggaagaatgggctggttttagagctagagaaactgctgctttggaagctgttactgctccatctactgttattgctactggtggtggtattatcttgaccgaattcaacagacacttcatgcaaaacaacggtatcgttgtctatttgtgtgctccagtttctgttttggtcaacagattgcaagctgctccagaagaagatctaagaccaactttgactggtaagccattgtctgaagaagttcaagaagtcttggaagaaagagatgccttgtatagagaagttgcccatattatcattgacgctaccaatgaaccatctcaggttatctctgaaattagatctgctttggcccaaaccattaactgctaa

SEQ ID NO: 10 - protein, 174 aa, Escherichia coli
MTQPLFLIGPRGCGKTTVGMALADSLNRRFVDTDQWLQSQLNMTVAEIVEREEWAGFRARETAALEAVTAPSTVIATGGGIILTEFNRHFMQNNGIVVYLCAPVSVLVNRLQAAPEEDLRPTLTGKPLSEEVQEVLEERDALYREVAHIIIDATNEPSQVISEIRSALAQTINC

SEQ ID NO: 11 - DNA, 1131 bp, Saccharomyces cerevisiae
  source 1..1131: mol_type = other DNA
atgtcaacgtttgggaaactgttccgcgtcaccacatatggtgaatcgcattgtaagtctgtcggttgcattgtcgacggtgttcctccaggaatgtcattaaccgaagctgacattcagccacaattgaccagaagaagaccgggtcaatctaagctatcgacccctagagatgaaaaggatagagtggaaatccagtccggtaccgagttcggcaagactctaggtacacccatcgccatgatgatcaaaaacgaggaccaaagacctcacgactactccgacatggacaagttccctagaccttcccatgcggacttcacgtactcggaaaagtacggtatcaaggcctcctctggtggtggcagagcttctgctagagaaacgattggccgtgtcgcttcaggtgccattgctgagaagttcttagctcagaactctaatgtcgagatcgtagcctttgtgacacaaatcggggaaatcaagatgaacagagactctttcgatcctgaatttcagcatctgttgaacaccatcaccagggaaaaagtggactcaatgggtcctatcagatgtccagacgcctccgttgctggtttgatggtcaaggaaatcgaaaagtacagaggcaacaaggactctatcggtggtgtcgtcacttgtgtcgtgagaaacttgcctaccggcctcggtgagccatgctttgacaagttggaagccatgttggctcatgctatgttgtccattccagcatccaagggtttcgaaattggctcaggttttcagggtgtctctgttccagggtccaagcacaatgacccattttactttgaaaaagaaacaaacagattaagaacaaagaccaacaattcaggtggtgtacaaggtggtatctctaatggtgagaacatctatttctctgtcccattcaagtcagtggccactatctctcaagaacaaaaaaccgccacttacgatggtgaagaaggtatcttagccgctaagggtagacatgaccctgctgtcactccaagagctattcctattgtggaagccatgaccgctctggtgttggctgacgcgcttttgatccaaaaggcaagagatttctccagatccgtggttcattaa

SEQ ID NO: 12 - protein, 376 aa, Saccharomyces cerevisiae
MSTFGKLFRVTTYGESHCKSVGCIVDGVPPGMSLTEADIQPQLTRRRPGQSKLSTPRDEKDRVEIQSGTEFGKTLGTPIAMMIKNEDQRPHDYSDMDKFPRPSHADFTYSEKYGIKASSGGGRASARETIGRVASGAIAEKFLAQNSNVEIVAFVTQIGEIKMNRDSFDPEFQHLLNTITREKVDSMGPIRCPDASVAGLMVKEIEKYRGNKDSIGGVVTCVVRNLPTGLGEPCFDKLEAMLAHAMLSIPASKGFEIGSGFQGVSVPGSKHNDPFYFEKETNRLRTKTNNSGGVQGGISNGENIYFSVPFKSVATISQEQKTATYDGEEGILAAKGRHDPAVTPRAIPIVEAMTALVLADALLIQKARDFSRSVVH

SEQ ID NO: 13 - DNA, 1524 bp, Saccharomyces cerevisiae
  source 1..1524: mol_type = other DNA
atgaccgcttccatcaaaattcaaccggatattgactctctaaagcaattacagcagcaaaatgacgatagttccataaatatgtatcccgtgtatgcgtatttgccatcattggatctgactcctcacgtggcatatctaaaattggcacaattgaacaaccctgatagaaaggaatcatttctgttggaacgtgctaagacaaataatgaattagatcgttatctgttcataggtatctcgccacgcaagaccatcaaaaccggtcctactgaaggcattgaaacagatcctttggaaattttggaaaaggagatgtctacctttaaagtagccgaaaatgttcctggtttaccgaaattaagtggtggtgctattggttatatttcttatgactgtgttcgttatttcgagccaaaaacaagaaggcctttgaaagatgtcctaagacttccagaggcatatttaatgctttgtgataccattattgcctttgataatgtttttcagagatttcaaatcattcataacattaataccaatgaaacttcgttggaggaaggttaccaagctgcagcacaaataatcactgatatcgtatcaaagctaaccgacgattcctcgccaataccatatccagaacaacctcctattaaattgaatcaaacttttgaatcgaatgtgggcaaggaaggttacgaaaatcacgtgtccactttgaagaagcatattaagaaaggtgatattattcaaggtgtgccatcgcaaagagtggcaaggccaacttcgttacatcctttcaatatttacagacatttacgtacagtgaacccatctccttacctgttttatattgattgtttggatttccaaatcattggtgcatctccagaattgttgtgcaaatcggattccaaaaatagagtcattacccatccaattgctggtactgtcaaacgtggggctactactgaagaggatgatgctttagcggaccaattacgtggctcgttaaaagaccgtgcagaacatgttatgctggtagatttagcaagaaacgatattaacagaatttgtgacccattaacaacaagtgtcgataaactgttaactattcaaaaattttctcatgtccaacatctggtttctcaagtcagcggtgttctccgcccagaaaagacaagatttgatgcattcagatcgattttccctgcaggtactgtcagtggtgctccaaaggttagagccatggaattgattgccgaactagaaggagaaaggcgtggggtttatgcaggcgccgtaggtcattggtcatacgacggtaaaacaatggacaattgtatcgctttaaggactatggtctataaagatggcattgcttacttgcaagctggcggtggtattgtttacgattcagatgagtacgatgaatatgtcgaaaccatgaataaaatgatggccaatcacagtactattgtgcaagcagaagaattgtgggccgatatcgtaggatcagcttaa

SEQ ID NO: 14 - protein, 507 aa, Saccharomyces cerevisiae
MTASIKIQPDIDSLKQLQQQNDDSSINMYPVYAYLPSLDLTPHVAYLKLAQLNNPDRKESFLLERAKTNNELDRYLFIGISPRKTIKTGPTEGIETDPLEILEKEMSTFKVAENVPGLPKLSGGAIGYISYDCVRYFEPKTRRPLKDVLRLPEAYLMLCDTIIAFDNVFQRFQIIHNINTNETSLEEGYQAAAQIITDIVSKLTDDSSPIPYPEQPPIKLNQTFESNVGKEGYENHVSTLKKHIKKGDIIQGVPSQRVARPTSLHPFNIYRHLRTVNPSPYLFYIDCLDFQIIGASPELLCKSDSKNRVITHPIAGTVKRGATTEEDDALADQLRGSLKDRAEHVMLVDLARNDINRICDPLTTSVDKLLTIQKFSHVQHLVSQVSGVLRPEKTRFDAFRSIFPAGTVSGAPKVRAMELIAELEGERRGVYAGAVGHWSYDGKTMDNCIALRTMVYKDGIAYLQAGGGIVYDSDEYDEYVETMNKMMANHSTIVQAEELWADIVGSA

SEQ ID NO: 15 - DNA, 954 bp, Bacillus subtilis
  source 1..954: mol_type = other DNA
atgtctaatcagtacggtgacaagaacctgaagattttctctttgaactctaacccagaactggccaaagaaattgctgatatagttggtgtccaattgggtaaatgttccgttactagattctccgatggtgaagttcaaatcaacatcgaagaatccattagaggttgcgattgctatatcatccaatctacatctgatccagtcaacgaacacatcatggaactgttgattatggttgatgctttgaagagagcttctgccaagactattaacatcgttattccttactacggttacgccagacaagatagaaaagctagatcaagagaacctattaccgctaagttgttcgctaacttgttggaaactgctggtgctactagagttattgctttggacttgcatgctccacaaattcaaggtttcttcgatatcccaatcgatcacttgatgggtgttccaattttgggtgaatacttcgaaggtaagaacttggaagatatcgtcatcgtttctccagatcatggtggtgttacaagagctagaaaattggctgatagattgaaggctccaattgccattattgataagagaaggccaagaccaaatgttgccgaagttatgaatatcgtcggtaacattgaaggtaaaaccgccattttgatcgatgacattatagataccgctggcactattactttggctgctaatgctttggttgaaaacggtgctaaagaagtttacgcttgttgtactcatccagttttgtctggtccagctgttgaaagaatcaacaactctaccatcaaagaattggtcgtcaccaactctattaagttgccagaagaaaagaagatcgagaggttcaagcaattgtctgttggtccattattggccgaagccattataagagttcacgaacaacaatccgtgtcctacttgttctcttag

SEQ ID NO: 16 - protein, 317 aa, Bacillus subtilis
MSNQYGDKNLKIFSLNSNPELAKEIADIVGVQLGKCSVTRFSDGEVQINIEESIRGCDCYIIQSTSDPVNEHIMELLIMVDALKRASAKTINIVIPYYGYARQDRKARSREPITAKLFANLLETAGATRVIALDLHAPQIQGFFDIPIDHLMGVPILGEYFEGKNLEDIVIVSPDHGGVTRARKLADRLKAPIAIIDKRRPRPNVAEVMNIVGNIEGKTAILIDDIIDTAGTITLAANALVENGAKEVYACCTHPVLSGPAVERINNSTIKELVVTNSIKLPEEKKIERFKQLSVGPLLAEAIIRVHEQQSVSYLFS

SEQ ID NO: 17 - DNA, 1143 bp, Saccharomyces cerevisiae
  source 1..1143: mol_type = other DNA
atgtccgaggcgactttgctatcttacaccaagaaattattggcttctccgccgcaattgagtagcacagacctacacgatgcgttgctggttatattaagtcttttgcaaaaatgtgatacaaatagcgatgagagtctttccatctataccaaagtttcgagttttctcacggccttgagagttactaaacttgatcacaaggctgaatacattgcggaagctgcaaaggctgtgctcagacattccgaccttgttgatctacctttacccaagaaggacgaattacacccggaagatggaccagtaatcttagatattgtaggtactggtggtgacggacagaatacttttaatgtttccacgtctgctgctatcgttgcctccggaattcagggcctaaaaatttgtaagcacggtggtaaagcttctacatccaatagtggagctggtgacctaattggaactttaggctgtgacatgttcaaggttaattcatcgacagtgcccaaactttggcctgataatacgttcatgtttctacttgctcctttttttcatcatggaatgggccacgtttctaagatacgcaaatttcttggaattccgactgttttcaacgtactgggaccacttctacatccagttagccacgtaaacaagagaatattgggcgtttactcaaaggaacttgcgcctgaatatgccaaggcagccgctttggtatatccaggaagcgaaacttttatagtttggggacatgttgggttagacgaagtatcacctataggcaaaactactgtctggcatattgatccgacatcgtccgaacttaaattgaagaccttccaattagaaccttctatgtttggtttagaagaacacgagttgtcgaagtgtgcttcatacggccctaaagagaatgcgagaattctaaaagaagaagtcttgtccggcaagtaccaccttggcgacaataatcctatttatgactacatcttgatgaacaccgccgtgttatattgtttaagccaaggtcaccagaactggaaggaagggatcattaaggcagaagaaagcatacattctggtaatgcattacgttctttagaacactttatagatagtgtgagctccttgtag

SEQ ID NO: 18 - protein, 380 aa, Saccharomyces cerevisiae
MSEATLLSYTKKLLASPPQLSSTDLHDALLVILSLLQKCDTNSDESLSIYTKVSSFLTALRVTKLDHKAEYIAEAAKAVLRHSDLVDLPLPKKDELHPEDGPVILDIVGTGGDGQNTFNVSTSAAIVASGIQGLKICKHGGKASTSNSGAGDLIGTLGCDMFKVNSSTVPKLWPDNTFMFLLAPFFHHGMGHVSKIRKFLGIPTVFNVLGPLLHPVSHVNKRILGVYSKELAPEYAKAAALVYPGSETFIVWGHVGLDEVSPIGKTTVWHIDPTSSELKLKTFQLEPSMFGLEEHELSKCASYGPKENARILKEEVLSGKYHLGDNNPIYDYILMNTAVLYCLSQGHQNWKEGIIKAEESIHSGNALRSLEHFIDSVSSL

SEQ ID NO: 19 - DNA, 675 bp, Saccharomyces cerevisiae
  source 1..675: mol_type = other DNA
atgtctgttattaatttcacaggtagttctggtccattggtgaaagtttgcggcttgcagagcacagaggccgcagaatgtgctctagattccgatgctgacttgctgggtattatatgtgtgcccaatagaaagagaacaattgacccggttattgcaaggaaaatttcaagtcttgtaaaagcatataaaaatagttcaggcactccgaaatacttggttggcgtgtttcgtaatcaacctaaggaggatgttttggctctggtcaatgattacggcattgatatcgtccaactgcatggagatgagtcgtggcaagaataccaagagttcctcggtttgccagttattaaaagactcgtatttccaaaagactgcaacatactactcagtgcagcttcacagaaacctcattcgtttattcccttgtttgattcagaagcaggtgggacaggtgaacttttggattggaactcgatttctgactgggttggaaggcaagagagccccgaaagcttacattttatgttagctggtggactgacgccagaaaatgttggtgatgcgcttagattaaatggcgttattggtgttgatgtaagcggaggtgtggagacaaatggtgtaaaagactctaacaaaatagcaaatttcgtcaaaaatgctaagaaatag

SEQ ID NO: 20 - protein, 224 aa, Saccharomyces cerevisiae
MSVINFTGSSGPLVKVCGLQSTEAAECALDSDADLLGIICVPNRKRTIDPVIARKISSLVKAYKNSSGTPKYLVGVFRNQPKEDVLALVNDYGIDIVQLHGDESWQEYQEFLGLPVIKRLVFPKDCNILLSAASQKPHSFIPLFDSEAGGTGELLDWNSISDWVGRQESPESLHFMLAGGLTPENVGDALRLNGVIGVDVSGGVETNGVKDSNKIANFVKNAKK

SEQ ID NO: 21 - DNA, 1455 bp, Saccharomyces cerevisiae
  source 1..1455: mol_type = other DNA
atgtctgtgcacgctgcaacaaacccaatcaataagcatgtggttctaattgacaactacgattcctttacctggaacgtttacgagtacttgtgccaggagggcgccaaagtgagcgtctaccgtaacgatgcaattacagttccagaaattgccgccttgaatcccgacacattgcttatctcgcctggaccaggccacccaaagacagattctggcatttcaagagactgtatccggtactttactgggaaaattcctgtatttggaatctgtatgggccagcaatgcatgtttgacgtatttggtggtgaagttgcctacgctggtgagattgtccacggtaaaacgtccccaatctctcacgacaactgtggaattttcaagaacgtgccgcaaggtattgctgtgacaagataccattcattggccgggacagaatcgtccctaccatcctgcttgaaggttactgcgagtaccgaaaatggaattatcatgggtgtaagacacaagaagtacactgtagaaggtgtgcaatttcatccggaatccatcttgaccgaggaaggtcatctgatgatcaggaacattttaaacgtcagtggaggcacttgggaggaaaacaaatcatctccttcaaattctattttggaccgtatctatgctcggcgtaaaatagacgtcaatgagcagtctaaaatcccaggtttcacctttcaagacttacaatctaactatgatttaggtcttgccccaccgttacaggatttctacacggtgttgtcatcatcccataaaagagccgttgttcttgctgaagtcaagcgtgcctctccatcgaagggacccatttgtttaaaagctgttgctgctgaacaggctctcaaatacgcagaggctggtgcatccgcaatttccgtattgaccgaacctcattggtttcacggttcgttacaggatttagtaaatgtgaggaaaatcctagatttgaaatttcctcccaaggagaggccttgtgttttgagaaaagaatttattttcagcaagtatcaaatactagaagcaagattagctggagctgacactgtccttcttatagtcaagatgctatctcaacccttattgaaggaactgtacagctacagtaaagatttgaacatggaacctctcgttgaggtgaactccaaagaggaattacaaagggctctagaaattggtgctaaagttgtaggtgtcaataatagggacctgcattcattcaacgtagacctaaataccaccagtaacttggtagaatctattccaaaggatgttcttctaattgctctatcgggaattaccaccagggacgatgctgaaaaatacaaaaaagaaggtgtccatggatttttagtgggtgaagccctaatgaaatcaaccgatgtgaagaagttcattcatgaattatgcgaataa

SEQ ID NO: 22 - protein, 484 aa, Saccharomyces cerevisiae
MSVHAATNPINKHVVLIDNYDSFTWNVYEYLCQEGAKVSVYRNDAITVPEIAALNPDTLLISPGPGHPKTDSGISRDCIRYFTGKIPVFGICMGQQCMFDVFGGEVAYAGEIVHGKTSPISHDNCGIFKNVPQGIAVTRYHSLAGTESSLPSCLKVTASTENGIIMGVRHKKYTVEGVQFHPESILTEEGHLMIRNILNVSGGTWEENKSSPSNSILDRIYARRKIDVNEQSKIPGFTFQDLQSNYDLGLAPPLQDFYTVLSSSHKRAVVLAEVKRASPSKGPICLKAVAAEQALKYAEAGASAISVLTEPHWFHGSLQDLVNVRKILDLKFPPKERPCVLRKEFIFSKYQILEARLAGADTVLLIVKMLSQPLLKELYSYSKDLNMEPLVEVNSKEELQRALEIGAKVVGVNNRDLHSFNVDLNTTSNLVESIPKDVLLIALSGITTRDDAEKYKKEGVHGFLVGEALMKSTDVKKFIHELCE

SEQ ID NO: 23 - DNA, 2124 bp, Saccharomyces cerevisiae
  source 1..2124: mol_type = other DNA
atgtcagaacaactcagacaaacatttgctaacgctaaaaaagaaaacaggaacgccttggtcacatttatgaccgcaggttacccaacagtcaaagacactgtccctattctcaagggtttccaggatggtggtgtagatatcatcgaattgggtatgcccttctctgatccaattgcagatggtcctacaattcaattatctaatactgtggctttgcaaaacggtgttaccttgcctcaaactctagaaatggtctcccaagctagaaatgaaggtgttaccgtacccataatcctaatgggttactataaccctattctaaactacggtgaagaaagatttattcaggacgctgccaaggctggtgctaatggttttatcatcgtcgatttgccaccagaggaggcgttgaaggtcagaaactacatcaatgataatggtttgagcctgatcccactagtggctccttctaccaccgacgaaagattggaattactatcgcatattgccgattcgtttgtctacgttgtgtctagaatgggtactactggtgttcaaagttctgtggccagtgatttggatgaactcatctctagagtcagaaagtacaccaaggatactcctttggccgttgggtttggtgtctctaccagagaacatttccaatcagttggtagtgttgctgacggtgtagtgattggttccaaaatcgtcacattatgtggagatgctccagagggcaaaaggtacgacgttgctaaggaatatgtacagggaattctaaatggtgctaagcataaggttctgtccaaggacgaattctttgcctttcaaaaagagtccttgaagtccgcaaacgttaagaaggaaatactggacgaatttgatgaaaatcacaagcacccaattagatttggggactttggtggtcagtatgtcccagaagctcttcatgcatgtctaagagagttggaaaagggttttgatgaagctgtcgccgatcccacattctgggaagacttcaaatccttgtattcttatattggccgtccttcttcactacacaaagctgagagattaactgagcattgtcaaggtgctcaaatctggttgaagagagaagatcttaaccacacgggatctcacaagatcaacaatgctttagcacaagttcttctagctaaaagattaggcaagaagaacgttattgctgaaaccggtgctggtcaacacggtgttgccactgccactgcatgtgctaaatttggcttaacctgtactgtgttcatgggtgcagaagatgttcgtcgccaagctttaaacgtcttcagaatgagaattctcggtgctaaagtaattgctgttactaatggtacaaagactctaagagacgctacttcagaggcattcagattttgggttactaacttgaaaactacttactacgtcgtcggttctgccattggtcctcacccatatccaactttggttagaactttccaaagtgtcattggtaaagaaaccaaggaacagtttgctgccatgaacaatggtaaattacctgacgcagttgttgcatgtgttgggggtggttccaactctacaggtatgttttcaccattcgagcatgatacttccgttaagttattgggtgtggaagccggtggtgatggtgtagatacaaagttccactctgctactctaactgccggtagacctggtgtcttccatggtgtcaagacttatgtcttgcaagatagtgatggtcaagtccatgatactcattctgtttctgctgggttagactacccaggtgtcggtccagaattggcatattggaaatctactggccgtgctcaattcattgcagctactgacgctcaggctctgcttggctttaaattattatctcaattagaaggtattattcccgctttggaatcttctcatgctgtttatggcgcttgcgaattggctaagacgatgaagcctgatcaacatttggttatcaatatttctggtagaggtgataaagatgtccaaagtgtcgctgaagtcttgccgaaattaggtccaaagataggttgggatttgagattcgaagaagacccatctgcctaa

SEQ ID NO: 24 - protein, 707 aa, Saccharomyces cerevisiae
MSEQLRQTFANAKKENRNALVTFMTAGYPTVKDTVPILKGFQDGGVDIIELGMPFSDPIADGPTIQLSNTVALQNGVTLPQTLEMVSQARNEGVTVPIILMGYYNPILNYGEERFIQDAAKAGANGFIIVDLPPEEALKVRNYINDNGLSLIPLVAPSTTDERLELLSHIADSFVYVVSRMGTTGVQSSVASDLDELISRVRKYTKDTPLAVGFGVSTREHFQSVGSVADGVVIGSKIVTLCGDAPEGKRYDVAKEYVQGILNGAKHKVLSKDEFFAFQKESLKSANVKKEILDEFDENHKHPIRFGDFGGQYVPEALHACLRELEKGFDEAVADPTFWEDFKSLYSYIGRPSSLHKAERLTEHCQGAQIWLKREDLNHTGSHKINNALAQVLLAKRLGKKNVIAETGAGQHGVATATACAKFGLTCTVFMGAEDVRRQALNVFRMRILGAKVIAVTNGTKTLRDATSEAFRFWVTNLKTTYYVVGSAIGPHPYPTLVRTFQSVIGKETKEQFAAMNNGKLPDAVVACVGGGSNSTGMFSPFEHDTSVKLLGVEAGGDGVDTKFHSATLTAGRPGVFHGVKTYVLQDSDGQVHDTHSVSAGLDYPGVGPELAYWKSTGRAQFIAATDAQALLGFKLLSQLEGIIPALESSHAVYGACELAKTMKPDQHLVINISGRGDKDVQSVAEVLPKLGPKIGWDLRFEEDPSA

SEQ ID NO: 25 - DNA, 1257 bp, Chromobacterium violaceum
  source 1..1257: mol_type = other DNA
atgaagcactcttctgatatctgtatagttggtgctggtatttctggtttgacttgtgcttctcatttgttggattctccagcttgtagaggtttgtccttgagaattttcgatatgcaacaagaggctggtggtagaatcagatctaaaatgttggatggtaaggcctccattgaattaggtgcaggtagatattctccacaattgcatccacatttccaatccgctatgcaacattactctcaaaagtctgaagtctacccattcacgcaattgaagttcaagtctcacgttcagcaaaaattgaagagagccatgaacgaattgtccccaagattgaaagaacacggtaaagaatctttcttgcagttcgtgtctagataccaaggtcatgattctgctgttggtatgattagatctatgggttacgatgctttgttcttgccagatatttctgctgaaatggcctatgatatcgttggtaaacatccagaaatccaatccgttactgataacgatgctaatcaatggtttgctgctgaaactggtttcgctggtttgattcaaggtattaaggctaaagttaaggctgctggtgctagattttctttgggttacagattattgtccgttagaactgatggtgacggttacttgttgcaattggctggtgatgatggttggaaattggaacatagaaccagacatttgatcttggctattccaccatctgctatggctggtttaaatgttgattttcctgaagcttggtctggtgcaagatatggttctttgcctttgtttaagggtttcttgacttatggtgaaccatggtggttggattacaagttggatgatcaagttttgatcgtcgacaacccattgagaaagatctactttaagggcgacaagtacctgttcttctacactgattcagaaatggccaattattggagaggttgtgttgctgaaggtgaagatggttacttggaacaaattagaactcatttggcttccgctttgggtatcgttagagaaagaattccacaaccattggctcatgttcataagtattgggctcatggtgttgaattctgtagagattccgatatcgatcatccatctgctttgtctcatagagactctggtattattgcttgctctgatgcttacactgaacattgcggatggatggaaggtggtttgttgtctgctagagaagcttcaagattgctgttgcaaagaattgctgcttaa

SEQ ID NO: 26 - protein, 418 aa, Chromobacterium violaceum
MKHSSDICIVGAGISGLTCASHLLDSPACRGLSLRIFDMQQEAGGRIRSKMLDGKASIELGAGRYSPQLHPHFQSAMQHYSQKSEVYPFTQLKFKSHVQQKLKRAMNELSPRLKEHGKESFLQFVSRYQGHDSAVGMIRSMGYDALFLPDISAEMAYDIVGKHPEIQSVTDNDANQWFAAETGFAGLIQGIKAKVKAAGARFSLGYRLLSVRTDGDGYLLQLAGDDGWKLEHRTRHLILAIPPSAMAGLNVDFPEAWSGARYGSLPLFKGFLTYGEPWWLDYKLDDQVLIVDNPLRKIYFKGDKYLFFYTDSEMANYWRGCVAEGEDGYLEQIRTHLASALGIVRERIPQPLAHVHKYWAHGVEFCRDSDIDHPSALSHRDSGIIACSDAYTEHCGWMEGGLLSAREASRLLLQRIAA

SEQ ID NO: 27 - DNA, 2997 bp, Chromobacterium violaceum
  source 1..2997: mol_type = other DNA
atgtccatcttggatttcccaagaatccattttagaggttgggctagagttaatgctccaactgctaatagagatccacatggtcatattgatatggcctctaatactgttgctatggctggtgaaccatttgatttggctagacatccaactgaattccacagacatttgagatctttgggtccaagatttggtttggatggtagagctgatccagaaggtccattttctttggctgaaggttataatgctgctggtaacaatcatttctcttgggaatctgctaccgtttctcatgttcaatgggatggtggtgaagctgatagaggtgatggtttggttggtgctagattggctttgtggggtcattacaatgattacttgagaaccactttcaacagagccagatgggttgattctgatccaactagaagagatgctgctcaaatctatgctggtcaattcactatttctccagctggtgctggtccaggtactccatggttgtttactgctgatattgatgattctcatggtgccagatggactagaggtggtcatatagcagaaagaggtggacatttcttggatgaagaatttggtttagccaggttgttccaattctctgttccaaaagatcacccacactttttgtttcatccaggtccattcgattctgaagcttggagaagattgcaattggcattggaagatgatgatgttttgggtttgactgttcaatacgccttgttcaatatgtctactccaccacaacctaactctccagtttttcatgatatggttggtgttgttggtttatggcgtagaggtgaattggcttcttacccagctggtagattattaagaccaagacaaccaggtttgggtgatttgactttgagagtttctggtggtagagttgctttgaatttggcttgtgctattccattctctactagagctgctcaaccatctgctccagatagattgactccagatttgggtgctaaattgccattaggtgatttgttgttgagggatgaagatggtgctttgttagctagagttccacaagccttgtatcaagattactggactaatcacggtatcgttgatttgccattattgagagaacctagaggttctttgaccttgtcatctgaattagctgaatggcgtgaacaagattgggttactcaatctgatgcttccaacttgtatttggaagctcctgatagaaggcatggtagatttttcccagaatctattgccttgaggtcttacttcagaggtgaagctagagctagaccagatattccacatagaattgaaggtatgggtttagtcggtgtcgaatctagacaagatggtgatgctgcagaatggcgtttgactggtttaagaccaggtccagctagaatagttttggatgatggtgcagaagctattcctttaagagttttgccagatgattgggctttagatgatgctactgttgaagaagttgattacgccttcttgtacagacatgttatggcttactacgaattggtctaccctttcatgtctgataaggttttctcattggctgatagatgtaagtgtgaaacctacgctagattgatgtggcaaatgtgtgatccacaaaaccgtaacaagtcttactacatgccatccactagagaattgtcagctccaaaagctaggttgttcttgaaatacttggcacacgttgaaggtcaagctagattgcaagctccaccaccagccggtcctgcaagaattgaatcaaaagctcaattggctgctgaattgagaaaggctgttgatttggaattgtccgtcatgttgcaatacttgtatgctgcttactccattccaaattacgctcaaggtcaacaaagggttagagatggtgcatggactgctgaacaattacaattagcttgtggttctggtgacagacgtagagatggcggtattagagcagctttgttggaaattgctcacgaagaaatgatccattacttggtcgttaacaacttgttgatggctttgggtgaacctttttatgctggtgttccattgatgggtgaagcagctagacaagctttcggtttagatactgaatttgccttggaaccattctccgaatctacattggctagattcgttagattggaatggccacattttattccagctccaggtaaatcaattgctgattgctatgctgctattagacaagcctttttggatttgcctgatttgtttggtggtgaggcaggtaagagaggtggcgaacatcatttgtttttgaacgaattgactaacagagcccatccaggttatcaattggaagttttcgatagagattccgctttgttcggtattgctttcgttactgatcaaggtgaaggtggtgcattggattctccacattatgaacattcccacttccaaagattgagggaaatgtctgctagaattatggctcaatcagctccatttgaaccagctttgccagctttaagaaatccagttttggacgaatctccaggttgtcaaagagttgctgacggtagagcaagagctttgatggcattatatcaaggtgtgtacgaactgatgtttgctatgatggctcaacattttgctgttaagccattgggttcattgagaagatccagattgatgaacgctgccattgatttgatgaccggtttgttaagaccactgtcttgtgctttgatgaacttgccatctggtattgctggtagaacagctggtccaccattgccaggtcctgttgataccagatcttatgatgattacgctttgggttgtagaatgttggctagaagatgcgaaagattgcttgaacaagcttctatgttggaaccaggttggttaccagatgctcaaatggaattattggacttctacagacgtcagatgttggatttggcatgtggtaaattgtctagagaagcctga

SEQ ID NO: 28 - protein, 998 aa, Chromobacterium violaceum
MSILDFPRIHFRGWARVNAPTANRDPHGHIDMASNTVAMAGEPFDLARHPTEFHRHLRSLGPRFGLDGRADPEGPFSLAEGYNAAGNNHFSWESATVSHVQWDGGEADRGDGLVGARLALWGHYNDYLRTTFNRARWVDSDPTRRDAAQIYAGQFTISPAGAGPGTPWLFTADIDDSHGARWTRGGHIAERGGHFLDEEFGLARLFQFSVPKDHPHFLFHPGPFDSEAWRRLQLALEDDDVLGLTVQYALFNMSTPPQPNSPVFHDMVGVVGLWRRGELASYPAGRLLRPRQPGLGDLTLRVSGGRVALNLACAIPFSTRAAQPSAPDRLTPDLGAKLPLGDLLLRDEDGALLARVPQALYQDYWTNHGIVDLPLLREPRGSLTLSSELAEWREQDWVTQSDASNLYLEAPDRRHGRFFPESIALRSYFRGEARARPDIPHRIEGMGLVGVESRQDGDAAEWRLTGLRPGPARIVLDDGAEAIPLRVLPDDWALDDATVEEVDYAFLYRHVMAYYELVYPFMSDKVFSLADRCKCETYARLMWQMCDPQNRNKSYYMPSTRELSAPKARLFLKYLAHVEGQARLQAPPPAGPARIESKAQLAAELRKAVDLELSVMLQYLYAAYSIPNYAQGQQRVRDGAWTAEQLQLACGSGDRRRDGGIRAALLEIAHEEMIHYLVVNNLLMALGEPFYAGVPLMGEAARQAFGLDTEFALEPFSESTLARFVRLEWPHFIPAPGKSIADCYAAIRQAFLDLPDLFGGEAGKRGGEHHLFLNELTNRAHPGYQLEVFDRDSALFGIAFVTDQGEGGALDSPHYEHSHFQRLREMSARIMAQSAPFEPALPALRNPVLDESPGCQRVADGRARALMALYQGVYELMFAMMAQHFAVKPLGSLRRSRLMNAAIDLMTGLLRPLSCALMNLPSGIAGRTAGPPLPGPVDTRSYDDYALGCRMLARRCERLLEQASMLEPGWLPDAQMELLDFYRRQMLDLACGKLSREA

SEQ ID NO: 29 - DNA, 576 bp, Chromobacterium violaceum
  source 1..576: mol_type = other DNA
atggaaaacagagaaccaccattattgccagctagatggtcatctgcttatgtttcttattggtcaccaatgttgccagacgatcaattgacttctggttactgttggttcgattacgaaagagatatctgcagaatcgacggtttgtttaatccatggtctgaaagagacactggttacagattgtggatgtctgaagttggtaatgctgcttctggtagaacttggaaacaaaaagttgcttacggtagagaaagaaccgctttgggtgaacaattgtgtgaaagaccattggatgacgaaactggtccatttgctgaattattcttgccaagagatgtcttgagaagattgggtgctagacatattggtagaagagttgttttgggtagagaagctgatggttggagatatcaaagaccaggtaaaggtccatctaccttgtatttggatgctgcatcaggtactccattgagaatggttactggtgatgaagcttctagagcatctttgagagatttcccaaatgtttccgaagccgaaattccagatgctgtttttgctgctaagaggtga

SEQ ID NO: 30 - protein, 191 aa, Chromobacterium violaceum
MENREPPLLPARWSSAYVSYWSPMLPDDQLTSGYCWFDYERDICRIDGLFNPWSERDTGYRLWMSEVGNAASGRTWKQKVAYGRERTALGEQLCERPLDDETGPFAELFLPRDVLRRLGARHIGRRVVLGREADGWRYQRPGKGPSTLYLDAASGTPLRMVTGDEASRASLRDFPNVSEAEIPDAVFAAKR

SEQ ID NO: 31 - DNA, 1122 bp, Chromobacterium violaceum
  source 1..1122: mol_type = other DNA
atgaagatcttggttattggtgctggtccagctggtttggtttttgcttctcaattgaaacaagctagaccattgtgggccattgatatcgttgaaaagaacgatgaacaagaggttttaggttggggtgttgttttgccaggtagaccaggtcaacatccagctaatccattgtcttatttggatgctccagaaagattgaacccacaattcttggaagatttcaagttggttcatcacaacgagccatctttgatgtctactggtgttttgttgtgtggtgttgagagaagaggtttggttcatgctttgagagataagtgtagatcccaaggtattgccattagatttgaatctcccttgttggaacatggtgaattgccattggctgattacgatttggttgttttggctaatggtgttaaccataagactgctcatttcaccgaagctttggttccacaagttgattatggtaggaacaagtacatttggtacggtacttctcagttgttcgatcaaatgaacttggttttcagaacccacggtaaggatattttcattgctcatgcttacaagtactccgataccatgtctactttcatcgttgaatgttccgaagaaacttacgctagagctagattgggtgaaatgtctgaagaggcttctgctgaatatgttgctaaggttttccaagctgaattaggtggtcatggtttagtttctcaaccaggtctaggttggagaaactttatgactttgtctcatgatagatgccacgacggtaaattggttttgttgggtgatgctttacaatccggtcatttctctataggtcatggtactactatggctgttgttgttgctcaattattggttaaggctttgtgtaccgaagatggtgttccagctgctttgaaaagatttgaagaaagagctttgcctctggtccaattattcagaggtcatgctgataattccagagtttggttcgaaactgtcgaagaaagaatgcatttgtcctccgctgaatttgtccaatcttttgatgctagacgtaagtctttgccaccaatgccagaagctttagctcaaaatttgagatacgccttgcagaggtaa

SEQ ID NO: 32 - protein, 373 aa, Chromobacterium violaceum
MKILVIGAGPAGLVFASQLKQARPLWAIDIVEKNDEQEVLGWGVVLPGRPGQHPANPLSYLDAPERLNPQFLEDFKLVHHNEPSLMSTGVLLCGVERRGLVHALRDKCRSQGIAIRFESPLLEHGELPLADYDLVVLANGVNHKTAHFTEALVPQVDYGRNKYIWYGTSQLFDQMNLVFRTHGKDIFIAHAYKYSDTMSTFIVECSEETYARARLGEMSEEASAEYVAKVFQAELGGHGLVSQPGLGWRNFMTLSHDRCHDGKLVLLGDALQSGHFSIGHGTTMAVVVAQLLVKALCTEDGVPAALKRFEERALPLVQLFRGHADNSRVWFETVEERMHLSSAEFVQSFDARRKSLPPMPEALAQNLRYALQR

SEQ ID NO: 33 - DNA, 1290 bp, Chromobacterium violaceum
  source 1..1290: mol_type = other DNA
atgaagagggctattatagttggtggtggtttagctggtggtttgactgctatctatttggctaaaaggggttacgaagttcacgttgttgaaaagagaggtgatcctttgagagacttgtcatcttacgttgatgttgtttcctctagagccattggtgtttctatgactgttagaggtatcaagtctgttttggctgctggtattccaagagctgaattggatgcttgtggtgaacctatagttgctatggctttttctgttggtggtcagtatagaatgagagagttgaaaccattggaagatttcaggccattgtctttgaatagagctgctttccaaaagctgttgaacaagtatgctaatttggctggtgttaggtactacttcgaacataagtgtttggatgttgatttggatggcaagtccgttttgattcaaggtaaagatggtcaaccacaaagattgcaaggtgatatgattattggtgctgatggtgctcattctgctgttagacaagctatgcaatctggtttgagaagattcgaattccaacagacctttttcagacatggttacaagactttggttttgccagatgctcaagctttgggttatagaaaagataccttgtacttcttcggtatggattccggtggtttgtttgctggtagagctgcaactattccagatggttctgtttctattgctgtttgcttgccatattctggttctccatctttgactactactgatgaacctactatgagagccttcttcgatagatatttcggtggtttaccaagagatgctagagatgagatgttgagacaatttttggctaagccatccaacgatttgatcaacgttagatcttctaccttccactacaagggtaacgttttgttgttgggtgatgctgctcatgctactgctccatttttaggtcaaggtatgaatatggctttggaagatgccagaacctttgttgaattattggatagacaccaaggtgatcaagataaggcttttccagaattcaccgaattgagaaaggttcaagctgatgctatgcaagatatggctagagctaattacgatgtcttgtcttgttctaaccccatctttttcatgagagctagatacaccagatatatgcactctaaatttccaggcttgtacccacctgatatggctgaaaagttgtactttacttccgaaccatacgacagattgcaacaaatccaaagaaagcaaaacgtctggtacaagatcggtagagttaactga

SEQ ID NO: 34 - protein, 429 aa, Chromobacterium violaceum
MKRAIIVGGGLAGGLTAIYLAKRGYEVHVVEKRGDPLRDLSSYVDVVSSRAIGVSMTVRGIKSVLAAGIPRAELDACGEPIVAMAFSVGGQYRMRELKPLEDFRPLSLNRAAFQKLLNKYANLAGVRYYFEHKCLDVDLDGKSVLIQGKDGQPQRLQGDMIIGADGAHSAVRQAMQSGLRRFEFQQTFFRHGYKTLVLPDAQALGYRKDTLYFFGMDSGGLFAGRAATIPDGSVSIAVCLPYSGSPSLTTTDEPTMRAFFDRYFGGLPRDARDEMLRQFLAKPSNDLINVRSSTFHYKGNVLLLGDAAHATAPFLGQGMNMALEDARTFVELLDRHQGDQDKAFPEFTELRKVQADAMQDMARANYDVLSCSNPIFFMRARYTRYMHSKFPGLYPPDMAEKLYFTSEPYDRLQQIQRKQNVWYKIGRVN

SEQ ID NO: 35 - DNA, 1503 bp, Catharanthus roseus
  source 1..1503: mol_type = other DNA
atgggttctattgattctaccaacgtcgctatgtctaattctccagttggtgaatttaagccattggaagccgaagaatttagaaagcaagctcacagaatggttgatttcattgccgattactacaagaacgttgaaacctacccagttttgtctgaagttgaaccaggttacttgagaaagagaataccagaaactgctccatatttgccagaaccattggatgatattatgaaggacatccaaaaggacatcattccaggtatgactaattggatgtctccaaacttttacgctttcttccctgctactgtttcttctgctgcttttttgggtgaaatgttgtctactgctttgaactctgttggtttcacttgggtttcttcaccagctgctactgaattggaaatgatagttatggattggttggcccaaatcttgaagttgccaaagtcttttatgttctctggtactggtggtggtgttattcaaaacactacctccgaatctattttgtgcaccattattgctgctagagaaagagctttggaaaagttgggtccagattccattggtaaattggtttgttacggttctgatcaaacccataccatgtttccaaagacttgtaaattggctggtatctacccaaacaacatcagattgattccaaccactgttgaaaccgatttcggtatttctccacaagttttaagaaagatggtcgaagatgatgttgctgctggttatgttcctttgtttttgtgtgctactttgggtactacttctactactgctacagatccagttgactccttgtctgaaattgctaacgaatttggtatctggattcatgttgatgctgcttacgctggttctgcttgtatttgtcctgaatttagacactacttggacggtatcgaaagagtcgattctttgtctttgtctccacataagtggttgttggcttatttggattgcacttgtttgtgggttaagcaaccacatttgttgttgagagctttgactaccaatccagaatacttgaagaacaagcaatccgatttggataaggtcgttgactttaagaactggcaaattgctactggtagaaagttcagatccttgaaattgtggttgatcttgagatcctacggtgttgttaacttgcaatcccacattagatcagatgttgctatgggtaagatgtttgaagaatgggttagatccgactccagattcgaaatagttgttccaagaaacttctcattggtctgctttagattgaagccagatgtttcctcattgcacgttgaagaagtcaacaaaaagttgttggacatgttgaactctacaggtagagtctacatgactcatacaatagtcggtggtatctatatgttgagattggctgttggttcttcattgaccgaagaacatcatgttagaagagtttgggacttgatccaaaagttgaccgatgacttgttgaaagaagcctga

SEQ ID NO: 36 - protein, 500 aa, Catharanthus roseus
MGSIDSTNVAMSNSPVGEFKPLEAEEFRKQAHRMVDFIADYYKNVETYPVLSEVEPGYLRKRIPETAPYLPEPLDDIMKDIQKDIIPGMTNWMSPNFYAFFPATVSSAAFLGEMLSTALNSVGFTWVSSPAATELEMIVMDWLAQILKLPKSFMFSGTGGGVIQNTTSESILCTIIAARERALEKLGPDSIGKLVCYGSDQTHTMFPKTCKLAGIYPNNIRLIPTTVETDFGISPQVLRKMVEDDVAAGYVPLFLCATLGTTSTTATDPVDSLSEIANEFGIWIHVDAAYAGSACICPEFRHYLDGIERVDSLSLSPHKWLLAYLDCTCLWVKQPHLLLRALTTNPEYLKNKQSDLDKVVDFKNWQIATGRKFRSLKLWLILRSYGVVNLQSHIRSDVAMGKMFEEWVRSDSRFEIVVPRNFSLVCFRLKPDVSSLHVEEVNKKLLDMLNSTGRVYMTHTIVGGIYMLRLAVGSSLTEEHHVRRVWDLIQKLTDDLLKEA

SEQ ID NO: 37 - DNA, 1008 bp, Saccharomyces cerevisiae
  source 1..1008: mol_type = other DNA
atgtctgaaccagctcaaaagaaacaaaaggttgctaacaactctctagaacaattgaaagcctccggcactgtcgttgttgccgacactggtgatttcggctctattgccaagtttcaacctcaagactccacaactaacccatcattgatcttggctgctgccaagcaaccaacttacgccaagttgatcgatgttgccgtggaatacggtaagaagcatggtaagaccaccgaagaacaagtcgaaaatgctgtggacagattgttagtcgaattcggtaaggagatcttaaagattgttccaggcagagtctccaccgaagttgatgctagattgtcttttgacactcaagctaccattgaaaaggctagacatatcattaaattgtttgaacaagaaggtgtctccaaggaaagagtccttattaaaattgcttccacttgggaaggtattcaagctgccaaagaattggaagaaaaggacggtatccactgtaatttgactctattattctccttcgttcaagcagttgcctgtgccgaggcccaagttactttgatttccccatttgttggtagaattctagactggtacaaatccagcactggtaaagattacaagggtgaagccgacccaggtgttatttccgtcaagaaaatctacaactactacaagaagtacggttacaagactattgttatgggtgcttctttcagaagcactgacgaaatcaaaaacttggctggtgttgactatctaacaatttctccagctttattggacaagttgatgaacagtactgaacctttcccaagagttttggaccctgtctccgctaagaaggaagccggcgacaagatttcttacatcagcgacgaatctaaattcagattcgacttgaatgaagacgctatggccactgaaaaattgtccgaaggtatcagaaaattctctgccgatattgttactctattcgacttgattgaaaagaaagttaccgcttaa

SEQ ID NO: 38 - protein, 335 aa, Saccharomyces cerevisiae
MSEPAQKKQKVANNSLEQLKASGTVVVADTGDFGSIAKFQPQDSTTNPSLILAAAKQPTYAKLIDVAVEYGKKHGKTTEEQVENAVDRLLVEFGKEILKIVPGRVSTEVDARLSFDTQATIEKARHIIKLFEQEGVSKERVLIKIASTWEGIQAAKELEEKDGIHCNLTLLFSFVQAVACAEAQVTLISPFVGRILDWYKSSTGKDYKGEADPGVISVKKIYNYYKKYGYKTIVMGASFRSTDEIKNLAGVDYLTISPALLDKLMNSTEPFPRVLDPVSAKKEAGDKISYISDESKFRFDLNEDAMATEKLSEGIRKFSADIVTLFDLIEKKVTA

SEQ ID NO: 39 - DNA, 2043 bp, Saccharomyces cerevisiae
  source 1..2043: mol_type = other DNA
atgactcaattcactgacattgataagctagccgtctccaccataagaattttggctgtggacaccgtatccaaggccaactcaggtcacccaggtgctccattgggtatggcaccagctgcacacgttctatggagtcaaatgcgcatgaacccaaccaacccagactggatcaacagagatagatttgtcttgtctaacggtcacgcggtcgctttgttgtattctatgctacatttgactggttacgatctgtctattgaagacttgaaacagttcagacagttgggttccagaacaccaggtcatcctgaatttgagttgccaggtgttgaagttactaccggtccattaggtcaaggtatctccaacgctgttggtatggccatggctcaagctaacctggctgccacttacaacaagccgggctttaccttgtctgacaactacacctatgttttcttgggtgacggttgtttgcaagaaggtatttcttcagaagcttcctccttggctggtcatttgaaattgggtaacttgattgccatctacgatgacaacaagatcactatcgatggtgctaccagtatctcattcgatgaagatgttgctaagagatacgaagcctacggttgggaagttttgtacgtagaaaatggtaacgaagatctagccggtattgccaaggctattgctcaagctaagttatccaaggacaaaccaactttgatcaaaatgaccacaaccattggttacggttccttgcatgccggctctcactctgtgcacggtgccccattgaaagcagatgatgttaaacaactaaagagcaaattcggtttcaacccagacaagtcctttgttgttccacaagaagtttacgaccactaccaaaagacaattttaaagccaggtgtcgaagccaacaacaagtggaacaagttgttcagcgaataccaaaagaaattcccagaattaggtgctgaattggctagaagattgagcggccaactacccgcaaattgggaatctaagttgccaacttacaccgccaaggactctgccgtggccactagaaaattatcagaaactgttcttgaggatgtttacaatcaattgccagagttgattggtggttctgccgatttaacaccttctaacttgaccagatggaaggaagcccttgacttccaacctccttcttccggttcaggtaactactctggtagatacattaggtacggtattagagaacacgctatgggtgccataatgaacggtatttcagctttcggtgccaactacaaaccatacggtggtactttcttgaacttcgtttcttatgctgctggtgccgttagattgtccgctttgtctggccacccagttatttgggttgctacacatgactctatcggtgtcggtgaagatggtccaacacatcaacctattgaaactttagcacacttcagatccctaccaaacattcaagtttggagaccagctgatggtaacgaagtttctgccgcctacaagaactctttagaatccaagcatactccaagtatcattgctttgtccagacaaaacttgccacaattggaaggtagctctattgaaagcgcttctaagggtggttacgtactacaagatgttgctaacccagatattattttagtggctactggttccgaagtgtctttgagtgttgaagctgctaagactttggccgcaaagaacatcaaggctcgtgttgtttctctaccagatttcttcacttttgacaaacaacccctagaatacagactatcagtcttaccagacaacgttccaatcatgtctgttgaagttttggctaccacatgttggggcaaatacgctcatcaatccttcggtattgacagatttggtgcctccggtaaggcaccagaagtcttcaagttcttcggtttcaccccagaaggtgttgctgaaagagctcaaaagaccattgcattctataagggtgacaagctaatttctcctttgaaaaaagctttctaa

SEQ ID NO: 40 - protein, 680 aa, Saccharomyces cerevisiae
MTQFTDIDKLAVSTIRILAVDTVSKANSGHPGAPLGMAPAAHVLWSQMRMNPTNPDWINRDRFVLSNGHAVALLYSMLHLTGYDLSIEDLKQFRQLGSRTPGHPEFELPGVEVTTGPLGQGISNAVGMAMAQANLAATYNKPGFTLSDNYTYVFLGDGCLQEGISSEASSLAGHLKLGNLIAIYDDNKITIDGATSISFDEDVAKRYEAYGWEVLYVENGNEDLAGIAKAIAQAKLSKDKPTLIKMTTTIGYGSLHAGSHSVHGAPLKADDVKQLKSKFGFNPDKSFVVPQEVYDHYQKTILKPGVEANNKWNKLFSEYQKKFPELGAELARRLSGQLPANWESKLPTYTAKDSAVATRKLSETVLEDVYNQLPELIGGSADLTPSNLTRWKEALDFQPPSSGSGNYSGRYIRYGIREHAMGAIMNGISAFGANYKPYGGTFLNFVSYAAGAVRLSALSGHPVIWVATHDSIGVGEDGPTHQPIETLAHFRSLPNIQVWRPADGNEVSAAYKNSLESKHTPSIIALSRQNLPQLEGSSIESASKGGYVLQDVANPDIILVATGSEVSLSVEAAKTLAAKNIKARVVSLPDFFTFDKQPLEYRLSVLPDNVPIMSVEVLATTCWGKYAHQSFGIDRFGASGKAPEVFKFFGFTPEGVAERAQKTIAFYKGDKLISPLKKAF

SEQ ID NO: 41 - DNA, 1038 bp, Saccharomyces cerevisiae
  source 1..1038: mol_type = other DNA
atgaccatagataactacgacaacagtaaacaggatagcagcaaatacgaggttagtggtacgggtgatggcaggaacggcgatggcggcttgcctctagtacaatgtgtcgcaagagctcgtatcccaaccacacagggtccggatatctttttacatctttacagtaacaacagggacaacaaggaacatctagccattgtgtttggtgaagacatacggtcgcgctcgctattccgtagaagacagtgcgagacgcaacaagatagaatgatcaggggcgcttatattggcaaactgtatcccggcagaactgtggcagacgaagacgatagactcggattagctctggagtttgatgatagtacaggtgagttattagcttccaaagccactacatgggacgcccataacgacacgctggtacggatccattctgaatgttacaccggtgaaaacgcatggagcgcccgttgtgattgtggtgaacaattcgatagggccggtaggcttatcgcttgcgaccacgaacccacaagcaacatcaaaggtggaaacggccatggtgttatcgtgtatctaagacaagagggtcgtggcatcgggttaggtgagaaactcaaggcctacaacctgcaagacttaggtgctgacacagtgcaggccaacttaatgctgaaacatcccgtggatgctagggacttctcgctgggtaaggctattttgctggatcttggcatcggtaatgtcaggctgctgacaaataacccggaaaagataaaacaggtagatcatgcgccttaccttaagtgcgttgaacgagtgcctatggtacccatacactggacaaactccagtgaaggcatagactccaaggagatagaaggttatctcaggaccaagatagaaagaatgggtcatttactaacggagcctctgaaacttcatacaaaccctcaacctactgagacaagtgaagcccaaaaccaaaaccgtatgaactctgcgttgtcatcaacatcgacgctggcaatataa

SEQ ID NO: 42 - protein, 345 aa, Saccharomyces cerevisiae
MTIDNYDNSKQDSSKYEVSGTGDGRNGDGGLPLVQCVARARIPTTQGPDIFLHLYSNNRDNKEHLAIVFGEDIRSRSLFRRRQCETQQDRMIRGAYIGKLYPGRTVADEDDRLGLALEFDDSTGELLASKATTWDAHNDTLVRIHSECYTGENAWSARCDCGEQFDRAGRLIACDHEPTSNIKGGNGHGVIVYLRQEGRGIGLGEKLKAYNLQDLGADTVQANLMLKHPVDARDFSLGKAILLDLGIGNVRLLTNNPEKIKQVDHAPYLKCVERVPMVPIHWTNSSEGIDSKEIEGYLRTKIERMGHLLTEPLKLHTNPQPTETSEAQNQNRMNSALSSTSTLAI

SEQ ID NO: 43 - DNA, 936 bp, Saccharomyces cerevisiae
  source 1..936: mol_type = other DNA
atggtcgatcaccagtggacgccactgcagaaggaggttatctcaggtctgtccgcgggatccgtcaccacattggtagtgcatccattagatttactgaaggttcgtttgcaattatccgctacaagcgcacaaaaggcccattacggcccatttatggtaatcaaagagattatacgttcgtcggcgaatagtggacgtagtgttacgaacgaactgtatcgcggattaagcataaatttgtttggaaatgccattgcatggggagtttatttcggtttatatggtgtcacgaaagaattgatatacaagtcagtggcaaagccaggggaaacacagcttaaaggtgttgggaacgaccacaaaatgaattcgcttatttatttgtctgccggtgcttccagtgggttgatgacagctatcctaacaaaccctatctgggttataaagaccaggataatgtctacaagtaaaggcgcccaaggagcgtacacttctatgtacaacggtgtccaacaactgttacgaactgatggcttccagggcctatggaagggccttgtacccgcactgtttggtgtttcgcaaggtgccttatattttgcagtttacgataccttaaagcaaagaaaattgcgacggaagagggaaaatgggcttgacatccatttaacaaatcttgaaaccattgaaatcacatcgttgggaaaaatggtctccgtcactttggtttacccttttcaattattgaaatcaaacttacaaagtttcagggccaatgagcaaaaatttaggctctttcctttgataaaattaatcattgctaatgatggttttgttggactttataagggcctatctgcaaacttagtacgggccattccatccacttgcattaccttttgtgtatacgagaaccttaagcataggctttag

SEQ ID NO: 44 - protein, 311 aa, Saccharomyces cerevisiae
MVDHQWTPLQKEVISGLSAGSVTTLVVHPLDLLKVRLQLSATSAQKAHYGPFMVIKEIIRSSANSGRSVTNELYRGLSINLFGNAIAWGVYFGLYGVTKELIYKSVAKPGETQLKGVGNDHKMNSLIYLSAGASSGLMTAILTNPIWVIKTRIMSTSKGAQGAYTSMYNGVQQLLRTDGFQGLWKGLVPALFGVSQGALYFAVYDTLKQRKLRRKRENGLDIHLTNLETIEITSLGKMVSVTLVYPFQLLKSNLQSFRANEQKFRLFPLIKLIIANDGFVGLYKGLSANLVRAIPSTCITFCVYENLKHRL

SEQ ID NO: 45 - DNA, 954 bp, Saccharomyces cerevisiae
  source 1..954: mol_type = other DNA
atggaggacagtagcaatacaatcataccctcacccactgacgtgggggcgctagcaaacagaatcaactttcaaaccagagatgcccacaataaaatcaataccttcatgggcataaagatggccatcgccatgagacatggctttatatacagacagggtattctggcgtactattatgtgttcgatgccatcgagcaagagatagatcgcctactgaatgaccccgtaacggaggaggagctgcaaacttcgaccattctgaagcagttttggctcgaagattttagaagatctacgcagatctataaggacctgaagctgctatactcaaacacgtttaaaagcacagaatcattaaacgaattcctggctacgttccagaagccaccgctactacagcagtttatcaataacatccacgaaaacatacacaaggagccatgcaccattctttcttactgtcacgttctgtacttggcgcttttcgccggcggcaagctaatacgatcgaatttgtacagaagactggggctcttccccaacttcgagaagctatcacagaaggaactggtcaaaaagggcacaaacttcttcaccttcagcgatctgggtcccactgaagaaacacgcttgaaatgggaatacaagaagaactatgagctggccaccaggacggaattgaccgaagcacaaaagttgcagatcattagcgtcgcagaaggcatttttgattggaacttcaacatcgttgcagaaattggagagttgaatcgtcgcgagttaatgggcaagttcagcttcaagtgtattacgtacttgtacgaagaatggatgttcaacaaggattctgctactagaagagcactccacacggtcatgctgctggtgctttctattatcgcgatctgggttctttacttcttggtaaagagttttcttagcatagtataa

SEQ ID NO: 46 - DNA, 984 bp, Saccharomyces cerevisiae
  source 1..984: mol_type = other DNA
atgggccctgaaactctacatattggtgggagaaaatcgaaattggcggtaatacaatccaaccatgttttaaaactgatcgaagaaaagtatccggactacgactgcaaggttttcactttgcaaactcttggtgaccagattcaattcaaacctttgtactcatttggcggtaaagctttatggacaaaggagttggaagaccatctttaccatgacgatccctcaaagaagcttgacttgatcgttcattctctgaaggacatgcccactttactaccagagggtttcgagctggggggtatcactaagcgggtcgatccaacagattgtcttgtcatgcccttttactctgcttataagtctctggatgaccttccagacggggggattgtgggaacctcatccgtgagaagatctgctcagctaaaaagaaaatacccacatttgaaatttgaaagtgtcagaggaaatatacaaactagattacaaaaactagacgacccaaaatctccgtaccaatgcatcatcttggcgtctgctgggttgatgcgtatggggttggaaaacagaattacgcagcgattccattcggatacaatgtaccatgcagttggacaaggcgccctgggtatagaaattagaaagggtgacaccaagatgatgaagattcttgacgaaatttgcgatctaaatgcaactatatgttgcctttcggagcgtgctttgatgagaactttagaggggggttgttccgttcctattggtgtggaatctaaatacaatgaagagactaaaaaattactattaaaggccattgtagttgacgttgaaggcacagaagcagtagaagacgaaattgaaatgctaatagaaaatgttaaagaagattccatggcgtgtggtaagatactagctgaaagaatgattgccgatggcgcaaagaaaattctggatgaaattaatttagacagaatcaaatga

SEQ ID NO: 47 - protein, 327 aa, Saccharomyces cerevisiae
MGPETLHIGGRKSKLAVIQSNHVLKLIEEKYPDYDCKVFTLQTLGDQIQFKPLYSFGGKALWTKELEDHLYHDDPSKKLDLIVHSLKDMPTLLPEGFELGGITKRVDPTDCLVMPFYSAYKSLDDLPDGGIVGTSSVRRSAQLKRKYPHLKFESVRGNIQTRLQKLDDPKSPYQCIILASAGLMRMGLENRITQRFHSDTMYHAVGQGALGIEIRKGDTKMMKILDEICDLNATICCLSERALMRTLEGGCSVPIGVESKYNEETKKLLLKAIVVDVEGTEAVEDEIEMLIENVKEDSMACGKILAERMIADGAKKILDEINLDRIK

SEQ ID NO: 48 - DNA, 1023 bp, Saccharomyces cerevisiae
  source 1..1023: mol_type = other DNA
atgtctgaaataaccttaggtaaatatttatttgaaagattgagccaagtcaactgtaacaccgtcttcggtttgccaggtgactttaacttgtctcttttggataagctttatgaagtcaaaggtatgagatgggctggtaacgctaacgaattgaacgctgcctatgctgctgatggttacgctcgtatcaagggtatgtcctgtattattaccaccttcggtgttggtgaattgtctgctttgaatggtattgccggttcttacgctgaacatgtcggtgttttgcacgttgttggtgttccatccatctcttctcaagctaagcaattgttgttgcatcataccttgggtaacggtgacttcactgttttccacagaatgtctgccaacatttctgaaaccactgccatgatcactgatattgctaacgctccagctgaaattgacagatgtatcagaaccacctacactacccaaagaccagtctacttgggtttgccagctaacttggttgacttgaacgtcccagccaagttattggaaactccaattgacttgtctttgaagccaaacgacgctgaagctgaagctgaagttgttagaactgttgttgaattgatcaaggatgctaagaacccagttatcttggctgatgcttgtgcttctagacatgatgtcaaggctgaaactaagaagttgatggacttgactcaattcccagtttacgtcaccccaatgggtaagggtgctattgacgaacaacacccaagatacggtggtgtttacgttggtaccttgtctagaccagaagttaagaaggctgtagaatctgctgatttgatattgtctatcggtgctttgttgtctgatttcaataccggttctttctcttactcctacaagaccaaaaatatcgttgaattccactctgaccacatcaagatcagaaacgccaccttcccaggtgttcaaatgaaatttgccttgcaaaaattgttggatgctattccagaagtcgtc

SEQ ID NO: 49 - DNA, 1023 bp, Saccharomyces cerevisiae
  source 1..1023: mol_type = other DNA
atggcacctgttacaattgaaaagttcgtaaatcaagaagaacgacaccttgtttccaaccgatcagcaacaattccgtttggtgaatacatatttaaaagattgttgtccatcgatacgaaatcagttttcggtgttcctggtgacttcaacttatctctattagaatatctctattcacctagtgttgaatcagctggcctaagatgggtcggcacgtgtaatgaactgaacgccgcttatgcggccgacggatattcccgttactctaataagattggctgtttaataaccacgtatggcgttggtgaattaagcgccttgaacggtatagccggttcgttcgctgaaaatgtcaaagttttgcacattgttggtgtggccaagtccatagattcgcgttcaagtaactttagtgatcggaacctacatcatttggtcccacagctacatgattcaaattttaaagggccaaatcataaagtatatcatgatatggtaaaagatagagtcgcttgctcggtagcctacttggaggatattgaaactgcatgtgaccaagtcgataatgttatccgcgatatttacaagtattctaaacctggttatatttttgttcctgcagattttgcggatatgtctgttacatgtgataatttggttaatgttccacgtatatctcaacaagattgtatagtatacccttctgaaaaccaattgtctgacataatcaacaagattactagttggatatattccagtaaaacacctgcgatccttggagacgtactgactgataggtatggtgtgagtaactttttgaacaagcttatctgcaaaactgggatttggaatttttccactgttatgggaaaatctgtaattgatgagtcaaacccaacttatatgggtcaatataatggtaaagaaggtttaaaacaagtctatgaacattttgaactgtgcgacttggtcttgcattttggagtcgacatcaatgaaattaataatgggcattatact

SEQ ID NO: 50 - DNA, 1245 bp, Saccharomyces cerevisiae
  source 1..1245: mol_type = other DNA
atgtttgtcagggttaaattgaataaaccagtaaaatggtataggttctatagtacgttggattcacattccctaaagttacagagcggctcgaagtttgtaaaaataaagccagtaaataacttgaggagtagttcatcagcagatttcgtgtccccaccaaattccaaattacaatctttaatctggcagaaccctttacaaaatgtttatataactaaaaaaccatggactccatccacaagagaagcgatggttgaattcataactcatttacatgagtcataccccgaggtgaacgtcattgttcaacccgatgtggcagaagaaatttcccaggatttcaaatctcctttggagaatgatcccaaccgacctcatatactttatactggtcctgaacaagatatcgtaaacagaacagacttattggtgacattgggaggtgatgggactattttacacggcgtatcaatgttcggaaatacgcaagttcctccggttttagcatttgctctgggcactctgggctttctatcaccgtttgattttaaggagcataaaaaggtctttcaggaagtaatcagctctagagccaaatgtttgcatagaacacggctagaatgtcatttgaaaaaaaaggatagcaactcatctattgtgacccatgctatgaatgacatattcttacataggggtaattcccctcatctcactaacctggacattttcattgatggggaatttttgacaagaacgacagcagatggtgttgcattggccactccaacgggttccacagcatattcattatcagcaggtggatctattgtttccccattagtccctgctattttaatgacaccaatttgtcctcgctctttgtcattccgaccactgattttgcctcattcatcccacattaggataaagataggttccaaattgaaccaaaaaccagtcaacagtgtggtaaaactttctgttgatggtattcctcaacaggatttagatgttggtgatgaaatttatgttataaatgaggtcggcactatatacatagatggtactcagcttccgacgacaagaaaaactgaaaatgactttaataattcaaaaaagcctaaaaggtcagggatttattgtgtcgccaagaccgagaatgactggattagaggaatcaatgaacttttaggattcaattctagctttaggctgaccaagagacagactgataatgattaa

SEQ ID NO: 51 - protein, 414 aa, Saccharomyces cerevisiae
MFVRVKLNKPVKWYRFYSTLDSHSLKLQSGSKFVKIKPVNNLRSSSSADFVSPPNSKLQSLIWQNPLQNVYITKKPWTPSTREAMVEFITHLHESYPEVNVIVQPDVAEEISQDFKSPLENDPNRPHILYTGPEQDIVNRTDLLVTLGGDGTILHGVSMFGNTQVPPVLAFALGTLGFLSPFDFKEHKKVFQEVISSRAKCLHRTRLECHLKKKDSNSSIVTHAMNDIFLHRGNSPHLTNLDIFIDGEFLTRTTADGVALATPTGSTAYSLSAGGSIVSPLVPAILMTPICPRSLSFRPLILPHSSHIRIKIGSKLNQKPVNSVVKLSVDGIPQQDLDVGDEIYVINEVGTIYIDGTQLPTTRKTENDFNNSKKPKRSGIYCVAKTENDWIRGINELLGFNSSFRLTKRQTDND

SEQ ID NO: 52 - DNA, 1257 bp, Chromobacterium violaceum
  source 1..1257: mol_type = other DNA
atgaaacattcatcggacatctgcatcgttggcgcgggaatctctggcctgacgtgtgcctcccacttgttggactcgcctgcttgccgtggcctttcgttacgtatcttcgatatgcaacaggaagctggagggcgtatccgctcgaagatgcttgacgggaaggcatctatcgagcttggagctggacgttattcgccccagcttcatccgcactttcagtcagctatgcaacactactcacaaaagtccgaggtttaccctttcacccaactgaagttcaaaagccacgtacagcaaaagcttaaacgcgccatgaatgagttatcgccccgtcttaaagagcacggtaaggaaagctttttacaattcgtgagccgctaccagggtcatgattcagcggtaggaatgattcgttcaatgggctatgatgccctgtttttgcctgatatctccgctgaaatggcatatgacattgtgggtaaacacccggaaatccaatccgtgaccgataacgatgcaaaccagtggtttgccgcggagacgggcttcgcaggcttaatccaaggtattaaggcgaaagtgaaggcagcgggcgcacgcttttcattaggttaccgtctgctgagcgtgcgtacggatggtgacgggtacttgcttcagttggcgggggacgatgggtggaagttggagcatcgcactcgtcaccttatcttagctattccgccctctgcgatggccgggctgaacgtagactttcctgaagcttggagtggagcgcgttatggtagtttgccccttttcaagggattccttacttacggagaaccatggtggcttgactacaaactggacgaccaagtcttaatcgtcgataaccctctgcgtaagatttattttaagggagataaatacctgtttttctacaccgattccgagatggccaattactggcgtgggtgcgtggcagagggcgaagacggttacttggaacagatccgtacccatttggcgtctgccttgggcatcgtgcgtgagcgcatcccccagccgttagcccacgtgcacaaatactgggcgcacggggtggaattctgtcgtgatagtgatattgaccacccatcggcgctgtcgcatcgtgactccggtattatcgcgtgctcagatgcctacacagagcactgtggatggatggaggggggactgctttctgctcgcgaggcttcgcgcttgcttttacaacgcatcgcagcatga

SEQ ID NO: 53 - DNA, 2997 bp, Chromobacterium violaceum
  source 1..2997: mol_type = other DNA
atgtcgattttggacttcccacgtatccattttcgtggatgggcgcgtgttaacgcgccaactgcgaatcgtgatccccatgggcacatcgatatggcgtcgaatacggtcgccatggccggcgagcccttcgacctggctcgtcatccgaccgagttccatcgccacttgcgctcattgggcccccgtttcggcttagatggtcgtgctgatccggaagggccatttagcctggcggaaggctacaatgccgcaggcaacaatcacttttcttgggagagcgcgaccgtcagccacgtgcaatgggacggtggggaagcagaccgtggcgatggtctggtgggagcacgcttggccctgtggggacattataatgattatctgcgtacgacttttaatcgtgcacgttgggttgacagtgatccaacacgtcgtgatgctgctcaaatctatgcggggcagtttaccatcagtcctgcaggggcaggccctggtacgccctggttattcaccgcagacatcgacgattctcacggagcacgctggactcgcggtggacatattgcagagcgtggcggccatttccttgacgaggagttcggtttggcccgtttatttcagttttccgtgcctaaagaccaccctcattttctttttcacccggggccattcgattccgaggcatggcgtcgtctgcagctggccttggaagacgacgacgttttaggtttgacggttcagtatgcactgttcaatatgtctactcctccccagcctaattccccagtatttcatgatatggtaggtgtagttggcctgtggcgccgtggagaattggcgtcttatccggctgggcgtttgttacgtccccgtcaaccaggccttggggatctgaccctgcgtgttagcggagggcgtgtcgccttaaatcttgcttgtgcgatccccttttccacgcgtgcagcgcagccatcggcccctgatcgcttgacgccagacttgggcgccaaattacccctgggcgatcttcttctgcgtgacgaagatggcgccttacttgcccgtgtaccgcaagccttatatcaggattactggacgaaccatggcatcgtagatcttccgttactgcgcgaaccacgtggaagccttaccttatcctctgaactggccgaatggcgtgagcaagactgggttacgcaatctgacgcctctaacctgtatttggaggccccagaccgtcgtcacggccgtttttttcctgagagtattgcattacgctcgtattttcgtggcgaggcgcgtgctcgtccagatatcccgcaccgcattgagggaatgggactggtcggagtggaatcccgtcaggatggagacgccgcggagtggcgcctgaccgggttgcgccccggcccagcacgcattgtattggatgatggggctgaggcgatcccattgcgtgtactgccagacgattgggcacttgatgacgcaaccgtcgaggaggttgattatgcgttcctttaccgccacgtaatggcttactatgagctggtctaccctttcatgtcagataaagtcttctcacttgccgaccgttgtaagtgcgaaacatacgcacgtcttatgtggcagatgtgcgatccgcaaaaccgcaacaagtcgtattatatgccgtcgacgcgtgagctttctgcaccgaaagctcgccttttcttgaagtacttagcacatgttgaaggccaggctcgtttacaagcacctccacccgctggacccgcccgcattgaatcgaaagcccaacttgccgcagagttgcgtaaagctgtagaccttgagcttagcgtaatgcttcagtacctgtacgccgcttactcgattcctaattacgctcagggccaacaacgcgtgcgcgacggggcgtggactgcagaacaacttcagctggcctgcggttccggtgatcgtcgccgcgacggtggaatccgtgccgcgctgcttgagattgctcacgaggagatgatccattatttggtcgtaaacaatctgctgatggccttaggagagccattctacgccggagttcctcttatgggagaagcggcgcgtcaggcattcggattagatacagagttcgctctggaaccatttagtgaaagtactctggcgcgttttgtgcgcttagaatggccacactttatccccgcgccggggaaaagtatcgcggattgttacgcggctattcgccaagcgtttctggatttacccgacctgtttggaggtgaagcgggcaaacgtgggggcgaacaccatttatttttgaatgagcttactaatcgcgcccatcccggatatcagcttgaggtgttcgatcgtgactccgccctttttggcattgcctttgtcaccgaccagggagaaggaggagctctggatagtccgcattatgaacattctcatttccaacgcttacgtgagatgtcggcccgtattatggcccagtctgcaccgtttgaaccagctttacctgcgttgcgtaacccagtcctggacgagtcacccggttgccaacgtgtggcagacgggcgcgctcgcgcacttatggccttgtaccaaggggtgtatgagttgatgttcgcaatgatggcacagcacttcgcggtcaagcctcttggatcgcttcgtcgctcccgcttgatgaacgcggccattgatcttatgacgggacttcttcgtccattgtcatgtgcgctgatgaatttaccttcggggatcgccggacgcacggcgggaccgccgttgcctggccccgtcgacacacgttcctacgatgactatgctttgggttgccgtatgttagctcgtcgttgtgagcgccttttagagcaagcctcgatgcttgaaccgggttggttgcctgatgcgcagatggaactgttggacttctaccgccgccaaatgctggacttggcgtgcggtaaattaagccgtgaagcatga

SEQ ID NO: 54 - DNA, 1290 bp, Chromobacterium violaceum
  source 1..1290: mol_type = other DNA
atgaagcgcgcgatcattgtcggtggtgggctggccggagggcttaccgcaatctacttagccaaacgtggttatgaggtacatgtagttgagaagcgtggagacccgcttcgcgacttaagcagttacgttgatgtggtttcttctcgtgctattggagtaagcatgaccgtccgtggcattaaaagtgtgctggcagccgggatcccccgcgcggagttggatgcatgtggagaacctattgtggcaatggccttctccgtaggaggtcagtatcgtatgcgtgagcttaagccgttggaggacttccgtccactttcgcttaatcgcgctgcctttcagaagttattgaataagtacgcaaacttagcaggggtgcgctattactttgagcataaatgtcttgacgttgacttagacggtaaatcagttttgatccaaggtaaagacggccagccccaacgtctgcagggtgatatgattattggcgccgatggagctcactcagcagtgcgtcaggcgatgcaaagcgggcttcgccgcttcgagtttcagcaaacgttcttccgtcacgggtacaagaccctggtactgcccgacgcacaggcgttaggatatcgtaaggatacgctttattttttcggcatggattcgggcgggttatttgctggtcgcgcggcgacgattcctgacggcagcgtaagtattgctgtttgccttccatacagtggtagccctagtcttacaacaactgacgagcccactatgcgcgcatttttcgatcgttactttggagggctgccacgcgacgcacgcgacgaaatgttgcgtcagtttttggcaaaaccttcgaatgatctgattaacgtgcgtagcagcacattccactataaggggaacgttcttttgctgggcgacgctgcacatgctacagctccgttcctggggcagggtatgaacatggctctggaagacgcccgcacattcgttgagctgttagatcgccaccaaggcgaccaggataaggcttttccggaattcacggagcttcgtaaggtgcaagcagatgcaatgcaggacatggcgcgcgcgaactacgatgtgctgtcatgcagcaaccctatcttctttatgcgcgctcgctacacacgttacatgcactctaaattccccggcctttatccacctgacatggcggaaaagctttattttactagtgaaccctacgaccgccttcagcagattcaacgcaaacagaatgtgtggtacaaaatcggccgcgtgaattga

SEQ ID NO: 55 - DNA, 1122 bp, Chromobacterium violaceum
  source 1..1122: mol_type = other DNA
atgaaaattttagtgattggcgctggaccggcaggactggttttcgcgtctcagctgaagcaggctcgtcctttatgggcaatcgatatcgtcgaaaagaatgacgaacaagaagtattgggatggggcgtagtgcttccggggcgtcctggtcaacatcccgccaatccactttcctatttggatgcaccggaacgtctgaacccacaatttttagaagattttaagttggtacatcacaatgagccatccttaatgagcacaggtgtacttctgtgcggagtggaacgtcgcggtttggtacacgcgttacgcgacaaatgtcgtagtcagggaatcgccattcgtttcgagtctccgttactggagcacggagaacttcccttagctgattatgatttggtcgtattagccaatggtgtgaatcataagacggcgcatttcaccgaagcccttgtccctcaggtcgattatggacgcaacaaatatatctggtacggtacatctcaattattcgatcagatgaatctggtgttccgtacacatggaaaagatattttcattgcacacgcttataagtactcggacacgatgtcgacgttcattgtggagtgttccgaggaaacgtatgcacgtgctcgtctgggcgagatgagtgaagaagcgtcggctgagtatgtagccaaagtgttccaagcagaacttggtgggcacggtttagtctctcaaccaggtcttggctggcgcaactttatgactttatcgcatgatcgctgtcacgatggtaaactggttttgctgggagacgctctgcaaagtggccatttttcaatcggtcacggcaccacaatggcggttgttgttgctcagctgttggtgaaggccttatgtactgaagacggagttcccgcagcgctgaagcgtttcgaggaacgtgctctgccactggttcagctttttcgcgggcacgcagacaattctcgtgtctggttcgagaccgtagaagagcgcatgcacctttcatctgcagagtttgttcaatcctttgatgctcgccgtaaatcgcttccacctatgccagaggcgcttgctcagaacttacgctacgctcttcagcgttga

SEQ ID NO: 56 - DNA, 576 bp, Chromobacterium violaceum
  source 1..576: mol_type = other DNA
atggaaaaccgcgaaccccccctgcttccagcacgctggagttcggcatacgtttcatactggtcaccgatgcttccagacgaccaactgacatcgggttattgctggttcgactatgagcgcgacatttgccgtattgacggattatttaatccatggagcgaacgtgataccggataccgcctgtggatgtctgaagtggggaacgcagcgtcagggcgtacttggaaacaaaaagtagcttatggtcgtgaacgcactgcccttggagaacagctttgtgagcgcccattggacgatgagacaggtccctttgccgagctgtttttacctcgcgacgtcttgcgccgtttgggcgcacgtcacattggacgccgtgttgtgcttggtcgcgaggcggacgggtggcgttatcaacgtcctggaaagggtccttccactctgtatttagatgctgcctccggaacccccttgcgtatggtgacaggcgatgaggcttcacgtgcttccttacgcgattttcctaacgtaagtgaagcggagattcccgacgctgtctttgcggcgaagcgttga

SEQ ID NO: 57 - DNA, 2739 bp, Saccharomyces cerevisiae
  source 1..2739: mol_type = other DNA
atgaacatgcaagacgctaacaaagaagagtcgtactcgatgtacccgaaaacctcttctccaccaccacctacgccaaccaatatgcagattcctatttatcaagcgcctttgcagatgtacggctacactcaggccccatatctataccccacacaaatacctgcctattcgtttaatatggtcaaccaaaaccagccaatctaccatcaaagtggcagcccacatcacttgcctccgcaaaacaatattaacggcggaagcactaccaataacaacaacattaacaagaagaagtggcactctaatggcattaccaataacaatggaagcagcggtaatcaaggcgccaactctagcggtagcggcatgagctacaacaaatcccacacctaccatcacaattactctaacaatcatatccccatgatggcctctccaaacagtggcagcaatgcgggcatgaaaaaacagaccaactcttccaacggcaacggttcttcggctacttcaccatcgtactcttcctacaactcttcttcacagtatgatttatacaagtttgatgtcactaaattaaagaatctcaaggaaaattcatcaaacttgattcaattgccactgttcataaacactacggaagcagaatttgctgcggcaagtgtccaaaggtacgaattaaacatgaaggctttgaacctaaactctgaaagcttagagaactcatctgtagaaaagagctctgcccatcatcacacaaaaagccatagtataccaaagcataatgaggaagtaaagacagaaacacatggggaagaagaagatgctcatgataaaaaaccacatgcgagcaaagatgcgcacgagcttaaaaagaaaactgaagtaaagaaagaggatgctaagcaagaccgtaacgaaaaagttatacaggaacctcaagctactgttttacctgtagtggataagaaggaaccagaggaatctgttgaagaaaatacttccaagacatcttcaccttcaccatctcctccagcagcaaaatcctggtccgccatagcatcagatgcgattaaaagtagacaagctagtaacaaaacagtctccggatcgatggtcactaaaacaccaatttctggtacgaccgcaggcgtttcatcaacaaacatggctgcggcgactataggtaaatccagctctcccctgttgtccaagcagcctcagaaaaaggataaaaaatacgttccaccttctacaaagggtattgagccactgggttcgattgcgttaagaatgtgttttgatcccgatttcattagttacgttttacggaataaagatgttgaaaacaaaataccagtccattccattattccaagaggcataattaacagagccaacatttgttttatgagttctgtgttacaagtgttactctactgtaagccatttattgatgtaattaacgttctcagtacacggaataccaattcaagagtcggcacatcatcctgtaaattattagatgcttgtttgactatgtataagcaattcgataaggaaacctatgagaaaaaattcctagagaatgctgatgatgctgaaaaaaccacggaaagtgatgcaaaaaaatcatcaaaatccaagagtttccaacactgcgccactgccgatgctgtcaaacctgacgaattttacaaaactttgtctactataccgaagttcaaagacttgcaatggggccatcaggaagacgcagaagaatttttgacccacttattggaccaattacacgaggaattaatttctgcaattgatggcttaaccgataatgaaattcaaaatatgctgcaaagtattaatgatgaacaattgaaagttttctttattagaaatttgtcacgttatggaaaagcagagtttatcaaaaatgctagtcctagactgaaggagttgatagaaaaatatggcgtgatcaatgatgactctaccgaagaaaatggttggcatgaagtgagcggatctagcaaaagaggcaagaaaactaagaccgctgccaagaggactgtcgagattgttccatcaccaatctccaaacttttcggtggccagttcagatctgtgttagatataccgaacaataaggaatctcaatcgattacactcgatccgttccaaacaattcaattggacatttcagatgctggtgtgaatgatctagaaactgcattcaaaaaatttagtgaatacgaattgctaccctttaagtcctcgtcagggaatgatgtcgaggccaagaagcagacttttattgataaattgccgcaagttcttttaatccaattcaaaagattctcattcataaataatgtgaacaaagacaacgcaatgacgaactataacgcgtacaatggacgtattgagaagatcaggaaaaaaattaaatatggtcacgagttaatcatacctgaagaatcaatgtcttccataacattgaaaaacaacacctcagggattgatgatagaagatataagctaaccggagttatataccatcatggggtaagttccgatggcggtcattacacagcggatgtttatcatagcgagcacaacaaatggtatagaatagatgatgtaaatattaccgaactagaggacgatgacgttttgaaaggtggcgaagaagcttctgattcgaggactgcctatattttaatgtatcaaaagagaaattaa

SEQ ID NO: 58 - DNA, 3171 bp, Saccharomyces cerevisiae
  source 1..3171: mol_type = other DNA
atgttcatcaaacagtctgaaaaaaatacacctaaatgcctctacaaaaaaaaaggtaaggtaagagtgttattgaccggaagctgtaagaaactgaacacgtggaaaatgcacttatggccagtcagtccgccccaattgcttagaattccaccccgcaacgccgaattaggtgagggtactaaaatagatgattgcaatattcttcaatcaatgacactaccccaggccaatgttcttatcatgctaaccccaacaagagtgctcatttacaacttcaaaccaatggcactagtagctagccatgaacgaacaatggcttctcttaaggaatttggcgataacagatcaatgaaacgatcagctccatataatgatatcatcgaagggcttatttcaaaaaaggactcgcaatacttattgtggcaccagggaaagctgatattttacgtaatgactgataaaaacttccttttaacataccaaattttaaaaaactgcacaaatgaaataattttcaaagaatacggcatacccgtcatagagcctcttttaatgagtgaagaagaagcgaacagtgctgaatatgactataacaacgatgatgatactctcacggtatttgataaaaatagttcttcaagaattatacagaatggatttggcataactaaggaaaaagggtttttacactttctttctaatcaagaaaacattgatgaactccccgtgaaaaaactagaacttcgtttaaaagtagttcttaaatttgactatgaaatcatagacatgattggcatcaagacattttccaaagtcggggatgggcgttatgaagaagttttgatagttttattccctcatggattacaaatacttacaatatcggattttaaagttagcaagagctcgttggttgaagtgaagaagggatccaagacaattgtgtgtaataaacaattaatggttttatctcatgattccgtcgaaaagcagactattgtaagcattatagacattgaaaagcaagctgttgaagcaataccgttaacggacacaccagatgaacttttgacttgcctagaagttaatggatacctggtggtcgtttacaaagaaaaaataatctgctttgatacaagaattaaaaaagtaagccattcttggaaaccaccatttgtgataaaactttgcgataaaattaatgataaaatattacttttggtttctgaggatagtgttaacattcatttttacacagaatttgggaatctgctattcgctacatattttgacgaagatgattataatggtgataacaataatgacaacagcaaggataaaaatgagaaaaaggcggctgaatataaaatttctgattttgtatgcttggataaatcactaataacagtgtctcattccggaaaatatcaagtttggaaattatgggaagaaataaaacaaacacaatttgatttcagaaacccaaaatgttacgtattaaccaatacgaacaatgatgtaatcatatattcacctgtgacaagttcttcaatcaataatgataatttacaagtcataaaacttcctacaaagactttcaataatcatattgcttttgtaaaaattaattcttccttgaggttatttgctacttatgtgtcgaacaaaaacatactgcttatccacaatctagagaccaatatgtggtctagttttgctgaccaaaacgtgttggatttacattggctaggtgataattatttggtttgtcatatgaagaatgatgatggctcaacaaatctgaaatgtctgcagattccactgcaagaagcgaatccggacgtggaactatcagattatgtcatgtgggaatacaatgttccagagaatacgattgttttcagtttacatgttaatactttatcccggtataagttgttgaaaatgaaatccaagaatcacaatgcttctgaaaaacaacctgatgcattgcttaaaactgccgagattattttagttactgatacacagacaattgtcttcgatgtaatttctactgtacatccatgtggattaaacatcatcaaaaaattctaccagtatctcaaaattaacattccaattgatgttttacccaacaaaattgagtggataattaacatgaaggagggcttgttattttttgccgataggaaatttataaagcttggcaaagtggatggagggggctggcaaaccttaactttgttggataacatagaaaaaataatcgatgttatcagggacgaaatatttgtagttcaaggccataattacgtcgtatactctttggaagatttatgggatgataaaaagccactagtttccattccaatcgaagaagatttgtatccaatatcaaccaccccagaaacagctacaacacatacacttcactgcatttttaacgcgcgattttccaaattggtggtgaaacaccaaatatacctcgaccaattaatcttagccaaattagaagacaacacagatttggaggatatttcacacaattatcgttttttaaagccttacaaatttgcactcgaaaagattttgtctactaaaatactgcgaagcgactctttggatgatatcctgaaattaattaagatgtatgacaacaccgatcctgagcataatatttcaccaccaacccacagtggtatgttagaaattattagtaattgtttaaggaagatcgaaactaaatactggaaccatctattcactaatctaaagatgacaccaagagatttattagcactctgtatcgaagaaaatgaggctaaaatgcttggcgtacttctgttagtgtttttaaattacgatgagaaagatttgggagatgatctacattttaagaaatcagatttagggactgaggaaagcaaagctctaaatgataactctacaaaaaagtcagagaaatctgtcactaatttattgaaggatgaagaattaatgcttaaagtcttagagcttttggtgacaagtgctgcaaacgccacagacccaattaaagctaccgattcgtgggacatgtgtttccaattaatacgcttactcaaggaattagacagagaaaacaacacacaattggttcaaaaagcactcgagagattcaaataa

SEQ ID NO: 59 - DNA, 753 bp, Saccharomyces cerevisiae
  source 1..753: mol_type = other DNA
atgcctttgaccacaaaacctttatctttgaaaatcaacgccgctctattcgatgttgacggtaccatcatcatctctcaaccagccattgctgctttctggagagatttcggtaaagacaagccttacttcgatgccgaacacgttattcacatctctcacggttggagaacttacgatgccattgccaagttcgctccagactttgctgatgaagaatacgttaacaagctagaaggtgaaatcccagaaaagtacggtgaacactccatcgaagttccaggtgctgtcaagttgtgtaatgctttgaacgccttgccaaaggaaaaatgggctgtcgccacctctggtacccgtgacatggccaagaaatggttcgacattttgaagatcaagagaccagaatacttcatcaccgccaatgatgtcaagcaaggtaagcctcacccagaaccatacttaaagggtagaaacggtttgggtttcccaattaatgaacaagacccatccaaatctaaggttgttgtctttgaagacgcaccagctggtattgctgctggtaaggctgctggctgtaaaatcgttggtattgctaccactttcgatttggacttcttgaaggaaaagggttgtgacatcattgtcaagaaccacgaatctatcagagtcggtgaatacaacgctgaaaccgatgaagtcgaattgatctttgatgactacttatacgctaaggatgacttgttgaaatggtaa

SEQ ID NO: 60 - DNA, 1107 bp, Saccharomyces cerevisiae
  source 1..1107: mol_type = other DNA
atgaatcctaaatcctctacacctaagattccaagacccaagaacgcatttattctgttcagacagcactaccacaggatcttaatagacgaatggaccgctcaaggtgtggaaataccccataattcaaacatttctaaaattattggtacgaagtggaagggcttacaaccggaagataaggcacactgggaaaatctagcggagaaggagaaactagaacatgaaaggaagtatcctgaatacaaatacaagccggtaagaaagtctaagaagaagcaactacttttgaaggaaatcgagcaacagcagcagcaacaacagaaagaacagcagcagcagaaacagtcacaaccgcaattacaacagccctttaacaacaatatagttcttatgaaaagagcacattctctttcaccatcttcctcggtgtcaagctcgaacagctatcagttccaattgaacaatgatcttaagaggttgcctattccttctgttaatacttctaactatatggtctccagatctttaagtggactacctttgacgcatgataagacggcaagagacctaccacagctgtcatctcaactaaattctattccatattactcagctccacacgacccttcaacgagacatcattacctcaacgtcgctcaagctcaaccaagggctaactcgacccctcaattgccctttatttcatccattatcaacaacagcagtcaaacaccggtaactacaactaccacatccacaacaactgcgacatcttctcctgggaaattctcctcttctccgaactcctctgtactggagaacaacagattaaacagtatcaacaattcaaatcaatatttacctccccctctattaccttctctgcaagattttcaactggatcagtaccagcagctaaagcagatgggaccaacttatattgtcaaaccactgtctcacaccaggaacaatctattgtccacaactacccctacgcatcatcacattcctcatataccaaaccaaaacattcctctacatcaaattataaactcaagcaacactgaggtcaccgctaaaactagcctagtttctccgaaatga

SEQ ID NO: 61 - DNA, 4509 bp, Saccharomyces cerevisiae
  source 1..4509: mol_type = other DNA
atgtcgaataccccttataattcatctgtgccttccattgcatccatgacccagtcttcggtctcaagaagtcctaacatgcatacagcaactacgcccggtgccaacaccagctctaactctccacccttgcacatgtcttcagattcgtccaagatcaagaggaagcgtaacagaattccgctcagttgcaccatttgtcggaaaaggaaagtcaaatgtgacaaactcagaccacactgccagcagtgcactaaaactggggtagcccatctctgccactacatggaacagacctgggcagaagaggcagagaaagaattgctgaaggacaacgaattaaagaagcttagggagcgcgtaaaatctttagaaaagactctttctaaggtgcactcttctccttcgtctaactccttgaaaagttacaacactcccgagagcagcaacctgtttatgggtagcgatgaacacaccacccttgttaatgcaaatacaggctctgcttcctctgcctcgcatatgcatcagcaacaacagcaacagcagcaacaggaacaacaacaagacttttccagaagtgcgaacgccaacgcgaattcctcgtccctttctatctcaaataaatatgacaacgatgagctggacttaactaaggactttgatcttttgcatatcaaaagtaacggaaccatccacttaggtgccacccactggttgtctatcatgaaaggtgacccgtacctaaaacttttgtggggtcatatcttcgctatgagggaaaagttaaatgaatggtactaccaaaaaaattcgtactctaagctgaagtcaagcaaatgtcccatcaatcacgcgcaagcgccgccttctgccgctgccgccgctaccagaaaatgtcctgttgatcactccgcgttttcgtctggcatggtggccccaaaggaggagactcctcttcctaggaaatgtccagttgaccacaccatgttctcttcgggaatgattcctcccagagaggacacttcgtcccagaagaggtgtcccgttgaccacaccatgtattccgcaggaatgatgccgcccaaggacgagacaccttccccattttccactaaagctatgatagaccataacaagcatacaatgaatccgcctcagtcaaaatgtcctgtggaccatagaaactatatgaaggattatccctctgacatggcaaattcttcttcgaacccggcaagtcgttgccccattgaccattcaagcatgaaaaatacagcggccttaccagcttcaacgcacaataccatcccacaccaccaaccacagtccggatctcatgctcgttcgcatcccgcacaaagcaggaaacatgattcctacatgacagaatctgaagtcctcgcaacactttgtgagatgttgccaccaaagcgcgtcatcgcattattcatcgagaaattcttcaaacatttataccctgccattccaatcttagatgaacagaatttcaaaaatcacgtgaatcaaatgctttcgttgtcttcgatgaatcccacagttaacaactttggtatgagcatgccatcttcatctacactagagaaccaacccataacacaaatcaatcttccaaaactttccgattcttgtaacttaggtattctgataataatcttgagattgacatggctatccataccttctaattcctgcgaagtcgacctgggagaagaaagtggctcatttttagtgcccaacgaatctagcaatatgtctgcatctgcattgacctcgatggctaaagaagaatcacttctgctaaagcatgagacaccggtcgaggcactggagctatgtcaaaaatacttgattaaattcgatgaactttctagtatttccaataacaacgttaatttaaccacggtgcagtttgccattttttacaacttctatatgaaaagtgcctctaatgatttgactaccttgacaaataccaacaacactggcatggccaatcctggtcacgattccgagtctcaccagatcctattgtccaatattactcaaatggcctttagttgtgggttacacagagaccctgataattttcctcaattaaacgctaccattccagcaaccagccaggacgtgtctaacaacgggagcaaaaaggcaaaccctagcaccaatccaactttgaataacaacatgtctgctgccactaccaacagcagtagcagatctggcagtgctgattcaagaagtggttctaaccctgtgaacaagaaggaaaatcaggttagtatcgaaagatttaaacacacttggaggaaaatttggtattacattgttagcatggatgttaaccaatctctttccctggggagccctcgactactaagaaatctgagggatttcagcgatacaaagctaccaagtgcgtcaaggattgattatgttcgcgatatcaaagagttaatcattgtgaagaattttactctttttttccaaattgatttgtgtattattgctgtattaaatcacattttgaatgtttctttagcaagaagcgtgagaaaatttgaactggattcattgattaatttattgaaaaatctgacctatggtactgagaatgtcaatgatgtagtgagctccttgatcaacaaagggttattaccaacttcggaaggtggttctgtagattcaaataatgatgaaatttacggtctaccgaaactacccgatattctaaaccatggtcaacataaccaaaacttgtatgctgatggaagaaatacttctagtagtgatatagataagaaattggaccttcctcacgaatctacaacgagagctctattcttttccaagcatatgacaattagaatgttgctgtacttattgaactacattttgtttactcattatgaaccaatgggcagtgaagatcctggtactaatatcctagctaaggagtacgctcaagaggcattaaattttgccatggatggctacagaaactgcatgattttcttcaacaatatcagaaacaccaattcactattcgattacatgaatgttatcttgtcttacccttgtttggacattggacatcgttctttacaatttatcgtttgtttgatcctgagagctaaatgtggcccattgactggtatgcgtgaatcatcgatcattactaatggtacatcaagtggatttaatagttcggtagaagatgaggacgtcaaagttaaacaagaatcttctgatgaattgaaaaaagacgatttcatgaaagatgtaaatttggattcaggcgattcattagcagagattctaatgtcaagaatgctgctatttcaaaaactaacaaaacaactatcaaagaagtacaactacgctattcgtatgaacaaatccactggattctttgtctctttactagatacaccttcaaagaaatcagactcgaaatcgggtggtagttcattcatgttgggtaattggaaacatccaaaggtttcaaacatgagcggatttcttgctggtgacaaagaccaattacagaaatgccccgtgtaccaagatgcgctggggtttgttagtccaaccggtgctaatgaaggttctgctccgatgcaaggcatgtccttacagggctctactgctaggatgggagggacccagttgccaccaattagatcatacaaacctatcacgtacacaagtagtaatctacgtcgtatgaatgaaacgggtgaggcagaagctaagagaagaagatttaatgatggctatattgataataatagtaacaacgatatacctagaggaatcagcccaaaaccttcaaatgggctatcatcggtgcagccactattatcgtcattttccatgaaccagctaaacgggggtaccattccaacggttccatcgttaaccaacattacttcacaaatgggagctttaccatctttagataggatcaccactaatcaaataaatttgccagacccatctagagatgaagcatttgacaactccatcaagcaaatgacgcctatgacaagtgcattcatgaatgctaatactacaattccaagttcaactttaaacgggaatatgaacatgaatggagctggaactgcgaatacagatacaagtgccaacggcagtgctttatcgacactgacaagcccacaaggctcagacttagcatccaattctgctacacagtataaacctgacttagaagactttttgatgcaaaattctaactttaatgggctaatgataaatccttccagtctggtagaagttgttggtggatacaacgatcctaataaccttggaagaaatgacgcggttgattttctacccgttgataatgttgaaattgatggtgttggaataaaaatcaactatcatctactaactagtatttacgttactagtatattatcatatacggtgttagaagatgacgcaaatgatgagaaatag

SEQ ID NO: 62 - DNA, 2103 bp, Psilocybe cubensis
  source 1..2103: mol_type = other DNA
atggaagccatcaaaaaagtgttcgagaacaaaaaagcagaaggcattccggttctggttacctttgttaccgcaggttatccgcgtccggaagataccgttccgattctgctggcaatggaagccggtggtgcagatattattgaactgggtatgccgtttagcgatccgattgcagatggtccggttattcaagaaaccaataccattgcagttgccaaccaggttgattataccaccgttctgggtcagctgcgtgaagcacgtaaacagggtctgaaagcaccggttctgctgatgggctattacaacccgattctggcatatggtgaagatcgtagcattcaggatgcagccgaagcgggtgcaaatggctttattatggttgatctgcctccggaagaggcagttgcatttcgtgaaaaatgcattaaaagcaacctgagctatgttccgctgattgcaccgagcaccacactgagccgtatcaaatttctgagcaccattgccgatacctttatctatgttgttagcaaaatgggtacaaccggtagcagcgaaaaagttgcaatgaataatgcactgccgaccattattgatcgtattcgtgaatatgcagaagttccgctggcagttggttttggtgttgcaacccgtgcacattttaactatgttgcagatagcggtgccgatggtgttgttattggcaccaaactggttaacgtgattaaagaaagtccgcagggtgaagcaccgaaaaatgttgaagcatattgtcgtgaaatgagccagaaaggtgaaaccaatcgtgttaaaagccctccgaccgcacgtgcagcaagcagcgaatcaattccggttgttgttccgagcgttctgcctgcacgttttggtgaatttggtggtcagtatgttccggaagcactggttgattgtctggttgaactggaagaagcacataaaagcgcaatggcagatccggaatttcagaaagaactgcagagccatgcaggttatgcaaatcgtccgagccagatttatctggcggaaaatctgaccaaagatgcaggcggtgcgaatatttggctgaaacgtgaagatctgaatcataccggtagccacaaaattaacaatgcactgggtcagatcctgctggcacgtcgtattggtaaaacccgtattattgccgaaaccggtgcaggtcagcatggtgttgccaccgcaaccgtttgtgcaaaatttggtatggaatgcgtgatttatatgggtgcagaagatgttcgtcgtcaggcactgaatgtttttcgtattgaaatgctgggtgccaaagttgttccggttaccagcggtagctgtaccctgaaagatgccgttaatgaagcctttcgtgattgggttaccaatctgagcacaacccattatctggttggtagcgttattggtccgcatccgtttccgaccattgttcgtgatttccagaaagtgattggccaagaaattaaagcacagatgctggcagcacgtggtaaactgccggatgttgttgttgcatgtgttggtggtggtagcaatgcaattggcaccttttatgatttcatcggcgataaaagcgttcgtctggtgggtgtagaagctggcggtgaaggtattgatggtgatcgtcatagcgcaaccctgagcatgggtcagccaggtgttctgcatggtgtgcgtacctatattctgcaggataaagcaggtcagattatcgaaacccatagcattagcgcaggtctggattatcctggtgttggtcctgaacatgcatggctgaaagatagcaaacgtgccgaatatgttgttgcgaccgatgaagaggcactgcgtggttttcgcatgctgacacagcgcgaaggtattattccggcactggaaagcagccatgccatttgggaagcagttcgtattgcacgtaccatgagcaaagatcaggatctggttgtttgcctgagcggtcgtggtgataaagatgttgagcagattagccagctgctgccgaaatgggcagatattctggattggcatgttagcagtcatgcagttggtcatacaaccaaattctaa

SEQ ID NO: 63 - protein, 700 aa, Psilocybe cubensis [sequence omitted]

SEQ ID NO: 64 - DNA, 1458 bp, Populus trichocarpa
  source 1..1458: mol_type = other DNA
atgggtagcctgggtcatcagctgcatatcttttttctgccgttttttgcacatggccatatgattccgagcgttgatatggcaaaactgtttgcaagccgtggtattaaaaccaccattattaccacaccgctgaacgcaccgttttttagcaaaaccattcagaaaaccaaagagctgggcttcgatattaacatcctgaccatcaaatttccggcagcagaagcaggtctgccggaaggttatgaaaataccgatgcatttatcttcagcgaaaatgcacgtgagatgacgatcaaattcattaaagcaaccacctttctgcaggcaccgtttgaaaaagttctgcaagaatgtcatccggattgtattgttgccgatgttttttttccgtgggcaaccgatgcagcagcaaaatttggtattccgcgtctggtttttcatggcaccagcaattttgcactgagcgcaagcgaatgtgttcgtctgtatgaaccgcataaaaaagttagcagcgatagcgaaccgtttgttgttccggatctgcctggtgatattaaactgaccaaaaaacagctgccggatgatgttcgtgaaaatgtggaaaatgacttcagcaaattcctgaaagcaagcaaagaagcagaactgcgtagctttggtgttgttgtgaatagcttttatgaactggaaccggcatatgcggactactacaaaaaagtgctgggtcgtcgtgcatggaatgttggtccggttagcctgtgtaatcgtgataccgaagataaagcaggtcgtggtaaagaaaccagcattgatcatcatgaatgtctgaaatggctggacagcaaaaaaccgaatagcgttgtgtatatttgctttggtagcaccacgaattttagcgatagccagctgaaagaaattgcagccggtctggaagcaagcggtcagcagtttatttgggttgttcgtcgtaacaaaaaaggccaagaggataaagaagattggctgcctgaaggctttgaagaacgtatggaaggtgttggtctgattattcgtggttgggcaccgcaggttctgattctggatcatgaagcaattggtgcatttgttacccattgtggttggaatagcaccctggaaggtattaccgcaggtaaaccgatggttacctggccgatttttgcagaacagttctataatgaaaaactggtgaccgatgtgctgaaaaccggtgttggtgtgggtgttaaagaatggtttcgtgttcatggtgatcacgttaaaagcgaagcagtggaaaaaaccattacgcagattatggttggtgaagaggccgaagaaatgcgtagccgtgccaaaaaactgggtgaaaccgcacgtaaagcagttgaagaaggtggtagcagctatagtgattttaatgccctgattgaagaactgcgctggcgtcgtccgtaa

SEQ ID NO: 65 - DNA, 1458 bp, Populus trichocarpa
  source 1..1458: mol_type = other DNA
atgggttctttgggtcatcagttgcacattttctttttgccatttttcgcccatggtcacatgattccatctgttgatatggctaagttgttcgcttctagaggtattaagaccaccattattactaccccattgaacgctccattcttctcaaagactatccaaaagaccaaagagttgggtttcgacattaacatcttgaccattaagtttccagctgctgaagctggtttgccagaaggttacgaaaacactgatgctttcatcttctctgaaaacgctagagaaatgaccatcaagttcattaaggctaccactttcttgcaagctccatttgaaaaggtgttgcaagaatgtcatccagattgcatagttgccgatgttttctttccatgggctactgatgctgctgctaaatttggtattccaagattggttttccatggcacttctaactttgctttgtctgcttctgaatgcgttaggttgtatgaaccacataagaaggttagctctgactctgaaccatttgttgttccagatttgccaggtgatattaagttgaccaaaaagcaattgccagacgacgttagagaaaacgttgaaaatgacttctccaagttcctgaaggcttctaaagaagctgaattgagatctttcggtgttgtcgttaactccttctatgaattggaaccagcttacgctgattactacaaaaaggttttgggtagacgtgcttggaatgttggtccagtttctttgtgtaacagagacactgaagataaggctggtagaggtaaagaaacctccattgatcatcatgaatgcttgaagtggttggactctaagaagccaaattccgttgtttacatttgcttcggttctaccactaacttctccgactctcaattgaaagaaattgctgctggtttggaagcttctggtcaacaattcatttgggttgtcagaagaaacaagaaaggccaagaggacaaagaagattggctacctgaaggttttgaagaaagaatggaaggtgttggtttgattattagaggttgggctccacaagttttgattttggatcatgaagctattggtgccttcgttactcattgtggttggaattctactttggaaggtattactgctggtaagccaatggttacttggccaatttttgctgaacagttctacaacgaaaagttggttaccgatgtcttgaaaactggtgttggtgtaggtgtcaaagaatggtttagagttcatggtgatcacgttaagtctgaagctgttgaaaagaccatcactcaaatcatggttggtgaagaggctgaagaaatgagatctagagctaaaaagttgggtgaaactgctagaaaggctgttgaagaaggtggttcttcttactctgatttcaacgccttgattgaagaactaagatggcgtagaccataa

SEQ ID NO: 66 - protein, 485 aa, Populus trichocarpa
MGSLGHQLHIFFLPFFAHGHMIPSVDMAKLFASRGIKTTIITTPLNAPFFSKTIQKTKELGFDINILTIKFPAAEAGLPEGYENTDAFIFSENAREMTIKFIKATTFLQAPFEKVLQECHPDCIVADVFFPWATDAAAKFGIPRLVFHGTSNFALSASECVRLYEPHKKVSSDSEPFVVPDLPGDIKLTKKQLPDDVRENVENDFSKFLKASKEAELRSFGVVVNSFYELEPAYADYYKKVLGRRAWNVGPVSLCNRDTEDKAGRGKETSIDHHECLKWLDSKKPNSVVYICFGSTTNFSDSQLKEIAAGLEASGQQFIWVVRRNKKGQEDKEDWLPEGFEERMEGVGLIIRGWAPQVLILDHEAIGAFVTHCGWNSTLEGITAGKPMVTWPIFAEQFYNEKLVTDVLKTGVGVGVKEWFRVHGDHVKSEAVEKTITQIMVGEEAEEMRSRAKKLGETARKAVEEGGSSYSDFNALIEELRWRRP

SEQ ID NO: 67 - DNA, 1176 bp, Bacillus subtilis
  source 1..1176: mol_type = other DNA
atgaaaaagtaccacatctcgatgattaacatccctgcgtacggacatgtcaaccctaccttggctttggttgagaaattgtgcgaaaaagggcatcgtgtaacctatgctaccacagaagaattcgcgcccgcagttcagcaagcaggtggcgaagccttgctgtaccacacttctttgaacatcgatcctaaacaaattcgcgaaatgatggaaaagaatgacgctcccttgagtttactgaaggagtctctttcaatcctgccccagcttgaagaattatataaggatgaccaacccgaccttattatttatgacttcgttgctctggcggggaagttatttgccgagaagttaaatgtgcctgtcattaaactgtgctcgtcatatgcacagaatgagtcatttcagttaggtaacgaagatatgttgaagaagattcgtgaggcggaggcagaattcaaggcatatctggagcaggagaagcttccagccgtgagctttgagcagcttgcagtgccggaagcacttaatattgtcttcatgcctaaatcattccaaattcagcacgaaacgtttgatgaccgtttttgttttgtaggacctagcttgggtgaacgtaaagaaaaagagtcattgttaattgagaaagacgatcgtccgctgatgctgatttcactggggacggcttttaacgcatggcccgaattttacaagatgtgcatcaaggcgtttcgtgactcctcttggcaggtaattatgagtgtcggtaaggcaatcgacccagagtcacttgaagacattcctgcgaatttcacaatccgccaatcagtaccccagttggaagtgctggaaaaagctgacctttttatttcgcatgggggtatgaattcgaccatggaggcaatgaatgcaggggtacctttggtggtgatcccccagatgtacgagcaagaattgacggctaaccgtgtggatgaacttggattaggggtgtacctgccaaaggaggaagtcactgtttcatcacttcaggaagccgtacaagcagtgtccagcgaccaagaattgttgagtcgcgttaagaatatgcagaaagacgtcaaggaagcgggtggtgcagaacgtgccgccgctgagatcgaggcgtttatgaagaaatccgcagtccctcag

SEQ ID NO: 68 - protein, 392 aa, Bacillus subtilis
MKKYHISMINIPAYGHVNPTLALVEKLCEKGHRVTYATTEEFAPAVQQAGGEALLYHTSLNIDPKQIREMMEKNDAPLSLLKESLSILPQLEELYKDDQPDLIIYDFVALAGKLFAEKLNVPVIKLCSSYAQNESFQLGNEDMLKKIREAEAEFKAYLEQEKLPAVSFEQLAVPEALNIVFMPKSFQIQHETFDDRFCFVGPSLGERKEKESLLIEKDDRPLMLISLGTAFNAWPEFYKMCIKAFRDSSWQVIMSVGKAIDPESLEDIPANFTIRQSVPQLEVLEKADLFISHGGMNSTMEAMNAGVPLVVIPQMYEQELTANRVDELGLGVYLPKEEVTVSSLQEAVQAVSSDQELLSRVKNMQKDVKEAGGAERAAAEIEAFMKKSAVPQ

SEQ ID NO: 69 - DNA, 1176 bp, Bacillus subtilis
  source 1..1176: mol_type = other DNA
atgaagaagcaccacatcagtatgatcaatattccggcgtatggccatgtaaaccctacgttggcacttgtcgaaaagttatgtgaaaaaggtcatcgcgtcacgtatgcaacgaccgaagagttcgcccccgcagtccaacaggccggcggtgaggccctgatttatcacacaagtctgaacatcgatccaaagcagattcgcgagatgatggaaaagaacgacgctaccctgtccttacttaaagaatcattgagtatcctgccacagttagaggagctgtacaaagatgaccagcctgacttgattatctacgatttcgtggcattagcggggaagctgtttgcggataaactgaacgtacccgtaatcaagctttgctcctcctatgcccagaatgagtcttttcagcttgggaatgaggatatgctgaaaaagatcaaggaggccgaggccgagtttaaggcatacttggagcaagagcaattaccggctgtcagctttgaacaactggccgttcctgaagccttaaatatcgtgtttatgcccaagtcattccaaattcagcatgagacgttcgacgaccgtttttgctttgttggtccttcgctgggcaagcgcaccgaacaggaatcgctgcttatcgataaaggagaccgcccccttatgctgatttctttaggtactgcatttaacgcctggccagagttttacaagatgtgtatcgatgcgtttcgcgatagctcgtggcaagttattatgagtgtcggcaaaagcattgatcccgaatcacttgacgacacaccggcgaattttaccattcgccagagtgtgccgcaattagaagtgcttgcgaaggctgatttatttatctcacacggtggaatgaactccacgatggaggccatgaacgcgggcgtgcctcttgttgtaattccccagatgtacgagcaggaacttaccgcaaagcgtgtcgatgagcttggtttaggcgtctaccttcagcgtgaagaagtaacagtaagtaaactgcaggaagccgtccaggctgtgtctggagatcaggagcttctgagtcgtgttaaatccatgcagaaagatgtgaaggaggccggaggagcagaacgtgccgcagcagaaatcgaagcctttatgaaaaaatcagctgtcccgcag

SEQ ID NO: 70 - protein, 392 aa, Bacillus subtilis
MKKHHISMINIPAYGHVNPTLALVEKLCEKGHRVTYATTEEFAPAVQQAGGEALIYHTSLNIDPKQIREMMEKNDATLSLLKESLSILPQLEELYKDDQPDLIIYDFVALAGKLFADKLNVPVIKLCSSYAQNESFQLGNEDMLKKIKEAEAEFKAYLEQEQLPAVSFEQLAVPEALNIVFMPKSFQIQHETFDDRFCFVGPSLGKRTEQESLLIDKGDRPLMLISLGTAFNAWPEFYKMCIDAFRDSSWQVIMSVGKSIDPESLDDTPANFTIRQSVPQLEVLAKADLFISHGGMNSTMEAMNAGVPLVVIPQMYEQELTAKRVDELGLGVYLQREEVTVSKLQEAVQAVSGDQELLSRVKSMQKDVKEAGGAERAAAEIEAFMKKSAVPQ

SEQ ID NO: 71 - DNA, 1488 bp, Carica papaya
  source 1..1488: mol_type = other DNA
atgggtagcgaagttctgcatcatgattatagccagctgaacatctttttctttccgtttatggcacatggtcatatgattccgacactggatatggcaaaactgtttgcaacccatggtgcaaaaaccagcattattaccacaccgctgaatctgccgttttttagcaaaagcattgaacgctttagcaaacagacaggtctggaaattggtgtgaaactgctgaattttccgagcgttgaagttggtctgccgagcggttgtgaaaatgcagatagcctgcctgccggtgaaccgctgattgtgaataaattctttgcagcagcaggcatgctgaaagatccgctggaacgtctgctgcaagagtttaaaccggattgtctgattgccgatatgttttttccgtggaccaccgatgcagcagccaaatttgatattccgcgtctggtttttcatggcaccagcttttttgcactgagcgcaagcgaatgtattcgtctgtataccccgtttaataacgttagcagcgatagcgaaccgtttctggtgccgacactgccggatgaaattcgtctgacccgtaatcagctggcagattttgcaatgaaagaaggtgacgaaaacggtattcatcgtctgattaaagaagccaaagaaagcgagctgaaaagctatggtgttgtggtgaatagcttttatgaactggaaccggcatatgcggatcattatcgtaattttctgaaacgcaaagcctggcatattggtccggttagcctgtgtaataaaaccgttgaagataaagccgaacgtggtaaacgtgcaagcattgatgaagatgaatgtctgaaatggctgaatagcaaagcaccgaatagcgtgatttatatctgctttggtagcatggccaattttaacagcgcacagctgatggaaattgcaaccgcactggatgcaagcggtcaagaattcatttgggttgttcgtcgcgaaaaaaacgaaaacaatcaagaagattggctgccggaaggttttgaacagcgtaccgaaggtaaaggtctgattattcgtggttgggcaccgcaggttctgattctggaacatgaagcagttggtggttttgttacccattgtggttggaatagcaccctggaaggtgttaccgcaggtatgccgatggttacctggcctgttagcgcagaacagttttataacgaaaaactggttaccgaggtgctgaaaattggtctgagcgtgggtgtgaaaaaatgggttcgtagcgaaggtgattttgtgagccgtgaaaaagttgaacaggcagttcgtgaaattatggttggtagtgaagccgttgaacgtcgtatgcgtgcaaaagcaatggcagatatggcacgtgcagcagttgaaaaaggtggtagcagctataatgatctgaatgcactgctgcgtgaagttagcctgatgcgtcgtcagcagagtcagaatcagtaa

SEQ ID NO: 72 - protein, 495 aa, Carica papaya
MGSEVLHHDYSQLNIFFFPFMAHGHMIPTLDMAKLFATHGAKTSIITTPLNLPFFSKSIERFSKQTGLEIGVKLLNFPSVEVGLPSGCENADSLPAGEPLIVNKFFAAAGMLKDPLERLLQEFKPDCLIADMFFPWTTDAAAKFDIPRLVFHGTSFFALSASECIRLYTPFNNVSSDSEPFLVPTLPDEIRLTRNQLADFAMKEGDENGIHRLIKEAKESELKSYGVVVNSFYELEPAYADHYRNFLKRKAWHIGPVSLCNKTVEDKAERGKRASIDEDECLKWLNSKAPNSVIYICFGSMANFNSAQLMEIATALDASGQEFIWVVRREKNENNQEDWLPEGFEQRTEGKGLIIRGWAPQVLILEHEAVGGFVTHCGWNSTLEGVTAGMPMVTWPVSAEQFYNEKLVTEVLKIGLSVGVKKWVRSEGDFVSREKVEQAVREIMVGSEAVERRMRAKAMADMARAAVEKGGSSYNDLNALLREVSLMRRQQSQNQ

SEQ ID NO: 73 - DNA, 3615 bp, Chromobacterium violaceum
  source 1..3615: mol_type = other DNA
atgtccatcttggatttcccaagaatccattttagaggttgggctagagttaatgctccaactgctaatagagatccacatggtcatattgatatggcctctaatactgttgctatggctggtgaaccatttgatttggctagacatccaactgaattccacagacatttgagatctttgggtccaagatttggtttggatggtagagctgatccagaaggtccattttctttggctgaaggttataatgctgctggtaacaatcatttctcttgggaatctgctaccgtttctcatgttcaatgggatggtggtgaagctgatagaggtgatggtttggttggtgctagattggctttgtggggtcattacaatgattacttgagaaccactttcaacagagccagatgggttgattctgatccaactagaagagatgctgctcaaatctatgctggtcaattcactatttctccagctggtgctggtccaggtactccatggttgtttactgctgatattgatgattctcatggtgccagatggactagaggtggtcatatagcagaaagaggtggacatttcttggatgaagaatttggtttagccaggttgttccaattctctgttccaaaagatcacccacactttttgtttcatccaggtccattcgattctgaagcttggagaagattgcaattggcattggaagatgatgatgttttgggtttgactgttcaatacgccttgttcaatatgtctactccaccacaacctaactctccagtttttcatgatatggttggtgttgttggtttatggcgtagaggtgaattggcttcttacccagctggtagattattaagaccaagacaaccaggtttgggtgatttgactttgagagtttctggtggtagagttgctttgaatttggcttgtgctattccattctctactagagctgctcaaccatctgctccagatagattgactccagatttgggtgctaaattgccattaggtgatttgttgttgagggatgaagatggtgctttgttagctagagttccacaagccttgtatcaagattactggactaatcacggtatcgttgatttgccattattgagagaacctagaggttctttgaccttgtcatctgaattagctgaatggcgtgaacaagattgggttactcaatctgatgcttccaacttgtatttggaagctcctgatagaaggcatggtagatttttcccagaatctattgccttgaggtcttacttcagaggtgaagctagagctagaccagatattccacatagaattgaaggtatgggtttagtcggtgtcgaatctagacaagatggtgatgctgcagaatggcgtttgactggtttaagaccaggtccagctagaatagttttggatgatggtgcagaagctattcctttaagagttttgccagatgattgggctttagatgatgctactgttgaagaagttgattacgccttcttgtacagacatgttatggcttactacgaattggtctaccctttcatgtctgataaggttttctcattggctgatagatgtaagtgtgaaacctacgctagattgatgtggcaaatgtgtgatccacaaaaccgtaacaagtcttactacatgccatccactagagaattgtcagctccaaaagctaggttgttcttgaaatacttggcacacgttgaaggtcaagctagattgcaagctccaccaccagccggtcctgcaagaattgaatcaaaagctcaattggctgctgaattgagaaaggctgttgatttggaattgtccgtcatgttgcaatacttgtatgctgcttactccattccaaattacgctcaaggtcaacaaagggttagagatggtgcatggactgctgaacaattacaattagcttgtggttctggtgacagacgtagagatggcggtattagagcagctttgttggaaattgctcacgaagaaatgatccattacttggtcgttaacaacttgttgatggctttgggtgaacctttttatgctggtgttccattgatgggtgaagcagctagacaagctttcggtttagatactgaatttgccttggaaccattctccgaatctacattggctagattcgttagattggaatggccacattttattccagctccaggtaaatcaattgctgattgctatgctgctattagacaagcctttttggatttgcctgatttgtttggtggtgaggcaggtaagagaggtggcgaacatcatttgtttttgaacgaattgactaacagagcccatccaggttatcaattggaagttttcgatagagattccgctttgttcggtattgctttcgttactgatcaaggtgaaggtggtgcattggattctccacattatgaacattcccacttccaaagattgagggaaatgtctgctagaattatggctcaatcagctccatttgaaccagctttgccagctttaagaaatccagttttggacgaatctccaggttgtcaaagagttgctgacggtagagcaagagctttgatggcattatatcaaggtgtgtacgaactgatgtttgctatgatggctcaacattttgctgttaagccattgggttcattgagaagatccagattgatgaacgctgccattgatttgatgaccggtttgttaagaccactgtcttgtgctttgatgaacttgccatctggtattgctggtagaacagctggtccaccattgccaggtcctgttgataccagatcttatgatgattacgctttgggttgtagaatgttggctagaagatgcgaaagattgcttgaacaagcttctatgttggaaccaggttggttaccagatgctcaaatggaattattggacttctacagacgtcagatgttggatttggcatgtggtaaattgtctagagaagccggtggaggaggttctggaggcggtggaagtggtggcggaggtagcatggaaaacagagaaccaccattattgccagctagatggtcatctgcttatgtttcttattggtcaccaatgttgccagacgatcaattgacttctggttactgttggttcgattacgaaagagatatctgcagaatcgacggtttgtttaatccatggtctgaaagagacactggttacagattgtggatgtctgaagttggtaatgctgcttctggtagaacttggaaacaaaaagttgcttacggtagagaaagaaccgctttgggtgaacaattgtgtgaaagaccattggatgacgaaactggtccatttgctgaattattcttgccaagagatgtcttgagaagattgggtgctagacatattggtagaagagttgttttgggtagagaagctgatggttggagatatcaaagaccaggtaaaggtccatctaccttgtatttggatgctgcatcaggtactccattgagaatggttactggtgatgaagcttctagagcatctttgagagatttcccaaatgtttccgaagccgaaattccagatgctgtttttgctgctaagaggtga

SEQ ID NO: 74 - protein, 1204 aa, Chromobacterium violaceum
MSILDFPRIHFRGWARVNAPTANRDPHGHIDMASNTVAMAGEPFDLARHPTEFHRHLRSLGPRFGLDGRADPEGPFSLAEGYNAAGNNHFSWESATVSHVQWDGGEADRGDGLVGARLALWGHYNDYLRTTFNRARWVDSDPTRRDAAQIYAGQFTISPAGAGPGTPWLFTADIDDSHGARWTRGGHIAERGGHFLDEEFGLARLFQFSVPKDHPHFLFHPGPFDSEAWRRLQLALEDDDVLGLTVQYALFNMSTPPQPNSPVFHDMVGVVGLWRRGELASYPAGRLLRPRQPGLGDLTLRVSGGRVALNLACAIPFSTRAAQPSAPDRLTPDLGAKLPLGDLLLRDEDGALLARVPQALYQDYWTNHGIVDLPLLREPRGSLTLSSELAEWREQDWVTQSDASNLYLEAPDRRHGRFFPESIALRSYFRGEARARPDIPHRIEGMGLVGVESRQDGDAAEWRLTGLRPGPARIVLDDGAEAIPLRVLPDDWALDDATVEEVDYAFLYRHVMAYYELVYPFMSDKVFSLADRCKCETYARLMWQMCDPQNRNKSYYMPSTRELSAPKARLFLKYLAHVEGQARLQAPPPAGPARIESKAQLAAELRKAVDLELSVMLQYLYAAYSIPNYAQGQQRVRDGAWTAEQLQLACGSGDRRRDGGIRAALLEIAHEEMIHYLVVNNLLMALGEPFYAGVPLMGEAARQAFGLDTEFALEPFSESTLARFVRLEWPHFIPAPGKSIADCYAAIRQAFLDLPDLFGGEAGKRGGEHHLFLNELTNRAHPGYQLEVFDRDSALFGIAFVTDQGEGGALDSPHYEHSHFQRLREMSARIMAQSAPFEPALPALRNPVLDESPGCQRVADGRARALMALYQGVYELMFAMMAQHFAVKPLGSLRRSRLMNAAIDLMTGLLRPLSCALMNLPSGIAGRTAGPPLPGPVDTRSYDDYALGCRMLARRCERLLEQASMLEPGWLPDAQMELLDFYRRQMLDLACGKLSREAGGGGSGGGGSGGGGSMENREPPLLPARWSSAYVSYWSPMLPDDQLTSGYCWFDYERDICRIDGLFNPWSERDTGYRLWMSEVGNAASGRTWKQKVAYGRERTALGEQLCERPLDDETGPFAELFLPRDVLRRLGARHIGRRVVLGREADGWRYQRPGKGPSTLYLDAASGTPLRMVTGDEASRASLRDFPNVSEAEIPDAVFAAKR

SEQ ID NO: 75 - DNA, 3615 bp, Chromobacterium violaceum
  source 1..3615: mol_type = other DNA
atgtccatcttggatttcccaagaatccattttagaggttgggctagagttaatgctccaactgctaatagagatccacatggtcatattgatatggcctctaatactgttgctatggctggtgaaccatttgatttggctagacatccaactgaattccacagacatttgagatctttgggtccaagatttggtttggatggtagagctgatccagaaggtccattttctttggctgaaggttataatgctgctggtaacaatcatttctcttgggaatctgctaccgtttctcatgttcaatgggatggtggtgaagctgatagaggtgatggtttggttggtgctagattggctttgtggggtcattacaatgattacttgagaaccactttcaacagagccagatgggttgattctgatccaactagaagagatgctgctcaaatctatgctggtcaattcactatttctccagctggtgctggtccaggtactccatggttgtttactgctgatattgatgattctcatggtgccagatggactagaggtggtcatatagcagaaagaggtggacatttcttggatgaagaatttggtttagccaggttgttccaattctctgttccaaaagatcacccacactttttgtttcatccaggtccattcgattctgaagcttggagaagattgcaattggcattggaagatgatgatgttttgggtttgactgttcaatacgccttgttcaatatgtctactccaccacaacctaactctccagtttttcatgatatggttggtgttgttggtttatggcgtagaggtgaattggcttcttacccagctggtagattattaagaccaagacaaccaggtttgggtgatttgactttgagagtttctggtggtagagttgctttgaatttggcttgtgctattccattctctactagagctgctcaaccatctgctccagatagattgactccagatttgggtgctaaattgccattaggtgatttgttgttgagggatgaagatggtgctttgttagctagagttccacaagccttgtatcaagattactggactaatcacggtatcgttgatttgccattattgagagaacctagaggttctttgaccttgtcatctgaattagctgaatggcgtgaacaagattgggttactcaatctgatgcttccaacttgtatttggaagctcctgatagaaggcatggtagatttttcccagaatctattgccttgaggtcttacttcagaggtgaagctagagctagaccagatattccacatagaattgaaggtatgggtttagtcggtgtcgaatctagacaagatggtgatgctgcagaatggcgtttgactggtttaagaccaggtccagctagaatagttttggatgatggtgcagaagctattcctttaagagttttgccagatgattgggctttagatgatgctactgttgaagaagttgattacgccttcttgtacagacatgttatggcttactacgaattggtctaccctttcatgtctgataaggttttctcattggctgatagatgtaagtgtgaaacctacgctagattgatgtggcaaatgtgtgatccacaaaaccgtaacaagtcttactacatgccatccactagagaattgtcagctccaaaagctaggttgttcttgaaatacttggcacacgttgaaggtcaagctagattgcaagctccaccaccagccggtcctgcaagaattgaatcaaaagctcaattggctgctgaattgagaaaggctgttgatttggaattgtccgtcatgttgcaatacttgtatgctgcttactccattccaaattacgctcaaggtcaacaaagggttagagatggtgcatggactgctgaacaattacaattagcttgtggttctggtgacagacgtagagatggcggtattagagcagctttgttggaaattgctcacgaagaaatgatccattacttggtcgttaacaacttgttgatggctttgggtgaacctttttatgctggtgttccattgatgggtgaagcagctagacaagctttcggtttagatactgaatttgccttggaaccattctccgaatctacattggctagattcgttagattggaatggccacattttattccagctccaggtaaatcaattgctgattgctatgctgctattagacaagcctttttggatttgcctgatttgtttggtggtgaggcaggtaagagaggtggcgaacatcatttgtttttgaacgaattgactaacagagcccatccaggttatcaattggaagttttcgatagagattccgctttgttcggtattgctttcgttactgatcaaggtgaaggtggtgcattggattctccacattatgaacattcccacttccaaagattgagggaaatgtctgctagaattatggctcaatcagctccatttgaaccagctttgccagctttaagaaatccagttttggacgaatctccaggttgtcaaagagttgctgacggtagagcaagagctttgatggcattatatcaaggtgtgtacgaactgatgtttgctatgatggctcaacattttgctgttaagccattgggttcattgagaagatccagattgatgaacgctgccattgatttgatgaccggtttgttaagaccactgtcttgtgctttgatgaacttgccatctggtattgctggtagaacagctggtccaccattgccaggtcctgttgataccagatcttatgatgattacgctttgggttgtagaatgttggctagaagatgcgaaagattgcttgaacaagcttctatgttggaaccaggttggttaccagatgctcaaatggaattattggacttctacagacgtcagatgttggatttggcatgtggtaaattgtctagagaagccgaggctgctgcgaaggaagcggcggcgaaagaggcggcggcgaagatggaaaacagagaaccaccattattgccagctagatggtcatctgcttatgtttcttattggtcaccaatgttgccagacgatcaattgacttctggttactgttggttcgattacgaaagagatatctgcagaatcgacggtttgtttaatccatggtctgaaagagacactggttacagattgtggatgtctgaagttggtaatgctgcttctggtagaacttggaaacaaaaagttgcttacggtagagaaagaaccgctttgggtgaacaattgtgtgaaagaccattggatgacgaaactggtccatttgctgaattattcttgccaagagatgtcttgagaagattgggtgctagacatattggtagaagagttgttttgggtagagaagctgatggttggagatatcaaagaccaggtaaaggtccatctaccttgtatttggatgctgcatcaggtactccattgagaatggttactggtgatgaagcttctagagcatctttgagagatttcccaaatgtttccgaagccgaaattccagatgctgtttttgctgctaagaggtga

SEQ ID NO: 76 - protein, 1204 aa, Chromobacterium violaceum
MSILDFPRIHFRGWARVNAPTANRDPHGHIDMASNTVAMAGEPFDLARHPTEFHRHLRSLGPRFGLDGRADPEGPFSLAEGYNAAGNNHFSWESATVSHVQWDGGEADRGDGLVGARLALWGHYNDYLRTTFNRARWVDSDPTRRDAAQIYAGQFTISPAGAGPGTPWLFTADIDDSHGARWTRGGHIAERGGHFLDEEFGLARLFQFSVPKDHPHFLFHPGPFDSEAWRRLQLALEDDDVLGLTVQYALFNMSTPPQPNSPVFHDMVGVVGLWRRGELASYPAGRLLRPRQPGLGDLTLRVSGGRVALNLACAIPFSTRAAQPSAPDRLTPDLGAKLPLGDLLLRDEDGALLARVPQALYQDYWTNHGIVDLPLLREPRGSLTLSSELAEWREQDWVTQSDASNLYLEAPDRRHGRFFPESIALRSYFRGEARARPDIPHRIEGMGLVGVESRQDGDAAEWRLTGLRPGPARIVLDDGAEAIPLRVLPDDWALDDATVEEVDYAFLYRHVMAYYELVYPFMSDKVFSLADRCKCETYARLMWQMCDPQNRNKSYYMPSTRELSAPKARLFLKYLAHVEGQARLQAPPPAGPARIESKAQLAAELRKAVDLELSVMLQYLYAAYSIPNYAQGQQRVRDGAWTAEQLQLACGSGDRRRDGGIRAALLEIAHEEMIHYLVVNNLLMALGEPFYAGVPLMGEAARQAFGLDTEFALEPFSESTLARFVRLEWPHFIPAPGKSIADCYAAIRQAFLDLPDLFGGEAGKRGGEHHLFLNELTNRAHPGYQLEVFDRDSALFGIAFVTDQGEGGALDSPHYEHSHFQRLREMSARIMAQSAPFEPALPALRNPVLDESPGCQRVADGRARALMALYQGVYELMFAMMAQHFAVKPLGSLRRSRLMNAAIDLMTGLLRPLSCALMNLPSGIAGRTAGPPLPGPVDTRSYDDYALGCRMLARRCERLLEQASMLEPGWLPDAQMELLDFYRRQMLDLACGKLSREAEAAAKEAAAKEAAAKMENREPPLLPARWSSAYVSYWSPMLPDDQLTSGYCWFDYERDICRIDGLFNPWSERDTGYRLWMSEVGNAASGRTWKQKVAYGRERTALGEQLCERPLDDETGPFAELFLPRDVLRRLGARHIGRRVVLGREADGWRYQRPGKGPSTLYLDAASGTPLRMVTGDEASRASLRDFPNVSEAEIPDAVFAAKR

SEQ ID NO: 77 - DNA, 3042 bp, Lentzea aerocolonigenes
  source 1..3042: mol_type = other DNA
atgtccgttttcgatttgccaagattgcattttgctggtactgctactactagattgccaactggtccaagaaatggtttggttgatttgtctacccactccgttgttatggatggtgaaagatttccagcttctagaccagctgctgaatatcatgcttatttggatagagttggtggtaagggtactgctttcgctggtaatggttattttgctattgatgctggtattaccgctgttgaaagagctgctggcgaagttgatactggtgatttgttggttggtagagctgttgatgtttggggtcattacaatgaatacttggctactaccttcaacagagccagaatttttgatgttgacccatcttcttcatggacctctactgttatgattggtcaatttggtttcggtaggttgggtagatctcatgatgttggttatgttttcactggtggtgttcatggtatgcaaccaccaagatggcatgaagatggtagagttttacatcaattcactgttccagctggtgaagatatgacatggttcggttctgctgctgattctcctgctgctgctagattgagagaattggttgaatctggtgaagccgatggtttagttgttcaattggctttgtcagatgctggtccagctccaatgccacatgctcaacaatggcgtttgagaggtactattgctccatggcatgctggtgaacctagaacttgtccagcaggtagattattgactccacataatttgaccgctgacttgagaggtgatcatgtttctttgaacttgatctcttttagaccaccaaccggtatttctggtttggaattgagaactgctgataccgatagattcattgctagagttccagcagatgatccacatggtgttgttactgtaccagcagctgaaggtggtgatgaagctttgtgtgttgttggtactactgctgccggtgaaagaatagttgtttctagagaaagggaagttaccgttcatgttgatgatgcttcagttttcttggaacatccaagaggtccaggtgattctgatcaagatgctgaaattgctgttagaacctacgttagaggtgaaccagctgccgctactattcatataggtcaatactttaacccaagggctttcccattggatgaacatgctacagctgcttcagctactcctgaagatttggatgttgttgctttatgcgttgatggtacaagatggtctagacattgtgttatttctaccgacgaaaacggtgatggtagattcttgttaagaggtgctagaccaggtgcaactagattactattgtctgcagaaggtgctactccatttgatggtttgactgcagctgctgcttatgataacgatgattctttaggtttgtggtccggtttggcttctgttgcagttagagttttgccagatcattggtggatggatgatattccaagagataaggttaccttcgacttgttgtacagagaagtttttgctttctacgagctgctgtactcttttatgggtgaagaagtattctctttggccgacagattcagagttgaaactcatcctagattgatttggcaaatgtgcgatcctagaaatagagctaagacttattacatgccaccaaccagagatttgactggtccacaagctagattgcttttggcttatttgagagcccaaaactctgatgttgtcgttccagttattgaaccatctcatacaagatccggtactccaatttctactagaaccgatttggttagagctttgagacacggtgttgcaattgaattggctgttatgttgcaatacttgtacgctgctttctccattccaactcatggtgctggtcaagaattagtttcaagaggtgattggactccagaacaattgagattgatgtgtggtgatggtggtgaaactactgacggtggtgttagaggttctttgttgggtgttgctagagaagaaatgatccacttcttggttgtcaacaacgttttgatggccgttggtgaaccatttcatgttccagatttggacttcggtactatcaacgatactttgatggtgccattggacttttcattggaagccttaggtttaggttccgtccaaagattcattcaaatcgaacaacctgaaggtttgacaggtgccgttagattgggtgatttgccagttcctgttagagaagccgaagattttcattacgcttcattgtccgaactgtacggtgatattagagaaggtttacaaagagtcccaggtttgtttttggttgaacgtggtagaggtggtggtgagcatcatttgtttttaagagaatccgttaacgccgttcatccagattatcaattggaagttgatgacttgtcctctgctttgttcgctattgatttcgttactgaacaaggtgaaggtcacgttttgactgatgaagatacaggtgaagaatcccattacgatacctttgttagagttgccgatctgttgatgaaggaaagattgacagctgcagatacaagacgtgctcaatggtcaccagcttatccagttgctagaaatccaacagttcacggtggtggtcaatctaaagaattggtaacttctccagttgccagggaattgatggtcttgtttaacaagtcttacttcatgatgctgcagctgatggtccaacattttggtggttctccagatgcttctttgagaagatctaaattgatgaacgccgccattgatgttatgactggtgttatgaggccattggctgaattattggttacagttccatctggtagacatggtagaacagctggtccatcttttgaattggacgaaaagccagcttttatcccaagagctgatgttgcaagaagggctatttctttgaggtttagacatttggcagaatctgctagaacatgtgctttggttccagacaaggttgttagaaacttagatttcttggctgatcaattcgctactgaaggtccaagataa

SEQ ID NO: 78 - protein, 1013 aa, Lentzea aerocolonigenes
MSVFDLPRLHFAGTATTRLPTGPRNGLVDLSTHSVVMDGERFPASRPAAEYHAYLDRVGGKGTAFAGNGYFAIDAGITAVERAAGEVDTGDLLVGRAVDVWGHYNEYLATTFNRARIFDVDPSSSWTSTVMIGQFGFGRLGRSHDVGYVFTGGVHGMQPPRWHEDGRVLHQFTVPAGEDMTWFGSAADSPAAARLRELVESGEADGLVVQLALSDAGPAPMPHAQQWRLRGTIAPWHAGEPRTCPAGRLLTPHNLTADLRGDHVSLNLISFRPPTGISGLELRTADTDRFIARVPADDPHGVVTVPAAEGGDEALCVVGTTAAGERIVVSREREVTVHVDDASVFLEHPRGPGDSDQDAEIAVRTYVRGEPAAATIHIGQYFNPRAFPLDEHATAASATPEDLDVVALCVDGTRWSRHCVISTDENGDGRFLLRGARPGATRLLLSAEGATPFDGLTAAAAYDNDDSLGLWSGLASVAVRVLPDHWWMDDIPRDKVTFDLLYREVFAFYELLYSFMGEEVFSLADRFRVETHPRLIWQMCDPRNRAKTYYMPPTRDLTGPQARLLLAYLRAQNSDVVVPVIEPSHTRSGTPISTRTDLVRALRHGVAIELAVMLQYLYAAFSIPTHGAGQELVSRGDWTPEQLRLMCGDGGETTDGGVRGSLLGVAREEMIHFLVVNNVLMAVGEPFHVPDLDFGTINDTLMVPLDFSLEALGLGSVQRFIQIEQPEGLTGAVRLGDLPVPVREAEDFHYASLSELYGDIREGLQRVPGLFLVERGRGGGEHHLFLRESVNAVHPDYQLEVDDLSSALFAIDFVTEQGEGHVLTDEDTGEESHYDTFVRVADLLMKERLTAADTRRAQWSPAYPVARNPTVHGGGQSKELVTSPVARELMVLFNKSYFMMLQLMVQHFGGSPDASLRRSKLMNAAIDVMTGVMRPLAELLVTVPSGRHGRTAGPSFELDEKPAFIPRADVARRAISLRFRHLAESARTCALVPDKVVRNLDFLADQFATEGPR

SEQ ID NO: 79 - DNA, 1515 bp, Streptomyces sp. TP-A0274
  source 1..1515: mol_type = other DNA
atgactgctccattgcaagattctgatggtccagatgatgctattggtggtccaaaacaagttactgttattggtgctggtattgctggtttggttactgcttatgaattggaaagattgggtcaccacgttcaaattatcgaaggttctgatgatatcggtggtagaattcatacccatagattttcaggtgctggtggtcctggtccatttgctgaaatgggtgctatgagaattccagctggtcatagattgaccatgcattacattgcagaattgggcttgcaaaaccaggttagagaattcagaactttgttctctgatgatgctgcttacttgccatcttctgctggttatttgagagttagagaagctcatgataccttggttgatgaatttgctactggtttgccatctgctcattacagacaagatactttgttgtttggtgcttggttggatgcttccattagagctattgctccaagacaattttacgatggcttgcataacgatattggtgtcgagttgttgaacttggttgacgatattgatttgaccccatacagatgtggtactgccagaaatagaattgacttgcatgctttgtttgccgatcatccaagagtaagagcttcatgtccaccaagattagaaagattcttggacgatgttctggacgaaacttcttcatctatcgttagattgaaggacggtatggatgaattgccaagaagattggcttctagaatcagaggtaagatctctttgggtcaagaggttactggtatcgatgttcatgatgatactgttaccttgaccgttagacaaggtttgagaactgttactagaacctgtgattacgttgtctgtactattccattcaccgttttgagaaccttgagattgactggtttcgatcaagataagttggatatcgtccacgaaactaagtattggccagctactaagattgctttccattgcagagaaccattctgggaaaaagatggtatttctggtggtgcttcttttactggtggtcatgttagacaaacttattacccaccagctgaaggtgatccagctttgggtgctgttttgttggcttcttatacaattggtcctgatgctgaagctttggctagaatggatgaagctgaaagagatgctttggtcgctaaagaattgtctgttatgcatccagaattgagaaggccaggtatggttttggctgttgctggtagagattggggtgctagaagatggtctagaggtgctgctactgttagatggggacaagaagctgctttgagagaagctgagagaagagaatgtgctagaccacaaaaaggtttgtttttcgctggtgaacactgttcttctaaaccagcttggattgaaggtgctattgaatctgctattgatgctgcacacgaaattgaatggtatgaacctagagcttctagagtttttgctgcctctagattgtctagatctgatagatcagcttaa

SEQ ID NO: 80 - protein, 504 aa, Streptomyces sp. TP-A0274
MTAPLQDSDGPDDAIGGPKQVTVIGAGIAGLVTAYELERLGHHVQIIEGSDDIGGRIHTHRFSGAGGPGPFAEMGAMRIPAGHRLTMHYIAELGLQNQVREFRTLFSDDAAYLPSSAGYLRVREAHDTLVDEFATGLPSAHYRQDTLLFGAWLDASIRAIAPRQFYDGLHNDIGVELLNLVDDIDLTPYRCGTARNRIDLHALFADHPRVRASCPPRLERFLDDVLDETSSSIVRLKDGMDELPRRLASRIRGKISLGQEVTGIDVHDDTVTLTVRQGLRTVTRTCDYVVCTIPFTVLRTLRLTGFDQDKLDIVHETKYWPATKIAFHCREPFWEKDGISGGASFTGGHVRQTYYPPAEGDPALGAVLLASYTIGPDAEALARMDEAERDALVAKELSVMHPELRRPGMVLAVAGRDWGARRWSRGAATVRWGQEAALREAERRECARPQKGLFFAGEHCSSKPAWIEGAIESAIDAAHEIEWYEPRASRVFAASRLSRSDRSA

SEQ ID NO: 81 - DNA, 3291 bp, Streptomyces sp. TP-A0274
  source 1..3291: mol_type = other DNA
atgtccgttttcgatttgccaagattgcattttgctggtactgctactactcatttgccaactggtgctagatctggtttggttgatttggctactaatactgctttgactgctgatggtagaccatttccagctcatagaccaccagctgaatatcatgaacatttggatagattgggtccaagatacgatacaacaggtagaccagctccagatggtccttttactgaagctaaaggtgaagatttcgctggtaatggtcatttcgctgttgatgctagaatagttggtgttgaatgtagagctggtgaattggatactgctgatccagttgttggtagatctgttgatttgtggggtcattacaatgaatacttggctaccactgttaatagagccagagtttttgatttggatccagcttctgaatggactactactttgatggttggtcaatttggtttcggtagagatggtagatcacatgatgttggttatatgttgaccggtactgttcatggttttcaaccaccaagatggcataatccaggtcatgttagagatttgggtgaagctgaaggtcattggttggctgaaagattgagaagatcagttgttcatcaattcgtcgcctctgaagatgatgaattgacttggtttgatggtactgatgcttctccagctgttagaagattaagagatactgttgctactgaaggtgttggtggtttagttgttcaattcgctttgtctcatagagctgctccagaagctcctgatagaccatctagattcagattgagaggtactattgctccatggttgccacacgaaactagaacttatccagctggtagattattgactccaggtccaggtcctcatcatagaactgttagaggtttacacaacttgaccgttagattggctgatgatcatgctaccttcaacatgattactgcattgccaacatctgatagaactactggtgacggtagaactggtgttgctccattggacttgggtgatttggaattgagaactgcagatactgataggttggttgctagattgccaagacatgcttacttaggtgatgcttatgctttggctggtggtttggtaactgttccagttgaaatgccagctcatgaagctgcacatgaagctttgagattattaggttctggtcctgatggtcctgttgttcatttgagagaaaaagaaatcaacgtccaagttgatgatgcctccttggttttggaacatccaagagatacagctgatgctgaacacgatgttgaagttttggttagatctttcgttagaggtagacctgctgctgttgacgaaattggtgttagacaatttttcaacccaagagccttgccaagagatccagtagcttctgctcctgaagcaagatcttcagatttggatattgtcagagttagagccggtagaagagatgatgatggtccatggtcatctggttgtttgttgtctactgatgaagatggaagaggttggttaactttgagaggtgctactgcaggtacagctagagttttgttatctacaggtccagatgatatgccatgtgatccagatgctccaggttctgctgctttgggtcatgatcaagatgatactttaggttattggccaggtgctggtcatttgtcagttagagttttaccagatgattggagattagctggtgtcggtgaagatgaagttacattcgatttggtttacagggaagttttcgcctactatgaacacgttcattctttcatgaaggccgaggtttttagtttggccgatagatgtaaagttgaaacctacgccaagctaatctggcaaatgtgtgatccaagaaacaaggctaagacttattacatgccaccaaccagagaattgtcagctccaaaagctagactgttgttgacatttttgagagcccaacaaaaacctgatgccgttgttttgactacaccagcagttggtagaggtggaagaagaattactacaagaggtagattggttgaagtcttgcatgatgctgctgctattgaattggctgttatgttgcaatacttgtacgctgcttattccgttccaactcatggtgctggtgctgaatacgttagaagaggtttgtggactccagatcaattgagattggcttgtggtgatggtggtgaaactttggatgaaggtattaggtctatgttgctgaccattgctagagaagaaatgatccacttcttgctggttaacaacattttgatggctgttggtgaaccatttcatgccccaagaattgatttcggtactgtcaatcaaagattggccgttccattggattttgccttggaaagattaggtcctggttctgttgaaagattcgttcaaattgaaagaccagaggatttggtcgacgaagtgagaagaggtgatgccccagctccaccagcagcttatgacgaaagacatccatatgctagcttgtccgaattatacgccgatattagagaaggtttggaatccattccaggtttgttcttggttgacaaaggtagaggcggtggtgaacatcatttgttcttaagagaatccttgaacagaaggcacccagattatcaattggaagttgacgatttgtcctctgctttgttcgccattgatgttattactgaacaaggtgaaggtggtgttttagatccaagagctgacgaaacttctcattaccatgctttcttgagaatctccgatttgttaagaggtgctccaactattggtggtaggccaggtggtgagagatggaatccagcttacccagttgttagaaatccaactttaggtcatggtaacccagctatggaaactgttactgatcctgatgcaagagccgttatggaattatttgatagagcttacttcatggccttgcaattgatggctcaacatttttgtgaatgcccagatgcttctttgagaaggtctgatttgatgaacggtgccattgatatgatgactggtttaatgaggccattggccgaattattggttactttgccatctggtagaaggggtatgactgctggtccatcttttgaattgccagaacaaccagctcctgtttctagaccagatgttgcaagaagaggtattgctagaagattggatgacttggctgctcaatgtgctaaacatccattggttccaccaagagttgctgaaatgtctactttttgggctgatcattttaggccaccttctagataa

SEQ ID NO: 82 - protein, 1096 aa, Streptomyces sp. TP-A0274
MSVFDLPRLHFAGTATTHLPTGARSGLVDLATNTALTADGRPFPAHRPPAEYHEHLDRLGPRYDTTGRPAPDGPFTEAKGEDFAGNGHFAVDARIVGVECRAGELDTADPVVGRSVDLWGHYNEYLATTVNRARVFDLDPASEWTTTLMVGQFGFGRDGRSHDVGYMLTGTVHGFQPPRWHNPGHVRDLGEAEGHWLAERLRRSVVHQFVASEDDELTWFDGTDASPAVRRLRDTVATEGVGGLVVQFALSHRAAPEAPDRPSRFRLRGTIAPWLPHETRTYPAGRLLTPGPGPHHRTVRGLHNLTVRLADDHATFNMITALPTSDRTTGDGRTGVAPLDLGDLELRTADTDRLVARLPRHAYLGDAYALAGGLVTVPVEMPAHEAAHEALRLLGSGPDGPVVHLREKEINVQVDDASLVLEHPRDTADAEHDVEVLVRSFVRGRPAAVDEIGVRQFFNPRALPRDPVASAPEARSSDLDIVRVRAGRRDDDGPWSSGCLLSTDEDGRGWLTLRGATAGTARVLLSTGPDDMPCDPDAPGSAALGHDQDDTLGYWPGAGHLSVRVLPDDWRLAGVGEDEVTFDLVYREVFAYYEHVHSFMKAEVFSLADRCKVETYAKLIWQMCDPRNKAKTYYMPPTRELSAPKARLLLTFLRAQQKPDAVVLTTPAVGRGGRRITTRGRLVEVLHDAAAIELAVMLQYLYAAYSVPTHGAGAEYVRRGLWTPDQLRLACGDGGETLDEGIRSMLLTIAREEMIHFLLVNNILMAVGEPFHAPRIDFGTVNQRLAVPLDFALERLGPGSVERFVQIERPEDLVDEVRRGDAPAPPAAYDERHPYASLSELYADIREGLESIPGLFLVDKGRGGGEHHLFLRESLNRRHPDYQLEVDDLSSALFAIDVITEQGEGGVLDPRADETSHYHAFLRISDLLRGAPTIGGRPGGERWNPAYPVVRNPTLGHGNPAMETVTDPDARAVMELFDRAYFMALQLMAQHFCECPDASLRRSDLMNGAIDMMTGLMRPLAELLVTLPSGRRGMTAGPSFELPEQPAPVSRPDVARRGIARRLDDLAAQCAKHPLVPPRVAEMSTFWADHFRPPSR

SEQ ID NO: 83 - DNA, 1461 bp, Nonomuraea longicatena
  source 1..1461: mol_type = other DNA
atgttctccagattgccaaacgttagagaacctagaagagttactgttttaggtgctggtgttgctggtttggttgctgcttatgaattggaaagattgggtcaccaagtcgaaattattgaagctgctgatagagttggtggtagagttcatactcatagatttggttctgctccaggtgctccatttgctgatttgggtgctatgagattgagaactgatcatacaagaaccttgcactacgttgttgaattgggcttgcataacgatatcagagaattcagaactttgttcgccgatgatggtaacttgttgtctattcatgacgaaagacacatcagagttagagaagctccagatgttttgactggtagattggctgctagattgggtgatcattcttatagaccagctactttgttgtttggtgcttggttacatgcttgcttggaagctattgctccaagagattttaatgattggccagaagttactaccgagttgttggatttggttgatggtattgatttggagccatacttgcatccagctggttctaaaccagacttgtatgctgttgttaaggatcacccacaaattagatctggtccttttagaggtagagaaaggttgttggacgatgttttggacgaaacttctccagcattatacagattgagaggtggtatggaaactttgactaatgctttggctgcaagaattcaaggtccaatctggttgaatcaagaagttacaggtattgccgttcatgatgatggtgttgcagttggtgttagaagaggtggaagaattagatacagaaactacgatcatgtcctgtgcactattccattcactgttttgagaggtttgagattggatggtttcgatgctgataagttagctgctgttcacgaaactcaatattggccagctacaaagattgctttgagatgtagagaaccattttgggctgctgatggtatagctggtggtgcttcttttactggtggtttagttagacaaacttactacccaccagttgaaggtgatccagctcaaggtgctgttttgattgcttcttacactattggtccagatgctgaagctttgggtagattagatccagctgctagaaggcaagttgttttggatgaagttgctagaatgcatcctggtttgagagaaagaggtatgattttggatactgctggtagagcttggggtgaacatagatggtcattgggtgctgctactattagatggggtcaagatgctggtactagaaaagaacaacaatgggctgcagctagaccacaaggtagattattctttgctggtgaacactgttcttctatgccagcttggattgaaggtgctattgaatctgttactgatgccttgagagatatggaaacaagagatccacatgagttgatgagattagatttgggcagataa

SEQ ID NO: 84 - protein, 486 aa, Nonomuraea longicatena
MFSRLPNVREPRRVTVLGAGVAGLVAAYELERLGHQVEIIEAADRVGGRVHTHRFGSAPGAPFADLGAMRLRTDHTRTLHYVVELGLHNDIREFRTLFADDGNLLSIHDERHIRVREAPDVLTGRLAARLGDHSYRPATLLFGAWLHACLEAIAPRDFNDWPEVTTELLDLVDGIDLEPYLHPAGSKPDLYAVVKDHPQIRSGPFRGRERLLDDVLDETSPALYRLRGGMETLTNALAARIQGPIWLNQEVTGIAVHDDGVAVGVRRGGRIRYRNYDHVLCTIPFTVLRGLRLDGFDADKLAAVHETQYWPATKIALRCREPFWAADGIAGGASFTGGLVRQTYYPPVEGDPAQGAVLIASYTIGPDAEALGRLDPAARRQVVLDEVARMHPGLRERGMILDTAGRAWGEHRWSLGAATIRWGQDAGTRKEQQWAAARPQGRLFFAGEHCSSMPAWIEGAIESVTDALRDMETRDPHELMRLDLGR

SEQ ID NO: 85 - DNA, 3096 bp, Nonomuraea longicatena
  source 1..3096: mol_type = other DNA
atgtccgttttctcattgccaagattgcattttgctggtttggctactactcaattgccaactggtccaagacaaggtttgactgatttggcaactaatggtgctttgtgtgctgatggtactccatttccaccaggtagaccaccagctgaatatgatggtcctagaggtgaacatttcgctggtaatggtcattttgccgttccatctgctgctattactggtgttgaattgagagctggtgaagttgatactgctgatccagttgttggtagacaagttgatttgtggggtcattacaacgaatacttgcaaactaccgttaacagaggtagagttttcgatgttgatccagcttctaattggactaccactgttatggttggtcaatttggttttggtagagctggtagatctcatgatactggttatgctttggttggtgatgttcatggttatcaaccaccaagatggcaagatttgagaagaggtactgttgttcatcaattcgctgttgccagagaagaaattgcttggccagctccagttgctggttctgaagctgttgctgctttagctagagctttggctggttcagatggtttggttgtacaatttgctttgttggctccagacggtccaagattaccagatgctccaagatcatgggaacttagaggtactattgctccatggtggcgtgctgaacctagaacttttccagccggtagattgatgttgccagttgaaccaggtgttagagtcttttctgttgatgttggtgctgaatacgttaccttgaatttggttactgctgctgcttctttgccaccaggtgaagtagaattgagaactgctagaactggtaaattggttgctgctgttcctgttagatcttgtagagaatcagctagacatggtggtttagtttgtgctactagattgggtgaagctgaagatgaagctttggttttagttgctgctagagatggtagagtattggctagagaacaagaagttgttctgcaagctgatgatgcatgtcaagttttggatcatccaagacatgctggtgatgattcttgtgatgctgaagttggtttcagatctttcgttagaggtagacctgctcctgttgctgttgttagagttagacaatttccaaatccaagagctttgccattagctccagatgcagctactgctgaaatagttcatgttgctgatcctgaaagaacaggtccagaatctacagatactttgtctactgataccttgtccacagacacattatccaccgataccttatctattgatccaccatccattgataccgtttctactgatgctggtggtttcggtagattaactttgagaggtttaagaccaggttctaccagattgctattaggtggtgatgctacaggtgttcattacgataacgatgacgaattaggtttttgggctggtgccggtggtgttcacgttagagttttgccagatgattggagattggctgatttgagagatgatgaagtcactttcgaaaccgtttacagagaagtttttgctccatacgaagcccaatactcattcatgagagaagaagtgttttccttggctgatagatgtaaggttgaaacctacgcaaaattggtttggcaaatgtgtgatccaagaaacaaagctactacctattacatgccaccaactagagatttgactgctccaaaagctgctctgttgttgagatacttaagaagagttcaaagaccagaccacgttttgttaagagctgaaactccagctgcaagacctggtttagctacaagagcagaattagttgcagctttaagagatgccgttaagttggaattggccgttatgattcaatacttgtacgctgcttataccgttccagatcatggtgctggtagagaatatgttagaagaggtgattggactccagaacaattggctttggcttgtggtgatggtggtgaaacattggctggtggtatcagaggtactttgattagagtagctagggaagaaatgatccacttcttgctggctaacaacattttgatggctattggtgaaccattccatttgccacatttggatttcggtacattgaatgccgaattgccaattgccttggacttttcattggaaagattggatgtagcctctgttgctagattcgttagaatagaagaacctgacggtttggttacaccagttagattaggtactagaacttctactggtacttctacttctgctgatgcagatgctgacgctgatggtgacggtggcgaaccaggttacgcttctatttctgaattatacggtgatatcagaagggccttgactactattccaggtttgtttttgactgaacctggtagaggtggtggtgagcatcatttgtttttaagaaagagagtcaacgaccaccatcctgattatcaattggaagttgatgacttggcttctgctttgttcgccattgatgttattactgaacaaggtgaaggcggtgaattgaccgatggtgatgaaccttctgaagaatcacatcatgctgcatttttgggtgttgcagaagccttgagaagatctgattgggttccatcttatccagctgctagaaatccaactttaagaaggggtgatagagccagacagtatattccagatcctgatgctagaagagttatgaggttgtttaatgctgcttacgctgttgcattgcaattgatggttcaacatttcggtgctggtccagatacttctttgagaaggtctaagttaatgaacgctgctatcgatgttatgactggtgttttaagacctttgggtgagttgttaactactatgccatctggtttaagaggtagaacagctggtccatcttttgaattggatgctccaccagcttttattgctagaccagatgttgctgcattgagagttgctagacaattggatgatgttgcagctgctgctagtgcttgtccaccagttccagctagagttgctgaaatttctactttgttagccgaacaattgaggggttctgttagatga

SEQ ID NO: 86 - protein, 1031 aa, Nonomuraea longicatena
MSVFSLPRLHFAGLATTQLPTGPRQGLTDLATNGALCADGTPFPPGRPPAEYDGPRGEHFAGNGHFAVPSAAITGVELRAGEVDTADPVVGRQVDLWGHYNEYLQTTVNRGRVFDVDPASNWTTTVMVGQFGFGRAGRSHDTGYALVGDVHGYQPPRWQDLRRGTVVHQFAVAREEIAWPAPVAGSEAVAALARALAGSDGLVVQFALLAPDGPRLPDAPRSWELRGTIAPWWRAEPRTFPAGRLMLPVEPGVRVFSVDVGAEYVTLNLVTAAASLPPGEVELRTARTGKLVAAVPVRSCRESARHGGLVCATRLGEAEDEALVLVAARDGRVLAREQEVVLQADDACQVLDHPRHAGDDSCDAEVGFRSFVRGRPAPVAVVRVRQFPNPRALPLAPDAATAEIVHVADPERTGPESTDTLSTDTLSTDTLSTDTLSIDPPSIDTVSTDAGGFGRLTLRGLRPGSTRLLLGGDATGVHYDNDDELGFWAGAGGVHVRVLPDDWRLADLRDDEVTFETVYREVFAPYEAQYSFMREEVFSLADRCKVETYAKLVWQMCDPRNKATTYYMPPTRDLTAPKAALLLRYLRRVQRPDHVLLRAETPAARPGLATRAELVAALRDAVKLELAVMIQYLYAAYTVPDHGAGREYVRRGDWTPEQLALACGDGGETLAGGIRGTLIRVAREEMIHFLLANNILMAIGEPFHLPHLDFGTLNAELPIALDFSLERLDVASVARFVRIEEPDGLVTPVRLGTRTSTGTSTSADADADADGDGGEPGYASISELYGDIRRALTTIPGLFLTEPGRGGGEHHLFLRKRVNDHHPDYQLEVDDLASALFAIDVITEQGEGGELTDGDEPSEESHHAAFLGVAEALRRSDWVPSYPAARNPTLRRGDRARQYIPDPDARRVMRLFNAAYAVALQLMVQHFGAGPDTSLRRSKLMNAAIDVMTGVLRPLGELLTTMPSGLRGRTAGPSFELDAPPAFIARPDVAALRVARQLDDVAAAASACPPVPARVAEISTLLAEQLRGSVR

SEQ ID NO: 87 - DNA, 1437 bp, Actinomadura melliaura
  source 1..1437: mol_type = other DNA
atgtccaacagaatcactgttttaggtgctggtattgctggtttggttgctgcttgggaattagaacaattgggttactctgttgaagtcttggaaggttcttctagatgtggtggtagaatctacactcatagatttgctgatgctcaaccagctccattggttgaattgggtgctatgagaattccaactaatcacttgcataccagagcctacattgataagttgggtttgaacgatagagtcagagatttcagaaccttgttctctgaagatggtgcttaccatacaacatctgctggttttgttagagttggtgaagctgctcaagttttggttgaagaattcagaacaggtatggttgctgaaagatatggtgaacctactattttgtttggtgcttggttgatggctatggctgatgctattgctccaccagattttagaatcaccttgcagaaagaattgggcagagatttgttggatgaattggaaggtctagatttggctccattcagatatggtagaggtagagatagaattgacttgcatgctttctttgctgctcatccacaagttagaactacttggaatggtagattgaaccacttcatcgacgatgttttgaacgaaacttccccagaattgattagactagaaggtggtatggatcaattggttaccggtttagttgaaagaatcagaggtccaattacctacggtcaagaggttgttggtattcacgttagagataacgatgtcttgttggagattagagatggtgccggtgttactgttagaagatgtgatcaagttctgtgtaccgttccattctccattttgagaagaatgtctttgtctggtttcgacggtgacaaattggatgttgttcatgctgttaagtactggtctgctactaaggttgcttttcattgcagagaagctttttgggaacatgcaggtatttctggtggtgcttcttattctggtggaagaattagacaaacttactacccagctgctgaaggtgttccaggtggtggtgcagttttgttggcttcttatacaatgggtgatgatgctgatagattgggtagaatgccaccagctgcttgtagatctgttgttttagatgagttgtccaagatgcatcctgaattgagaaggcctggtatggttatggattctgcttctttggcttggggtcaacatagatggtctgaaggtgctggcgttactagatggggtatgtctgttgctgcatctgaagaacaaagaagtcaagctgctagaccacaaggtagattattcttcgctggtgaacactgttcttctactccagcatggattgatggtgctattgaatctgctttggaagctgttggtcaagtcagattaggtactagagaaggtgcagttgctgttggtgctagataa

SEQ ID NO: 88 - protein, 478 aa, Actinomadura melliaura
MSNRITVLGAGIAGLVAAWELEQLGYSVEVLEGSSRCGGRIYTHRFADAQPAPLVELGAMRIPTNHLHTRAYIDKLGLNDRVRDFRTLFSEDGAYHTTSAGFVRVGEAAQVLVEEFRTGMVAERYGEPTILFGAWLMAMADAIAPPDFRITLQKELGRDLLDELEGLDLAPFRYGRGRDRIDLHAFFAAHPQVRTTWNGRLNHFIDDVLNETSPELIRLEGGMDQLVTGLVERIRGPITYGQEVVGIHVRDNDVLLEIRDGAGVTVRRCDQVLCTVPFSILRRMSLSGFDGDKLDVVHAVKYWSATKVAFHCREAFWEHAGISGGASYSGGRIRQTYYPAAEGVPGGGAVLLASYTMGDDADRLGRMPPAACRSVVLDELSKMHPELRRPGMVMDSASLAWGQHRWSEGAGVTRWGMSVAASEEQRSQAARPQGRLFFAGEHCSSTPAWIDGAIESALEAVGQVRLGTREGAVAVGAR

SEQ ID NO: 89 - DNA, 3063 bp, Actinomadura melliaura
  source 1..3063: mol_type = other DNA
atgtccttgttcgatttgccaagattgcattttgctggtgttgctactactagattgccaactggtttgagaaacggtttgattgatttggctaccaacactgttcatactcacgaaggtccatttccagttgatagaccagctcatgattatcatgcttacttggatgaacacggtactagatatggtgctgatggtagagctgatcctgaaggtccttttaatgctttgaaaggttggaatttcgctggtaatggtcatttcgctgttgattctgaaatcgttggtgttgaaactccagatggttttgatactggtgatttgttggttggtagaaaggttgatatgtggggtcattacaatgaatacttggctactactgctaacagagccagagtttttgatgttgatccagcttctgatttgaccactactttgatggttggtcaattgtctttcggtagacaaggtagatctcatgatgttggttatttggtttctggtggtgtttctggtttggctccaccaagatggcaaaatgatagacatgttcaaggtgttactggtcattggttagctggtcaattaggtagagctaccttgtaccaatttgttgttccagctgatgaagaattgagatggggtgaagaagctgctggttctccagctttgagattattgagacagttgatcgatgaaggtagagccgatggtttggttgttcaatttgctttggctggtatggctcctccagctgctccaggtccaccagatcaatggggtttgagaggtactattgctccttggagagctggtgaacctagaactgctccagctggtagattattggttcctgttgatgatggttctttgaggactttgtcattgagattggatgctgaacatgtgacgttgaacatgattactgctgttccagttgctcatagattgggtccaccattggatttgggtgatttggaattgagaactgcttctggtgatagattggttgctgttattccatctagagtttacacagcttctgctggtacttctggtgttgttacagttgctagagaagccggtgttgctgctgaaaatgaagctttggttattacagctggtggtagacctttgttgagagaacaagaaaccgttttggttgtagatgatgctgctgttttgttggactacgaagaagaaactgaagttgaagtctcttgcttcgttagaggtgaaccagctccagccggtgctattagagttagacaatttgctaatccaagagccagaccattagatccaccaggtgatgttgaaatagttgaagttgctactggtgagggtagattgactttaagaggtgttagaccaggtactacttgcgttttgttagctgctgatccagcagaattgccagaaccaggtcatgctttacatgatggtgatgaagcagctttgtggcgtgctagattaggtgctattgctgttagagttttgccagatcatagacacttggatgctattccacaacaagatgtaggtttcgaagtcttgtacagacatattttcgcctactacgaactgactaacacgttcatgaagtccgaagtttttagtttggccgatagattcaaggttgaaacccatccaagactgatttggactatgtgtgatccaaagaacaagtccaagacttattacatgccaccaaccagagatatgtctgctccacaagctagactgttgttgaaatttttgagaaggcaacacactcaatctccagctccaactttggttagagctgaagaaagaagagatggcggtattactacaagacaacaattgattaccgctttgagacaagctgctactattgaattggctgtcatgttgcaatacttgtacgctgcttattccgttccattatacggtgctggtttggatagagtaagatctggtgaatggactccagaacaattgagactattgtgtggttcaggtggtgaatctttggataatggtattagaggtactttgatcaccgttgccagagaagaaatgattcacttcttggttgtcaacaacgttttgatggcttgtggtgctccattttacattccagatgttgatttcaccaggatcaacgatagattagctgttcctttggatttcgctttggaaccattgtctttgggttctgttcatagattcattgccatcgaacaaccagaatcaatgattccagctgttggtccacaaactggtgctactcatccatacggttctttgtctgaattatacgccgatattagacaaggcttgcaaagagttccagatttgttcatggttgacaaaggtagaggtggtggtgaacatcatttgttcttgagaagatctgttaacgatgcccatccagattaccaattggaagttgatgacttggcttctgctttgttcgctattgatgttgttactgaacaaggcgaaggtaacattttgagtgaagctgttgaaggtgaaccttctcattttgatgctttcgttggtattgctgaccaattgtctgttcaacctagagttggtagagcaccatggtcaccagcttatccagttgttagaaatccaacattgagagcaggtagagcaacaggtgaacaagttactgaccctgaagctagacaagttatgggtttgttcaacaggtcttaccacatgatgtttcaattgatggctcaacactttggtgaaagaccagattcttcattgagaaggtccgatttgatgaatgctgccttggatattatggttggtgctatgaggccattgggtgaattattagttactatgccatctggtagaagaggtaaaactgctggtccatcttttgaattggaagaaattccagccgctatctctagacctgatgttgctagaagggctatgtctttaagattcgatcatttggctaaggcttgccatgcttctcaagttgtaccagaaagagttggcgataactttgctttgttggctgatttttacggcagagccggtatggcttaa

SEQ ID NO: 90 - protein, 1020 aa, Actinomadura melliaura
MSLFDLPRLHFAGVATTRLPTGLRNGLIDLATNTVHTHEGPFPVDRPAHDYHAYLDEHGTRYGADGRADPEGPFNALKGWNFAGNGHFAVDSEIVGVETPDGFDTGDLLVGRKVDMWGHYNEYLATTANRARVFDVDPASDLTTTLMVGQLSFGRQGRSHDVGYLVSGGVSGLAPPRWQNDRHVQGVTGHWLAGQLGRATLYQFVVPADEELRWGEEAAGSPALRLLRQLIDEGRADGLVVQFALAGMAPPAAPGPPDQWGLRGTIAPWRAGEPRTAPAGRLLVPVDDGSLRTLSLRLDAEHVTLNMITAVPVAHRLGPPLDLGDLELRTASGDRLVAVIPSRVYTASAGTSGVVTVAREAGVAAENEALVITAGGRPLLREQETVLVVDDAAVLLDYEEETEVEVSCFVRGEPAPAGAIRVRQFANPRARPLDPPGDVEIVEVATGEGRLTLRGVRPGTTCVLLAADPAELPEPGHALHDGDEAALWRARLGAIAVRVLPDHRHLDAIPQQDVGFEVLYRHIFAYYELTNTFMKSEVFSLADRFKVETHPRLIWTMCDPKNKSKTYYMPPTRDMSAPQARLLLKFLRRQHTQSPAPTLVRAEERRDGGITTRQQLITALRQAATIELAVMLQYLYAAYSVPLYGAGLDRVRSGEWTPEQLRLLCGSGGESLDNGIRGTLITVAREEMIHFLVVNNVLMACGAPFYIPDVDFTRINDRLAVPLDFALEPLSLGSVHRFIAIEQPESMIPAVGPQTGATHPYGSLSELYADIRQGLQRVPDLFMVDKGRGGGEHHLFLRRSVNDAHPDYQLEVDDLASALFAIDVVTEQGEGNILSEAVEGEPSHFDAFVGIADQLSVQPRVGRAPWSPAYPVVRNPTLRAGRATGEQVTDPEARQVMGLFNRSYHMMFQLMAQHFGERPDSSLRRSDLMNAALDIMVGAMRPLGELLVTMPSGRRGKTAGPSFELEEIPAAISRPDVARRAMSLRFDHLAKACHASQVVPERVGDNFALLADFYGRAGMA

SEQ ID NO: 91 - DNA, 1443 bp, Crocus sativus
  source 1..1443: mol_type = other DNA
atgggttctgaaggtagacaattgcacattttcatgttcccattcatggctcatggtcatatgattccaatagttgatatggctaagttgttcgcctcaagaggtattaagattaccatcgttactacgcccttgaactccatttctatctctaagtcattgcacaactgctccccaaattctttgattcagttgctgattttgaagttcccagctgctgaagctggtttgccagatggttgtgaaaatgctgattctatcccatctatggacttgttgccaaagtttttcgaagccgtttctttgttgcaaccaccatttgaagaagccttgcataacaatagaccagactgcttgatttccgatatgttttttccatggaccaacgatgttgctgatagagttggtattccaagattgatcttccatggcacctcttgcttttctttgtgttcttctgaattcatgaggctgcataagccataccaacatgtttcttcagatactgagccattcaccattccatatttgccaggtgatattaagctgaccaaaatgaagttgccaatcttcgtcagagaaaactccgaaaacgaattctccaagttcatcaccaaggtcaaagaatctgaatctttctgctacggtgttgtcgttaactctttctatgaattggaagccgaatacgttgattgctacaaagatgttttgggtagaaagacttggactatcggtccattgtctttgactaacactaagacccaagaaatcaccttgagaggtagagaatctgccattgatgaacatgaatgtttgaagtggttggactctcaaaagccaaactctgttgtttacgtttgctttggttctttggccaagtttaactccgctcagttgaaagaaattgctattggtttggaagcctccggtaagaagtttatttgggttgttagaaaaggtaagggcgaagaagaagaggaagaacaaaattggttgccagaaggttacgaagaaagaatggaaggtactggtttgattattagaggttgggctccacaagttttgattttggatcatccatctgttggtggtttcgttactcattgtggttggaattctactttggaaggtgttgctgctggtgttccaatggttacttggccagttggtgctgaacaattttacaacgaaaagttggttaccgaggtcttgaaaactggtgttggtgtaggtgttcaaaaatgggctccaggtgtcggtgattttattgaatctgaagctgttgagaaggccatcagacgtattatggaaaaagaaggtgaagagatgagaaacagagccattgaattgggtaaaaaagctaaatgggctgtcggtgaagaaggttcttcttactctaatttggatgccttgatcgaagagttgaagtctttggctttctaa

SEQ ID NO: 92 - protein, 480 aa, Crocus sativus
MGSEGRQLHIFMFPFMAHGHMIPIVDMAKLFASRGIKITIVTTPLNSISISKSLHNCSPNSLIQLLILKFPAAEAGLPDGCENADSIPSMDLLPKFFEAVSLLQPPFEEALHNNRPDCLISDMFFPWTNDVADRVGIPRLIFHGTSCFSLCSSEFMRLHKPYQHVSSDTEPFTIPYLPGDIKLTKMKLPIFVRENSENEFSKFITKVKESESFCYGVVVNSFYELEAEYVDCYKDVLGRKTWTIGPLSLTNTKTQEITLRGRESAIDEHECLKWLDSQKPNSVVYVCFGSLAKFNSAQLKEIAIGLEASGKKFIWVVRKGKGEEEEEEQNWLPEGYEERMEGTGLIIRGWAPQVLILDHPSVGGFVTHCGWNSTLEGVAAGVPMVTWPVGAEQFYNEKLVTEVLKTGVGVGVQKWAPGVGDFIESEAVEKAIRRIMEKEGEEMRNRAIELGKKAKWAVGEEGSSYSNLDALIEELKSLAF

SEQ ID NO: 93 - DNA, 1404 bp, Helianthus annuus
  source 1..1404: mol_type = other DNA
atggaatcctctaccgttgttatgtatccatctccaggtattggtcacttggtttctatggttgaattgggcaagttgattcatacccatcatccatctttgtccgtcatcattttgattttgactgctccatacgaaactggtgctactggtaagtatattaacactgtttctgctactaccccagccattacttttcatcatttgccagctattgctttgccaccagatttttcttccgaattcattgatttggctttcggtttgccagagttgtacaattccgttgttcataacaccttggttgccatctctcaaaagtctactattaaggccgttatcctggactttttctctaatgctgctttccaagtttccaccaatttgtctttgccaacctactactttttcacttctggtacatttggtctgtgcgctttcttgtatttgactaccttgcataagaccacctccaagtctattaaggatttgaacaccttgttggatttcccaggtgttccacctattcattcttctcatatgccaaccgctatcttcgatagagaatctaactcttacaagaatttcatgaagacctccaacaacatggctaagtgttctggtattatcgtcaacagcttcttggaattagaagaaagagctgttgctactttgcgtgatggtaaatgtattactgatggtccaactccaccaatctactttattggtccattgattgcttccggttctcaagttgatccaaacgaaaatgaatgcctgaagtggttgaaaacccaaccatctaaatccgttgttttcttgtgtttcggttccatgggtgtgttcgaaaaagaacagttgaaagaaattgccgtcggtttggaaagatctggtcaaagatttttgtgggttgttagaaaccctccactggaatcttcatctggtgctaaagaatttgaactggatgacattttgcccgaaggtttcttgactagaacaaaggataagggtttagtcgttaagaattgggctccacaaccagctattttgggtcatgaatctgttggtggtttcgtttctcattgtggttggaattcttcattggaagcagttgtttctggtgttccaatggttgcttggccattatatgctgaacaacaaatgaacagagtctacttggtcgaagaaatcaaagttgctttgtggttaagaatgtccgccgatggttttgttggtgctgaagccgttgaagaaactgttagaaaattgatggaaggtgaagaaggtagagccgttagagaacaaattttggaaatgtctggtggtgcaaaagctgctgttgaagatggtggttcttctagattggatttcttgaagttgactagaccatggaccgatcaatga

SEQ ID NO: 94 - protein, 467 aa, Helianthus annuus
MESSTVVMYPSPGIGHLVSMVELGKLIHTHHPSLSVIILILTAPYETGATGKYINTVSATTPAITFHHLPAIALPPDFSSEFIDLAFGLPELYNSVVHNTLVAISQKSTIKAVILDFFSNAAFQVSTNLSLPTYYFFTSGTFGLCAFLYLTTLHKTTSKSIKDLNTLLDFPGVPPIHSSHMPTAIFDRESNSYKNFMKTSNNMAKCSGIIVNSFLELEERAVATLRDGKCITDGPTPPIYFIGPLIASGSQVDPNENECLKWLKTQPSKSVVFLCFGSMGVFEKEQLKEIAVGLERSGQRFLWVVRNPPLESSSGAKEFELDDILPEGFLTRTKDKGLVVKNWAPQPAILGHESVGGFVSHCGWNSSLEAVVSGVPMVAWPLYAEQQMNRVYLVEEIKVALWLRMSADGFVGAEAVEETVRKLMEGEEGRAVREQILEMSGGAKAAVEDGGSSRLDFLKLTRPWTDQ

SEQ ID NO: 95 - DNA, 1458 bp, Populus trichocarpa; identical to SEQ ID NO: 65
  source 1..1458: mol_type = other DNA

SEQ ID NO: 96 - protein, 485 aa, Populus trichocarpa; identical to SEQ ID NO: 66